SEQUENCE LISTING (10 sequences)

SEQ ID NO: 1 - protein, 195 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 2 - protein, 195 aa, Homo sapiens; identical to SEQ ID NO: 1

SEQ ID NO: 3 - protein, 170 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEMRKNQKMRVPGKMIKAFSGSISIFVFCKIICNSPLCL

SEQ ID NO: 4 - protein, 143 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPVKYHHVGKLLKEGEEPTVYSDEEEPKDESARKND

SEQ ID NO: 5 - protein, 223 aa, Rattus norvegicus
MAAEDVVATGADPSELEGGGLLQEIFTSPLNLLLLGLCIFLLYKIVRGDQPGASGDNDDDEPPPLPRLKPRDFTPAELRRYDGVQDPRILMAINGKVFDVTKGRKFYGPEGPYGVFAGRDASRGLATFCLDKEALKDEYDDLSDLTPAQQETLNDWDSQFSSPSSTITWGKLLEGAEEPIVYSDDEEQKMRLLGRVTEAVSGAYLFLYFAKSFVTFQSVFTTW

SEQ ID NO: 6 - protein, 195 aa, Rattus norvegicus
MAAEDVVATGADPSELEGGGLLQEIFTSPLNLLLLGLCIFLLYKIVRGDQPGASGDNDDDEPPPLPRLKPRDFTPAELRRYDGVQDPRILMAINGKVFDVTKGRKFYGPEGPYGVFAGRDASRGLATFCLDKEALKDEYDDLSDLTPAQQETLNDWDSQFTFKYHHVGKLLKEGEEPTVYSDDEEPKDEAARKSD

SEQ ID NO: 7 - protein, 11 aa, Artificial sequence
  Synthetic peptide
EPKDESARKND

SEQ ID NO: 8 - protein, 223 aa, Homo sapiens
MQWAVGRRWAWAALLLAVAAVLTQVVWLWLGTQSFVFQREEIAQLARQYAGLDHELAFSRLIVELRRLHPGHVLPDEELQWVFVNAGGWMGAMCLLHASLSEYVLLFGTALGSRGHSGRYWAEISDTIISGTFHQWREGTTKSEVFYPGETVVHGPGEATAVEWGPNTWMVEYGRGVIPSTLAFALADTVFSTQDFLTLFYTLRSYARGLRLELTTYLFGQDP

SEQ ID NO: 9 - protein, 45 aa, Homo sapiens
MAAEDVVATGADPSDLESGGLLHEIFTSPLNLLLLGLCIFLLYKI

SEQ ID NO: 10 - protein, 50 aa, Homo sapiens
TAAQQETLSDWESQFTFKYHHVGKLLKEGEEPTVYSDEEEPKDESARKND

(19) United States
(12) Patent Application Publication
(10) Pub. No.: US 2017/0349554 A1
(43) Pub. Date: Dec. 7, 2017

(54) COMPOSITIONS AND METHODS FOR TREATING NEURODEGENERATIVE DISEASE

(71) Applicant: Cognition Therapeutics, Inc., Pittsburgh, PA (US)

(72) Inventors: Susan M. CATALANO, Pittsburgh, PA (US); Gilbert RISHTON, Los Angeles, CA (US); Nicholas J. IZZO, Pittsburgh, PA (US)

(21) Appl. No.: 15/493,979

(22) Filed: Apr. 21, 2017

Related U.S. Application Data
(63) Continuation of application No. 14/241,026, filed on Sep. 10, 2014, now abandoned.
(60) Provisional application No. 61/527,584, filed on Aug. 25, 2011.
(60) Provisional application No. 61/527,963, filed on Aug. 26, 2011.

(51) Int. Cl. C07D 235/04; A61K 31/351; G01N 33/50; C07D 317/46; C07D 211/22; C07C 267/00; C07C 233/91; A61K 31/135; C07C 43/23; A61K 31/5377; A61K 31/495; A61K 31/47; A61K 31/454; A61K 31/423; C07C 211/15

(52) U.S. Cl. CPC C07D 235/04; C07C 211/15; A61K 31/351; G01N 33/5058; C07D 317/46; C07D 211/22; C07C 267/00; C07C 233/91; A61K 31/135; C07C 43/23; A61K 31/5377; A61K 31/495; A61K 31/47; A61K 31/454; A61K 31/423; G01N 2333/705; G01N 2500/10

(57) ABSTRACT

This invention relates to the use sigma-2 receptor antagonists, and of pharmaceutical compositions comprising such compounds, in methods for inhibiting Abeta-associated synapse loss or synaptic dysfunction in neuronal cells, modulating an Abeta-associated membrane trafficking change in neuronal cells, and treating cognitive decline associated with Abeta pathology and more broadly treating with such compounds and compositions neurodegenerative diseases and disorders associated with Abeta pathology. This invention also relates to methods for screening compounds for activity in inhibiting cognitive decline on the basis of their ability to bind to a sigma-2 receptor.

This application is being filed on 27 Aug. 2012, as a PCT International Patent application in the name of Cognition Therapeutics, Inc., a U.S. national corporation, applicant for the designation of all countries except the US, and Susan M. Catalano, Gilbert Rishton and Nicholas J. Izzo, Jr., citizens of the U.S., applicants for the designation of the US only, and claims priority to U.S. Provisional Patent Application Ser. No. 61/527,584, filed Aug. 25, 2011 and U.S. Provisional Patent Application No. 61/527,963, filed Aug. 26, 2011, which applications are hereby incorporated by reference in its entirety.

### BACKGROUND OF THE INVENTION

Overproduction and accumulation of amyloid beta is a pathologic feature of Alzheimer's disease. Human amyloid beta (Abeta) is the main component of insoluble amyloid plaques-deposits found in the brain of patients with Alzheimer's disease. The plaques are composed of fibrillar aggregates of Abeta. Amyloid beta fibrils have been associated with the advanced stages of Alzheimer's disease.

The cognitive hallmark of early Alzheimer's disease (AD) is an extraordinary inability to form new memories. Early memory loss is considered a synapse failure caused by soluble Aβ oligomers. These oligomers block long-term potentiation, a classic experimental paradigm for synaptic plasticity, and they are strikingly elevated in AD brain tissue and transgenic AD models. It has been hypothesized that early memory loss stems from synapse failure before neuron death and that synapse failure derives from actions of soluble Aβ oligomers rather than fibrils. Lacor et al., Synaptic targeting by Alzheimer's-related amyloid β oligomers, J. Neurosci. 2004, 24(45):10191-10200.

Abeta is a cleavage product of an integral membrane protein, amyloid precursor protein (APP), found concentrated in the synapses of neurons. Soluble forms of Abeta are present in the brains and tissues of Alzheimer's patients, and their presence correlates with disease progression. Yu et al., 2009, Structural characterization of a soluble amyloid beta-peptide oligomer, Biochemistry, 48(9):1870-1877. Soluble amyloid β oligomers have been demonstrated to induce changes in neuronal synapses that block learning and memory.

Smaller, soluble Aβ oligomers interfere with a number of signaling pathways critical for normal synaptic plasticity, ultimately resulting in spine and synapse loss. Selkoe et al., 2008, Soluble oligomers of the amyloid beta-protein impair synaptic plasticity and behavior, Behav Brain Res 192(1): 106-113. Alzheimer's begins and persists as a synaptic plasticity disease.

The presence of soluble Aβ oligomers is believed to be to be responsible for early cognitive decline in the pre-Alzheimer's diseased brain. It is known that amyloid beta oligomers bind at neuronal synapses and that sigma-2 receptors are present in significant amounts in neurons and glia.

One approach to development of AD therapeutics involves generation of anti-Aβ monoclonal antibodies, several of which are in various phases of clinical development including bapineuzumab (AAB-00; Janssen, Elan, Pfizer), solanezumab (LY2062430; Eli Lilly); PF-04360365 (Pfizer); MABT5102A (Genentech); GSK933776 (GlaxoSmithKline) and gantenerumab (R1450, RO4909832, Hoffman-LaRoche). However, thus far no intravenous amyloid beta specific monoclonal antibody has yet been approved for the treatment of AD. Recently, development of intravenous bapineuzumab was ended due to lack of efficacy in two late-stage trials in patients who had mild to moderate Alzheimer's disease. However, solanezumab in secondary analysis of Phase III clinical trial results was recently reported to show statistically significant slowing of cognitive decline in patients with mild AD, but not in patient's with moderate AD. One problem with this approach may be related to lack of adequate brain penetrability.

There are only five medications currently FDA-approved for the treatment of Alzheimer's Disease (AD). Four are cholinesterase inhibitors: tacrine (COGNEX®; Sciele), donepezil (ARICEPT®; Pfizer), rivastigmine (EXELON®; Novartis), and galantamine (RAZADYNE®; Ortho-McNeil-Janssen). Donepezil, rivastigmine, and galantamine are successors to tacrine, a first generation compound rarely prescribed because of the potential for hepatotoxicity; they are roughly equally efficacious at providing symptomatic improvement of cognition and function at all stages of AD. The fifth approved medication is memantine (NAMENDA®; Forest), a low-affinity, use dependent N-methyl-D-aspartate glutamate receptor antagonist that offers similar benefits, but only in moderate to severe AD. The clinical effects of these compounds are small and impermanent, and currently available data are inconclusive to support their use as disease modifying agents. See, e.g., Kerchner et al, 2010, Bapineuzumab, Expert Opin Biol Ther., 10(7):1121-1130. Clearly, alternative approaches to treatment of AD are required.

The present invention is based, in part, on the broad finding that sigma-2 receptor antagonists, meeting certain requirements, inhibit the deleterious effects of soluble Aβ oligomers. In some embodiments, sigma-2 receptor antagonist compounds and compositions are used to treat or prevent synaptic dysfunction in a subject.

### FIELD OF THE INVENTION

This invention relates to the use of selective sigma-2 receptor antagonist compounds, and pharmaceutical compositions comprising them, in methods for inhibiting amyloid beta (Aβ)-associated synapse loss and synaptic dysfunction in neuronal cells. In some embodiments, the compositions are useful for modulating an Aβ-associated membrane trafficking change in neuronal cells, and treating cognitive decline associated with Aβ pathology in a patient in need thereof. In some embodiments, the compounds and compositions are used for treating neurodegenerative diseases and disorders associated with Abeta pathology. This invention also relates to methods for screening compounds for activity in inhibiting cognitive decline, on the basis of their ability to bind to and act as antagonists at a sigma-2 receptor, as well as to methods for refining such screening methods based in the first instance on whether the compounds block Aβ-induced membrane trafficking deficits, and block Aβ-induced synapse loss, but do not affect trafficking or synapse number in the absence of Aβ oligomers. The sigma-2 receptor antagonist compound is selected from a small molecule, or an antibody or fragment thereof, selective for the sigma-2 receptor.

### SUMMARY OF THE INVENTION

The invention is based, in part, on the broad finding that a sigma-2 receptor antagonist, preferably one that also exhibits other aspects of a particular therapeutic phenotype, participates in inhibition and inhibits deleterious effects of soluble amyloid-beta (“Abeta”, “Aβ”) peptides and oligomers and other soluble species thereof on neuronal cells, as defined below, and, consequently, can be used to treat conditions, including diseases and disorders, associated with Abeta oligomer-induced pathology, such as Alzheimer's disease. Soluble Abeta oligomers behave like reversible pharmacological ligands that bind to specific receptors and interfere with signaling pathways critical for normal synaptic plasticity, ultimately resulting in spine and synapse loss. It has been discovered that compounds that bind to the sigma-2 receptor and that behave as functional neuronal antagonists exhibit pharmacological competition with Abeta oligomers. Sigma-2 antagonist compounds as described herein thus can decrease or prevent Abeta oligomer effects such as Abeta induced cellular toxicity. Excluded are certain compounds of the prior art which were not known to be sigma-2 receptor antagonists and either (i) were known to bind to sigma-2 receptor and to reduce or eliminate Abeta induced pathologies such as a defect in membrane trafficking or synapse reduction in neuronal cells or (ii) were known to have activity against symptoms of Alzheimer's disease without implication of sigma-2 receptor interaction. The present invention also encompasses methods for inhibiting effects of Abeta oligomers or other soluble Abeta species on a neuronal cell and more generally amyloid beta pathologies comprising contacting the cell with a sigma-2 antagonist according to the present invention. In some embodiments, methods are provided for treating early stages of Alzheimer's disease comprising administering a therapeutically effective amount of a sigma-2 functional antagonist.

In some embodiments, the sigma-2 antagonists of the present invention bind to a sigma-2 receptor and inhibit the binding of Aβ oligomers to neurons, and particularly to synapses. In some embodiments, the sigma-2 antagonist competes with Aβ oligomer binding to neurons and specifically synapses, or otherwise disrupts the ability of Aβ oligomer to bind to neurons, such as by interfering with Aβ oligomer formation or binding to Aβ oligomer or possibly interfering with the ability of Aβ oligomer to set in motion signal transduction mechanisms attendant to its binding to neurons. In certain embodiments, the sigma-2 antagonists thus inhibit a non-lethal Aβ pathologic effect (“non-lethal Aβ pathology” or “non-lethal amyloid beta pathology), including a defect in membrane trafficking, synaptic dysfunction, a memory and learning defect in an animal, reduction in synapse number, change in dendritic spine length or spine morphology, or a defect in long term potentiation (LTP), among others. In other words, the present inventors observed that the sigma-2 antagonists of the invention that are active in other assays as illustrated herein, possess an ability to restore neurons to a normal state or interfere with Aβ oligomer-induced synaptic dysfunction. Without being bound by theory, sigma-2 antagonists of the invention interfere with one or more of Aβ oligomer structure, Aβ oligomer binding to neurons or Aβ oligomer-induced molecular signaling mechanisms which is useful in counteracting the nonlethal effects of Aβ oligomers and in treating early stages of soluble Aβ oligomer-associated pathologies.

In one embodiment, the sigma-2 antagonists of the present invention are functional neuronal antagonists and are used in a method of inhibiting synapse loss in a neuronal cell, the loss being associated with exposure of the cell to one or more Abeta oligomers or other Abeta complexes or, more generally, Abeta species including Abeta peptides in monomeric or oligomeric or otherwise soluble complexed form (as defined below), the method comprising contacting said cell with an amount of one or more sigma-2 antagonists in an amount effective to avert or reduce said loss or to partially or completely restore synapse number in said cell to pre-exposure levels.

In another embodiment, the sigma-2 antagonists of the present invention are used in a method for modulating a membrane trafficking change in a neuronal cell, said change being associated with exposure of said cell to one or more Abeta species, the method comprising contacting said cell with an amount of one or more sigma-2 antagonists in an amount effective to avert or reduce said membrane trafficking change, or have it remain at or closer to levels observed prior to exposure of said cell to said Abeta species.

In another embodiment, the sigma-2 antagonists of the present invention are used in a method for treating cognitive decline comprising administering to a subject one or more of the sigma-2 antagonists of the present invention.

In yet another embodiment, the sigma-2 antagonists of the present invention are functional neuronal sigma-2 antagonists used in a method for treating a cognitive decline or neurodegenerative disorder or a defect in synapse function and/or number comprising administering to a subject one or more of the sigma-2 antagonists of the present invention.

The present invention also provides a method for screening for compounds that inhibit cognitive decline or treat a neurodegenerative disease, the method comprising selecting one or more compounds for testing on the basis of their ability to bind to a sigma-2 receptor in preference to other, non-sigma classes of CNS receptors. The sigma-2 antagonists may or may not also bind to sigma-1 receptor.

In some embodiments, the disclosure provides compositions and methods comprising sigma-2 receptor antagonists for inhibiting amyloid beta oligomer-induced synaptic dysfunction of a neuronal cell; and for inhibiting suppression of hippocampal long term potention caused by exposure of neurons to Abeta oligomers.

The present invention provides a method of identifying a compound that inhibits cognitive decline or treats a neurodegenerative disease, the method comprising contacting a cell with a compound that binds to a sigma-2 receptor and determining whether said compound has at least one of the following additional properties:
(a) it inhibits synapse loss in a central neuron, said loss being associated with exposure of the neuron to Abeta oligomer;
(b) it inhibits membrane trafficking abnormalities in a central neuron, the abnormalities being associated with exposure of said cell to one or more Abeta oligomers;
(c) it inhibits Abeta oligomer-mediated cognitive effects in an animal model of Alzheimer's disease; or
(d) it inhibits hippocampal-based spatial learning and memory decline in an animal model of Alzheimer's disease.

In some embodiments, an in vitro assay platform method is disclosed that is predictive of behavioral efficacy for screening a selective, sigma-2 antagonist compound for the ability to inhibit cognitive decline or to treat a neurodegenerative disease, the method comprising contacting a cell with a compound that binds and acts as an antagonist at a sigma-2 receptor and wherein said compound has each of the following properties:
(a) it inhibits synapse loss in a central neuron, said loss being associated with exposure of the neuron to Abeta oligomer;
(b) it inhibits membrane trafficking abnormalities in a central neuron, the abnormalities being associated with exposure of said cell to one or more Abeta oligomers; and
(c) it does not affect trafficking or synapse number in the absence of Abeta oligomer.

The present invention also provides methods of identifying compounds that inhibit cognitive decline or treat a neurodegenerative disease. In some embodiments, the method comprises contacting a cell with a compound that binds a sigma-2 receptor. In some embodiments, the method also comprises identifying an additional compound that binds a sigma-2 receptor. In some embodiments, a method of identifying a compound that binds to a sigma-2 receptor comprises a competitive binding assay wherein a test compound is contacted with a sigma-2 receptor in the presence of a known sigma-2 ligand, wherein a test compound that competitively inhibits the binding of the known ligand is identified as a sigma-2 receptor ligand. Such methods may be carried out using an animal model, which can be any animal model but it is preferably a rodent model. Any appropriate binding assay can be used to determine whether a compound binds a sigma-2 receptor (or the compound can have already been determined or even been known to do so).

### BRIEF DESCRIPTION OF THE DRAWINGS

FIG. 1A is a photomicrograph showing primary hippocampal and cortical cultures maintained in vitro for 21 days with intracellular vesicles containing formazan resulting from endocytosis and chemical reduction of cargo tetrazolium salt dye in the membrane trafficking assay.

FIG. 1B is a photomicrograph showing sister cultures with extracellular formazan crystals formed outside of the cellular membrane of neurons and glia upon exocytosis of formazan wherein the cell has been exposed to Abeta oligomer in the membrane trafficking assay. This figure shows that human Abeta 1-42 oligomers alter the phenotype of the cargo dye product formazan (intracellular vesicles vs. extracellular crystals) and therefore causes cellular membrane trafficking deficits.

FIG. 1C is a photomicrograph showing intracellular vesicles, wherein the cell has been exposed to both Abeta oligomer and to compound II, a selective, high affinity sigma-2 antagonist compound according to the invention. This figure shows that compound II is able to block the membrane trafficking deficits produced by Abeta oligomers, and restores the membrane trafficking phenotype to normal.

FIG. 1D shows quantification of the membrane trafficking assay where the y-axis represents the amount of formazan product contained in the intracellular vesicles at a given point in time after administration of the cargo tetrazolium salt dye, normalized to vehicle-treated values. Red circles represent Abeta oligomer-treated cultures, blue squares represent vehicle-treated control cultures and black or gray squares represent values from cultures treated with various concentrations of cpd II+Abeta, and cpd IXa,IXb+Abeta, when compounds are added before Abeta oligomers (prevention). The concentration log of the compounds is used in the abscissa. This figure shows that the compounds inhibit Abeta oligomer effects on membrane trafficking in a dose-dependent manner.

FIG. 1E shows membrane trafficking assay dose-response curves in the same type of plot as FIG. 1D but when compounds are added after Abeta oligomers (treatment). The concentration log of the compounds is used in the abscissa. This figure shows that the compounds inhibit Abeta oligomer effects on membrane trafficking in a dose-dependent manner.

FIG. 1F shows a membrane trafficking assay in the same type of plot as FIG. 1D in the presence of various concentrations of synthetic Abeta oligomer alone (EC50 820 nM), and with various concentrations of compound II, and resulting vesicles (as % vehicle) at each concentration. A rightward shift in the EC 50 (Schild slope=−0.75) was exhibited by the presence of increasing concentrations of compound II. This figure demonstrates that cpd II pharmacologically competes with oligomers for access to molecular targets that mediate membrane trafficking, and therefore the presence of compound II made synthetic Abeta oligomers less synaptotoxic.

FIG. 1G shows a membrane trafficking assay in the same type of plot as FIG. 1D in the presence of various concentrations of synthetic Abeta oligomer alone, and with various concentrations of compound mixture IXa,IXb, and resulting vesicles (as % vehicle) at each concentration. A rightward shift in the EC 50 (Schild slope=−0.51) was exhibited by the presence of increasing concentrations of compound mixture IXa,IXb. This figure demonstrates that cpd mixture IXa,IXb pharmacologically competes with oligomers for access to molecular targets that mediate membrane trafficking, and therefore the presence of compound mixture IXa,IXb made synthetic Abeta oligomers less synaptotoxic.

FIG. 1H shows a membrane trafficking assay in the same type of plot as FIG. 1D in the presence of various concentrations of Abeta oligomers derived from human Alzheimer's patients alone, and with various concentrations of compound II, and resulting vesicles (as % vehicle) at each concentration. A rightward shift in the EC 50 was exhibited by the presence of increasing concentrations of compound II. This figure demonstrates that cpd II pharmacologically competes with oligomers for access to molecular targets that mediate membrane trafficking, and therefore the presence of compound II made human Alzheimer's disease-relevant Abeta oligomers less synaptotoxic.

FIG. 1I shows a membrane trafficking assay in the same type of plot as FIG. 1D in the presence of various concentrations of Abeta oligomers derived from human Alzheimer's patients alone, and with various concentrations of compound mixture IXa,IXb, and resulting vesicles (as % vehicle) at each concentration. A rightward shift in the EC 50 was exhibited by the presence of increasing concentrations of compound mixture IXa,IXb. This figure demonstrates that cpd mixture IXa,IXb pharmacologically competes with oligomers for access to molecular targets that mediate membrane trafficking, and therefore the presence of compound mixture IXa,IXb made human Alzheimer's disease-relevant Abeta oligomers less synaptotoxic.

FIG. 1J shows a membrane trafficking assay in the same type of plot as FIG. 1D in the presence of various concentrations of synthetic Abeta oligomer alone, and with various concentrations of compound CF, and resulting vesicles (as % vehicle) at each concentration. A rightward shift in the EC 50 was exhibited by the presence of increasing concentrations of compound CF. This figure demonstrates that cpd CF pharmacologically competes with oligomers for access to molecular targets that mediate membrane trafficking, and therefore the presence of compound CF made synthetic Abeta oligomers less synaptotoxic.

FIG. 1K shows a membrane trafficking assay in the same type of plot as FIG. 1D in the presence of various concentrations of synthetic Abeta oligomer alone, and with various concentrations of compound W, and resulting vesicles (as % vehicle) at each concentration. A rightward shift in the EC 50 was exhibited by the presence of increasing concentrations of compound W. This figure demonstrates that cpd W pharmacologically competes with oligomers for access to molecular targets that mediate membrane trafficking, and therefore the presence of compound W made synthetic Abeta oligomers less synaptotoxic.

FIG. 1L shows membrane trafficking assay results using Abeta oligomers isolated from Alzheimer's disease patients. Compound CF (20 microMolar concentration) exhibited pharmacological competition with Abeta oligomers isolated from AD patients for access to molecular targets that mediate membrane trafficking and therefore the presence of compound CF made human Alzheimer's disease-relevant Abeta oligomers less synaptotoxic.

FIG. 1M is a bar graph of trafficking assay results with percent formazan-filled vesicles of a neuron identified (and quantitated) in the presence of (i) vehicle alone (1^{st} bar); (ii) an Abeta oligomer preparation from human Alzheimer's disease patient brains (2^{nd} bar, significantly reduced compared to 1^{st} bar); (ii) compound II as disclosed herein plus Abeta oligomer (3^{rd} bar, significantly higher than the 2^{nd} bar); and (iv) compound II without Abeta oligomer (4^{th} bar, not significantly different from the first bar). This figure demonstrates that compound II blocks the membrane trafficking deficits produced by human Alzheimer's disease-relevant Abeta oligomers, and restores the membrane trafficking phenotype to normal, but does not affect membrane trafficking when dosed on its own in the absence of Abeta oligomers.

FIG. 1N is a bar graph identical in type to that of Figure J but depicting data generated using an Abeta oligomer preparation isolated from age-matched histologically normal human brains. This figure demonstrates that Abeta oligomers derived from normal human brain do not significantly affect membrane trafficking, and that cpd II does not further affect membrane trafficking in the presence or absence of such oligomers.

FIG. 2A is a plot of pharmacokinetic data in which the concentration of compound II obtained in plasma (left ordinate, ng/mL) upon a single subcutaneous (open triangles) and intravenous (i.v.) (open circles) administration of Compound II and in brain (right ordinate, ng/g) upon a single i.v. administration (filled circles) of Compound II. Compound II was known to be subject to first pass metabolism and thus was dosed subcutaneously; nevertheless Compound II was highly brain penetrant following acute dosing. This figure demonstrates that cpd II is highly brain penetrant upon acute subcutaneous dosing.

FIG. 2B is a plot of pharmacokinetic data in which the concentration of compound II obtained in plasma (left ordinate) upon once daily subcutaneous administration for 5 days of different amounts of Compound II (0.5 mg/kg/day: downward pointing filled triangles; 0.35 mg/kg/day: upward pointing filled triangles; and 0.1 mg/day filled squares) and in brain (right ordinate) upon subcutaneous administration of the same amounts (respectively downward pointing open triangle, upward pointing open triangle and open square) of Compound II. Compound II was known to be subject to first pass metabolism and thus was dosed subcutaneously; nevertheless Compound II was highly brain penetrant following chronic dosing. This figure demonstrates that cpd II is highly brain penetrant upon chronic subcutaneous dosing.

FIG. 2C is a plot of pharmacokinetic data in which the concentration of compound CB obtained following single acute oral dosing obtained in plasma (left ordinate, closed triangles) and in brain (right ordinate, open triangles) upon single acute oral administration of Compound CB (10 mg/kg/day). Compound CB was highly brain penetrant following acute oral dosing and exhibits 50% bioavailability with a plasma half-life of 3.5 hours. This figure demonstrates that cpd CB is highly brain penetrant upon acute oral dosing.

FIG. 2D shows is a plot of pharmacokinetic data in which the concentration of compound CB obtained following chronic once daily oral dosing for 5 days obtained in plasma (left ordinate, closed triangles) and in brain (right ordinate, open triangles) upon once daily oral administration of Compound CB (10 mg/kg/day, upright triangles) or 30 mg/kg/day (inverted triangles). Compound CB was highly brain penetrant following chronic oral dosing and exhibits a brain/plasma ratio of 3 at up to 5 days of once daily oral administration. This figure demonstrates that cpd CB is highly brain penetrant upon chronic oral dosing.

FIG. 3A-Panel A is a fluoromicrograph of primary hippocampal and cortical cultures maintained in vitro for 21 days exposed to Abeta oligomer in the absence of Compound IXa,IXb; Abeta (visualized with monoclonal antibody 6E10 immunolabeling) is bound to cellular membranes including neuronal postsynaptic spines at synapses.

FIG. 3A-Panel B is the same field of view as seen in FIG. 3A-Panel A showing the number of synapses (visualized with synaptophysin immunolabeling) are reduced in the presence of Abeta oligomers compared to a negative control (not shown).

FIG. 3A-Panel C is a lower magnification fluoromicrograph of primary hippocampal and cortical cultures maintained in vitro for 21 days exposed to Abeta oligomer in the absence of Compound IXa,IXb; Abeta (visualized with monoclonal antibody 6E10 immunolabeling) is bound to cellular membranes including neuronal postsynaptic spines at synapses.

FIG. 3A-Panel D shows sister cultures of primary hippocampal and cortical cultures maintained in vitro for 21 days exposed to Abeta oligomer in the presence of Compound IXa,IXb; the amount of Abeta bound to cellular membranes including neuronal postsynaptic spines is visibly reduced.

FIG. 3B-Panel A is a fluoromicrograph of sister cultures of primary hippocampal and cortical cultures maintained in vitro for 21 days exposed to Abeta oligomer in the presence of Compound IXa,IXb; the amount of Abeta bound to cellular membranes including neuronal postsynaptic spines is visibly reduced. This figure demonstrates that the presence of Compound IXa,IXb (i) significantly reduced the amount of Abeta oligomer bound to cellular membranes including neuronal postsynaptic spines. Similar protection was seen in the presence of Compound II (data not shown).

FIG. 3B-Panel B is the same field of view as seen in FIG. 3A-Panel C showing the number of synapses (visualized with synaptophysin immunolabeling) are restored in the presence of Compound IXa,IXb with increased synaptophysin visualization compared to FIG. 3A-panel B. This figure demonstrates that compound mixture IXa,IXb significantly blocks Abeta oligomer-induced synaptic loss. Similar protection was seen in the presence of Compound II (data not shown).

FIG. 3C is a quantification of the data shown in FIG. 3A-panels A-D in a bar graph of a synapse loss assay experiment. Synapse loss provides the closest correlate to cognitive function. In the synapse loss assay, Abeta oligomers caused an 18.2% synapse loss vs. vehicle in vitro. The presence of compound II or compound mixture IXa,IXb completely eliminated this synaptic regression. No effect was seen when the compounds were dosed in vehicle alone, without Abeta oligomers. Specifically, synapse count was calculated by image processing-based quantification of the number, intensity and area of synaptophysin-immunolabeled areas of the fluoromicrographs expressed as percent of negative control (vehicle) in neurons exposed to vehicle alone (black first bar); vehicle and Compound IXa,IXb or Vehicle and Compound II (second and third bars, respectively, showing no effect on synapse number by Compounds); Abeta oligomer (fourth bar showing significant reduction in synapse count compared to first bar) and Abeta oligomer in the presence of either Compounds IXa,IXb or II (fifth and sixth bars) showing no reduction in synapse number compared to first bar. This figure demonstrates that the compounds IXa,IXb and II exhibited protective effects and blocked Abeta oligomer-induced reduction in synapse number.

FIG. 3D is a quantification of the data shown in FIG. 3A-Panels A-D in a bar graph of Abeta binding intensity calculated by image processing-based quantification of the number, intensity and area of 6E10-immunolabeled areas of the fluoromicrographs when Abeta alone is added to vehicle (first bar graph) and their significant reduction in the co-presence of Abeta and either Compound II or Compound mixture IXa,IXb. This figure demonstrates that compounds IXa,IXb and II lower the amount of Abeta bound to cellular membranes.

FIG. 4 is a bar graph of memory performance measured by percent freezing behavior in an in vivo fear conditioning assay measured at baseline training and 24 hours post-training for mice administered vehicle alone (first bar), vehicle plus Abeta oligomer (second bar) Compound II plus Abeta oligomer (third bar) and Compound II alone (fourth bar) and at 24 hours after administration of vehicle alone (first bar), vehicle plus Abeta oligomer (second, significantly reduced, bar), Compound II plus Abeta oligomer (third bar) and Compound II alone. Abeta oligomers (single 200 nanoMolar intrahippocampal injection) produced significant deficits in memory formation in 3-4 month old male wt C57BL/6 mice (N=16) compared to vehicle (N=18). Compound II (single 2 microMolar intrahippocanpal injection one hour prior to oligomers) eliminated memory deficits (N=1) produced by Abeta oligomers. There was no effect of compounds alone and no adverse behavioral effects were observed. This figure demonstrates that compound II can prevent Abeta oligomer-induced memory deficits, while have no effect on memory performance when dosed on its own.

FIG. 5A is a graph of the correlation between Sigma-2 binding affinity (from Table 2) and potency in the trafficking assay (from Table 5). Included are only compounds that were active in the trafficking assay: excluded are compounds that were also sigma 1 antagonists.

FIG. 5B is a graph of the same correlation between Sigma-2 binding affinity from Table 2) and potency in the trafficking assay (from Table 5) for the same compounds used to generate FIG. 5A but additionally including data points for compounds that are both sigma-2 ligands and sigma-1 antagonists (these outlier data point are clustered in the lower right hand quadrant of the graph and have not been used to calculate correlation coefficient.)

FIG. 5C is a graph showing the absence of a correlation between Sigma-1 binding affinity (from Table 2) and EC₅₀ in the trafficking assay (from Table 5), r²=0.06, p>0.05.

FIG. 5D is a graph showing the absence of correlation between Sigma 2 binding affinity (from Table 2) and maximum inhibition of Abeta in the trafficking assay (from Table 5). All data points are included in the analysis.

FIG. 6 is the same type of bar graph as FIG. 4 showing memory performance measured by freezing behavior in the same contextual fear conditioning assay as that which gave rise to FIG. 4 when animals were treated with (i) vehicle alone (first bar) (ii) Abeta oligomers (2^{nd} bar, showing a significant reduction in ability of test animals to acquire new memories)) (iii) a mixture of compounds IXa and IXb, (3rd bar, showing complete (and statistically significant) inhibition of Abeta oligomer-induced memory deficit); or (iv) a mixture of compounds IXa and IXb in the absence of Abeta oligomer (4^{th} bar, showing no effect on memory). There was no adverse behavioral effects observed. This figure demonstrates that compound mixture IXa,IXb can prevent Abeta oligomer-induced memory deficits, while have no effect on memory performance when dosed on its own.

FIG. 7A shows the membrane trafficking assay performed in primary hippocampal and cortical cultures used in the prevention mode where compound II, with or without threo-ifenprodil (TIF), is added before oligomers. Threo-ifenprodil (TIF) is a sigma-2 receptor ligand (Monassier et al., JPET, 322 (1):341-350, 2007) with affinity for other receptors (s2 0.9 nM, s1 59 nM, NR2B 222 nM, K+ch 88 nM, etc.), that does not cause apoptosis, affect trafficking or interfere with Abeta oligomer-induced trafficking deficits when dosed alone (data not shown), therefore high affinity sigma receptor binding is not sufficient to produce therapeutic phenotype. This figure demonstrates that TIF exhibits pharmacological competition with II (and IXa,IXb; not shown) in prevention format in neurons indicating that their binding sites on sigma receptors partially overlap. This is the first demonstration of functional competition in neurons by sigma ligands demonstrating that sigma receptors participate in Abeta oligomer-induced membrane trafficking processes.

FIG. 7B shows the membrane trafficking assay performed in primary hippocampal and cortical cultures used in the treatment mode where compound II, with or without threo-ifenprodil (TIF), is added after oligomers. This figure demonstrates that threo-ifenprodil (TIF) exhibits pharmacological competition with compound II (and IXa,IXb; not shown) in treatment format in neurons indicating that their binding sites on sigma receptors partially overlap.

FIG. 7C shows membrane trafficking assay performed in primary hippocampal and cortical cultures used in the treatment mode. The data show the amount of formazan contained inintracellular vesicles in the presence of vehicle (open square) and Abeta oligomer (open circle). The Abeta oligomer effect on membrane trafficking is attenuated by presence of Compound CF (closed squares) in a dose dependent manner. Addition of TIF significantly lowers maximum inhibition due to Compound CF, and shifts the EC50 value rightward; therefore TIF acts as a antagonist of same receptor bound by Compound CF in treatment format (compounds added after Abeta).

FIG. 7D shows membrane trafficking data in presence of vehicle (open square) and Abeta oligomer (open circle). Abeta effect is attenuated by presence of Compound II (closed squares) in a dose dependent manner. Addition of TIF significantly lowers maximum inhibition due to Compound II, and shifts EC50 value. Therefore TIF exhibits pharmacological competition with Compound II in treatment format.

FIG. 8A shows autoradiographic binding of [³H]-(+)-pentazocine (a sigma-1 receptor ligand) in (left panel) human frontal cortex tissue sections from normal patients, Lewy Body Dementia (DLB) patients, or Alzheimer's Disease (AD) patients, where BS is specific binding, and BNS is non-specific binding; and (right panel) shows a graph of average specific binding for [³H]pentazocine from the autoradiography experiments from the control (normal), DLB, or AD patients. The sigma-1 receptor is statistically lower in Alzheimer's disease brains compared to control age-matched brains in parallel with the degree of neuronal loss seen in AD. This figure demonstrates that sigma-1 receptor expression may remain constant in Alzheimer's disease brains.

FIG. 8B shows autoradiographic binding of [¹²⁵I]-RHM-4 (a sigma-2 receptor ligand) in (left panel) adjacent human frontal cortex tissue sections from normal patients, Lewy Body Dementia (DLB) patients, or Alzheimer's Disease (AD) patients; and (right panel) shows a graph of average specific binding for [¹²⁵I]RHM-4 from the autoradiography experiments from the control (normal), DLB, or AD patients. The sigma-2 receptor is not statistically lower in Alzheimer's disease and Lewy Body Dementia brains compared to control age-matched brains despite the neuronal loss seen in these diseases This figure demonstrates that sigma-2 receptor expression on surviving neurons and/or glia may be upregulated in DLB and Alzheimer's disease brains.

FIG. 8C shows (left panel) displacement of 18.4 nM [³H]-RHM-1 (a sigma-2 receptor ligand) in monkey frontal cortex, monkey hippocampus or human temporal cortex by sigma-2 ligands and (right panel) a graph of binding density of [³H]-RHM-1 with and without 1 uM each of siramesine and compounds IXa,IXb and II. This figure demonstrates that Compounds II and mixture IXa,IXb competitively displace known radiolabeled sigma-2 ligands such as [³H]-RHM-1 from the sigma-2 receptor in monkey and human brain tissue sections, and therefore both of these compounds bind to sigma-2 receptors.

FIG. 9A shows tumor cell cytotoxicity of sigma-2 receptor agonists as cell viability in MTS assay in SKOV-3 human ovarian cancer cell line treated with sigma compounds for 48 hours. Sigma-2 agonists (siramesine, SV-119, WC-26) kill tumor cells. Sigma-2 antagonists (RHM-1, IXa,IXb and II) do so only at a much higher concentration in the absence of agonists. This figure demonstrates that cpds II and IXa,IXb behave similarly to known sigma-2 antagonists in this assay, and therefore implies that they are sigma-2 antagonists in tumor cells.

FIG. 9B shows neuronal cell cytotoxicity of sigma-2 receptor agonists as nuclear intensity variation in neuronal cultures with sigma-2 compounds after 24 hours. Sigma-2 agonists (siramesine, SV-119, WC-26) cause abnormal nuclear morphology in neurons; Sigma-2 antagonists (RHM-1, IXa,IXb and II) do not. This figure demonstrates that cpds II and IXa,IXb behave similarly to known sigma-2 antagonists in this assay, and therefore implies that they are sigma-2 antagonists in primary hippocampal and cortical cells.

FIG. 10A shows caspase-3 activity in SKOV-3 hyman ovarian cancer cells induced by sigma-2 agonist siramesine whereas the sigma-2 receptor antagonists RHM-1, compounds II and IXa,IXb did not induce caspase-3 activity. Abeta oligomers cause low levels of caspase-3 activation and lead to LTD. High levels of oligomers and caspase-3 lead to cell death. Sigma-2 receptor agonists (SV-119, siramesine) activate caspase-3 in tumor cells and neurons; sigma-2 antagonists do not (FIGS. 10A and 10B). This figure demonstrates that cpds II and IXa,IXb behave similarly to known sigma-2 antagonists in this assay, and therefore implies that they are sigma-2 antagonists in tumor cells.

FIG. 10B shows caspase-3 activity in neurons induced by sigma-2 agonist siramesine whereas the sigma-2 receptor antagonists RHM-1, compounds II and IXa,IXb did not induce caspase-3 activity. This figure demonstrates that cpds II and IXa,IXb behave similarly to known sigma-2 antagonists in this assay, and therefore implies that they are sigma-2 antagonists in primary hippocampal and cortical cells.

FIG. 10C shows caspase-3 activation in SKOV-3 human ovarian tumor cells by sigma-2 receptor agonist SV-119. Sigma-2 receptor antagonists compounds IXa,IXb and II, RHM-1 do not block caspase-3 activation caused by sigma-2 receptor agonist SV-119 in tumor cells. This figure demonstrates that cpds II and IXa,IXb behave similarly to known sigma-2 antagonists in this assay, and therefore implies that they are sigma-2 antagonists in tumor cells.

FIG. 10D shows caspase-3 activation in neuronal cultures by sigma-2 receptor agonist SV-119 after 24 hours at various concentrations of agonist. This figure demonstrates that Sigma-2 receptor antagonists compounds IXa,IXb and II, but not RHM-1, blocked caspase-3 activation caused by sigma-2 receptor agonist SV-119 in primary hippocampal and cortical cells.

FIG. 11A shows the trafficking assay and trafficking deficits (reduction in vesicles compared to vehicle) due to the presence of Abeta oligomers. Addition of sigma-2 agonist siramesine blocks Abeta oligomer trafficking deficits at low concentrations, but causes cellular toxicity at high concentrations. Sigma-2 antagonist II blocks oligomer-induced trafficking deficits at all concentrations tested. This figure demonstrates that cmpd II exhibits a therapeutic phenotype, and does not behave similarly to known sigma-2 agonists.

FIG. 11B shows the trafficking assay and trafficking deficits (reduction in vesicles compared to vehicle) due to the presence of Abeta oligomers. Addition of sigma-2 agonist SV119 blocks Abeta oligomer trafficking deficits at low concentrations, but causes cellular toxicity at high concentrations. Sigma-2 antagonist RHM-1 blocks oligomer-induced trafficking deficits at all concentrations tested, but does not exhibit a therapeutic phenotype since its structure is not drug-like. This figure demonstrates that cmpd II behaves similarly to known sigma-2 antagonists.

FIG. 12A shows memory performance measured by percent freezing behavior in an in vivo fear conditioning assay measured at 24 hours post-training at 1-3 minutes in a 15 month old male transgenic Alzheimer's disease mouse model following oral administration of sigma-2 receptor antagonist compounds at various doses for 5.5 months. A significant improvement of memory deficits occurred in transgenic animals that were treated with 10 and 30 mg/kg/day of CB (p<0.05) and 30 mg/kg/day of CF (p<0.005) compared to Tg animals treated with vehicle (Mann-Whitney U test). This figure demonstrates that cmpds CB and CF reverse established memory deficits in transgenic Alzheimer's mice following chronic long-term administration.

FIG. 12B shows a bar graph of behavioral data for 9-month old female transgenic (Tg) Alzheimer's disease mice that exhibited significant memory deficits in the Y-maze (% alternation) when treated p.o. for 39 days with vehicle vs. vehicle treated non-transgenic littermates (i.e., vehicle treated Tg mice performed at chance, vehicle-treated non-Tg litter mates performed significantly better than chance-see asterisk and line next to each bar). Treatment of Tg animals with Cpd. CF at 30 mg/kG/day orally improved the deficits. No adverse behavioral effects were observed. This figure demonstrates that cmpd CF reverses established memory deficits in transgenic Alzheimer's mice following chronic short-term administration.

FIG. 13A shows a fluoromicrograph of Abeta oligomers binding to primary neuronal cultures 21 DIV visualized with 6E10 Abeta specific antibody immunolabeling.

FIG. 13B shows the same field as 13A in which neurons are selectively visualized via neuron-specific MAP2 immunolabeling.

FIG. 13C shows a fluoromicrograph of Abeta oligomers (visualized with 6E10 immunolabeling) binding to sister primary neuronal cultures pretreated with 78 nM anti-PGRMC1 C-terminal antibody 21 DIV. This figure demonstrates that the presence of anti-PGRMC C-terminal antibody resulted in significantly reduced Abeta oligomer binding that was 47% lower than control Abeta-only-treated cultures.

FIG. 13D shows the same field as 13C in which neurons are selectively visualized via neuron-specific MAP2 immunolabeling. A similar density of neurons are present in the culture as are seen in sister control cultures (FIG. 13A).

FIG. 13E shows a fluoromicrograph of Abeta oligomers (visualized with 6E10 immunolabeling) binding to sister primary neuronal cultures pretreated with 78 nM control antibody (non-immune IgG). This figure demonstrates that the presence of nonimmune IgG does not significantly change Abeta binding intensity from control cultures treated with Abeta only (FIG. 13A).

FIG. 13F shows the same field as 13E in which neurons are selectively visualized via neuron-specific MAP2 immunolabeling. A similar density of neurons are present in the culture as are seen in sister control cultures (FIG. 13A).

FIG. 13G shows a fluoromicrograph of Abeta oligomers (visualized with 6E10 immunolabeling) binding to sister primary neuronal cultures pretreated with 78 nM anti-PGRMC1 N-terminal antibody. This figure demonstrates that the presence of 78 nM anti-PGRMC1 N-terminal antibody does not significantly change Abeta binding intensity from control cultures treated with Abeta only (FIG. 13A).

FIG. 13H shows the same field as 13G in which neurons are selectively visualized via neuron-specific MAP2 immunolabeling. A similar density of neurons are present in the culture as are seen in sister control cultures (FIG. 13A). Scale bar=15 microns.

FIG. 14A shows a quantification of the data shown in FIG. 13 in a bar graph of Abeta binding intensity per neuron calculated by image processing-based quantification of the number, intensity and area of 6E10-immunolabeled areas of the fluoromicrographs. Abeta oligomer binding intensity per neuron in the absence of antibodies (open bar labeled “Control”) and with three concentrations each of C-terminal, and N-terminal specific anti-PGRMC1 antibodies, as well as nonimmune control IgG antibodies (respectively labeled bars). The C-terminal specific anti-PGRMC1 antibody is the only antibody that significantly decreased Abeta oligomer binding in a dose-dependent manner. Antibody induced changes to binding puncta number and binding area per neuron were very similar to those shown for intensity. This figure is consistent with the interpretation that a large percentage of Abeta oligomers present in these cultures bind to sigma-2 receptor.

FIG. 14B shows the nuclear area in the same neuronal cultures in FIGS. 13 and 14A. When treated with increasing concentration of antibodies, nuclear area, a measure of cellular toxicity, does not change. Therefore, the addition of antibodies does not affect the health of the neuronal cultures.

### DETAILED DESCRIPTION OF THE INVENTION

Before the compounds, compositions and methods of the invention are described in detail, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Definitions

The singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of a given value. For example, “about 50%” means in the range of 45%-55%.

“Sigma-2 ligand” refers to a compound that binds to a sigma-2 receptor and includes agonists, antagonists, partial agonists, inverse agonists and simply competitors for other ligands of this receptor or protein.

The term “agonist” refers to a compound, the presence of which results in a biological activity of a receptor that is the same as the biological activity resulting from the presence of a naturally occurring ligand for the receptor.

The term “partial agonist” refers to a compound the presence of which results in a biological activity of a receptor that is of the same type as that resulting from the presence of a naturally occurring ligand for the receptor, but of a lower magnitude.

The term “antagonist” refers to an entity, e.g., a compound, antibody or fragment, the presence of which results in a decrease in the magnitude of a biological activity of a receptor. In certain embodiments, the presence of an antagonist results in complete inhibition of a biological activity of a receptor. As used herein, the term “sigma-2 receptor antagonist” is used to describe a compound that acts as a “functional antagonist” at the sigma-2 receptor in that it blocks Abeta effects, for example, Abeta oligomer-induced synaptic dysfunction, for example, as seen in an in vitro assay, such as a membrane trafficking assay, or a synapse loss assay, or Abeta oligomer mediated sigma-2 receptor activation of caspase-3, or in a behavioral assay, or in a patient in need thereof. The functional antagonist may act directly by inhibiting binding of, for example, an Abeta oligomer to a sigma-2 receptor, or indirectly, by interfering with downstream signaling resultant from Abeta oligomer binding the sigma-2 receptor.

The term “sigma-2 receptor antagonist compound” refers to a small molecule, antibody, or active binding fragment thereof, that binds to a sigma-2 receptor in a measurable amount and acts as a functional antagonist with respect to Abeta effects oligomer induced synaptic dysfunction resultant from sigma-2 receptor binding.

The term “selectivity” or “selective” refers to a difference in the binding affinity of a compound (Kᵢ) for a sigma receptor, for example, a sigma-2 receptor, compared to a non-sigma receptor. The sigma-2 antagonists possess high selectivity for a sigma receptor in synaptic neurons. The Kᵢ for a sigma-2 receptor or both a sigma-2 and a sigma-1 receptor is compared to the Kᵢ for a non-sigma receptor. In some embodiments, the selective sigma-2 receptor antagonist, or sigma-1 receptor ligand, has at least 10-fold, 20-fold, 30-fold, 50-fold, 70-fold, 100-fold, or 500-fold higher affinity, or more, for binding to a sigma receptor compared to a non-sigma receptor as assessed by a comparison of binding dissociation constant Ki values, or IC₅₀ values, or binding constant, at different receptors. Any known assay protocol can be used to assess the Ki or IC₅₀ values at different receptors, for example, by monitoring the competitive displacement from receptors of a radiolabeled compound with a known dissociation constant, for example, by the method of Cheng and Prusoff (1973) (Biochem. Pharmacol. 22, 3099-3108), or specifically as provided herein. In some embodiments, the sigma-2 antagonist compound is an antibody, or active binding fragment thereof, specific for binding to a sigma-2 receptor compared to a non-sigma receptor. In the case of an antibody, or fragment, binding constants at a sigma-2 receptor, or fragment, can be calculated and compared to binding constants at a non-sigma receptor by any means known in the art, for example, by the method of Beatty et al., 1987, J Immunol Meth, 100(1-2):173-179, or the method of Chalquest, 1988, J. Clin. Microbiol. 26(12): 2561-2563. The non-sigma receptor is, for example, selected from a muscarinic M1-M4 receptor, serotonin (5-HT) receptor, alpha adrenergic receptor, beta adrenergic receptor, opioid receptor, serotonin transporter, dopamine transporter, adrenergic transporter, dopamine receptor, or NMDA receptor.

In the present application, the term “high affinity” is intended to mean a compound which exhibits a Kᵢ value of less than 600 nM, 500 nM, 400 nM, 300 nM, 200 nM, less than 150 nM, less than 100 nM, less than 80 nM, less than 60 nM, or preferably less than 50 nM in a sigma receptor binding assay, for example against [³H]-DTG, as disclosed by Weber et al., Proc. Natl. Acad. Sci (USA) 83: 8784-8788 (1986), incorporated herein by reference, which measures the binding affinity of compounds toward both the sigma-1 and sigma-2 receptor sites. Especially preferred sigma ligands exhibit Ki values of less than about 150 nM, preferably less than 100 nM, less than about 60 nM, less than about 10 nM, or less than about 1 nM against [³H]-DTG.

The term “therapeutic phenotype” is used to describe a pattern of activity for compounds in the in vitro assays that is predictive of behavioral efficacy. A compound that (1) selectively binds with high affinity to a sigma-2 receptor, and (2) acts as a functional antagonist with respect to Abeta oligomer-induced effects in a neuron, is said to have the “therapeutic phenotype” if (i) it blocks or reduces Aβ-induced membrane trafficking deficits; (ii) it blocks or reduces Aβ-induced synapse loss and (iii) it does not affect trafficking or synapse number in the absence of Abeta oligomer. This pattern of activity in the in vitro assays is termed the “therapeutic phenotype” and is predictive of behavioral efficacy.

The term “therapeutic profile” is used to describe a compound that meets the therapeutic phenotype, and also has good brain penetrability (the ability to cross the blood brain barrier), good plasma stability and good metabolic stability.

The term “drug-like properties” is used herein to describe the pharmacokinetic and stability characteristics of the sigma-2 receptor ligands upon administration; including brain penetrability, metabolic stability and/or plasma stability.

“Abeta species” or “Aβ” shall include compositions comprising soluble amyloid peptide-containing components such as Abeta monomers, Abeta oligomers, or complexes of Abeta peptide (in monomeric, dimeric or polymeric form) with other soluble peptides or proteins as well as other soluble Abeta assemblies, including any processed product of amyloid precursor protein. Soluble Aβ oligomers are known to be neurotoxic. Even Aβ₁₋₄₂ dimers are known to impair synaptic plasticity in mouse hippocampal slices. In one theory known in the art, native Aβ₁₋₄₂ monomers are considered neuroprotective, and self-association of Aβ monomers into soluble Abeta oligomers is required for neurotoxicity. However, certain Aβ mutant monomers (arctic mutation (E22G) are reported to be associated with familial AD. See, for example, Giuffrida et al., β-Amyloid monomers are neuroprotective. J. Neurosci. 2009 29(34):10582-10587. Nonlimiting examples of preparations comprising Abeta species are disclosed in U.S. patent application Ser. No. 13/021,872; U.S. Patent Publication 2010/0240868; International Patent Application WO/2004/067561; International Patent Application WO/2010/011947; U.S. Patent Publication 20070098721; U.S. Patent Publication 20100209346; International Patent Application WO/2007/005359; U.S. Patent Publication 20080044356; U.S. Patent Publication 20070218491; WO/2007/126473; U.S. Patent Publication 20050074763; International Patent Application WO/2007/126473, International Patent Application WO/2009/048631, and U.S. Patent Publication 20080044406, each of which is incorporated herein by reference.

“Administering,” when used in conjunction with the compounds of the present invention, means to administer a compound directly into or onto a target tissue or to administer a compound systemically or locally to a patient or other subject.

The term “animal” as used herein includes, but is not limited to, humans and non-human vertebrates such as wild, experimental, domestic and farm animals and pets.

As used herein, the terms “subject,” “individual,” and “patient,” are used interchangeably and refer to any animal, including mammals, mice, rats, other rodents, rabbits, dogs, cats, swine, cattle, sheep, horses, primates, non-human primates, humans, and the like.

As used herein, the term “contacting” refers to the bringing together or combining of molecules (or of a molecule with a higher order structure such as a cell or cell membrane) such that they are within a distance that allows for intermolecular interactions such as the non-covalent interaction between two peptides or one protein and another protein or other molecule, such as a small molecule. In some embodiments, contacting occurs in a solution in which the combined or contacted molecules are mixed in a common solvent and are allowed to freely associate. In some embodiments, the contacting can occur at or otherwise within a cell or in a cell-free environment. In some embodiments, the cell-free environment is the lysate produced from a cell. In some embodiments, a cell lysate may be a whole-cell lysate, nuclear lysate, cytoplasm lysate, and combinations thereof. In some embodiments, the cell-free lysate is lysate obtained from a nuclear extraction and isolation wherein the nuclei of a cell population are removed from the cells and then lysed. In some embodiments, the nuclei are not lysed, but are still considered to be a cell-free environment. The molecules can be brought together by mixing such as vortexing, shaking, and the like.

The term “improves” is used to convey that the present invention changes either the characteristics and/or the physical attributes of the tissue to which it is being provided, applied or administered. The term “improves” may also be used in conjunction with a disease state such that when a disease state is “improved” the symptoms or physical characteristics associated with the disease state are diminished, reduced, eliminated, delayed or averted.

The term “inhibiting” includes the blockade, aversion of a certain result or process, or the restoration of the converse result or process. In terms of prophylaxis or treatment by administration of a compound of the present invention, “inhibiting” includes protecting against (partially or wholly) or delaying the onset of symptoms, alleviating symptoms, or protecting against, diminishing or eliminating a disease, condition or disorder.

The term “inhibiting trafficking deficits” refers to the ability to block soluble Ab oligomer-induced membrane trafficking deficits in a cell, preferably a neuronal cell. A compound capable of inhibiting trafficking deficits has an EC50<20 μM, less than 15 μM, less than 10 μM, less than 5 μM, and preferably less than 1 μMin the membrane trafficking assay, and further is capable of at least 50%, preferably at least 60%, and more preferably at least 70% maximum inhibition of the Abeta oligomer effects of soluble Abeta oligomer-induced membrane trafficking deficits, for example, as described in Example 6.

At various places in the present specification, substituents of compounds of the invention are disclosed in groups or in ranges. It is specifically intended that embodiments of the invention include each and every individual subcombination of the members of such groups and ranges. For example, the term “C₁₋₆ alkyl” is specifically intended to individually disclose e.g. methyl (C₁ alkyl), ethyl (C₂ alkyl), C₃ alkyl, C₄ alkyl, C₅ alkyl, and C₆ alkyl as well as, e.g. C₁-C₂ alkyl, C₁-C₃ alkyl, C₁-C₄ alkyl, C₂-C₃ alkyl, C₂-C₄ alkyl, C₃-C₆ alkyl, C₄-C₅ alkyl, and C₅-C₆ alkyl.

For compounds of the invention in which a variable appears more than once, each variable can be a different moiety selected from the Markush group defining the variable. For example, where a structure is described having two R groups that are simultaneously present on the same compound, then the two R groups can represent different moieties selected from the Markush group defined for R.

The term “n-membered” where n is an integer typically describes the number of ring-forming atoms in a moiety where the number of ring-forming atoms is n. For example, pyridine is an example of a 6-membered heteroaryl ring and thiophene is an example of a 5-membered heteroaryl group.

As used herein, the term “alkyl” is meant to refer to a saturated hydrocarbon group which is straight-chained or branched. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylene” refers to a divalent alkyl linking group. An example of alkylene is methylene (CH₂).

As used herein, “alkenyl” refers to an alkyl group having one or more double carbon-carbon bonds. Example alkenyl groups include, but are not limited to, ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include, but are not limited to, ethynyl, propynyl, and the like. The term “alkynylenyl” refers to a divalent linking alkynyl group.

As used herein, “haloalkyl” refers to an alkyl group having one or more halogen substituents. Example haloalkyl groups include, but are not limited to, CF₃, C₂F₅, CHF₂, CCl₃, CHCl₂, C₂Cl₅, CH₂CF₃, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms. In some embodiments, aryl groups have from 6 to about 10 carbon atoms.

As used herein, “cycloalkyl” refers to non-aromatic cyclic hydrocarbons including cyclized alkyl, alkenyl, and alkynyl groups that contain up to 20 ring-forming carbon atoms. Cycloalkyl groups can include mono- or polycyclic (e.g., having 2, 3 or 4 fused rings) ring systems as well as spiro ring systems. A cycloalkyl group can contain from 3 to about 15, from 3 to about 10, from 3 to about 8, from 3 to about 6, from 4 to about 6, from 3 to about 5, or from 5 to about 6 ring-forming carbon atoms. Ring-forming carbon atoms of a cycloalkyl group can be optionally substituted by oxo or sulfido. Example of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl). Preferably, “cycloalkyl” refers to cyclized alkyl groups that contain up to 20 ring-forming carbon atoms. Examples of cycloalkyl preferably include cyclopropyl, cyclobutyl, cyclopentyl, cyclohexyl, cycloheptyl, adamantyl, and the like

As used herein, “heteroaryl” groups refer to an aromatic heterocycle having up to 20 ring-forming atoms and having at least one heteroatom ring member (ring-forming atom) such as sulfur, oxygen, or nitrogen. In some embodiments, the heteroaryl group has at least one or more heteroatom ring-forming atoms each independently selected from sulfur, oxygen, and nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 1 to about 5, from about 1 to about 4, from about 1 to about 3, from about 1 to about 2, carbon atoms as ring-forming atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles having up to 20 ring-forming atoms including cyclized alkyl, alkenyl, and alkynyl groups where one or more of the ring-forming carbon atoms is replaced by a heteroatom such as an O, N, or S atom. Heterocycloalkyl groups can be mono or polycyclic (e.g., both fused and spiro systems). Example “heterocycloalkyl” groups include morpholino, thiomorpholino, piperazinyl, tetrahydrofuraryl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. Ring-forming carbon atoms and heteroatoms of a heterocycloalkyl group can be optionally substituted by oxo or sulfido. For example, a ring-forming S atom can be substituted by 1 or 2 oxo [i.e., form a S(O) or S(O)₂]. For another example, a ring-forming C atom can be substituted by oxo (i.e., form carbonyl). Also included in the definition of heterocycloalkyl are moieties that have one or more aromatic rings fused (i.e., having a bond in common with) to the nonaromatic heterocyclic ring, for example pyridinyl, thiophenyl, phthalimidyl, naphthalimidyl, and benzo derivatives of heterocycles such as indolene, isoindolene, isoindolin-1-one-3-yl, 4,5,6,7-tetrahydrothieno[2,3-c]pyridine-5-yl, 5,6-dihydrothieno[2,3-c]pyridin-7(4H)-one-5-yl, and 3,4-dihydroisoquinolin-1(2H)-one-3yl groups. Ring-forming carbon atoms and heteroatoms of the heterocycloalkyl group can be optionally substituted by oxo or sulfido. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to an —O-haloalkyl group. An example haloalkoxy group is OCF₃. As used herein, “trihalomethoxy” refers to a methoxy group having three halogen substituents. Examples of trihalomethoxy groups include, but are not limited to, —OCF₃, —OCClF₂, —OCCl₃, and the like.

As used herein, “arylalkyl” refers to a C₁₋₆ alkyl substituted by aryl and “cycloalkylalkyl” refers to C₁₋₆ alkyl substituted by cycloalkyl.

As used herein, “heteroarylalkyl” refers to a C₁₋₆ alkyl group substituted by a heteroaryl group, and “heterocycloalkylalkyl” refers to a C₁₋₆ alkyl substituted by heterocycloalkyl.

As used herein, “amino” refers to NH₂.

As used herein, “alkylamino” refers to an amino group substituted by an alkyl group.

As used herein, “dialkylamino” refers to an amino group substituted by two alkyl groups.

As used here, C(O) refers to C(═O).

As used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valence of the designated atom or moiety is not exceeded, and that the substitution results in a stable compound. For example, if a methyl group (i.e., CH₃) is optionally substituted, then 3 hydrogen atoms on the carbon atom can be replaced with substituent groups, in indicated.

As used herein, an “amyloid beta effect”, for example, a “nonlethal amyloid beta effect”, or Abeta oligomer effect, refers to an effect, particularly a nonlethal effect, on a cell that is contacted with an Abeta species. For example, it has been found that when a neuronal cell is contacted with a soluble Amyloid-beta (“Abeta”) oligomer, the oligomers bind to a subset of synapses on a subset of neuronal cells in vitro. This binding can be quantified in an assay measuring Abeta oligomer binding in vitro for example. Another documented effect of Abeta species is a reduction in synapse number, which has been reported to be about 18% in the human hippocampus (Scheff et al, 2007) and can be quantified (for example, in an assay measuring synapse number). As another example, it has been found that, when a neuronal cell is contacted with an Amyloid-beta (“Abeta”) oligomer, membrane trafficking is modulated and alteration of membrane trafficking ensues. This abnormality can be visualized with many assays, including but not limited to, an MTT assay. For example, yellow tetrazolium salts are endocytosed by cells and the salts are reduced to insoluble purple formazan by enzymes located within vesicles in the endosomal pathway. The level of purple formazan is a reflection of the number of actively metabolizing cells in culture, and reduction in the amount of formazan is taken as a measure of cell death or metabolic toxicity in culture. When cells that are contacted with a yellow tetrazolium salt are observed through a microscope, the purple formazan is first visible in intracellular vesicles that fill the cell. Over time, the vesicles are exocytosed and the formazan precipitates as needle-shaped crystals on the outer surface of the plasma membrane as the insoluble formazan is exposed to the aqueous media environment. Still other effects of Abeta species include cognitive decline, such as a decline in the ability to form new memories and memory loss which can be measured in assays using animal models in vivo. In some embodiments, an Abeta effect is selected from Abeta oligomer-induced synaptic dysfunction, for example, as seen in an in vitro assay, such as a membrane trafficking assay, or a synapse loss assay, or Abeta oligomer mediated sigma-2 receptor activation of caspase-3, or Abeta induced neuronal dysfunction, Abeta mediated decrease in long term potentiation (LTP), or in cognitive decline in a behavioral assay, or in a patient in need thereof.

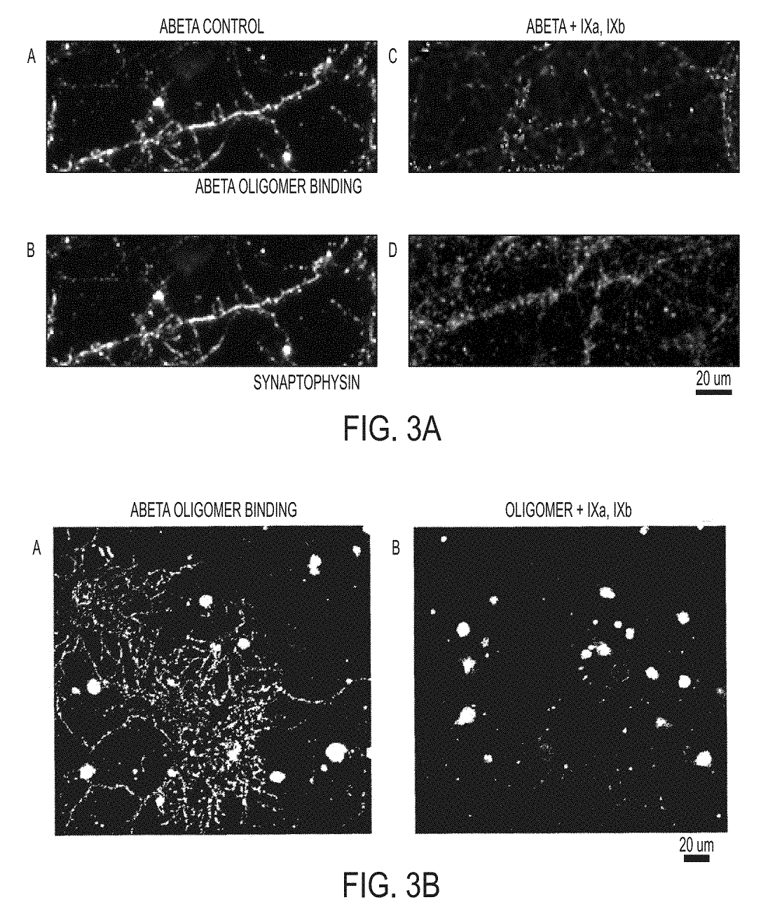

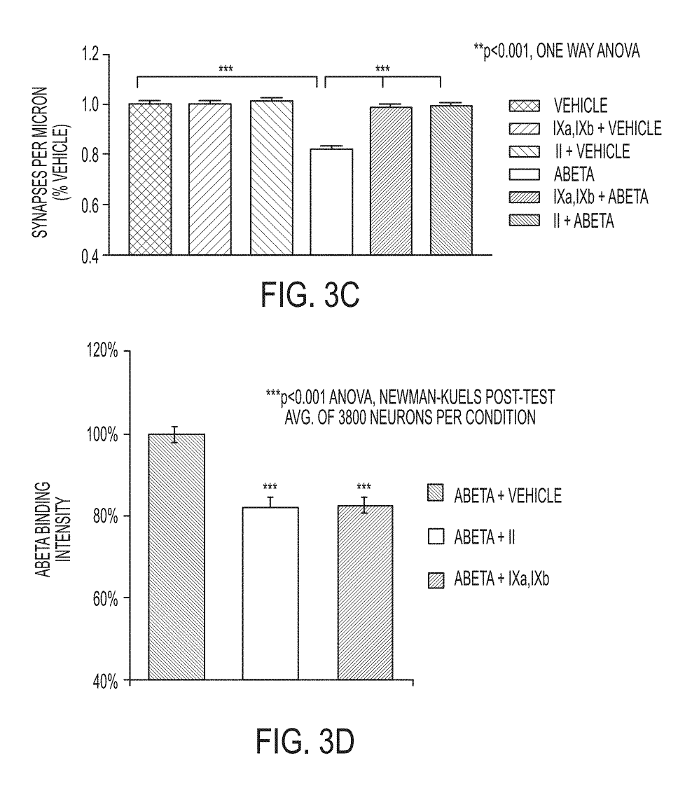

In some embodiments, a test compound is said to be effective to treat cognitive decline or a disease associated therewith when it can inhibit an effect associated with soluble Abeta oligomer species on a neuronal cell more than about 10%, preferably more than 15%, and preferably more than 20% as compared to a negative control. In some embodiments, a test agent is said to be effective when it can inhibit a processed product of amyloid precursor protein-mediated effect more than about 10%, preferably more than 15%, and preferably more than 20% as compared to a positive control. For example, as shown in the Examples below, inhibition of Abeta oligomer binding by only 18% inhibits synapse reduction completely. For example, see FIGS. 3C and 3D. Although the present specification focuses on inhibition of nonlethal effects of Abeta species, such as abnormalities in neuronal metabolism and synapse number reduction, these are shown to correlate with cognitive function and are furthermore expected, over time, to result in reduction (compared to untreated subjects) of downstream measurable symptoms of amyloid pathology, notably clinical symptoms such as 1) fibril or plaque accumulation measured by amyloid imaging agents such as fluorbetapir, PittB or any other imaging agent, 2) synapse loss or cell death as measured by glucose hypometabolism detected with FDG-PET, or 3) changes in protein expression or metabolite amount in the brain or body detectable by imaging or protein/metabolite detection in cerebrospinal fluid, brain biopsies or plasma obtained from patients by ELISA, (such as changes in levels and or ratios of Abeta 42, phosphorylated tau, total tau measured by ELISA, or patterns of protein expression changes detectable in an ELISA panel (see reference: Wyss-Coray T. et al. Modeling of pathological traits in Alzheimer's disease based on systemic extracellular signaling proteome. Mol Cell Proteomics 2011 Jul. 8, which is hereby incorporated by reference in its entirety), 4) cerebral vascular abnormalities as measured by the presence of vascular edema or microhemorrhage detectable by MRI and any other symptoms detectable by imaging techniques, and 5) cognitive loss as measured by any administered cognitive test such as ADAS-Cog, MMSE, CBIC or any other cognitive testing instrument.

As used herein, the term “a neuronal cell” can be used to refer to a single cell or to a population of cells. In some embodiments, the neuronal cell is a primary neuronal cell. In some embodiments, the neuronal cell is an immortalized or transformed neuronal cell or a stem cell. A primary neuronal cell is a neuronal cell that cannot differentiate into other types of neuronal cells, such as glia cells. A stem cell is one that can differentiate into neurons and other types of neuronal cells such as glia. In some embodiments, assays utilize a composition comprising at least one neuronal cell is free of glia cells. In some embodiments, the composition comprises less than about 30%, 25%, 20%, 15%, 10%, 5%, or 1% of glia cells, which are known to internalize and accumulate Abeta. The primary neuronal cell can be derived from any area of the brain of an animal. In some embodiments, the neuronal cell is a hippocampal or cortical cell. The presence of glia cells can be determined by any method. In some embodiments, glia cells are detected by the presence of GFAP and neurons can be detected by staining positively with antibodies directed against MAP2.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this invention are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable salt(s)”, as used herein, includes those salts of compounds of the invention that are safe and effective for use in mammals and that possess the desired biological activity. Pharmaceutically acceptable salts include salts of acidic or basic groups present in compounds of the invention or in compounds identified pursuant to the methods of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

As used herein, the term “therapeutic” means an agent utilized to treat, combat, ameliorate, protect against or improve an unwanted condition or disease of a subject.

As used herein, the term “effective amount” refers to an amount that results in measurable inhibition of at least one symptom or parameter of a specific disorder or pathological process. For example, an amount of a sigma-2 ligand of the present invention that provides a measurably lower synapse reduction in the presence of Abeta oligomer qualifies as an effective amount because it reduces a pathological process even if no clinical symptoms of amyloid pathology are altered, at least immediately.

A “therapeutically effective amount” or “effective amount” of a compound or composition of the invention is a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change). An effective amount of a compound of the invention may broadly range from about 0.01 mg/Kg to about 500 mg/Kg, about 0.1 mg/Kg to about 400 mg/Kg, about 1 mg/Kg to about 300 mg/Kg, about 0.05 to about 20 mg/Kg, about 0.1 mg/Kg to about 10 mg/Kg, or about 10 mg/Kg to about 100 mg/Kg. The effect contemplated herein includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The effective amount administered will be determined by the physician in the light of the foregoing relevant circumstances and the exercise of sound medical judgment. A therapeutically effective amount of a compound of this invention is typically an amount such that when it is administered in a physiologically tolerable excipient composition, it is sufficient to achieve an effective systemic concentration or local concentration in the tissue. The total daily dose of the compounds of this invention administered to a human or other animal in single or in divided doses can be in amounts, for example, from 0.01 mg/Kg to about 500 mg/Kg, about 0.1 mg/Kg to about 400 mg/Kg, about 1 mg/Kg to about 300 mg/Kg, about 10 mg/Kg to about 100 mg/Kg, or more usually from 0.1 to 25 mg/kg body weight per day. Single dose compositions may contain such amounts or submultiples thereof to make up the daily dose. In general, treatment regimens according to the present invention comprise administration to a patient in need of such treatment will usually include from about 1 mg to about 5000 mg, 10 mg to about 2000 mg of the compound(s), 20 to 1000 mg, preferably 20 to 500 mg and most preferably about 50 mg, of this invention per day in single or multiple doses.

The terms “treat”, “treated”, or “treating” as used herein refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this invention, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

Generally speaking, the term “tissue” refers to any aggregation of similarly specialized cells which are united in the performance of a particular function.

As used herein, “cognitive decline” can be any negative change in an animal's cognitive function. For example cognitive decline, includes but is not limited to, memory loss (e.g. behavioral memory loss), failure to acquire new memories, confusion, impaired judgment, personality changes, disorientation, or any combination thereof. A compound that is effective to treat cognitive decline can be thus effective by restoring long term neuronal potentiation (LTP) or long term neuronal depression (LTD) or a balance of synaptic plasticity measured electrophysiologically; inhibiting, treating, and/or abatement of neurodegeneration; inhibiting, treating, and/or abatement of general amyloidosis; inhibiting, treating, abatement of one or more of amyloid production, amyloid assembly, amyloid aggregation, and amyloid oligomer binding; inhibiting, treating, and/or abatement of a nonlethal effect of one or more of Abeta species on a neuron cell (such as synapse loss or dysfunction and abnormal membrane trafficking); and any combination thereof. Additionally, that compound can also be effective in treating Abeta related neurodegenerative diseases and disorders including, but not limited to dementia, including but not limited to Alzheimer's Disease (AD) including mild Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD), preclinical Alzheimer's Disease (PCAD); and Cognitive Impairment No Dementia (CIND).

As used herein, the term “natural ligand” refers to a ligand present in a subject that can bind to a protein, receptor, membrane lipid or other binding partner in vivo or that is replicated in vitro. The natural ligand can be synthetic in origin, but must also be present naturally and without human intervention in the subject. For example, Abeta oligomers are known to exist in human subjects. Therefore the Abeta oligomers found in a subject would be considered natural ligands. The binding of Abeta oligomers to a binding partner can be replicated in vitro using recombinant or synthetic techniques, but the Abeta oligomer would still be considered a natural ligand regardless of how the Abeta oligomer is prepared or manufactured. A synthetic small molecule that can also bind to the same binding partner is not a natural ligand if it does not exist in a subject. For example, Compound II, Compounds IXa and IXb, as well as all the other compounds which are described herein, are not normally present in a subject, and, therefore, would not be considered natural ligands.

Sigma-2 Receptors

The sigma receptors are multifunctional adapter/chaperone proteins that participate in several distinct protein signaling complexes in a tissue and state-related manner. The sigma-2 receptor is expressed in brain and various peripheral tissues at low levels. (Walker et al., 1990 Sigma receptors: biology and function. Pharmacol. Rev. 42:355-402). Sigma-2 receptors are present in human hippocampus and cortex. The sigma-2 receptor was also previously validated as a biomarker for tumor cell proliferation. (Mach et al., Sigma-2 receptors as potential biomarkers of proliferation in breast cancer. Cancer Res. 57:156-161, 1997).

Sigma-2 receptors are implicated in many signaling pathways such as heme binding, Cytochrome P450 metabolism, cholesterol synthesis, progesterone signaling, apoptosis and membrane trafficking. Only a subset of sigma receptor binding sites/signaling pathways are relevant to oligomer signaling in AD. No sigma-2 receptor knock-outs are currently available and human mutations in sigma-2 sequence have not been studied in a neurodegeneration context.

A sigma-2 receptor was recently identified as the progesterone receptor membrane component 1 (PGRMC1) in rat liver by use of a photoaffinity probe WC-21, which irreversibly labels sigma-2 receptors in rat liver. Xu et al. Identification of the PGRMC1 protein complex as the putative sigma-2 receptor binding site. Nature Communications 2, article number 380, Jul. 5, 2011, incorporated herein by reference. PGRMC1 (progesterone receptor membrane component 1) was identified as the critical 25 kDa component of sigma-2 receptor activity in August 2011 by Xu et al. PGRMC1 is a single transmembrane protein with no homology to sigma-1 protein; family members include PGRMC2 and neudesin. PGRMC1 contains a cytochrome b5 heme-binding domain. PGRMC1 is a single transmembrane protein with no homology to S1 protein; family members include PGRMC2 and neudesin. PGRMC1 contains a cytochrome b5 heme-binding domain. Endogenous PGRMC1 ligands include progesterone/steroids, cholesterol metabolites, glucocorticoids, and heme. PGRMC1 functions as chaperone/adapter associated with different protein complexes in different subcellular locations (Cahill 2007. Progesterone receptor membrane component 1: an integrative review. J. Steroid Biochem. Mol. Biol. 105:16-36). PGRMC1 binds heme with reducing activity, complexes with CYP450 proteins (regulated redox reactions), associates with PAIRBP1 and mediates progesterone block of apoptosis, and associates with Insig-1 and SCAP to induce SRE-related gene transcription in response to low cholesterol. The C. elegans homolog VEM1 associates with UNC-40/DCC to mediate axon guidance. PGRMC1 contains two SH2 target sequences, an SH3 target sequence, a tyrosine kinase site, two acidophilic kinase sites (CK2), and consensus binding sites for ERK1 and PDK1. PGRMC1 contains several ITAM sequences involved in membrane trafficking (vesicle transport, clathrin-dependent endocytosis of calveolin-containing pits).

Sigma-2 receptor therapeutics have reached human Phase II clinical trials for other CNS indications, but not for treatment of AD. Many of the sigma-2 receptor ligands are not very selective and have high affinity for other non-sigma CNS receptors. For example, Cyr-101/MT-210 (Cyrenaic Pharmaceuticals; Mitsubishi) is a sigma-2 receptor antagonist in phase IIa clinical trials for schizophrenia, but has multiple other receptor interactions including at 5HT2a, ADRA1, and histamine H1. Siramesine (Lundbeck, Forest Lu28179) is a sigma-2 receptor agonist that previously was in clinical trials for anxiety, but was discontinued. Sigma-1 receptor ligands are in clinical trials for various CNS indications. Cutamesine dihydrochloride (AGY SA4503, M's Science Corp.) is a sigma-1 receptor agonist that was in phase II clinical trials for stroke, and phase II trials for depression. Anavex 2-73 is a sigma-1 receptor agonist that also acts as at muscarinic cholinergic receptors as M2/3 antagonist, M1 agonist, and is an antagonist with respect to various ion channels (NMDAR, Na+, Ca++). Anavex 2-73 entered phase IIa clinical trials for patients with AD and mild cognitive impairment. There are no previous clinical trials with highly selective sigma-2 receptor ligand therapeutics in AD.

Sigma-2 Antagonists

While not being bound by theory, it is proposed that the sigma-2 receptor is a receptor for Abeta oligomer in neurons. Various receptors have been proposed in the literature for soluble Abeta oligomers including prion protein, insulin receptor, beta adrenergic receptor and RAGE (receptor for advanced glycation end products). Laurén, J. et al, 2009, Nature, 457(7233): 1128-1132; Townsend, M. et al, J. Biol. Chem. 2007, 282:33305-33312; Sturchler, E. et al, 2008, J. Neurosci. 28(20):5149-5158. Indeed many investigators believe that Abeta oligomer may bind to more than one receptor protein. Without being bound by theory, on the basis of evidence presented herein, the present inventors postulate an additional receptor for Abeta oligomer located (not necessarily exclusively) in neurons.

Without being bound by theory, Abeta oligomers are sigma receptor agonists that bind to sigma protein complexes and cause aberrant trafficking and synapse loss. It is demonstrated herein that high affinity sigma-2 ligands that antagonize this interaction and/or sigma receptor function in neurons will compete with Abeta oligomers and return neuronal responses to normal. Such ligands are considered functional sigma-2 receptor antagonists and are referred to as such or more simply as sigma-2 receptor antagonists or as sigma-2 antagonists.

In some embodiments, the sigma-2 receptor antagonist of the present invention acts as a functional antagonist in a neuronal cell with respect to inhibiting soluble Aβ oligomer induced synapse loss, and inhibiting soluble Aβ oligomer induced deficits in a membrane trafficking assay; exhibiting high affinity at a sigma-2 receptor; as well as having high selectivity for one or more sigma receptors compared to any other non-sigma receptor; and exhibiting good drug-like properties.

In some embodiments, a sigma-2 receptor functional antagonist meeting certain in vitro assay criteria detailed herein will exhibit behavioral efficacy, or be predicted to have behavioral efficacy, in one or more relevant animal behavioral models as disclosed in this specification. In some embodiments, behavioral efficacy is determined at 10 mg/kg p.o., or less.

In some embodiments, the disclosure provides an in vitro assay platform predictive of behavioral efficacy for high affinity sigma-2 receptor ligands. In accordance with the in vitro assay platform, the ligand binds with high affinity to a sigma-2 receptor; acts as a functional antagonist with respect to Abeta oligomer-induced effects in a neuron; inhibits Abeta oligomer-induced synapse loss in a central neuron or reduces Abeta oligomer binding to neurons to inhibit synapse loss; and does not affect trafficking or synapse number in the absence of Abeta oligomer. This pattern of activity in the in vitro assays is termed the “therapeutic phenotype”. The ability of a sigma-2 receptor antagonist to block Abeta oligomer effects in mature neurons without affecting normal function in the absence of Abeta oligomers meets the criteria for the therapeutic phenotype. It is now disclosed that a selective sigma-2 antagonist having a therapeutic phenotype, can block Abeta oligomer-induced synaptic dysfunction.

In some embodiments, high affinity, selective sigma-2 antagonists having the therapeutic phenotype that also possess the following characteristics are suitable as a therapeutic candidates for treating Abeta oligomer induced synaptic dysfunction in a patient in need thereof: high affinity at sigma receptors; high selectivity for sigma receptors compared to other non-sigma CNS receptors; higher affinity for a sigma-2 receptor, or comparable affinity, for example within an order of magnitude, at sigma-2 and sigma-1 receptors; selectivity for sigma receptors as opposed to other receptors relevant in the central nervous system and good drug-like properties. Drug-like properties include acceptable brain penetrability (the ability to cross the blood brain barrier), good stability in plasma and good metabolic stability, for example, as measured by exposure to liver microsomes. Without being bound by theory, high affinity sigma-2 receptor antagonists compete with Abeta oligomers, and/or stop pathological sigma receptor signaling, that leads to Alzheimer's disease.

In some embodiments, the antagonist of the invention may bind with greater affinity to sigma-1 receptor than to a sigma-2 receptor, but must still behave as a functional neuronal antagonist with respect to blocking or inhibiting an Abeta oligomer-induced effect (Abeta effect).

In some embodiments, a sigma-2 antagonist having the therapeutic phenotype that also possesses the following characteristics is suitable as a therapeutic candidate for treating Abeta oligomer induced synaptic dysfunction in a patient in need thereof: high affinity at sigma receptors; high selectivity for sigma receptors compared to other non-sigma CNS receptors; high affinity for a sigma-2 receptor, or comparable affinity at sigma-2 and sigma-1 receptors; and good drug-like properties. Drug-like properties include high brain penetrability, plasma stability, and metabolic stability.

In some embodiments, in the binding activity studies, an IC₅₀ or Ki value of at most about 600 nM, 500 nM, 400 nM, 300 nM, 200 nM, 150 nM, 100 nM, preferably at most about 75 nM, preferably at most about 60 nM, preferably at most about 40 nM, more preferably at most 10 nM, most preferably at most 1 nM indicates a high binding affinity with respect to the sigma receptor binding sites.

In some embodiments, a sigma-2 receptor antagonist with high affinity (preferably Ki less than about 600 nM, 500 nM, 400 nM, 300 nM, 200 nM, 150 nM, 100 nM, 70 nM, 60 nM, 50 nM, 30 nM, or 10 nM) at sigma-2 receptors that have greater than about 20-fold, 30-fold, 50-fold, 70-fold, or preferably greater than 100-fold selectivity for sigma receptors compared to other non-sigma CNS or target receptors, and have good drug-like properties including brain penetrability and good metabolic and/or plasma stability, and that possess the therapeutic phenotype, are predicted to have behavioral efficacy and can be used to treat Abeta oligomer-induced synaptic dysfunction in a patient in need thereof.

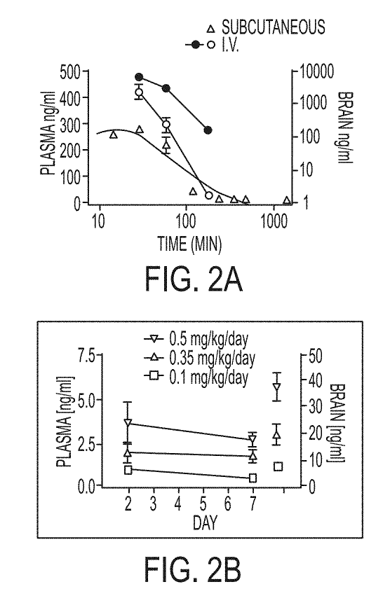

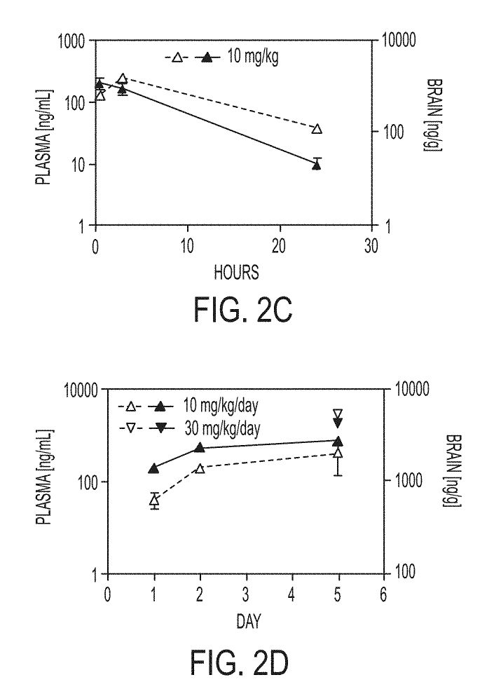

As used herein the term “brain penetrability” refers to the ability of a drug, antibody or fragment, to cross the blood-brain barrier. In some embodiments, an animal pharmacokinetic (pK) study, for example, a mouse pharmacokinetic/blood-brain barrier study can be used to determine or predict brain penetrability. In some embodiments various concentrations of drug can be administered, for example at 3, 10 and 30 mg/kg, for example p.o. for 5 days and various pK properties are measured, e.g., in an animal model. In some embodiments, dose related plasma and brain levels are determined. In some embodiments, brain Cmax>100, 300, 600, 1000, 1300, 1600, or 1900 ng/mL. In some embodiments good brain penetrability is defined as a brain/plasma ratio of >0.1, >0.3, >0.5, >0.7, >0.8, >0.9, preferably >1, and more preferably >2, >5, or >10. In other embodiments, good brain penetrability is defined as greater than about 0.1%, 1%, 5%, greater than about 10%, and preferably greater than about 15% of an administered dose crossing the BBB after a predetermined period of time. In certain embodiments, the dose is administered orally (p.o.). In other embodiments, the dose is administered intravenously (i.v.), prior to measuring pK properties. Assays and brain penetrability are described in Example 7 for and data for compound II are shown in FIGS. 2A and 2B, Compound II was known to be subject to first pass metabolism and thus was dosed subcutaneously; nevertheless Compound II was highly brain penetrant following both acute and chronic dosing. Brain/plasma ratio for compound II was >8.

As used herein the term “plasma stability” refers to the degradation of compounds in plasma, for example, by enzymes such as hydrolases and esterases. Any of a variety of in vitro assays can be employed. Drugs are incubated in plasma over various time periods. The percent parent compound (analyte) remaining at each time point reflects plasma stability. Poor stability characteristics can tend to have low bioavailability. Good plasma stability can be defined as greater than 50% analyte remaining after 30 min, greater than 50% analyte remaining after 45 minutes, and preferably greater than 50% analyte remaining after 60 minutes.

As used herein the term “metabolic stability” refers to the ability of the compound to survive first-pass metabolism (intestinal and hepatic degradation or conjugation of a drug administered orally). This can be assessed, for example, in vitro by exposure of the compounds to mouse or human hepatic microsomes. In some embodiments, good metabolic stability refers to a t_{1/2}>5 min, >10 min, >15 minutes, >20 minutes, and preferably >30 min upon exposure of a compound to mouse or human hepatic microsomes. In some embodiments, good metabolic stability refers to an Intrinsic Clearance Rate (Clint) of <300 uL/min/mg, preferably ≦200 uL/min/mg, and more preferably ≦100 uL/min/mg.

In some embodiments, excluded are certain compounds of the prior art which were not known to be sigma 2 antagonists and either (i) were known to bind to sigma 2 receptor and to reduce or eliminate Abeta induced pathologies such as a defect in membrane trafficking or synapse reduction in neuronal cells or (ii) were known to have activity against symptoms of Alzheimer's disease without implication of sigma 2 receptor interaction. In some embodiments, the compounds described in Table 1A are disclaimed with respect to compositions or methods of the disclosure, with respect to Table 1A, R, R₁, and R₂ can independently be alkyl, alkoxy, halo, halo alkyl, or halo alkoxy, and n=0-8.

TABLE 1A
Disclaimed Compounds.

| Disclaimed Compound | Reference |
|---|---|
| 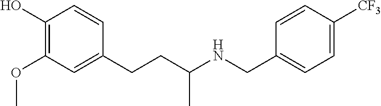 [SMILES: COc1cc(CCC(C)NCc2ccc(C(F)(F)F)cc2)ccc1O] | CogRx Rishton, Catalano WO 2010/118055 A1 Example 2, pp. 46-47 |
| 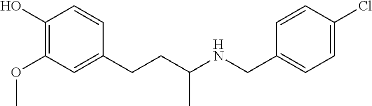 [SMILES: COc1cc(CCC(C)NCc2ccc(Cl)cc2)ccc1O] | CogRx Rishton, Catalano WO 2010/118055 A1 Example 1, pp. 43-44 |
| 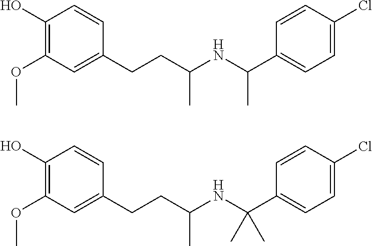 | CogRx Rishton, Catalano WO 2010/118055 A1 p. 32 |
| 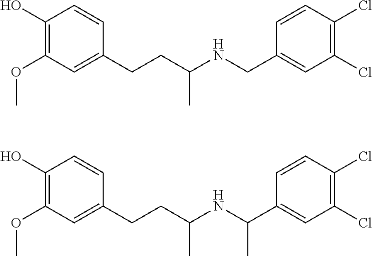 [SMILES: COc1cc(CCC(C)NCc2ccc(Cl)c(Cl)c2)ccc1O ; COc1cc(CCC(C)NC(C)c2ccc(Cl)c(Cl)c2)ccc1O] | CogRx Rishton, Catalano WO 2010/118055 A1 p. 32 |
| 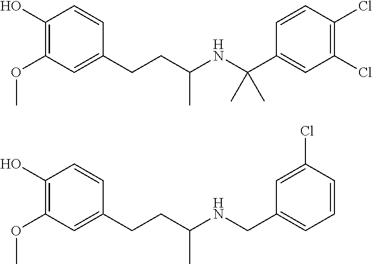 [SMILES: COc1cc(CCC(C)NC(C)(C)c2ccc(Cl)c(Cl)c2)ccc1O ; COc1cc(CCC(C)NCc2cccc(Cl)c2)ccc1O] | CogRx Rishton, Catalano WO 2010/118055 A1 p. 32 |
| 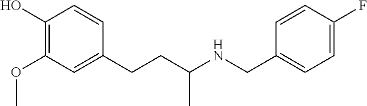 [SMILES: COc1cc(CCC(C)NCc2ccc(F)cc2)ccc1O] | CogRx Rishton, Catalano WO 2010/118055 A1 p. 32 |
| 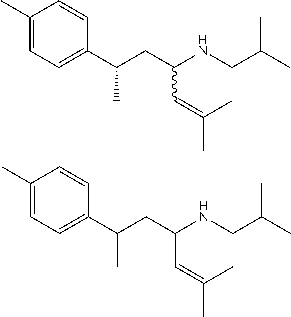 | CogRx Rishton, Catalano WO 2011/014880 A1 Example 1, pp. 41-42 |
| 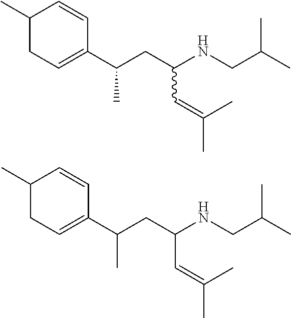 | CogRx Rishton, Catalano WO 2011/014880 A1 Example 1, p. 42 |
| 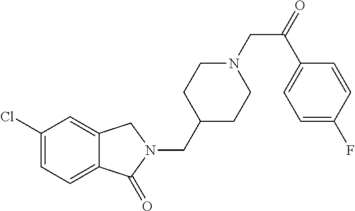 [SMILES: O=C(CN1CCC(CN2Cc3cc(Cl)ccc3C2=O)CC1)c1ccc(F)cc1] | Rocher, Yamabe WO 2001/64670 A1 Laurini, et al. Bioorganic and Medicinal Chemistry Letters 20 (2010) 2954-2957 |
| 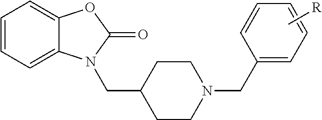 | Rocher, Yamabe WO 2001/64670 A1 Laurini, et al. Bioorganic and Medicinal Chemistry Letters 20 (2010) 2954-2957 |
| 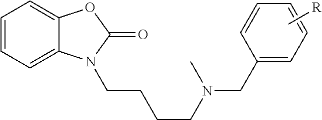 | Rocher, Yamabe WO 2001/64670 A1 Laurini, et al. Bioorganic and Medicinal Chemistry Letters 20 (2010) 2954-2957 |
| 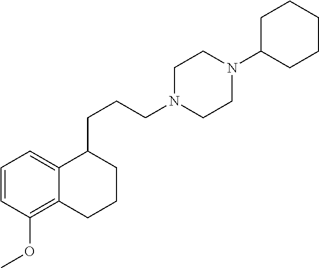 [SMILES: COc1cccc2c1CCCC2CCCN1CCN(C2CCCCC2)CC1] | Colabufo WO 2007/077543 A2 |
| 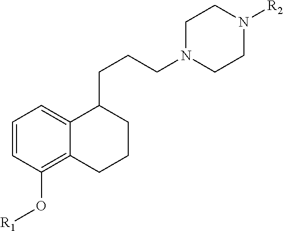 | Colabufo WO 2007/077543 A2 |
| 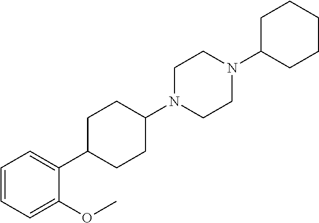 [SMILES: COc1ccccc1C1CCC(N2CCN(C3CCCCC3)CC2)CC1] | Colabufo WO 2007/077543 A2 |
| 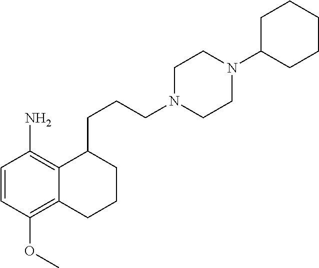 [SMILES: COc1ccc(N)c2c1CCCC2CCCN1CCN(C2CCCCC2)CC1] | Abate WO 2009/104058 A1 |
| 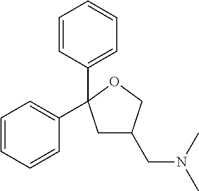 [SMILES: CN(C)CC1COC(c2ccccc2)(c2ccccc2)C1] | Anavex US 2010/0069484 A1 Anavex WO 2008/087458 A2 |
| 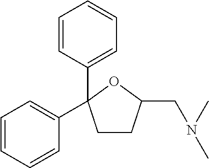 [SMILES: CN(C)CC1CCC(c2ccccc2)(c2ccccc2)O1] | Anavex US 2010/0069484 A1 Anavex WO 2008/087458 A2 |
| 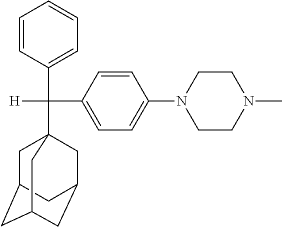 [SMILES: CN1CCN(c2ccc(C(c3ccccc3)C34CC5CC(CC(C5)C3)C4)cc2)CC1] | Anavex US 2010/0069484 A1 Anavex WO 2008/087458 A2 |
| 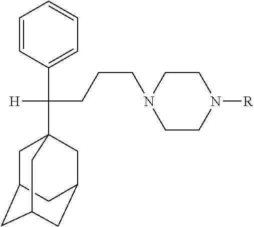 | Anavex US 2010/0069484 A1 Anavex WO 2008/087458 A2 |
| 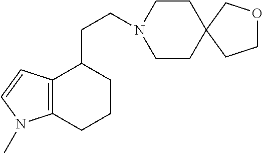 [SMILES: Cn1ccc2c1CCCC2CCN1CCC2(CCOC2)CC1] | Corbrera EP 1829862 A1 2007 |
| 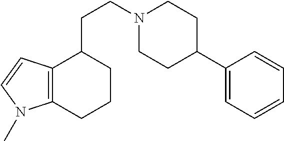 [SMILES: Cn1ccc2c1CCCC2CCN1CCC(c2ccccc2)CC1] | Corbrera EP 1829862 A1 2007 |
| 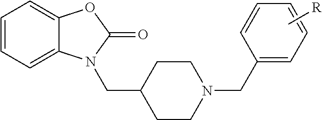 | Rocher, Yamabe WO 0164670 A1 |
| 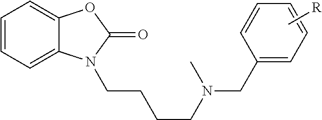 | Rocher, Yamabe WO 0164670 A1 |
| 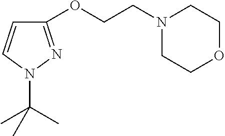 [SMILES: CC(C)(C)n1ccc(OCCN2CCOCC2)n1] | Cuberes EP 1829867 A1 2007 |
| 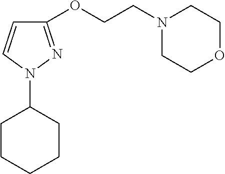 [SMILES: c1cn(C2CCCCC2)nc1OCCN1CCOCC1] | Cuberes EP 1829867 A1 2007 |
| 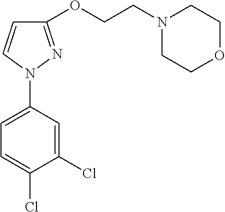 [SMILES: Clc1ccc(-n2ccc(OCCN3CCOCC3)n2)cc1Cl] | Cuberes EP 1829867 A1 2007 |
| 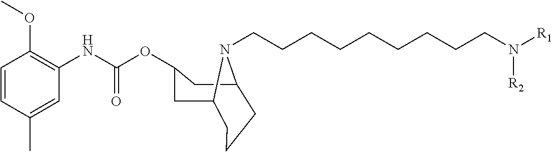 | Hawkins, Mach US 2009/0176705 A1 |
| 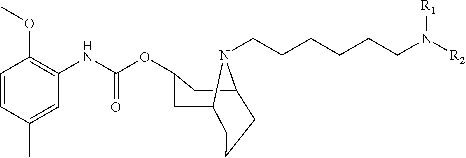 | Hawkins, Mach US 2009/0176705 A1 |
| 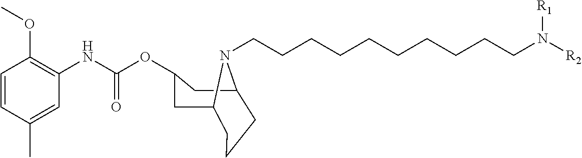 | Hawkins, Mach US 2009/0176705 A1 |
| 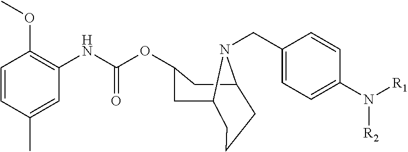 | Hawkins, Mach US 2009/0176705 A1 |
| 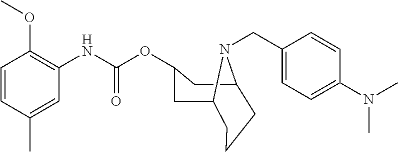 [SMILES: COc1ccc(C)cc1NC(=O)OC1CC2CCCC(C1)N2Cc1ccc(N(C)C)cc1] | Compound WC-26 Hawkins, Mach US 2009/0176705 A1 |
| 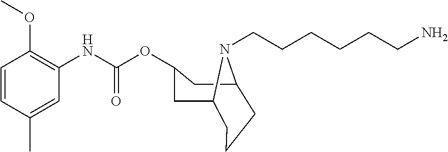 [SMILES: COc1ccc(C)cc1NC(=O)OC1CC2CCCC(C1)N2CCCCCCN] | Compound SV-119 Hawkins, Mach US 2009/0176705 A1 |
| 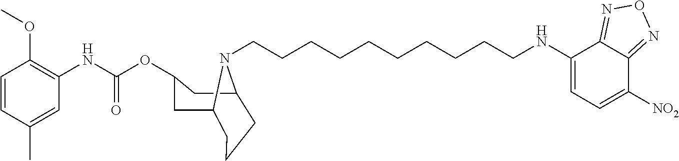 [SMILES: COc1ccc(C)cc1NC(=O)OC1CC2CCCC(C1)N2CCCCCCCCCCNc1ccc([N+](=O)[O-])c2nonc12] | Compound SW-120 Hawkins, Mach US 2009/0176705 A1 |
| 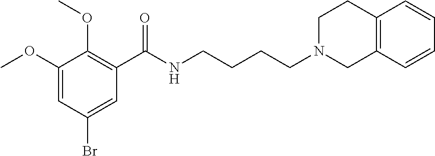 [SMILES: COc1cc(Br)cc(C(=O)NCCCCN2CCc3ccccc3C2)c1OC] | Mach US 2005/0107398 A1 |
| 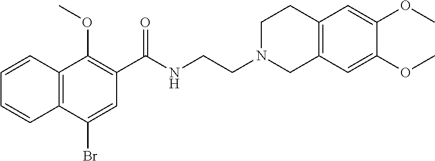 [SMILES: COc1cc2c(cc1OC)CN(CCNC(=O)c1cc(Br)c3ccccc3c1OC)CC2] | Mach US 2005/0107398 A1 |
| 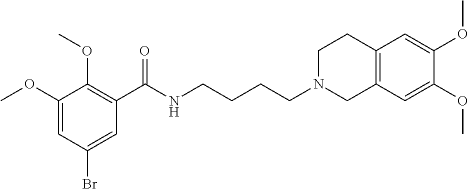 | Mach US 2005/0107398 A1 |
| 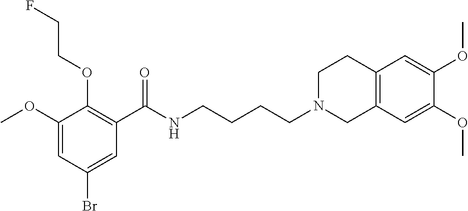 | Mach US 2005/0107398 A1 |
| 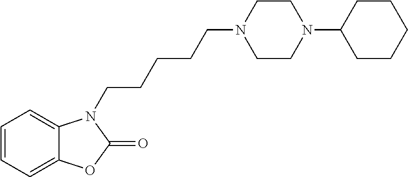 [SMILES: O=c1oc2ccccc2n1CCCCCN1CCN(C2CCCCC2)CC1] | McCurdy WO 2009/026227 A2 |
| 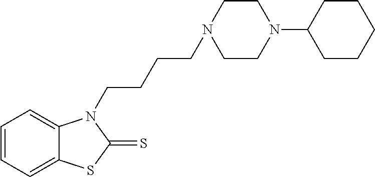 [SMILES: S=c1sc2ccccc2n1CCCCN1CCN(C2CCCCC2)CC1] | McCurdy JPET 333:491-500, 2010 US 2011/0280804 A1 |
| 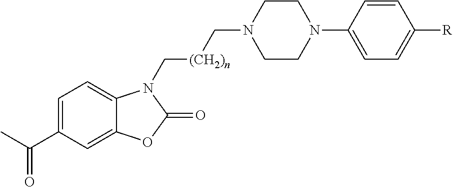 | McCurdy WO 2009/026227 A2 |
| 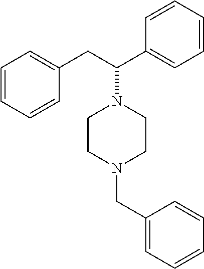 [SMILES: c1ccc(C[C@H](c2ccccc2)N2CCN(Cc3ccccc3)CC2)cc1] | Mita JP 2001/26584 |
| 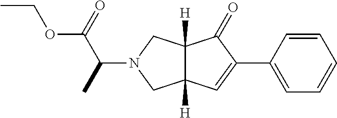 [SMILES: CCOC(=O)[C@H](C)N1C[C@H]2C=C(c3ccccc3)C(=O)[C@H]2C1] | Pericas WO 2007/128458 A1 |
| 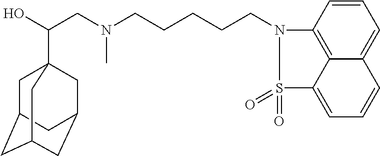 [SMILES: CN(CCCCCN1c2cccc3cccc(c23)S1(=O)=O)CC(O)C12CC3CC(CC(C3)C1)C2] | Rocher, Yamabe WO 1996/05185 |

In some embodiments, the compounds known as RHM-1, RHM-4, PB28, SM-21, M-14, NE100, BD1008, BD1047, fluvoxamine, PPBP, pentazocine or haloperidol are disclaimed with respect to compositions or methods of the disclosure. In certain assays, siramesine, SV-119 and WC-26 are functional sigma-2 receptor agonists. In some embodiments, the compositions and methods of the disclosure do not comprise siramesine, SV-119 or WC-26.

Sigma-2 Agonists Cause Cellular Toxicity

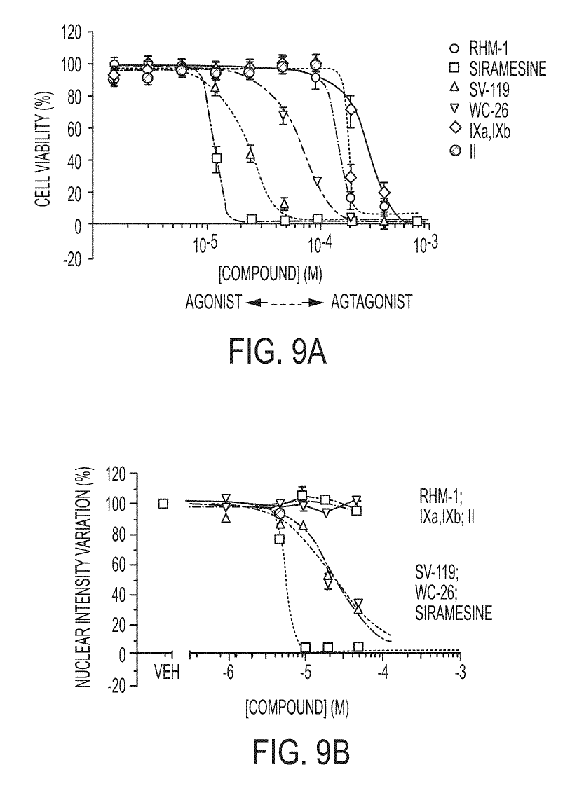

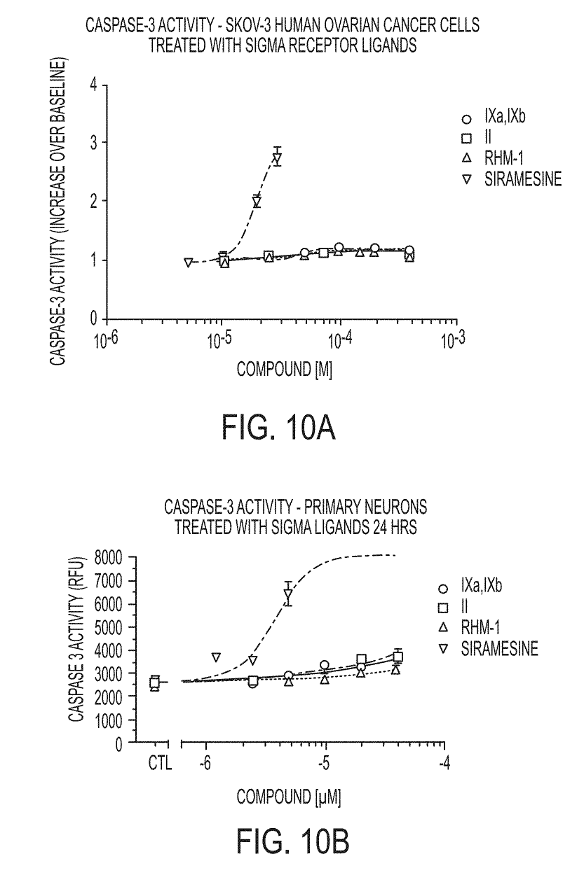

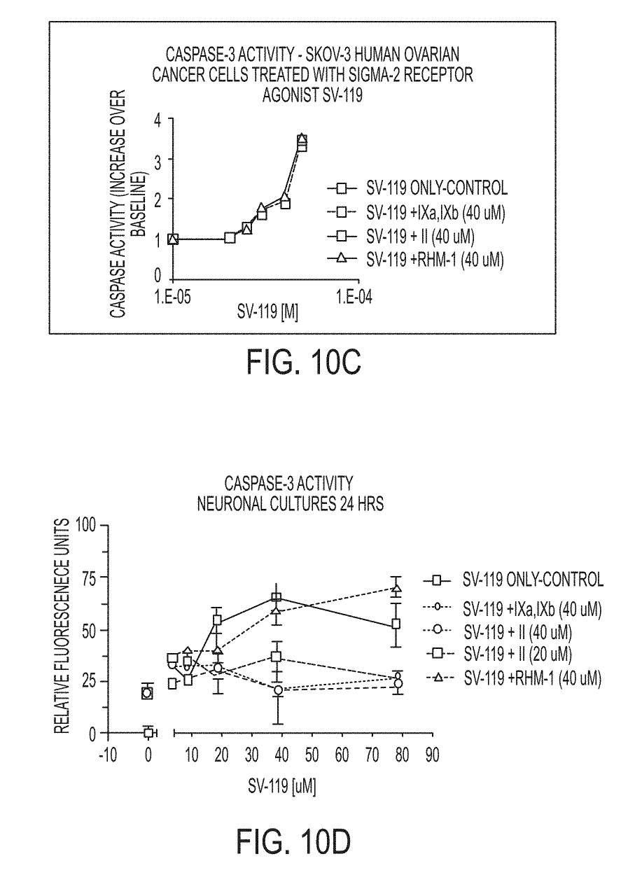

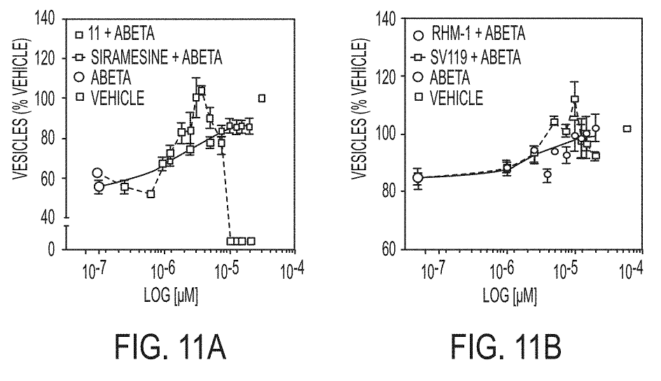

Without being bound by theory, it is proposed that Abeta oligomers are sigma-2 receptor agonists that bind to sigma protein complexes and can cause various deleterious Abeta effects such as neuronal toxicity, aberrant trafficking and synapse loss. It is demonstrated herein that known sigma-2 receptor agonists such as siramesine, SV-119, and WC-26, are cytotoxic to tumor cells and neurons, as exhibited by the ability to kill tumor cells and cause abnormal nuclear morphology in neurons (FIGS. 9A and 9B). Sigma-2 agonists (siramesine, SV-119), although capable of blocking oligomer-induced trafficking deficits at low concentrations, cause cellular toxicity and caspase-3 activation at higher concentrations (see agonist siramesine FIG. 10A and SV119 in 10B). Sigma-2 antagonists such as Compound II and IXa,IXb can block caspase-3 activation in neuronal cells caused by sigma-2 receptor agonists such as SV-119, as seen in FIG. 10D. Sigma-2 antagonists block Abeta oligomer-induced trafficking deficits at all tested concentrations without causing cellular toxicity, for example, see II in FIG. 11A and RHM-1 in FIG. 11B.

It is herein disclosed that a high affinity, selective sigma-2 functional antagonist having the therapeutic phenotype, and good drug-like properties, can be used to treat Abeta oligomer-induced synaptic dysfunction.

In certain embodiments, the compositions of the invention comprise selective sigma-2 functional antagonists that have high binding affinity to the sigma receptors. The sigma receptors include both the sigma-1 and sigma-2 subtypes. See Hellewell, S. B. and Bowen, W. D., Brain Res. 527: 224-253 (1990); and Wu, X.-Z. et al., J. Pharmacol. Exp. Ther. 257: 351-359 (1991). A sigma receptor binding assay which quantitates the binding affinity of a putative ligand for both sigma sites (against ³H-DTG, which labels both sites with about equal affinity) is disclosed by Weber et al., Proc. Natl. Acad. Sci (USA) 83: 8784-8788 (1986). Alternatively, [³H]pentozocine may be used to selectively label the sigma-1 binding site in a binding assay. A mixture of [³H]DTG and unlabeled (+)pentazocine is used to selectively label the sigma-2 site in a binding assay. The present invention is also directed to compositions comprising certain ligands which are selective for the sigma-1 and sigma-2 receptors and act as sigma-2 functional antagonists as well as use of these compositions to treat Abeta oligomer-induced synaptic dysfunction. The discovery of such ligands which are selective for one of the two sigma receptor subtypes may be an important factor in identifying compounds which are efficacious in treating central nervous system disorders with minimal side effects.

In some embodiments, the sigma-2 antagonist is selected from a small molecule or an antibody, or active binding fragment thereof, with high affinity for the sigma-2 receptor that has the ability to block soluble Abeta oligomer binding or Abeta oligomer-induced synaptic dysfunction.

Anti-Abeta Antibodies Several anti-Abeta monoclonal antibodies are in clinical development for the treatment of Alzheimer's disease. In some embodiments, the disclosure provides compositions comprising an sigma-2 receptor antagonist with an anti-Abeta antibody. For example, Bapineuzumab (AAB-00; Janssen, Elan, Pfizer) is an anti-β-amyloid humanized IgG₁ monoclonal antibody in Phase III clinical development for intravenous treatment of mild to moderate Alzheimer's disease. In a phase II clinical study, certain patients receiving the high dose of 2 mg/kg experienced reversible vasogenic edema. Although no significant differences were found in the primary efficacy analysis, meta-analysis showed potential treatment differences in APOE epsilon4 non-carriers. Salloway et al., 2009, A phase 2 ascending dose trial of bapineuzumab in mild to moderate Alzheimer's disease Neurol. 2009; 73(24):2061-70. Now undergoing phase III studies, bapineuzumab is administered at 0.5 or 1.0 mg/kg by intravenous infusion once about every 13 weeks with concurrent use of a cholinesterase inhibitor or memantidine allowed. Bapineuzumab recognizes an N-terminal epitope of Abeta: Abeta₁₋₅. Other anti-Aβ humanized monoclonal antibodies are in various phases of clinical development including solanezumab (LY2062430; Lilly) raised to Abeta₁₃₋₂₈, PF-04360365 (Pfizer) which targets Abeta₃₃₋₄₀; MABT5102A (Genentech); GSK933776 (GlaxoSmithKline) and gantenerumab (R1450, RO4909832, Hoffman-LaRoche). Solanezumab in secondary analysis of Phase III clinical trial results was recently reported to show statistically significant slowing of cognitive decline in patients with mild AD, but not in patient's with moderate AD. In one embodiment, a composition comprising a sigma-2 antagonist and solanezumab is used in a method for slowing cognitive decline in patients with mild AD.

It is acknowledged that peripherally administered antibodies may not have access to the tissue of interest, although passive immunization appeared to work in mice. One hypothesis was that circulating antibodies to Aβ shift the equilibrium of the Aβ peptide from the cerebrospinal fluid to the plasma, indirectly reducing the brain's Aβ burden. Kerchner at al, 2010, Bapineuzumab, Expert Opin Biol Ther., 10(7):1121-1130. Alternatively, it was proposed that it may be possible that intravenously-administered antibodies may bind Aβ directly in the brain. See, e.g., Yamada et al., 2009, Aβ immunotherapy: Intracerebral sequestration of Aβ by an anti-Ab monoclonal antibody 266 with high affinity to soluble Aβ. J Neurosci 29(36):11393-11398. Unfortunately, thus far intravenous amyloid beta specific monoclonal antibodies have not proven particularly efficacious; for example, recently, development of intravenous bapineuzumab was ended due to lack of efficacy in two late-stage trials in patients who had mild to moderate Alzheimer's disease.

Anti-Abeta polyclonal antibodies occur naturally in pooled preparations of intravenous immunoglobulin (IVIg or IGIV), which is already FDA-approved for the treatment of other neurological conditions. At least two clinical trials using IVIg in AD are underway by Baxter and Octpharma. Kerchner et al., 2010 infra. In some embodiments, the disclosure provides methods and compositions for the treatment of cognitive decline, or Alzheimer's disease, wherein the compositions comprise a sigma-2 receptor antagonist compound and an anti-Abeta antibody and a pharmaceutically acceptable carrier.

Sigma-2 Receptor Antibodies

In some embodiments, the sigma-2 receptor antagonist compound is a sigma-2 receptor specific antibody, or active binding fragment thereof, that has the ability to block soluble Abeta oligomer binding or Abeta oligomer-induced synaptic dysfunction. In preferred embodiments, the sigma-2 antagonist antibody or immunospecific fragment thereof for use in the methods disclosed herein will not elicit a deleterious immune response in the animal to be treated, e.g., in a human. In certain embodiments, the sigma-2 antagonist antibodies or active binding fragments thereof for use in the treatment methods disclosed herein may be modified to reduce their immunogenicity using art-recognized techniques. For example, antibodies can be humanized, primatized, deimmunized, synthetic or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, for example, but not limited to, (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; (c) transplanting the entire non-human variable domains, but “cloaking” them with a human-like section by replacement of surface residues or (d) use of genetically modified mice wherein the mouse engineered to express human repertoire, for example, human immunoglobulin heavy and light chain variable domains. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), Peterson, ILAR Journal 46(3): 314-319 (2005), Lonberg, Nat. Biotechnol. 23(9): 1119-1125 (2005) and U.S. Pat. Nos. 5,585,089, 5,693,761, 5,693,762, 6,190,370, and US2012/0021409, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, V_{H} and V_{L} sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative V_{H} and V_{L} sequences are designed comprising combinations of amino acid substitutions and these sequences are subsequently incorporated into a range of binding polypeptides, e.g., sigma-2 antagonist antibodies or immunospecific fragments thereof for use in the methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

Sigma-2 antagonist antibodies or fragments thereof for use in the methods of the present invention may be generated by any suitable method known in the art. Polyclonal antibodies can be produced by various procedures well known in the art. For example, a sigma-2 polypeptide fragment can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

In some embodiments, the sigma-2 antagonists, such as monoclonal antibodies, or active binding fragments thereof, specific for the sigma-2 receptor, can be engineered to enhance the ability to cross the blood-brain barrier using any available technique known in the art. Recombinant protein therapeutics cannot generally be employed for brain delivery since they will not cross the blood brain barrier; however, techniques are known in the art for brain delivery of biologic therapeutics. For example, see Pardridge and Boado, Reengineering biopharmaceuticals for targeted delivery across the blood-brain barrier, Methods Enzymol. 2012; 503:269-292, incorporated herein by reference. Pardridge and Boado report recombinant proteins can be reengineered as BBB-penetrating IgG fusion proteins, where the IgG part is a genetically engineered monoclonal antibody (MAb) against an endogenous BBB receptor, such as the human insulin receptor (HIR) or the transferrin receptor (TfR). The IgG binds the endogenous insulin receptor or TfR to trigger transport across the BBB and acts as a molecular Trojan horse (MTH) to ferry into brain the fused protein therapeutic. The pharmacokinetic (PK) properties of the IgG fusion proteins differ from that of typical MAb drugs and resemble the PK profiles of small molecules due to rapid uptake by peripheral tissues, as well as brain. The brain uptake of the IgG fusion proteins, 2-3% of injected dose/brain, is comparable to the brain uptake of small molecules. The IgG fusion proteins have been administered chronically in mouse models, and the immune response is low titer and has no effect on the fusion protein clearance from blood or brain uptake in vivo. For example, Zhou et al used the “Trojan horse” to re-engineer an anti-Abeta amyloid antibody (AAA) as a fusion protein with a blood-brain barrier (BBB) molecular Trojan horse. The AAA was engineered as a single-chain Fv (ScFv) antibody, and the ScFv was fused to the heavy chain of a chimeric monoclonal antibody (Mab) against the mouse transferrin receptor (TfR). The cTfRMAb-ScFv protein penetrates mouse brain from blood via transport on the BBB TfR, and the brain uptake is 3.5% of injected dose/gram brain following an intravenous administration. Zhou et al., Receptor-mediated Abeta Amyloid Antibody Targeting to Alzheimer's Disease Mouse Brain. Mol. Pharm. 2011, Feb. 7; 8(1):280-285. The BBB MTH technology enables the reengineering of a wide spectrum of recombinant protein therapeutics for targeted drug delivery to the brain.

In some embodiments, the antibodies can be engineered to enhance brain uptake by the method of Yu et al., 2011. Antibodies targeting the transferrin receptor, which is highly expressed by endothelial cells that make up the BBB, have been reported to cross the BBB by receptor-mediated transcytosis. One problem with this approach is that high affinity antibodies targeting the transferrin receptor might reduce the probability of antibody being released from the CNS vasculature. Yu et al designed antibodies with low affinity for transferrin to increase release of antibody from brain vascular endothelium and to enhance uptake and distribution to the brain. Yu reported that lower-affinity ant-TfR antibodies show increased brain uptake. In some embodiments, the anti-sigma-2 receptor antibody is a bispecific antibody with one arm comprising a low-affinity anti-transferrin receptor antibody and the other arm comprising a high-affinity anti-sigma-2 receptor antibody or anti-PGRMC1 antibody by the method of Y. Joy Yu et al., Sci Transl Med 3, 84ra44(2011), and US2012/0171120, each of which is incorporated herein by reference.

In some embodiments, the sigma-2 antagonist is selected from any anti-PGRMC1 antibody, or from any antibody, or fragment thereof, that is specific for binding the sigma-2 receptor and that also blocks Abeta oligomer binding or Abeta oligomer-induced synaptic dysfunction or that acts as a functional neuronal antagonist, or that blocks Abeta oligomer binding and Abeta effects.

In some embodiments, the sigma-2 receptor antibody or binding fragment thereof can be reengineered as BBB-penetrating IgG fusion protein, or conjugate, where the IgG part is a genetically engineered monoclonal antibody (MAb) against an endogenous BBB receptor, such as the human insulin receptor (HIR) or the transferrin receptor (TfR). Conjugates of anti-sigma-2 receptor antibodies, or fragments thereof, to HIR or TfR Mabs via, for example, chitosan nanoparticles, or poly-malic acid conjugates. See, e.g., Yemisci et al., Transport of a caspase inhibitor across the blood-brain barrier by chitosan nanoparticles, Methods Enzymol., 2012; 508:243-269; and Ding et al., Inhibition of brain tumor growth by intravenous poly(b-L-malic acid) nanobioconjugate with a pH-dependent drug release. PNAS, 2010 107(42) 18143-18148, each of which is incorporated herein by reference. In some embodiments, single domain antibodies that are raised against receptors that undergo cytosis across the blood-brain barrier are employed in fusion proteins or conjugates. See for example, Abulrob et al., J Neurochem 2005, 95(4):1201-121, incorporated herein by reference.

In some embodiments, the anti-sigma-2 receptor antibody is raised to an epitope of human membrane-associated progesterone receptor component 1 (human PGRMC1) or an isoform, homolog, variant, extracellular domain or fragment thereof. For example, one protein sequence of human PGRMC1 is a 195 amino acid (aa) protein; GI:48146103:
maaedvvatg adpsdlesgg llheiftspl nllllglcif llykivrgdq paasgdsddd

eppplprlkr rdftpaelrr fdgvqdpril maingkvfdv tkgrkfygpe gpygvfagrd

asrglatfcl dkealkdeyd dlsdltaaqq etlsdwesqf tfkyhhvgkl lkegeeptvy

sdeeepkdes arknd SEQ ID NO: 1.

For example, progesterone receptor membrane component 1, isoform CRAa_[Homo sapiens], a 195 aa protein; GI:119610285:

maaedvvatg adpsdlesgg llheiftspl nllllglcif llykivrgdq paasgdsddd
eppplprlkr rdftpaelrr fdgvqdpril maingkvfdv tkgrkfygpe gpygvfagrd
asrglatfcl dkealkdeyd dlsdltaaqq etlsdwesqf tfkyhhvgkl lkegeeptvy
sdeeepkdes arknd SEQ ID NO: 2.

For example, progesterone receptor membrane component 1, isoform CRA_b [Homo sapiens], a 170 aa protein; GI: 119610286:

maaedvvatg adpsdlesgg llheiftspl nllllglcif llykivrgdq paasgdsddd
eppplprlkr rdftpaelrr fdgvqdpril maingkvfdv tkgrkfygpe gpygvfagrd
asrglatfcl dkemrknqkm rvpgkmikaf sgsisifvfc kiicnsplcl

### SEQ ID NO: 3.

For example, progesterone receptor membrane component 1, isoform CRA_c [Homo sapiens], a 143 aa protein; GI:119610287:

maaedvvatg adpsdlesgg llheiftspl nllllglcif llykivrgdq paasgdsddd
eppplprlkr rdftpaelrr fdgvqdpril maingkvfdv tkgrkfygpv kyhhvgkllk
egeeptvysd eeepkdesar knd SEQ ID NO: 4.

Homologs of human PGRMC1 include, e.g., rat PGRMC1. For example a rat PGRMC1, a 243 aa protein; GI:11120720:

maaedvvatg adpseleggg llqeiftspl nllllglcif llykivrgdq pgasgdnddd
eppplprlkp rdftpaelrr ydgvqdpril maingkvfdv tkgrkfygpe gpygvfagrd
asrglatfcl dkealkdeyd dlsdltpaqq etlndwdsqf sspsstitwg kllegaeepi
vysddeeqkm rllgrvteav sgaylflyfa ksfvtfqsvf ttw SEQ ID NO: 5.

Another homolog is rat PGRMC1, a 195 aa protein; GI:38303845:

maaedvvatg adpseleggg llqeiftspl nllllglcif llykivrgdq pgasgdnddd
eppplprlkp rdftpaelrr ydgvqdpril maingkvfdv tkgrkfygpe gpygvfagrd
asrglatfcl dkealkdeyd dlsdltpaqq etlndwdsqf tfkyhhvgkl lkegeeptvy
sddeepkdea arksd SEQ ID NO: 6.

In some embodiments, the specific sigma-2 receptor antagonist compound is an anti-sigma-2 receptor antibody that blocks binding between soluble Abeta oligomers and a sigma-2 receptor. In some embodiments, the anti-sigma-2 receptor antibodies recognize an epitope corresponding to an amino acid sequence of a PGRMC1 protein. In some embodiments, the sigma-2 receptor specific antibody may be specific for an epitope corresponding to an amino acid sequence derived from an N-terminal sequence, C-terminal sequence, internal sequence, or full length protein corresponding to PGRMC1. In embodiments, the sigma-2 receptor specific antibody may be specific for binding to one or more of SEQ ID NOs: 1, 2, 3, 4, 5, 6, 7, 9, or 10. In some embodiments, the specific sigma-2 receptor antagonist compound is an anti-PGRMC1 antibody recognizing the synthetic peptide: EPKDESARKND (SEQ ID NO: 7), corresponding to C terminal amino acids 185-195 of human PGRMC1. In some embodiments, sigma-2 receptor antagonist compound is not an antibody specific for residues 1-46 at the N-terminus of human PGRMC1 protein (MAAEDVVATGADPSDLESGGLLHEIFTSPLNLLLLGLCIFLLYKI (SEQ ID NO: 9), #sc-98680, Santa Cruz),

In some embodiments, the anti-sigma-2 receptor antibodies include those raised against, or in any event recognizing, any known full length PGRMC1 protein, or any variant, fragment, immunogen or epitope thereof; including an N-terminal, central fragment, or C-terminal region of PGRMC1, or homolog, immunogen or variant thereof. Isolated, purified, or synthetic proteins or peptides can be employed as immunogens. The proteins or fragments are optionally adjuvanted and or conjugated by various means known in the art to enhance immunogenicity. Synonyms for PGRMC1 include progesterone binding protein, HPR6.6; HGNC:16090, progesterone receptor membrane binding component 1, and MPR. In one embodiment, the fragment or epitope is EPKDESARKND SEQ ID NO: 7, corresponding to C terminal amino acids 185-195 of Human PGRMC1. This fragment was used to raise commercially-available goat anti-human PGRMC1 polyclonal antibodies (e.g., Abcam ab48012; Sigma-Aldrich SAB2500782; and Everest Biotech, Ltd. EB07207). Another fragment consists of residues 50-150 of human PGRMC1, taaqq etlsdwesqf tfkyhhvgkl Ikegeeptvy sdeeepkdes arknd (SEQ ID NO: 10); this fragment was conjugated to KLH by means known in the art; rabbit anti-PGRMC1 polyclonal antibodies were generated; commercially available as Abcam ab88948. Other commercially-available antibodies include Santa Cruz Biotechnology sc-98680 (goat anti-human PGRMC1 polyclonal antibodies raised against N-terminus aa 1-46, (MAAEDVVATG ADPSDLESGG LLHEIFTSPL NLLLLGLCIF LLYKIV (SEQ ID NO: 9); sc-82694 (goat anti-human PGRMC1 polyclonal antibodies raised against an internal epitope); sc-133906 (rabbit anti-human PGRMC1 polyclonal antibodies raised against synthetic PGRMC1 peptide); sc-135720 (PGRMC1 (12B7) mouse Mab); sc-271275 (PGRMC1(c-3) mouse Mab raised against N-terminal 1-46); and Sigma-Aldrich HPA002877 anti-PGRMC1 rabbit polyclonal antibodies raised against Membrane-associated progesterone receptor component 1 recombinant protein epitope signature tag (PrEST).

Other antibodies raised against sigma-1 receptor (opioid receptor, sigma-1; Oprs 1) proteins, fragments, epitopes or immunogens are employed in the examples provided herein. Such anti-sigma-1 receptor antibodies include Thermo Scientific PAS-12326 (rabbit anti-sigma-1 receptor polyclonal antibodies raised to N-terminal region of OPRS1 conjugated to KLH); Santa Cruz Biotechnology, Inc. sigma receptor (L-20) sc-16203, goat anti-human raised to an internal region of sigma-1 receptor); Santa Cruz Biothechnology, Inc. sigma receptor (FL-223) sc-20935 raised to rabbit anti-human full length sigma receptor aa 1-223; Santa Cruz Biotechnology, Inc. sigma receptor (S-18) sc-22948 goat anti-human polyclonal antibodies raised against and internal region of human sigma receptor; Santa Cruz Biotechnology, Inc. sigma receptor (B-5) sc-137075 a mouse monoclonal antibody (Mab) specific for an epitope mapping between amino acids 136-169 of an internal region of human sigma-1 receptor; and Santa Cruz Biotechnology, Inc. sigma receptor (F-5) a mouse monoclonal antibody raised against amino acids 1-223 full length human sigma-1 receptor.

The human Sigma-1 receptor is a 223 aa protein; GI:74752153:

mqwavgrrwa waalllavaa vltqvvwlwl gtqsfvfqre eiaqlarqya gldhelafsr
livelrrlhp ghvlpdeelq wvfvnaggwm gamcllhasl seyvllfgta lgsrghsgry
waeisdtiis gtfhqwregt tksevfypge tvvhgpgeat avewgpntwm veygrgvips
tlafaladtv fstqdfltlf ytlrsyargl rlelttylfg qdp SEQ ID NO: 8.

In some embodiments, any sigma-1 receptor full length protein, homolog variant, or fragment, including N-terminal, C-terminal, central regions, can be employed to raise antibodies as sigma-1 receptor antagonists by any method known in the art.

In some embodiments, the sigma-2 antagonist is a small molecule compound with high affinity for the sigma-2 receptor.

Sigma-2 Receptor Ligands for Selection as Sigma-2 Receptor Antagonists

In some embodiments, sigma-2 receptor antagonists for use in the present invention are selected from among sigma-2 receptor ligand compounds that also meet additional selection criteria. Additional criteria are used to select sigma-2 receptor antagonists for use in the present invention from among sigma-2 receptor ligands. Additional selection criteria include: acting as a functional antagonist in a neuronal cell with respect to inhibiting soluble Aβ oligomer induced synapse loss, and inhibiting soluble Aβ oligomer induced deficits in a membrane trafficking assay; having high selectivity for one or more sigma receptors compared to any other non-sigma receptor; exhibiting high affinity at a sigma-2 receptor; and exhibiting good drug-like properties including good brain penetrability, good metabolic stability and good plasma stability. In some embodiments, the sigma-2 receptor antagonist is further selected on the basis of exhibiting one or more of the additional following properties: does not affect trafficking or synapse number in the absence of Abeta oligomer; does not induce caspase-3 activity in a neuronal cell; inhibits induction of caspase-3 activity by a sigma-2 receptor agonist; and/or decreases or protects against neuronal toxicity in a neuronal cell caused by a sigma-2 receptor agonist.

In some embodiments, certain sigma-2 receptor ligand compounds subject to further selection criteria are selected from compounds described herein and can be synthesized according to the methods described herein or in WO 2011/014880 (Application No. PCT/US2010/044136), WO 2010/118055 (Application No. PCT/US2010/030130), Application No. PCT/US2011/026530, and WO2012/106426, each of which is incorporated herein by reference in its entirety. Additional options for preparing these compounds are discussed in detail below.

In some embodiments, the sigma-2 ligand is an optionally substituted piperazine, phenyltetrahydrofuran-N,N-dimethylmethanamine, diphenyltetrahydrofuran-N,N-dimethylmethanamine, a 4-phenylpentyl-piperazine, benzylphenyl-piperazine, indole-oxa-azaspiro-decane, piperadine-indole, phenylpiperadine-indole, pyrazole-morpholine, pyrazole-piperadine, pyrazol-N,N-diethylethanamine, pyrazole-pyrrolidine, phenyl-pyrazol-morpholine, benzamide-quinoline compound, or derivatives thereof.

In some embodiments, the sigma-2 ligand is an optionally substituted, N-(4-(3,4-dihydroisoquinolin-2(1H)-yl)butyl)benzamide, 1-cyclohexyl-4-(3-(1,2,3,4-tetrahydronaphthalen-1-yl)propyl)piperazine, (5,5-diphenyltetrahydrofuran-3-yl)methanamine, 1-(5,5-diphenyltetrahydrofuran-3-yl)-N,N-dimethylmethanamine, N,N-dimethyl-1-(5-phenyltetrahydrofuran-3-yl)methanamine, 1-(4-phenylbutyl)piperazine, 1-(4-benzylphenyl)-4-methylpiperazine, cyclohexyl-4-(4-phenylcyclohexyl)piperazine, 1-cyclohexyl-4-(4-phenylcyclohexyl)piperazine, cyclohexyl-4-(3-(1,2,3,4-tetrahydronaphthalen-1-yl)propyl)piperazine, 1-cyclohexyl-4-(3-(1,2,3,4-tetrahydronaphthalen-1-yl)propyl)piperazine, 8-(2-(4,5,6,7-tetrahydro-1H-indol-4-yl)ethyl)-2-oxa-8-azaspiro[4.5]decane, 4-(2-(4-phenylpiperidin-1-yl)ethyl)-4,5,6,7-tetrahydro-1H-indole, 4-(2-(1H-pyrazol-3-yloxy)alkyl)morpholine, 4-(2-(1H-pyrazol-3-yloxy)ethyl)morpholine, 1-(2-(1H-pyrazol-3-yloxy)ethyl)piperidine, 1-(2-(1H-pyrazol-3-yloxy)alkyl)piperidine, 2-(1H-pyrazol-3-yloxy)-N,N-diethylethanamine, 2-(1H-pyrazol-3-yloxy)-N,N-dialkyl-alkanamine, 3-(2-(pyrrolidin-1-yl)ethoxy)-1H-pyrazole, 3-(2-(pyrrolidin-1-yl)alkoxy)-1H-pyrazole, 1-(1H-pyrazol-3-yloxy)-3-(piperazin-1-yl)propan-2-ol, 1-(1H-pyrazol-3-yloxy)-3-(piperazin-1-yl)alkan-2-ol, 1-(1-aryl-1H-pyrazol-3-yloxy)-3-(piperazin-1-yl)alkan-2-ol, 1-(1-phenyl-1H-pyrazol-3-yloxy)-3-(piperazin-1-yl)alkan-2-ol, 1-(1-aryl-1H-pyrazol-3-yloxy)-3-(piperazin-1-yl)propan-2-ol, 1-(1-heteroaryl-1H-pyrazol-3-yloxy)-3-(piperazin-1-yl)alkan-2-ol, 1-(1-(dihaloaryl)-1H-pyrazol-3-yloxy)-3-(piperazin-1-yl)alkan-2-ol, 1-(1-dihaloheteroaryl)-1H-pyrazol-3-yloxy)-3-(piperazin-1-yl)alkan-2-ol, 1-(1-(3,4-dihaloaryl)-1H-pyrazol-3-yloxy)-3-(piperidin-1-yl)alkan-2-ol, 1-(1-(3,4-dihaloheteroaryl)-1H-pyrazol-3-yloxy)-3-(piperidin-1-yl)alkan-2-ol, 1-(1-(dihaloaryl)-1H-pyrazol-3-yloxy)-3-(dialkylamino)alkan-2-ol, 1-(1-(dihaloheteroaryl)-1H-pyrazol-3-yloxy)-3-(dialkylamino)alkan-2-ol, 1-(1-(dichloroheteroaryl)-1H-pyrazol-3-yloxy)-3-(dialkylamino)alkan-2-ol, N-(4-(3,4-dihydroisoquinolin-2(1H)-yl)butyl)benzamide, N-(4-(3,4-dihydroisoquinolin-2(1H)-yl)alkyl)benzamide, N-(4-(3,4-dihydroisoquinolin-2(1H)-yl)alkyl)-2-hydroxybenzamide, N-(4-(6,7-dimethoxy-3,4-dihydroisoquinolin-2(1H)-yl)butyl)benzamide, N-(4-(6,7-dialkoxy-3,4-dihydroisoquinolin-2(1H)-yl)alkyl)benzamide, N-(4-(6,7-dimethoxy-3,4-dihydroisoquinolin-2(1H)-yl)butyl)-2-naphthamide, N-(4-(6,7-dialkoxy-3,4-dihydroisoquinolin-2(1H)-yl)alkyl)-2-naphthamide, N-(4-(3,4-dihydroisoquinolin-2(1H)-yl)butyl)-2-naphthamide, N-(4-(3,4-dihydroisoquinolin-2(1H)-yl)alkyl)-2-naphthamide, N-(2-(3,4-dihydroisoquinolin-2(1H)-yl)ethyl)-2,3-dimethoxybenzamide, N-(2-(3,4-dihydroisoquinolin-2(1H)-yl)alkyl)-2,3-dialkoxybenzamide, N-(2-(3,4-dihydroisoquinolin-2(1H)-yl)ethyl)benzamide, N-(2-(3,4-dihydroisoquinolin-2(1H)-yl)alkyl)benzamide, N-(2-(4-(2,3-dichlorophenyl)piperidin-1-yl)ethyl)-2-naphthamide, N-(2-(4-(2,3-dihaloaryl)piperidin-1-yl)alkyl)-2-naphthamide, 2,3-dimethoxy-N-(4-(4-phenylpiperidin-1-yl)butyl)benzamide, 2,3-dialkoxy-N-(4-(4-phenylpiperidin-1-yl)alkyl)benzamide, N-(4-(4-(2,3-dihalophenyl)piperidin-1-yl)alkyl)-2,3-dialkoxybenzamide, 5-halo-N-(4-(4-(2,3-dihalophenyl)piperidin-1-yl)alkyl)-2,3-dialkoxybenzamide (wherein the 2, 3, & 5 position halo are the same or independently F, Cl, Br, or I), N-(4-(6,7-dimethoxy-3,4-dihydroisoquinolin-2(1H)-yl)butyl)-2-(2-fluoroethoxy)-5-iodo-3-methoxybenzamide, N-(4-(6,7-dialkoxy-3,4-dihydroisoquinolin-2(1H)-yl)alkyl)-2-(2-haloethoxy)-5-halo-3-methoxybenzamide (wherein the halo substituents are the same or independently F, Cl, Br, or I), 9-benzyl-9-azabicyclo[3.3.1]nonan-3-yl phenylcarbamate, 9-benzyl-9-azabicyclo[3.3.1]nonan-3-yl 2-alkoxyphenylcarbamate, 9-(5-phenylalkyl)-9-azabicyclo[3.3.1]nonan-3-yl 2-methoxy-5-methylphenylcarbamate, 9-alkyl-9-azabicyclo[3.3.1]nonan-3-yl phenylcarbamate, 3-(2-(4-cyclohexylpiperazin-1-yl)alkyl)benzo[d]oxazol-2(3H)-one, 6-acetyl-3-(4-(4-cyclohexylpiperazin-1-yl)alkyl)benzo[d]oxazol-2(3H)-one, 1-benzyl-4-(1,2-diphenylethyl)piperazine, 1-aryl-4-(1,2-diphenylethyl)piperazine, ethyl 2-(6-oxo-5-phenyl-3,3a,6,6a-tetrahydrocyclopenta[c]pyrrol-2(1H)-yl)propanoate, ethyl 2-(5-alkyl-6-oxo-3,3a,6,6a-tetrahydrocyclopenta[c]pyrrol-2(1H)-yl)propanoate, 2-((3-(2H-naphtho[1,8-cd]isothiazol-2-yl)alkyl)(methyl)amino)alkanol, 2-((1-(2-(1-alkyl-1H-pyrrol-2-yl)-2-oxoethyl)piperidin-4-yl)alkyl)isoindolin-1-one, 2-((1-(2-oxoaklyl)piperidin-4-yl)alkyl)isoindolin-1-one, 1′-(4-(1-(4-haloaryl)-1H-indol-3-yl)alkyl)-3H-spiro[isobenzofuran-1,4′-piperidine], 1′-(4-(1-(4-haloheteroaryl)-1H-indol-3-yl)alkyl)-3H-spiro[isobenzofuran-1,4′-piperidine], 3-(3-alkylbut-2-alkynyl)-1,2,3,4,5,6-hexahydrobenzo[d]azocin-8-ol, pentazocine compound, or analogues or derivatives thereof.

In some embodiments, the sigma-2 ligand comprises a compound of Formula I:

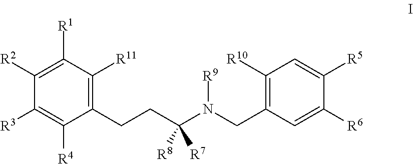

or a pharmaceutically acceptable salt thereof, wherein:

R¹, R², R3, R⁴, and R¹¹ are each independently selected from H, OH, halo, C₁₋₆ alkyl, C₁₋₆ haloalkyl, C₁₋₆ alkoxy, and NH(C₁₋₄ alkyl);

R⁵ and R⁶ are each independently selected from H, C₁₋₆ haloalkyl, C₁₋₆ alkyl, and C₃₋₇ cycloalkyl, and NH(C₁₋₄ alkyl);

R⁷ and R⁸ are each independently selected from H, C₁₋₆ alkyl, C₁₋₆ haloalkyl, and C₃₋₇ cycloalkyl;

or R² and R³ together with the C atom to which they are attached form a 4- to 8-membered cycloalkyl, aryl, heteroaryl, heteroarylalkyl, or heterocycloalkyl that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R² and R³ are each independently selected from a bond, C, N, S, and O;

or R⁹ and R¹⁰ together with the N and C atoms to which they are attached form a 4- to 8-membered heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O;

or R⁹ and R¹¹ together with the N and C atoms to which they are attached form a 6- to 8-membered heterocycloalkyl or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R⁹ and R¹¹ are each independently selected from a bond, C, N, S, and O;
or R¹ and R¹¹ together with the C atom to which they are attached form a 4-, 5-, 6-7- or 8-membered cycloalkyl, heterocycloalkyl, aryl, or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R¹ and R¹¹ are each independently selected from a bond, C, N, S, and O;

or R¹ and R² together with the C atom to which they are attached form a 4-, 5-, 6-7- or 8-membered cycloalkyl, heterocycloalkyl, aryl, or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R¹ and R² are each independently selected from a bond, C, N, S, and O;

or R³ and R⁴ together with the C atom to which they are attached form a 4-, 5-, 6-7- or 8-membered cycloalkyl, heterocycloalkyl, aryl, or heteroaryl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl and R³ and R⁴ are each independently selected from a bond, C, N, S, and O;

wherein each of the O, C₁₋₆ alkyl, C₁₋₆ haloalkyl, heteroaryl, aryl, heteroaryl, heterocycloalkyl, and cycloalkyl is optionally independently substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl.

In some embodiments, the sigma-2 ligand comprises a racemic mixture or an enantiomer of compound II

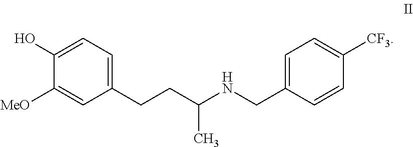 [SMILES: COc1cc(CCC(C)NCc2ccc(C(F)(F)F)cc2)ccc1O]

In some embodiments, the sigma-2 ligand is a compound or a pharmaceutically acceptable salt of Formula I, wherein R² and R³ are independently selected from OH and C₁₋₆ alkoxy.

In some embodiments, the sigma-2 ligand is a compound or a pharmaceutically acceptable salt of Formula I, wherein R² and R³ are independently selected from OH and NH(C₁₋₄ alkyl).

In some embodiments, the sigma-2 ligand is a compound or a pharmaceutically acceptable salt of Formula I, wherein R² and R³ are independently selected from H, halo, and C₁₋₆ haloalkyl.

In some embodiments, the sigma-2 ligand is a compound or a pharmaceutically acceptable salt of Formula I, wherein R² and R³ are each independently selected from OH and C₁₋₆ alkoxy and R⁷ and R⁸ are each independently C₁₋₆ alkyl. In some embodiments, R⁷ and R⁸ are each methyl.

In some embodiments, the sigma-2 ligand is a compound or a pharmaceutically acceptable salt of Formula I, wherein R⁵ and R⁶ are each independently selected from H and C₁₋₆ haloalkyl.

In some embodiments, the sigma-2 ligand is a compound or a pharmaceutically acceptable salt of Formula I, wherein R⁹ is H.

In some embodiments, the sigma-2 ligand is a compound or a pharmaceutically acceptable salt of Formula I, wherein R² and R³ or R³ and R⁴ together with the C atom to which they are attached form a 6-membered cycloalkyl, cycloheteroalkyl, aryl or heteroaryl ring. In some embodiments R² and R³ are O.

In some embodiments, the sigma-2 ligand is a compound or a pharmaceutically acceptable salt of Formula I, wherein R⁷ is C₁₋₆ alkyl and R⁸ is H.

In some embodiments, the sigma-2 ligand is a compound or a pharmaceutically acceptable salt of Formula I, wherein R⁷ is H and R⁸ is C₁₋₆ alkyl.

In some embodiments, the sigma-2 ligand is a compound or a pharmaceutically acceptable salt of Formula I, wherein R² and R³ are independently selected from H, OH, halo, C₁₋₆ alkoxy and C₁₋₆ haloalkyl.

In some embodiments, the sigma-2 ligand is a compound or a pharmaceutically acceptable salt of Formula I, wherein R² and R³ are independently selected from H, OH, Cl, F, —OMe, and —CF₃.

In some embodiments, the sigma-2 ligand is a compound or a pharmaceutically acceptable salt of Formula I, wherein R² and R³ are independently selected from H, OH, Cl, F, —OMe, and —CF₃, wherein R⁷ and R⁸ are each independently selected from H and C₁₋₆ alkyl, wherein R⁹ is H, and wherein R⁵ and R⁶ are each independently selected from H and C₁₋₆ haloalkyl.

In some embodiments, the compound of Formula I is a substantially pure (+) or (−) enantiomer of:

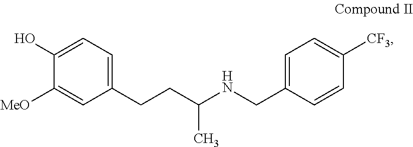 [SMILES: COc1cc(CCC(C)NCc2ccc(C(F)(F)F)cc2)ccc1O]

wherein the substantially pure enantiomer comprises at least 90, 91, 92, 93, 94, 95, 96, 97, 98, or 99% of one enantiomer of compound II. In some embodiments, a composition comprising a substantially pure enantiomer of compound II is at least 99.5% one enantiomer, and in other embodiments, the composition comprises only one enantiomer of compound II.

In one more specific embodiment, the sigma-2 ligands of the present invention are the novel compounds represented by Formula III:

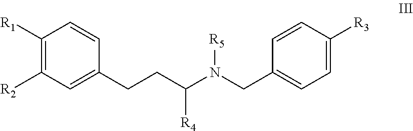

wherein

R₁ and R₂ are independently selected from H, OH, halo, CN, NO₂, NH₂, C₁₋₆ alkyl, C₁₋₆ alkoxy, C₁₋₆ haloalkyl, C₁₋₆ haloalkoxy, C₃₋₇ cycloalkyl, NH(C₁₋₄ alkyl), N(C₁₋₄ alkyl)₂, NH(C₃₋₇ cycloalkyl), NHC(O)(C₁₋₄ alkyl), (C₁₋₄ alkyl)₂N—C₁₋₄ methylene-O—, SH, S(C₁₋₆ alkyl), C(O)OH, C(O)O(C₁₋₄ alkyl), C(O) (C₁₋₄ alkyl), and C(O)NH(C₁₋₄ alkyl), or R1 and R2 are linked together to form a —O—C₁₋₄ methylene-O— group, and wherein at least one of R1 and R2 is not H;

R₃ is selected from H, OH, halo, CN, NO₂, NH₂, C₁₋₆ alkyl, C₁₋₆ alkoxy, C₁₋₆ haloalkyl, C₁₋₆ haloalkoxy, C₃₋₇ cycloalkyl, NH(C₁₋₄ alkyl), N(C₁₋₄ alkyl)₂, NH(C₃₋₇ cycloalkyl), NHC(O)(C₁₋₄alkyl), SH, S(C₁₋₆ alkyl), C(O)OH, C(O)O(C₁₋₄ alkyl), C(O)(C₁₋₄ alkyl), and C(O)NH(C₁₋₄ alkyl);

R₄ is C₁₋₆ alkyl; and

R₅ is H, C₁₋₆ alkyl, and C(O)O(C₁₋₄ alkyl), C(O)(C₁₋₄ alkyl), and C(O)(C₁₋₄haloalkyl).

In another more specific embodiment, the sigma-2 ligands of the present invention are the novel compounds represented by Formula IV:

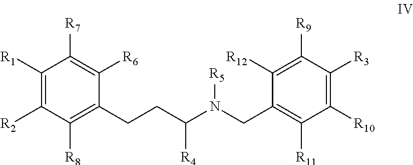

wherein

R₁, R₂, R₆, R₇ and R₈ are independently selected from H, OH, halo, CN, NO₂, NH₂, C₁₋₆ alkyl, C₁₋₆ alkoxy, C₁₋₆ haloalkyl, C₁₋₆ haloalkoxy, C₃₋₇ cycloalkyl, NH(C₁₋₄ alkyl), NH(C₁₋₄ alkyl)₂, NH(C₃₋₇ cycloalkyl), NHC(O)(C₁₋₄ alkyl), SH, S(C₁₋₆ alkyl), C(O)OH, C(O)O(C₁₋₄ alkyl), C(O) (C₁₋₄ alkyl), and C(O)NH(C₁₋₄ alkyl), or R1 and R2 are linked together to form a —O—C₁₋₄ methylene-O—, and wherein at least one of R₁, R₂, R₆, R₇ and R₈ is not H;

R₃ is selected from H, halo, and C₁₋₆ haloalkyl;
R₉, R₁₀, R₁₁, and R₁₂ are independently selected from H, C₁₋₆ alkoxy and halo.
R₄ is C₁₋₆ alkyl; and
R₅ is H, C₁₋₆ alkyl, and C(O)O(C₁₋₄ alkyl), C(O)(C₁₋₄ alkyl), C(O)(C₁₋₄haloalkyl).

In some embodiments, the sigma-2 ligands of the present invention are those of Formula Va

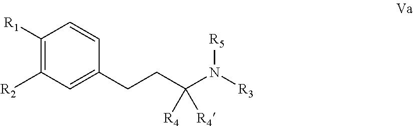

wherein

R₁ and R₂ are independently selected from H, OH, halo, C₁₋₆ alkoxy, C₁₋₆ haloalkyl, C₁₋₆ haloalkoxy, (R₁₆)(R₁₇)N—C₁₋₄ alkylene-O—, or R1 and R2 are linked together to form a —O—C₁₋₂ methylene-O— group, wherein
R₁₆ and R₁₇ are independently C₁₋₄ alkyl or benzyl, or R₁₆ and R₁₇ together with nitrogen form a ring selected from

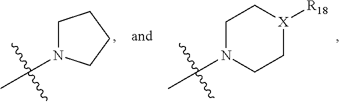

wherein
X is N or O and R₁₈ is H or unsubstituted phenyl; and

wherein at least one of R₁ and R₂ is not H;

R₃ is selected from

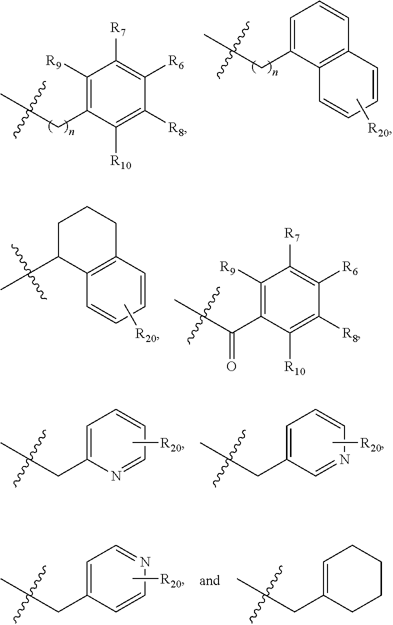

wherein
R₆, R₇, R₈, R₉, and R₁₀, are independently selected from H, halo, C₁₋₆ alkyl, C₁₋₆ alkoxy, C₁₋₆ haloalkyl, and S(O)₂—C₁₋₆ alkyl;
R₂₀ is H; and
n is 1-4

R₄ is C₁₋₆ alkyl;

R_{4′} is H or C₁₋₆ alkyl; and

R₅ is H, C₁₋₆ alkyl, and C(O)O(C₁₋₄ alkyl), C(O)(C₁₋₄ alkyl), or C(O)(C₁₋₄haloalkyl); or

R₃ and R₅ together with nitrogen form a ring selected from

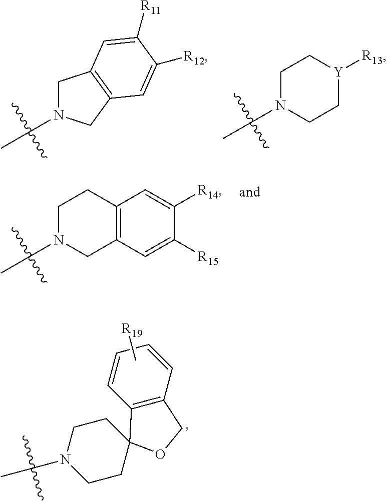

wherein
R₁₁ and R₁₂, are independently selected from H, halo, and C₁₋₆ haloalkyl, and
Y is CH or N;
R₁₃ is H, C₁₋₆ alkyl, C₃₋₆ cycloalkyl, unsubstituted phenyl or phenyl substituted with C₁₋₆ haloalkyl, or unsubstituted benzyl
R₁₄ and R₁₅ are independently selected from H and halo;
R₁₉ is H, or pharmaceutically acceptable salts thereof.

In some embodiments, the sigma-2 ligands of the present invention are those of Formula Va

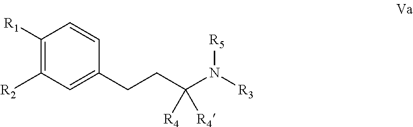

wherein

R₁ and R₂ are independently selected from H, OH, halo, C₁₋₆ alkoxy, C₁₋₆ haloalkyl, C₁₋₆ haloalkoxy, (R₁₆)(R₁₇)N—C₁₋₄ alkylene-O—, or R1 and R2 are linked together to form a —O—C₁₋₂ methylene-O— group, wherein
R₁₆ and R₁₇ are independently C₁₋₄ alkyl or benzyl, or R₁₆ and R₁₇ together with nitrogen form a ring selected from

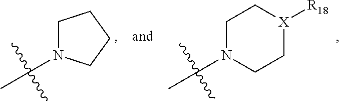

wherein
X is N or O and R₁₈ is absent or is H or unsubstituted phenyl; and

wherein at least one of R₁ and R₂ is not H;

R₃ is selected from

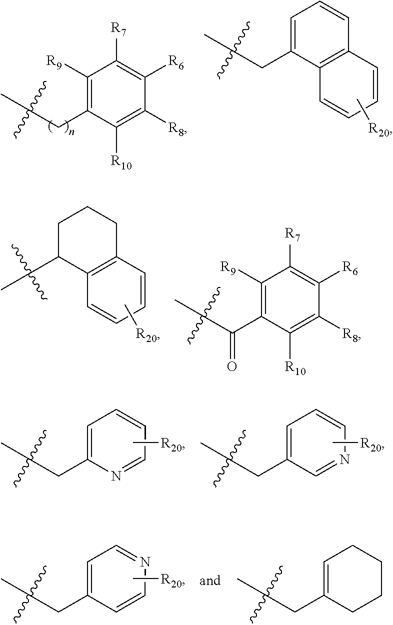

wherein
R₆, R₇, R₈, R₉, and R₁₀, are independently selected from H, halo, C₁₋₆ alkyl, C₁₋₆ alkoxy, C₁₋₆ haloalkyl, and S(O)₂—C₁₋₆ alkyl;
R₂₀ is H; and
n is 1-4

R₄ is C₁₋₆ alkyl;

R_{4′} is H or C₁₋₆ alkyl; and

R₅ is H, C₁₋₆ alkyl, and C(O)O(C₁₋₄ alkyl), C(O)(C₁₋₄ alkyl), or C(O)(C₁₋₄haloalkyl); or

R₃ and R₅ together with nitrogen form a ring selected from

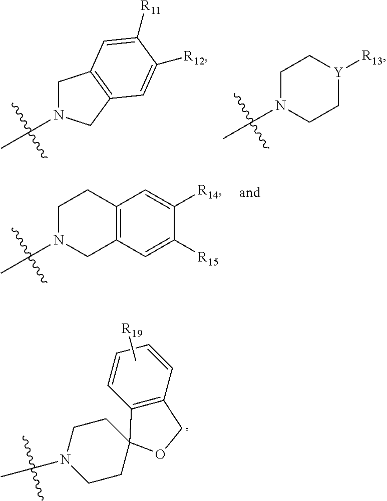

wherein
R₁₁ and R₁₂, are independently selected from H, halo, and C₁₋₆ haloalkyl, and
Y is CH or N;
R₁₃ is H, C₁₋₆ alkyl, C₃₋₆ cycloalkyl, unsubstituted phenyl or phenyl substituted with C₁₋₆ haloalkyl, or unsubstituted benzyl
R₁₄ and R₁₅ are independently selected from H and halo; and
R₁₉ is H, or pharmaceutically acceptable salts thereof.

In some embodiments, the sigma-2 ligands of the present invention are those of Formula Va

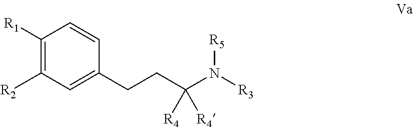

wherein

R₁ is selected from OH, OMe, F, Cl, CF₃, (R₁₆)(R₁₇)N-ethylene-O—, wherein
R₁₆ and R₁₇ are each methyl, isopropyl, n-butyl or benzyl, or R₁₆ and R₁₇ together with nitrogen form a ring selected from

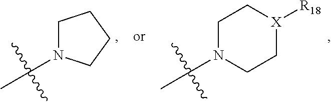

wherein
X is N or O and R₁₈ absent or is unsubstituted phenyl; and

R₂ is H, Cl, F, CF₃, OMe, OCF₃ or

R₁ and R₂ are linked together to form a —O—C₁₋₂ methylene-O— group

R₃ is selected from

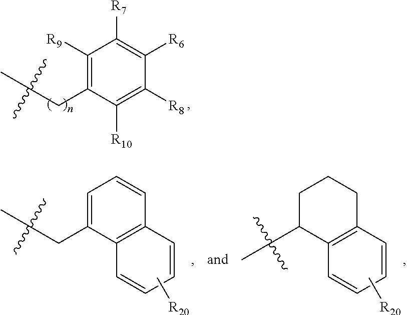

wherein
R₆ is H, F, Cl, Me, isopropyl, t-butyl, OMe, CF₃, or S(O)₂Me,
R₇ and R₈ are independently H, OMe, F, Cl, or CF₃,
R₉, and R₁₀ are independently selected from H, OMe, F, and Cl,
R₂₀ is H; and
n is 1

R₄ is Me;

R_{4′} is H or Me; and

R₅ is H; or

R₃ and R₅ together with nitrogen form a ring selected from

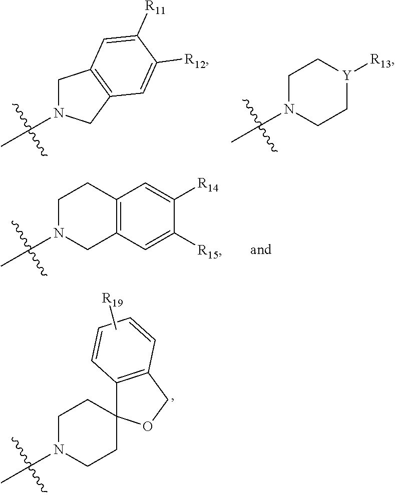

wherein
R₁₁ and R₁₂, are independently selected from H, Cl, and CF₃, and
Y is CH or N;
R₁₃ is H, Me, cyclohexyl, unsubstituted phenyl or phenyl substituted with CF₃, or unsubstituted benzyl
R₁₄ and R₁₅ are independently selected from H and Cl; and
R₁₉ is H, or pharmaceutically acceptable salts thereof.

In some embodiments, the sigma-2 ligands of the present invention are those of Formula Va

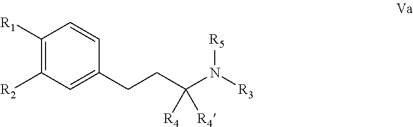

wherein

R₁ is selected from OH, OMe, F, Cl, CF₃, (R₁₆)(R₁₇)N-ethylene-O—, wherein
R₁₆ and R₁₇ are each methyl, isopropyl, n-butyl or benzyl, or R₁₆ and R₁₇ together with nitrogen form a ring selected from

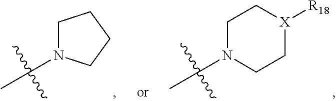

wherein
X is N or O and R₁₈ absent or is unsubstituted phenyl; and

R₂ is H, Cl, F, CF₃, OMe, OCF₃ or

R₁ and R₂ are linked together to form a —O—C₁₋₂ methylene-O— group

R₃ is selected from

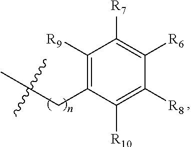

wherein
R₆ is H, F, Cl, Me, isopropyl, t-butyl, OMe, CF₃, or S(O)₂Me,
R₇ and R₈ are independently H, OMe, F, Cl, or CF₃,
R₉, and R₁₀ are independently selected from H, OMe, F, and Cl, and
n is 1

R₄ is Me;

R_{4′} is H; and

R₅ is H; or

R₃ and R₅ together with nitrogen form a ring selected from

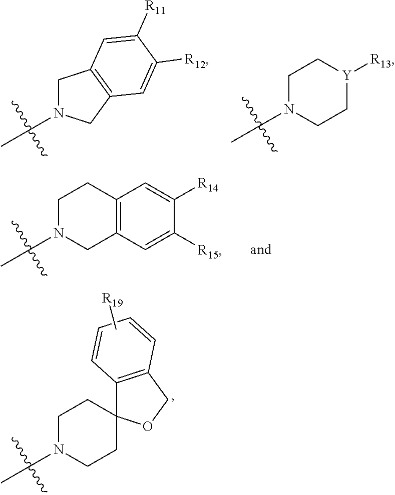

wherein
R₁₁ and R₁₂, are independently selected from H, Cl, and CF₃, and
Y is CH or N;
R₁₃ is H, Me, cyclohexyl, unsubstituted phenyl or phenyl substituted with CF₃, or unsubstituted benzyl
R₁₄ and R₁₅ are independently selected from H and Cl; and
R₁₉ is H, or pharmaceutically acceptable salts thereof.

In some more specific embodiments, the sigma-2 ligands of the present invention are those of Formula Vb

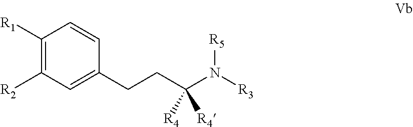

wherein R_{4′} is H and the remaining groups are as defined above for the compounds of Formula Va, or pharmaceutically acceptable salts thereof.

In some embodiments, the sigma-2 ligands of the present invention are those of Formula IIIa:

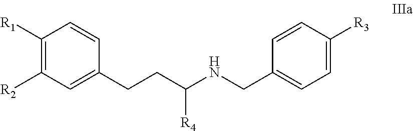

wherein
R₁=halo, C₁₋₆ haloalkyl, or OH;
R₂=H, halo or C₁₋₆ haloalkyl, or R₁ and R₂ are linked together to form a —O— methylene-O— group;
R₃=C₁₋₆ haloalkyl; and
R₄=C₁₋₆ alkyl, or pharmaceutically acceptable salts thereof.

In some more specific embodiments, the sigma-2 ligands of the present invention are those of Formula IIIa.

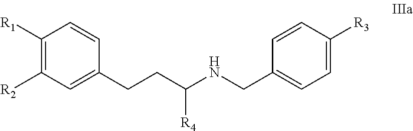

wherein
R₁=Cl, F, CF₃, or OH;
R₂=H, Cl, F, CF₃, or R₁ and R₂ are linked together to form a —O-ethylene-O— group;
R₃=CF₃; and
R₄=methyl, or pharmaceutically acceptable salts thereof.

In some more specific embodiments, the sigma-2 ligands of the present invention are those of Formula IIIb

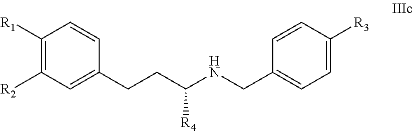

wherein R₁-R₄ are as defined above for the compounds of Formula IIIa, or pharmaceutically acceptable salts thereof.

Specific exemplary compounds of formulas III and IV and IIIa and IIIb as well as additional compounds are set forth in the following Table 1B.

TABLE 1B
Exemplary Compounds of Formulas III, IIIa, IIIb, IIIc, IV, Va, and Vb

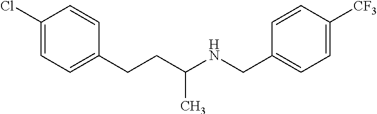 [SMILES: CC(CCc1ccc(Cl)cc1)NCc1ccc(C(F)(F)F)cc1]

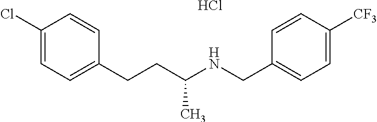 [SMILES: Cl ; C[C@H](CCc1ccc(Cl)cc1)NCc1ccc(C(F)(F)F)cc1]

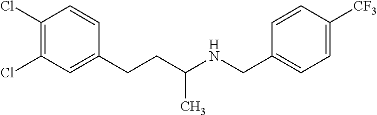 [SMILES: CC(CCc1ccc(Cl)c(Cl)c1)NCc1ccc(C(F)(F)F)cc1]

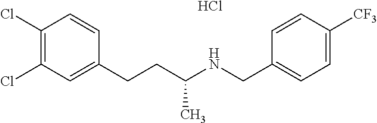 [SMILES: Cl ; C[C@H](CCc1ccc(Cl)c(Cl)c1)NCc1ccc(C(F)(F)F)cc1]

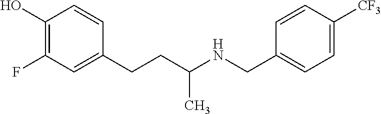 [SMILES: CC(CCc1ccc(O)c(F)c1)NCc1ccc(C(F)(F)F)cc1]

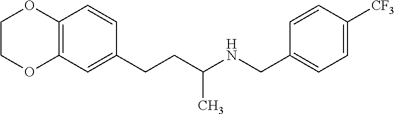 [SMILES: CC(CCc1ccc2c(c1)OCCO2)NCc1ccc(C(F)(F)F)cc1]

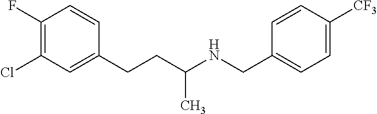 [SMILES: CC(CCc1ccc(F)c(Cl)c1)NCc1ccc(C(F)(F)F)cc1]

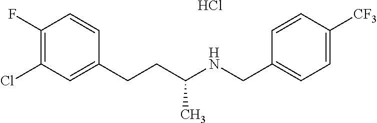 [SMILES: Cl ; C[C@H](CCc1ccc(F)c(Cl)c1)NCc1ccc(C(F)(F)F)cc1]

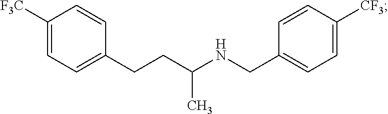 [SMILES: CC(CCc1ccc(C(F)(F)F)cc1)NCc1ccc(C(F)(F)F)cc1]

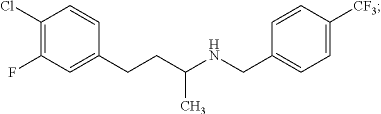 [SMILES: CC(CCc1ccc(Cl)c(F)c1)NCc1ccc(C(F)(F)F)cc1]

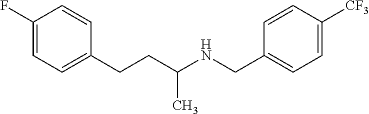 [SMILES: CC(CCc1ccc(F)cc1)NCc1ccc(C(F)(F)F)cc1]

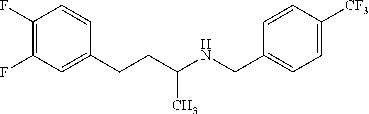 [SMILES: CC(CCc1ccc(F)c(F)c1)NCc1ccc(C(F)(F)F)cc1]

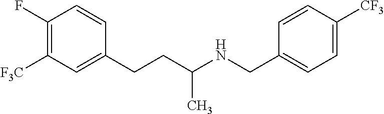 [SMILES: CC(CCc1ccc(F)c(C(F)(F)F)c1)NCc1ccc(C(F)(F)F)cc1]

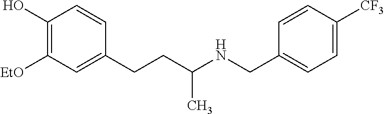 [SMILES: CCOc1cc(CCC(C)NCc2ccc(C(F)(F)F)cc2)ccc1O]

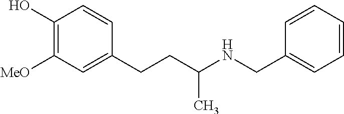 [SMILES: COc1cc(CCC(C)NCc2ccccc2)ccc1O]

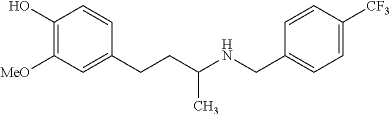 [SMILES: COc1cc(CCC(C)NCc2ccc(C(F)(F)F)cc2)ccc1O]

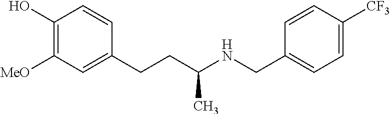 [SMILES: COc1cc(CC[C@H](C)NCc2ccc(C(F)(F)F)cc2)ccc1O]

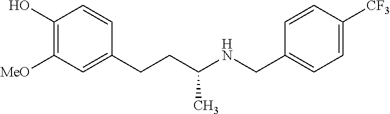 [SMILES: COc1cc(CC[C@@H](C)NCc2ccc(C(F)(F)F)cc2)ccc1O]

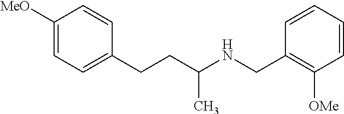 [SMILES: COc1ccc(CCC(C)NCc2ccccc2OC)cc1]

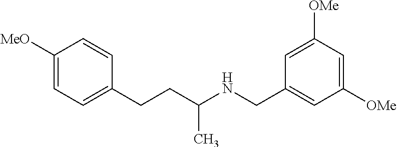 [SMILES: COc1ccc(CCC(C)NCc2cc(OC)cc(OC)c2)cc1]

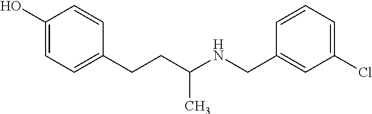 [SMILES: CC(CCc1ccc(O)cc1)NCc1cccc(Cl)c1]

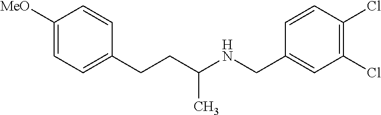 [SMILES: COc1ccc(CCC(C)NCc2ccc(Cl)c(Cl)c2)cc1]

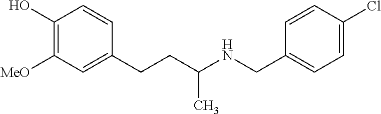 [SMILES: COc1cc(CCC(C)NCc2ccc(Cl)cc2)ccc1O]

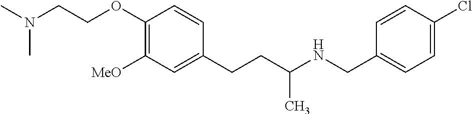 [SMILES: COc1cc(CCC(C)NCc2ccc(Cl)cc2)ccc1OCCN(C)C]

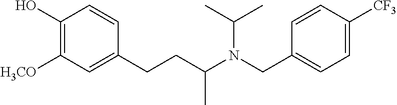 [SMILES: COc1cc(CCC(C)N(Cc2ccc(C(F)(F)F)cc2)C(C)C)ccc1O]

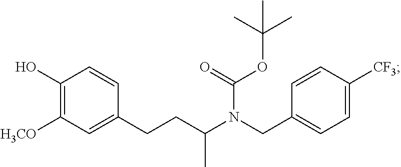 [SMILES: COc1cc(CCC(C)N(Cc2ccc(C(F)(F)F)cc2)C(=O)OC(C)(C)C)ccc1O]

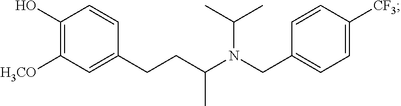 [SMILES: COc1cc(CCC(C)N(Cc2ccc(C(F)(F)F)cc2)C(C)C)ccc1O]

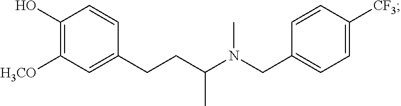 [SMILES: COc1cc(CCC(C)N(C)Cc2ccc(C(F)(F)F)cc2)ccc1O]

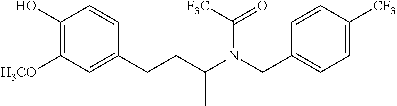 [SMILES: COc1cc(CCC(C)N(Cc2ccc(C(F)(F)F)cc2)C(=O)C(F)(F)F)ccc1O]

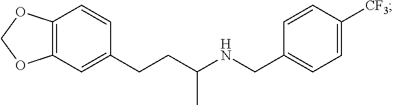 [SMILES: CC(CCc1ccc2c(c1)OCO2)NCc1ccc(C(F)(F)F)cc1]

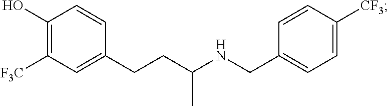 [SMILES: CC(CCc1ccc(O)c(C(F)(F)F)c1)NCc1ccc(C(F)(F)F)cc1]

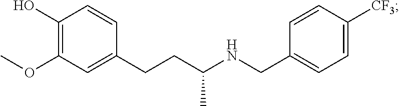 [SMILES: COc1cc(CC[C@@H](C)NCc2ccc(C(F)(F)F)cc2)ccc1O]

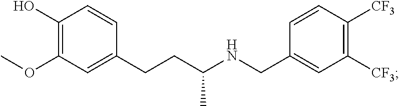 [SMILES: COc1cc(CC[C@@H](C)NCc2ccc(C(F)(F)F)c(C(F)(F)F)c2)ccc1O]

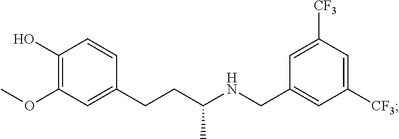 [SMILES: COc1cc(CC[C@@H](C)NCc2cc(C(F)(F)F)cc(C(F)(F)F)c2)ccc1O]

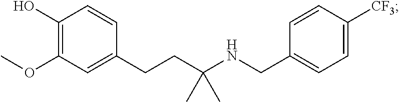 [SMILES: COc1cc(CCC(C)(C)NCc2ccc(C(F)(F)F)cc2)ccc1O]

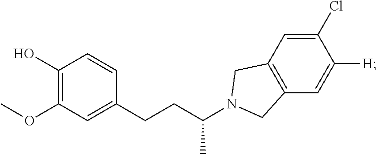

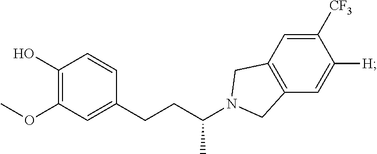

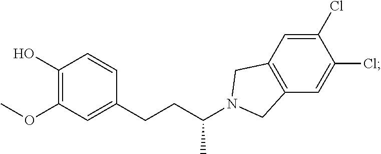 [SMILES: COc1cc(CC[C@@H](C)N2Cc3cc(Cl)c(Cl)cc3C2)ccc1O]

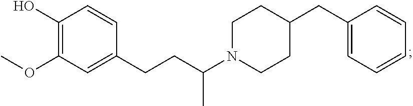 [SMILES: COc1cc(CCC(C)N2CCC(Cc3ccccc3)CC2)ccc1O]

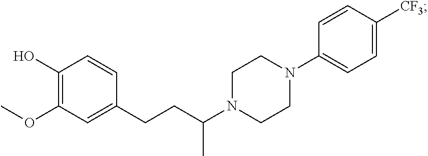 [SMILES: COc1cc(CCC(C)N2CCN(c3ccc(C(F)(F)F)cc3)CC2)ccc1O]

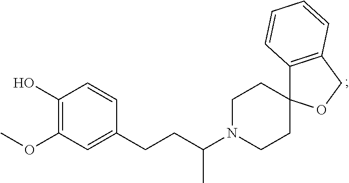 [SMILES: COc1cc(CCC(C)N2CCC3(CC2)OCc2ccccc23)ccc1O ; ";"]

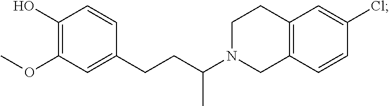 [SMILES: COc1cc(CCC(C)N2CCc3cc(Cl)ccc3C2)ccc1O]

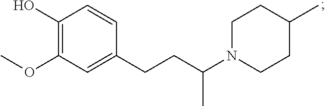

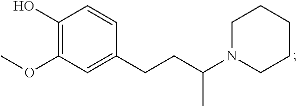 [SMILES: COc1cc(CCC(C)N2CCCCC2)ccc1O ; ";"]

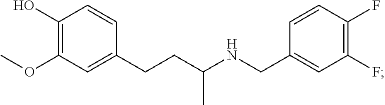 [SMILES: COc1cc(CCC(C)NCc2ccc(F)c(F)c2)ccc1O]

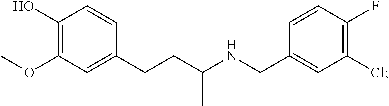 [SMILES: COc1cc(CCC(C)NCc2ccc(F)c(Cl)c2)ccc1O]

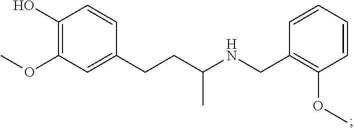

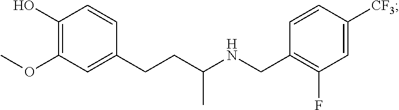 [SMILES: COc1cc(CCC(C)NCc2ccc(C(F)(F)F)cc2F)ccc1O]

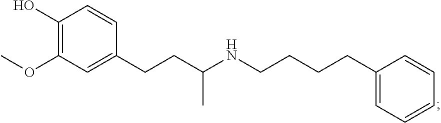 [SMILES: COc1cc(CCC(C)NCCCCc2ccccc2)ccc1O ; ";"]

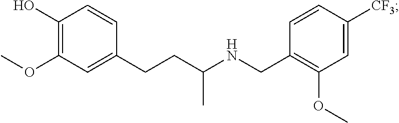 [SMILES: COc1cc(CCC(C)NCc2ccc(C(F)(F)F)cc2OC)ccc1O]

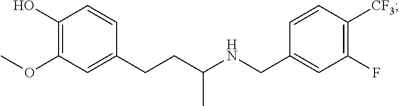 [SMILES: COc1cc(CCC(C)NCc2ccc(C(F)(F)F)c(F)c2)ccc1O]

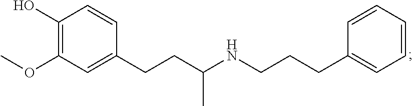 [SMILES: COc1cc(CCC(C)NCCCc2ccccc2)ccc1O ; ";"]

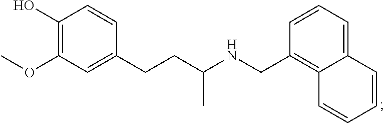 [SMILES: COc1cc(CCC(C)NCc2cccc3ccccc23)ccc1O ; ";"]

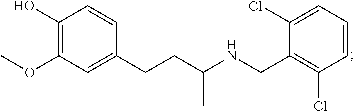 [SMILES: COc1cc(CCC(C)NCc2c(Cl)cccc2Cl)ccc1O ; ";"]

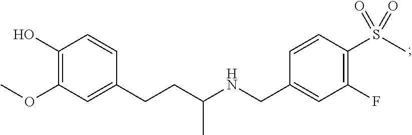

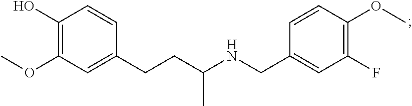

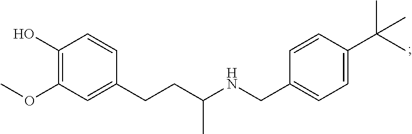 [SMILES: COc1cc(CCC(C)NCc2ccc(C(C)(C)C)cc2)ccc1O ; ";"]

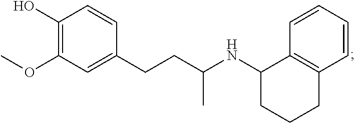 [SMILES: COc1cc(CCC(C)NC2CCCc3ccccc32)ccc1O ; ";"]

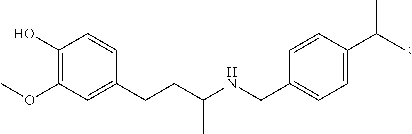 [SMILES: COc1cc(CCC(C)NCc2ccc(C(C)C)cc2)ccc1O ; ";"]

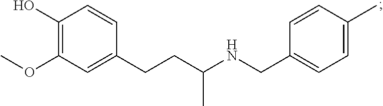

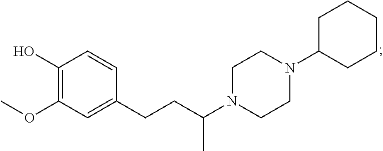 [SMILES: COc1cc(CCC(C)N2CCN(C3CCCCC3)CC2)ccc1O ; ";"]

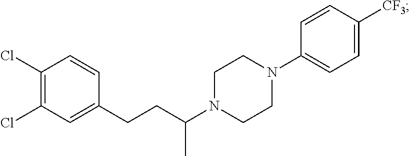 [SMILES: CC(CCc1ccc(Cl)c(Cl)c1)N1CCN(c2ccc(C(F)(F)F)cc2)CC1]

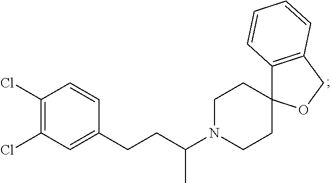 [SMILES: CC(CCc1ccc(Cl)c(Cl)c1)N1CCC2(CC1)OCc1ccccc12 ; ";"]

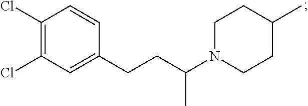 [SMILES: CC1CCN(C(C)CCc2ccc(Cl)c(Cl)c2)CC1 ; ";"]

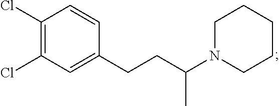 [SMILES: CC(CCc1ccc(Cl)c(Cl)c1)N1CCCCC1 ; ";"]

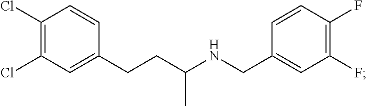 [SMILES: CC(CCc1ccc(Cl)c(Cl)c1)NCc1ccc(F)c(F)c1]

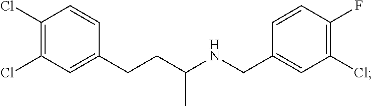 [SMILES: CC(CCc1ccc(Cl)c(Cl)c1)NCc1ccc(F)c(Cl)c1]

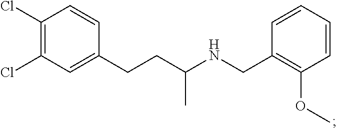 [SMILES: COc1ccccc1CNC(C)CCc1ccc(Cl)c(Cl)c1 ; ";"]

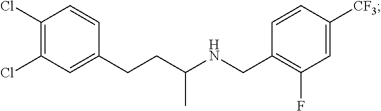 [SMILES: CC(CCc1ccc(Cl)c(Cl)c1)NCc1ccc(C(F)(F)F)cc1F]

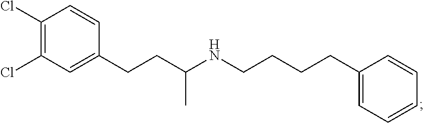 [SMILES: CC(CCc1ccc(Cl)c(Cl)c1)NCCCCc1ccccc1 ; ";"]

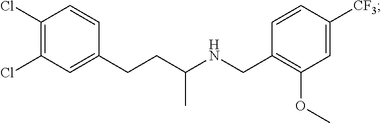 [SMILES: COc1cc(C(F)(F)F)ccc1CNC(C)CCc1ccc(Cl)c(Cl)c1]

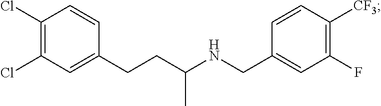 [SMILES: CC(CCc1ccc(Cl)c(Cl)c1)NCc1ccc(C(F)(F)F)c(F)c1]

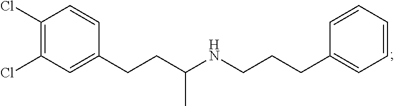 [SMILES: CC(CCc1ccc(Cl)c(Cl)c1)NCCCc1ccccc1 ; ";"]

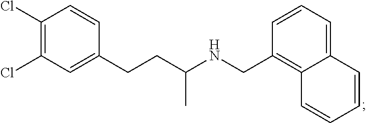 [SMILES: CC(CCc1ccc(Cl)c(Cl)c1)NCc1cccc2ccccc12 ; ";"]

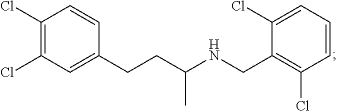 [SMILES: CC(CCc1ccc(Cl)c(Cl)c1)NCc1c(Cl)cccc1Cl ; ";"]

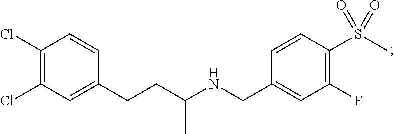 [SMILES: CC(CCc1ccc(Cl)c(Cl)c1)NCc1ccc(S(C)(=O)=O)c(F)c1 ; ";"]

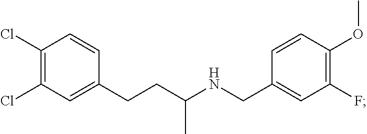 [SMILES: COc1ccc(CNC(C)CCc2ccc(Cl)c(Cl)c2)cc1F]

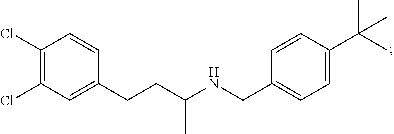 [SMILES: CC(CCc1ccc(Cl)c(Cl)c1)NCc1ccc(C(C)(C)C)cc1 ; ";"]

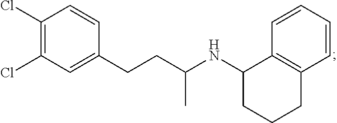 [SMILES: CC(CCc1ccc(Cl)c(Cl)c1)NC1CCCc2ccccc21 ; ";"]

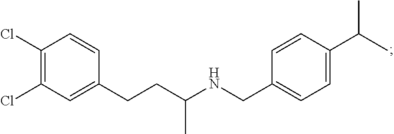 [SMILES: CC(CCc1ccc(Cl)c(Cl)c1)NCc1ccc(C(C)C)cc1 ; ";"]

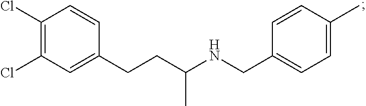 [SMILES: Cc1ccc(CNC(C)CCc2ccc(Cl)c(Cl)c2)cc1 ; ";"]

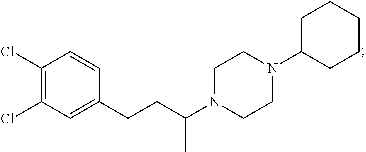 [SMILES: CC(CCc1ccc(Cl)c(Cl)c1)N1CCN(C2CCCCC2)CC1 ; ";"]

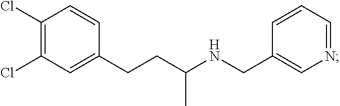 [SMILES: CC(CCc1ccc(Cl)c(Cl)c1)NCc1cccnc1]

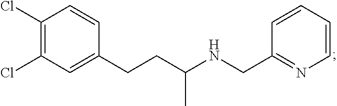 [SMILES: CC(CCc1ccc(Cl)c(Cl)c1)NCc1ccccn1 ; ";"]

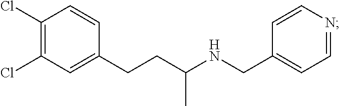 [SMILES: CC(CCc1ccc(Cl)c(Cl)c1)NCc1ccncc1]

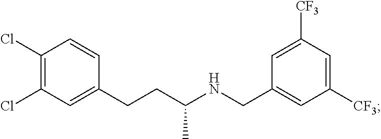 [SMILES: C[C@H](CCc1ccc(Cl)c(Cl)c1)NCc1cc(C(F)(F)F)cc(C(F)(F)F)c1]

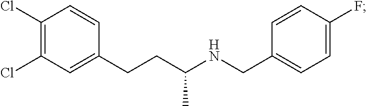 [SMILES: C[C@H](CCc1ccc(Cl)c(Cl)c1)NCc1ccc(F)cc1]

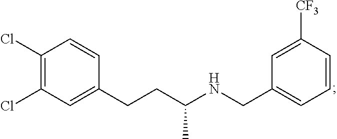 [SMILES: C[C@H](CCc1ccc(Cl)c(Cl)c1)NCc1cccc(C(F)(F)F)c1 ; ";"]

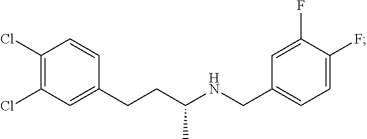 [SMILES: C[C@H](CCc1ccc(Cl)c(Cl)c1)NCc1ccc(F)c(F)c1]

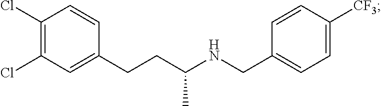 [SMILES: C[C@H](CCc1ccc(Cl)c(Cl)c1)NCc1ccc(C(F)(F)F)cc1]

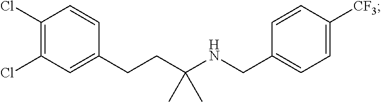 [SMILES: CC(C)(CCc1ccc(Cl)c(Cl)c1)NCc1ccc(C(F)(F)F)cc1]

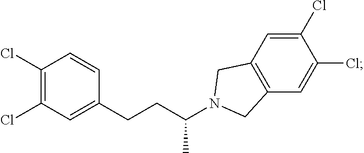 [SMILES: C[C@H](CCc1ccc(Cl)c(Cl)c1)N1Cc2cc(Cl)c(Cl)cc2C1]

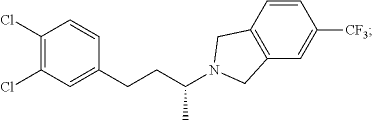 [SMILES: C[C@H](CCc1ccc(Cl)c(Cl)c1)N1Cc2ccc(C(F)(F)F)cc2C1]

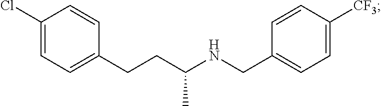 [SMILES: C[C@H](CCc1ccc(Cl)cc1)NCc1ccc(C(F)(F)F)cc1]

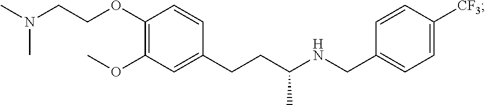 [SMILES: COc1cc(CC[C@@H](C)NCc2ccc(C(F)(F)F)cc2)ccc1OCCN(C)C]

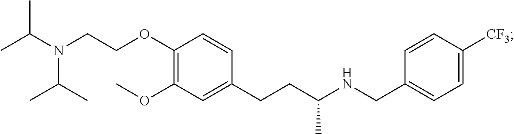 [SMILES: COc1cc(CC[C@@H](C)NCc2ccc(C(F)(F)F)cc2)ccc1OCCN(C(C)C)C(C)C]

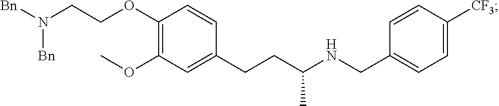 [SMILES: COc1cc(CC[C@@H](C)NCc2ccc(C(F)(F)F)cc2)ccc1OCCN(Cc1ccccc1)Cc1ccccc1]

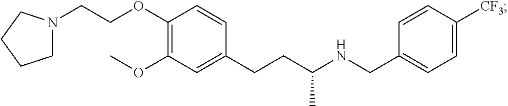 [SMILES: COc1cc(CC[C@@H](C)NCc2ccc(C(F)(F)F)cc2)ccc1OCCN1CCCC1]

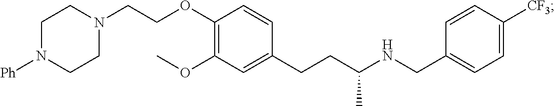 [SMILES: COc1cc(CC[C@@H](C)NCc2ccc(C(F)(F)F)cc2)ccc1OCCN1CCN(c2ccccc2)CC1]

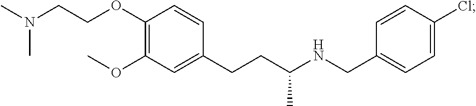 [SMILES: COc1cc(CC[C@@H](C)NCc2ccc(Cl)cc2)ccc1OCCN(C)C]

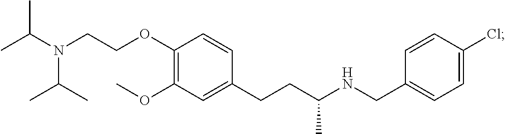 [SMILES: COc1cc(CC[C@@H](C)NCc2ccc(Cl)cc2)ccc1OCCN(C(C)C)C(C)C]

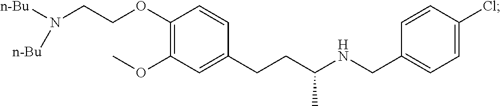

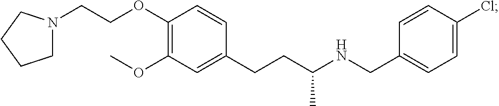 [SMILES: COc1cc(CC[C@@H](C)NCc2ccc(Cl)cc2)ccc1OCCN1CCCC1]

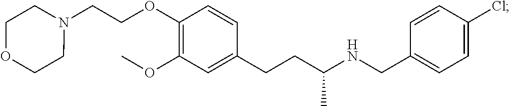 [SMILES: COc1cc(CC[C@@H](C)NCc2ccc(Cl)cc2)ccc1OCCN1CCOCC1]

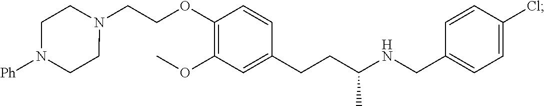 [SMILES: COc1cc(CC[C@@H](C)NCc2ccc(Cl)cc2)ccc1OCCN1CCN(c2ccccc2)CC1]

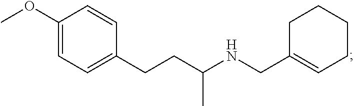 [SMILES: COc1ccc(CCC(C)NCC2=CCCCC2)cc1 ; ";"]

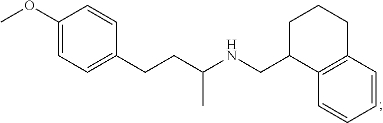 [SMILES: COc1ccc(CCC(C)NCC2CCCc3ccccc32)cc1 ; ";"]

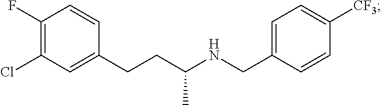 [SMILES: C[C@H](CCc1ccc(F)c(Cl)c1)NCc1ccc(C(F)(F)F)cc1]

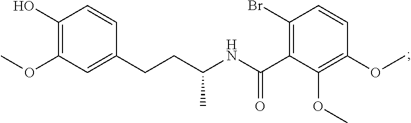

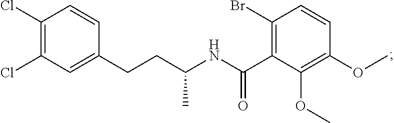 [SMILES: COc1ccc(Br)c(C(=O)N[C@H](C)CCc2ccc(Cl)c(Cl)c2)c1OC ; ";"]

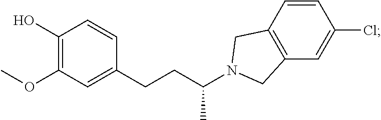 [SMILES: COc1cc(CC[C@@H](C)N2Cc3ccc(Cl)cc3C2)ccc1O]

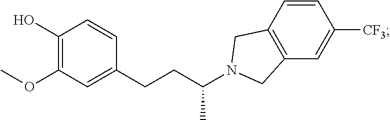 [SMILES: COc1cc(CC[C@@H](C)N2Cc3ccc(C(F)(F)F)cc3C2)ccc1O]

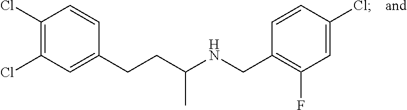 [SMILES: CC(CCc1ccc(Cl)c(Cl)c1)NCc1ccc(Cl)cc1F ; "and"]

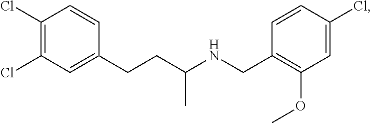 [SMILES: COc1cc(Cl)ccc1CNC(C)CCc1ccc(Cl)c(Cl)c1]

or pharmaceutically acceptable salts thereof.

Preferred salts for use in the present invention include the hydrochloride salts of the above compounds, including the following:

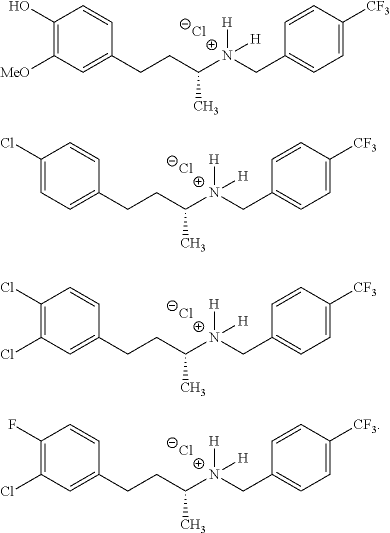

These have been synthesized in accordance with general methods provided herein and specific synthetic examples with any additional steps being well within the skill in the art. Several of these compounds have been tested in various assays as detailed herein and have been found active. Tested compounds also display increased bioavailability by reference to compounds disclosed in WO 2010/110855.

Compound II has the formula:

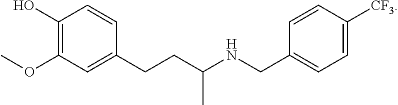 [SMILES: COc1cc(CCC(C)NCc2ccc(C(F)(F)F)cc2)ccc1O]

In some embodiments, each of the general formulae above may contain a proviso to remove the compound of Formula II.

In some embodiments, each of the general formulae above may contain a proviso to remove one or more of the following compounds:

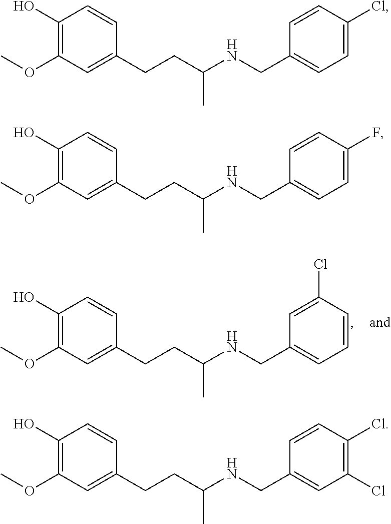

These have been synthesized in accordance with general methods provided herein and specific synthetic examples with any additional steps being well within the skill in the art. Several of these compounds have been tested in various assays as detailed herein and have been found active. Tested compounds also display increased bioavailability by reference to compounds disclosed in WO 2010/110855, incorporated herein by reference.

As used herein, the term “hydrogen bond acceptor group” refers to a group capable of accepting a hydrogen bond. Examples of hydrogen bond acceptor groups are known and include, but are not limited to, alkoxy groups, oxazolidin-2-one groups, —O—C(O)—N—; —C(O)—N—; —O—; the hetero atom (e.g. oxygen) in a cycloheteroalkyl; —N—SO₂— and the like. The groups can be bound in either direction and can be connected to another carbon or heteroatom. A hydrogen bond acceptor group can also be present in or near a hydrophobic aliphatic group. For example, a tetrahydrofuran group comprises both a hydrogen bond acceptor group and a hydrophobic aliphatic group. The oxygen present in the tetrahydrofuran ring acts as a hydrogen bond acceptor and the carbons in the tetrahydrofuran ring act as the hydrophobic aliphatic group.

As used herein, the term “hydrophobic aliphatic group” refers to a carbon chain or carbon ring. The carbon chain can be present in a cycloheteroalkyl, but the hydrophobic aliphatic group does not include the heteroatom. The tetrahydrofuran example provided above is one such example, but there are many others. In some embodiments, the hydrophobic aliphatic group is an optionally substituted C1-C6 alkyl. cycloalkyl, or C1-C6 carbons of a heterocycloalkyl. A “hydrophobic aliphatic group” is not a hydrophobic aromatic group.

As used herein, the term “positive ionizable group” refers to an atom or a group of atoms present in a structure that can be positively charged under certain conditions such as biological conditions present in solution or in a cell. In some embodiments, the positive ionizable group is a nitrogen. In some embodiments, the positive ionizable group is a nitrogen present in a cycloheteroalkyl ring. For example, in a piperazine group, the two nitrogens would be considered two positive ionizable groups. However, in some embodiments, the carbons linked to a positive ionizable group are not considered a hydrophobic aliphatic group. In some embodiments, the positive ionizable group is a nitrogen containing ring. Examples of nitrogen containing rings include, but are not limited to, piperazine, piperadine, triazinane, tetrazinane, and the like. In some embodiments with respect to the positive ionizable group, a nitrogen containing ring comprises 1, 2, 3, or 4 nitrogens. In some embodiments, the positive ionizable group is not the nitrogen present in a —N—SO₂— group

In some embodiments, a group comprises both a hydrogen bond acceptor and a positive ionizable group. For example, a morpholine group comprises both a hydrogen bond acceptor in the oxygen group and a positive ionizable group in the nitrogen.

As used herein, the term “hydrogen bond donor” refers to a group that is capable of donating a hydrogen bond. Examples of a hydrogen bond donor group include, but are not limited to, —OH, and the like.

In some embodiments, the sigma-2 receptor ligand is an optionally substituted piperazine, phenyltetrahydrofuran-N,N-dimethylmethanamine diphenyltetrahydrofuran-N,N-dimethylmethanamine, a 4-phenylpentyl-piperazine, benzylphenyl-piperazine, indole-oxa-azaspiro-decane, piperadine-indole, phenylpiperadine-indole, pyrazole-morpholine, pyrazole-piperadine, pyrazol-N,N-diethylethanamine, pyrazole-pyrrolidine, phenyl-pyrazol-morpholine, benzamide-quinoline compound, or derivatives thereof.

Additionally, the sigma-2 receptor ligand can be any compound described in WO 2011/014880 (Application No. PCT/US2010/044136), WO 2010/118055 (Application No. PCT/US2010/030130), and Application No. PCT/US2011/026530, and WO 2012/106426, each of which is hereby incorporated by reference in its entirety. For example, in some embodiments, the sigma-2 ligand is a compound of Formula V:

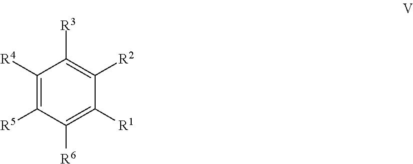

or pharmaceutically acceptable salts thereof wherein: R¹ is selected from (A1) and (A2):

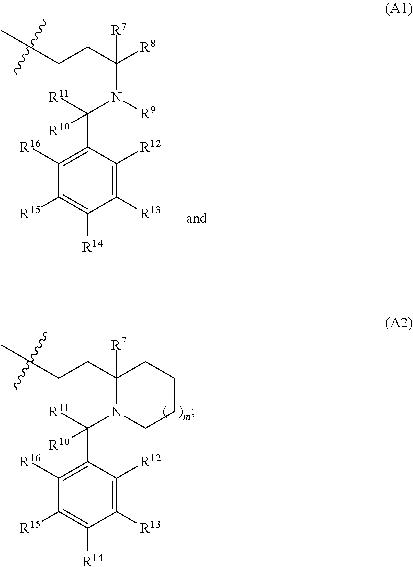

wherein in a compound of Formula V, R², R³, R⁴, R⁵, and R⁶ are each, independently, selected from H, OH, C₁₋₆ alkoxy, C₁₋₆ haloalkoxy, halo, CN, NO₂, C₁₋₆ alkyl, C₁₋₆ haloalkyl, C₃₋₇ cycloalkyl, NH₂, NH(C₁₋₄ alkyl), NH(C₃₋₇ cycloalkyl), N(C₁₋₄ alkyl)₂, NHC(O)(C₁₋₄ alkyl), SH, S(C₁₋₆ alkyl), C(O)OR^{a}, C(O)R^{b}, C(O)NR^{c}R^{d}, OC(O)R^{b}, OC(O)NR^{c}R^{d}, NR^{c}R^{d}, NR^{c}C(O)R^{b}, NR^{c}C(O)OR^{a}, NR^{c}S(O)₂R^{b}, NR^{c}S(O)₂NR^{c}R^{d}, S(O)R^{b}, S(O)₂R^{b}, and S(O)₂NR^{c}R^{d};
wherein in a compound of Formula V R⁷ is H, C₁₋₆ alkyl, C₁₋₆ haloalkyl, or C₃₋₇ cycloalkyl;
wherein in a compound of Formula V R⁸ is C₁₋₆ alkyl, C₁₋₆ haloalkyl, or C₃₋₇ cycloalkyl;
wherein in a compound of Formula V R⁹ is H, C₁₋₆ alkyl, C₁₋₆ haloalkyl, or C₃₋₇ cycloalkyl;
wherein in a compound of Formula V R¹⁰ is H, C₁₋₆ alkyl, C₁₋₆ haloalkyl, or C₃₋₇ cycloalkyl;
wherein in a compound of Formula V R¹¹ is H, C₁₋₆ alkyl, C₁₋₆ haloalkyl, or C₃₋₇ cycloalkyl;
wherein in a compound of Formula V R¹², R¹³, R¹⁴, R¹⁵, and R¹⁶ are each, independently, selected from H, OH, C₁₋₆ alkoxy, C₁₋₆ haloalkoxy, halo, CN, NO₂, C₁₋₆ alkyl, C₁₋₆ haloalkyl, C₃₋₇ cycloalkyl, NH₂, NH(C₁₋₄ alkyl), NH(C₃₋₇ cycloalkyl), N(C₁₋₄ alkyl)₂, NHC(O)(C₁₋₄ alkyl), SH, S(C₁₋₆ alkyl), C(O)OR^{a1}, C(O)R^{b1}, C(O)NR^{c1}R^{d1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}S(O)₂R^{b1}, NR^{c1}S(O)₂NR^{c1}R^{d1}, S(O)R^{b1}, S(O)₂R^{b1}, and S(O)₂NR^{c1}R^{d1};
wherein in a compound of Formula V each R^{a} is independently selected from H, C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl and heterocycloalkyl is optionally substituted with 1, 2, 3, 4, or 5 substituents independently selected from OH, CN, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
wherein in a compound of Formula V each R^{b} is independently selected from H, C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
wherein in a compound of Formula V R^{c} and R^{d} are independently selected from H, C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl and heterocycloalkyl;
or wherein in a compound of Formula V R^{c} and R^{d} together with the N atom to which they are attached form a 4-, 5-, 6- or 7-membered heterocycloalkyl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl;
wherein in a compound of Formula V each R^{a1} is independently selected from H, C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl and heterocycloalkyl is optionally substituted with 1, 2, 3, 4, or 5 substituents independently selected from OH, CN, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl;
wherein in a compound of Formula V each R^{b1} is independently selected from H, C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl;
wherein in a compound of Formula V R^{c1} and R^{d2} are independently selected from H, C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₆ alkyl, C₁₋₆ haloalkyl, arylalkyl, heteroarylalkyl, cycloalkylalkyl, heterocycloalkylalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl;
or wherein in a compound of Formula V R^{c1} and R^{d1} together with the N atom to which they are attached form a 4-, 5-, 6- or 7-membered heterocycloalkyl group that is optionally substituted with 1, 2, 3, 4, or 5 substituents independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl; and
m is 0, 1, or 2.

In some embodiments, in a compound of Formula V when R¹ is a moiety of (A1), then two of R², R³, R⁴, R⁵, and R⁶ are independently selected from OH, C₁₋₆ alkoxy, and C₁₋₆ haloalkoxy.

In some embodiments, in a compound of Formula V when R¹ is a moiety of (A1), then at least one of R¹², R¹³, R¹⁴, R¹⁵, and R¹⁶ is other than H.

In some embodiments, in a compound of Formula V two of R², R³, R⁴, R⁵, and R⁶ are independently selected from OH, C₁₋₆ alkoxy, and C₁₋₆ haloalkoxy. In some further embodiments, each of the rest of R², R³, R⁴, R⁵, and R⁶ is H.

In some embodiments, in a compound of Formula V R², R³, R⁴, R⁵, and R⁶ are each, independently, selected from H, OH, C₁₋₆ alkoxy, C₁₋₆ haloalkoxy, halo, CN, NO₂, C₁₋₆ alkyl, C₁₋₆ haloalkyl, C₃₋₇ cycloalkyl, NH₂, NH(C₁₋₄ alkyl), NH(C₃₋₇ cycloalkyl), N(C₁₋₄ alkyl)₂, NHC(O)(C₁₋₄ alkyl), SH, S(C₁₋₆ alkyl), C(O)OH, C(O)O(C₁₋₄ alkyl), C(O)(C₁₋₄ alkyl), and C(O)NH(C₁₋₄ alkyl).

In some embodiments, in a compound of Formula V one of R², R³, R⁴, R⁵, and R⁶ is OH; and one of R², R³, R⁴, R⁵, and R⁶ is OH, C₁₋₆ alkoxy, or C₁₋₆ haloalkoxy. In some further embodiments, each of the rest of R², R³, R⁴, R⁵, and R⁶ is H.

In some embodiments, in a compound of Formula V one of R², R³, R⁴, R⁵, and R⁶ is OH; and one of R², R³, R⁴, R⁵, and R⁶ is C₁₋₃ alkoxy or C₁₋₃ haloalkoxy (In some further embodiments, each of the rest of R², R³, R⁴, R⁵, and R⁶ is H). In some further embodiments, one of R², R³, R⁴, R⁵, and R⁶ is OH; and one of R², R³, R⁴, R⁵, and R⁶ is methoxy or trihalomethoxy (In some further embodiments, each of the rest of R², R³, R⁴, R⁵, and R⁶ is H.). In still further embodiments, one of R2, R³, R⁴, R⁵, and R⁶ is OH; and one of R², R³, R⁴, R⁵, and R⁶ is methoxy (In some further embodiments, each of the rest of R², R³, R⁴, R⁵, and R⁶ is H).

In some embodiments, in a compound of Formula V R⁴ is OH; and R⁵ is methoxy. In some further embodiments, R⁴ is OH; R⁵ is methoxy; and R², R³, and R⁶ are each H.

In some embodiments, in a compound of Formula V R⁷ is H or C₁₋₆ alkyl. In some further embodiments, R⁷ is H or C₁₋₃ alkyl.

In some embodiments, in a compound of Formula V R⁷ is C₁₋₃ alkyl. In some further embodiments, R⁷ is methyl or ethyl. In still further embodiments, R⁷ is methyl.

In some embodiments, in a compound of Formula V R⁷ is H.

In some embodiments, in a compound of Formula V R⁸ is C₁₋₆ alkyl. In some further embodiments, R⁸ is C₁₋₃ alkyl. In still further embodiments, R⁸ is methyl.

In some embodiments, in a compound of Formula V R⁹ is H or C₁₋₆ alkyl. In some further embodiments, R⁹ is H or C₁₋₃ alkyl.

In some embodiments, in a compound of Formula V R⁹ is H.

In some embodiments, in a compound of Formula V R⁹ is C₁₋₃ alkyl.

In some embodiments, in a compound of Formula V R¹⁰ is H or C₁₋₆ alkyl. In some further embodiments, R¹⁰ is H or C₁₋₃ alkyl. In still further embodiments, R¹⁰ is H. In other embodiments, R¹⁰ is C₁₋₃ alkyl.

In some embodiments, in a compound of Formula V R¹¹ is H or C₁₋₆ alkyl. In some further embodiments, R¹¹ is H or C₁₋₃ alkyl. In still further embodiments, R¹¹ is H. In other embodiments, R¹¹ is C₁₋₃ alkyl.

In some embodiments, in a compound of Formula V at least one of R¹², R¹³, R¹⁴, R¹⁵, and R¹⁶ is other than H.

In some embodiments, in a compound of Formula V R¹², R¹³, R¹⁴, R¹⁵, and R¹⁶ are each, independently, selected from H, OH, C₁₋₆ alkoxy, C₁₋₆ haloalkoxy, halo, CN, NO₂, C₁₋₆ alkyl, C₁₋₆ haloalkyl, C₃₋₇ cycloalkyl, NH₂, NH(C₁₋₄ alkyl), NH(C₃₋₇ cycloalkyl), N(C₁₋₄ alkyl)₂, NHC(O)(C₁₋₄ alkyl), SH, S(C₁₋₆ alkyl), C(O)OH, C(O)O(C₁₋₄ alkyl), C(O)(C₁₋₄ alkyl), and C(O)NH(C₁₋₄ alkyl).

In some embodiments, in a compound of Formula V R¹², R¹³, R¹⁴, R¹⁵, and R¹⁶ are each, independently, selected from H, halo, CN, NO₂, C₁₋₆ alkyl, C₁₋₆ haloalkyl, C₃₋₇ cycloalkyl, C(O)O(C₁₋₄ alkyl), C(O)(C₁₋₄ alkyl), and C(O)NH(C₁₋₄ alkyl).

In some embodiments, in a compound of Formula V at least one of R¹², R¹³, R¹⁴, R¹⁵, and R¹⁶ is selected from halo, CN, NO₂, C₁₋₆ haloalkyl, C(O)O(C₁₋₄ alkyl), C(O)(C₁₋₄ alkyl), and C(O)NH(C₁₋₄ alkyl).

In some embodiments, in a compound of Formula V at least one of R¹², R¹³, R¹⁴, R¹⁵, and R¹⁶ is selected from halo and C₁₋₆ haloalkyl. In some further embodiments, at least one of R¹², R¹³, R¹⁴, R¹⁵, and R¹⁶ is selected from halo and C₁₋₆ haloalkyl, and each of the rest is of R¹², R¹³, R¹⁴, R¹⁵, and R¹⁶ is H. In yet further embodiments, one or two of R¹², R¹³, R¹⁴, R¹⁵, and R¹⁶ are selected from halo and C₁₋₆ haloalkyl, and each of the rest is of R¹², R¹³, R¹⁴, R¹⁵, and R¹⁶ is H. In still further embodiments, one of R¹², R¹³, R¹⁴, R¹⁵, and R¹⁶ is selected from halo and C₁₋₆ haloalkyl, and each of the rest is of R¹², R¹³, R¹⁴, R¹⁵, and R¹⁶ is H.

In some embodiments, in a compound of Formula V R¹⁴ is halo or C₁₋₆ haloalkyl (In some further embodiments, each of R¹², R¹³, R¹⁵, and R¹⁶ is H.). In some further embodiments, R¹⁴ is halo or C₁₋₃ haloalkyl (In some further embodiments, each of R¹², R¹³, R¹⁵, and R¹⁶ is H). In still further embodiments, R¹⁴ is halo or C₁ haloalkyl (In some further embodiments, each of R¹², R¹³, R¹⁵, and R¹⁶ is H.).

In some embodiments, in a compound of Formula V R¹⁴ is halo (In some further embodiments, each of R¹², R¹³, R¹⁵, and R¹⁶ is H.). In some embodiments, R¹⁴ is Cl or F. In some embodiments, R¹⁴ is Cl. In some embodiments, R¹⁴ is F.

In some embodiments, in a compound of Formula V R¹⁴ is C₁₋₆ haloalkyl (In some further embodiments, each of R¹², R¹³, R¹⁵, and R¹⁶ is H.). In some further embodiments, R¹⁴ is C₁₋₃ haloalkyl. In still further embodiments, R¹⁴ is C₁ haloalkyl. In yet further embodiments, R¹⁴ is CF₃.

In some embodiments, in a compound of Formula V R¹⁵ is halo or C₁₋₆ haloalkyl (In some further embodiments, each of R¹², R¹³, R¹⁴, and R¹⁶ is H.). In some further embodiments, R¹⁵ is halo or C₁₋₃ haloalkyl (In some further embodiments, each of R¹², R¹³, R¹⁴, and R¹⁶ is H.). In still further embodiments, R¹⁵ is halo or C₁ haloalkyl (In some further embodiments, each of R¹², R¹³, R¹⁴, and R¹⁶ is H.).

In some embodiments, in a compound of Formula V R¹⁵ is halo. In some embodiments, R¹⁵ is Cl or F. In some embodiments, R¹⁵ is Cl. In some embodiments, R¹⁵ is F.

In some embodiments, in a compound of Formula V R¹⁵ is C₁₋₆ haloalkyl. In some further embodiments, R¹⁵ is C₁₋₃ haloalkyl. In still further embodiments, R¹⁵ is C₁ haloalkyl. In yet further embodiments, R¹⁵ is CF₃.

In some embodiments, in a compound of Formula V R¹⁴ and R¹⁵ are each independently halo or C₁₋₃ haloalkyl (In some further embodiments, each of R¹², R¹³, and R¹⁶ is H.). In some further embodiments, R¹⁴ and R¹⁵ are each independently halo or C₁ haloalkyl.

In some embodiments, in a compound of Formula V R¹⁴ and R¹⁵ are each independently halo.

In some embodiments, the compound of Formula V is a compound of Formula VI:

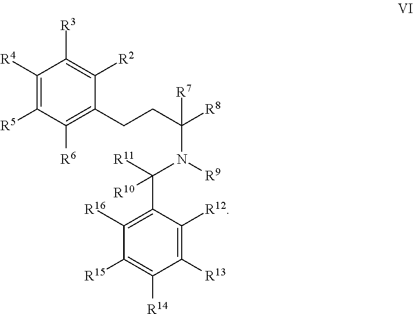

In some embodiments, the compound of Formula VI or pharmaceutically acceptable salt thereof is a compound of Formula VIa or VIb:

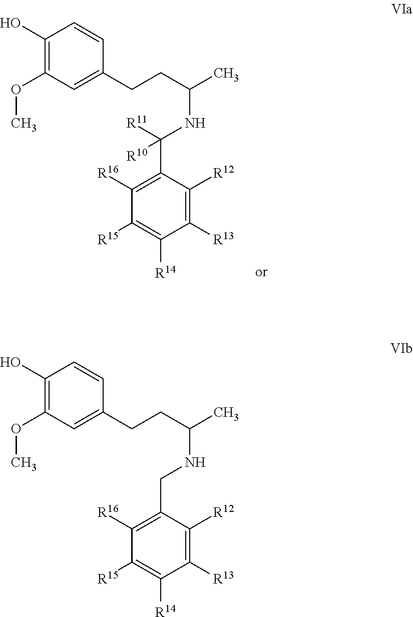

or pharmaceutically acceptable salt thereof.

In some embodiments, the compound of Formula VI is a compound of Formula VIa. In some further embodiments, R¹⁰ and R¹¹ are each, independently, selected from H and C₁₋₃ alkyl. In yet further embodiments, R¹⁰ and R¹¹ are each, independently, selected from H and methyl. In still further embodiments, R¹⁰ and R¹¹ are each H.

In some embodiments of the compound of Formula VIa or pharmaceutically acceptable salt thereof, one of R¹⁰ and R¹¹ is selected from H and C₁₋₃ alkyl and the other is H. In some further embodiments, one of R¹⁰ and R¹¹ is C₁₋₃ alkyl. In yet further embodiments, one of R¹⁰ and R¹¹ is methyl.

In some embodiments of the compound of Formula VIa or pharmaceutically acceptable salt thereof, both of R¹⁰ and R¹¹ are selected from C₁₋₃ alkyl. In some further embodiments, both R¹⁰ and R¹¹ are methyl.

In some embodiments of the compound of Formula VIa or pharmaceutically acceptable salt thereof, R¹², R¹³, R¹⁴, R¹⁵, and R¹⁶ are each, independently, selected from H, OH, C₁₋₆ alkoxy, C₁₋₆ haloalkoxy, halo, CN, NO₂, C₁₋₆ alkyl, C₁₋₆ haloalkyl, C₃₋₇ cycloalkyl, NH₂, NH(C₁₋₄ alkyl), NH(C₃₋₇ cycloalkyl), N(C₁₋₄ alkyl)₂, NHC(O)(C₁₋₄ alkyl), SH, S(C₁₋₆ alkyl), C(O)OH, C(O)O(C₁₋₄ alkyl), C(O)(C₁₋₄ alkyl), and C(O)NH(C₁₋₄ alkyl).

In some embodiments of the compound of Formula VIa or pharmaceutically acceptable salt thereof, R¹², R¹³, R¹⁴, R¹⁵, and R¹⁶ are each, independently, selected from H, halo, CN, NO₂, C₁₋₆ alkyl, C₁₋₆ haloalkyl, C₃₋₇ cycloalkyl, C(O)O(C₁₋₄ alkyl), C(O)(C1-4 alkyl), and C(O)NH(C₁₋₄ alkyl).

In some embodiments of the compound of Formula VIa or pharmaceutically acceptable salt thereof, R¹², R¹³, R¹⁴, R¹⁵, and R¹⁶ are each, independently, selected from H, halo, CN, NO₂, C₁₋₆ alkyl, C₁₋₆ haloalkyl, and C₃₋₇ cycloalkyl. In some further embodiments, R¹², R¹³, R¹⁴, R¹⁵, and R¹⁶ are each, independently, selected from H, halo, CN, C₁₋₆ alkyl, and C₁₋₆ haloalkyl. In yet further embodiments, R¹², R¹³, R¹⁴, R¹⁵, and R¹⁶ are each, independently, selected from H, halo, C₁₋₆ alkyl, and C₁₋₆ haloalkyl.

In some embodiments of the compound of Formula VIa or pharmaceutically acceptable salt thereof, at least one of R¹², R¹³, R¹⁴, R¹⁵, and R¹⁶ is selected from halo and C₁₋₆ haloalkyl, and each of the rest is of R¹², R¹³, R¹⁴, R¹⁵, and R¹⁶ is H. In some further embodiments, one or two of R¹², R¹³, R¹⁴, R¹⁵, and R¹⁶ are selected from halo and C₁₋₆ haloalkyl, and each of the rest is of R¹², R¹³, R¹⁴, R¹⁵, and R¹⁶ is H. In yet further embodiments, one of R¹², R¹³, R¹⁴, R¹⁵, and R¹⁶ is selected from halo and C₁₋₆ haloalkyl, and each of the rest is of R¹², R¹³, R¹⁴, R¹⁵ and R¹⁶ is H.

In some embodiments of the compound of Formula VIa or pharmaceutically acceptable salt thereof, at least one of R¹², R¹³, R¹⁴, R¹⁵, and R¹⁶ is selected from halo, CN, NO₂, C₁₋₆ haloalkyl, C(O)O(C₁₋₄ alkyl), C(O)(C₁₋₄ alkyl), and C(O)NH(C₁₋₄ alkyl).

In some embodiments of the compound of Formula VIa, at least one of R¹², R¹³, R¹⁴, R¹⁵, and R¹⁶ is selected from halo and C₁₋₆ haloalkyl.

In some embodiments of the compound of Formula VIa or pharmaceutically acceptable salt thereof, at least one of R¹², R¹³, R¹⁴, R¹⁵, and R¹⁶ is selected from halo and C₁₋₃ haloalkyl. In some further embodiments, at least one of R¹², R¹³, R¹⁴, R¹⁵, and R¹⁶ is selected from halo and C₁ haloalkyl.

In some embodiments of the compound of Formula VIa or pharmaceutically acceptable salt thereof, R¹⁴ is halo or C₁₋₆ haloalkyl. In some further embodiments, R¹⁴ is halo or C₁₋₃ haloalkyl. In still further embodiments, R¹⁴ is halo or C₁ haloalkyl.

In some embodiments of the compound of Formula VIa or pharmaceutically acceptable salt thereof, R¹⁴ is halo (In some further embodiments, each of R¹², R¹³, R¹⁵, and R¹⁶ is H.). In some embodiments, R¹⁴ is Cl or F. In some embodiments, R¹⁴ is Cl. In some embodiments, R¹⁴ is F.

In some embodiments of the compound of Formula IIa, R¹⁴ is C₁₋₆ haloalkyl (In some further embodiments, each of R¹², R¹³, R¹⁵, and R¹⁶ is H.). In some further embodiments, R¹⁴ is C₁₋₃ haloalkyl. In still further embodiments, R¹⁴ is C₁ haloalkyl. In yet further embodiments, R¹⁴ is CF₃.

In some embodiments of the compound of Formula VIa or pharmaceutically acceptable salt thereof, R¹⁴ is halo or C₁₋₆ haloalkyl and each of R¹², R¹³, R¹⁵, and R¹⁶ is H.

In some embodiments of the compound of Formula VIa or pharmaceutically acceptable salt thereof, R¹⁵ is halo or C₁₋₆ haloalkyl (In some further embodiments, each of R¹², R¹³, R¹⁴, and R¹⁶ is H.). In some further embodiments, R¹⁵ is halo or C₁₋₃ haloalkyl. In still further embodiments, R¹⁵ is halo or C₁ haloalkyl.

In some embodiments of the compound of Formula VIa or pharmaceutically acceptable salt thereof, R¹⁵ is halo. In some embodiments, R¹⁵ is Cl or F. In some embodiments, R¹⁵ is Cl. In some embodiments, R¹⁵ is F.

In some embodiments of the compound of Formula VIa or pharmaceutically acceptable salt thereof, R¹⁵ is C₁₋₆ haloalkyl. In some further embodiments, R¹⁵ is C₁₋₃ haloalkyl. In still further embodiments, R¹⁵ is C₁ haloalkyl. In yet further embodiments, R¹⁵ is CF₃.

In some embodiments of the compound of Formula VIa or pharmaceutically acceptable salt thereof, R¹⁴ and R¹⁵ are each independently halo or C₁₋₃ haloalkyl (In some further embodiments, each of R¹², R¹³, and R¹⁶ is H.). In some further embodiments, R¹⁴ and R¹⁵ are each independently halo or C₁ haloalkyl. In yet further embodiments, R¹⁴ and R¹⁵ are each independently halo.

In some embodiments, the compound of Formula VI or pharmaceutically acceptable salt thereof is a compound of Formula VIb or pharmaceutically acceptable salt thereof.

In some embodiments of the compound of Formula VIb or pharmaceutically acceptable salt thereof, R¹², R¹³, R¹⁴, R¹⁵, and R¹⁶ are each, independently, selected from H, OH, C₁₋₆ alkoxy, C₁₋₆ haloalkoxy, halo, CN, NO₂, C₁₋₆ alkyl, C₁₋₆ haloalkyl, C₃₋₇ cycloalkyl, NH₂, NH(C₁₋₄ alkyl), NH(C₃₋₇ cycloalkyl), N(C₁₋₄ alkyl)₂, NHC(O)(C₁₋₄ alkyl), SH, S(C₁₋₆ alkyl), C(O)OH, C(O)O(C₁₋₄ alkyl), C(O)(C₁₋₄ alkyl), and C(O)NH(C₁₋₄ alkyl).

In some embodiments of the compound of Formula VIb or pharmaceutically acceptable salt thereof, R¹², R¹³, R¹⁴, R¹⁵, and R¹⁶ are each, independently, selected from H, halo, CN, NO₂, C₁₋₆ alkyl, C₁₋₆ haloalkyl, C₃₋₇ cycloalkyl, C(O)O(C₁₋₄ alkyl), C(O)(C₁₋₄ alkyl), and C(O)NH(C₁₋₄ alkyl).

In some embodiments of the compound of Formula IIb or pharmaceutically acceptable salt thereof, R¹², R¹³, R¹⁴, R¹⁵, and R¹⁶ are each, independently, selected from H, halo, CN, NO₂, C₁₋₆ alkyl, C₁₋₆ haloalkyl, and C₃₋₇ cycloalkyl. In some further embodiments, R¹², R¹³, R¹⁴, R¹⁵, and R¹⁶ are each, independently, selected from H, halo, CN, C₁₋₆ alkyl, and C₁₋₆ haloalkyl. In yet further embodiments, R¹², R¹³, R¹⁴, R¹⁵, and R¹⁶ are each, independently, selected from H, halo, C₁₋₆ alkyl, and C₁₋₆ haloalkyl.

In some embodiments of the compound of Formula VIb or pharmaceutically acceptable salt thereof, at least one of R¹², R¹³, R¹⁴, R¹⁵, and R¹⁶ is selected from halo and C₁₋₆ haloalkyl, and each of the rest is of R¹², R¹³, R¹⁴, R¹⁵ and R¹⁶ is H. In some further embodiments, one or two of R¹², R¹³, R¹⁴, R¹⁵, and R¹⁶ are selected from halo and C₁₋₆ haloalkyl, and each of the rest is of R¹², R¹³, R¹⁴, R¹⁵, and R¹⁶ is H. In yet further embodiments, one of R¹², R¹³, R¹⁴, R¹⁵, and R¹⁶ is selected from halo and C₁₋₆ haloalkyl, and each of the rest is of R¹², R¹³, R¹⁴, R¹⁵, and R¹⁶ is H.

In some embodiments of the compound of Formula VIb or pharmaceutically acceptable salt thereof, at least one of R¹², R¹³, R¹⁴, R¹⁵, and R¹⁶ is selected from halo, CN, NO₂, C₁₋₆ haloalkyl, C(O)O(C₁₋₄ alkyl), C(O)(C₁₋₄ alkyl), and C(O)NH(C₁₋₄ alkyl).

In some embodiments of the compound of Formula VIb, at least one of R¹², R¹³, R¹⁴, R¹⁵, and R¹⁶ is selected from halo and C₁₋₆ haloalkyl.

In some embodiments of the compound of Formula VIb or pharmaceutically acceptable salt thereof, at least one of R¹², R¹³, R¹⁴, R¹⁵, and R¹⁶ is selected from halo and C₁₋₃ haloalkyl. In some further embodiments, at least one of R¹², R¹³, R¹⁴, R¹⁵, and R¹⁶ is selected from halo and C₁ haloalkyl.

In some embodiments of the compound of Formula VIb or pharmaceutically acceptable salt thereof, R¹⁴ is halo or C₁₋₆ haloalkyl. In some further embodiments, R¹⁴ is halo or C₁₋₃ haloalkyl. In still further embodiments, R¹⁴ is halo or C₁ haloalkyl.

In some embodiments of the compound of Formula VIb or pharmaceutically acceptable salt thereof, R¹⁴ is halo (In some further embodiments, each of R¹², R¹³, R¹⁵, and R¹⁶ is H.). In some embodiments, R¹⁴ is Cl or F. In some embodiments, R¹⁴ is Cl. In some embodiments, R¹⁴ is F.

In some embodiments of the compound of Formula VIb, R¹⁴ is C₁₋₆ haloalkyl (In some further embodiments, each of R¹², R¹³, R¹⁵, and R¹⁶ is H.). In some further embodiments, R¹⁴ is C₁₋₃ haloalkyl. In still further embodiments, R¹⁴ is C₁ haloalkyl. In yet further embodiments, R¹⁴ is CF₃.

In some embodiments of the compound of Formula VIb or pharmaceutically acceptable salt thereof, R¹⁴ is halo or C₁₋₆ haloalkyl and each of R¹², R¹³, R¹⁵, and R¹⁶ is H.

In some embodiments of the compound of Formula VIb or pharmaceutically acceptable salt thereof, R¹⁵ is halo or C₁₋₆ haloalkyl (In some further embodiments, each of R¹², R¹³, R¹⁴, and R¹⁶ is H.). In some further embodiments, R¹⁵ is halo or C₁₋₃ haloalkyl. In still further embodiments, R¹⁵ is halo or C₁ haloalkyl.

In some embodiments of the compound of Formula VIb or pharmaceutically acceptable salt thereof, R¹⁵ is halo. In some embodiments, R¹⁵ is Cl or F. In some embodiments, R¹⁵ is Cl. In some embodiments, R¹⁵ is F.

In some embodiments of the compound of Formula VIb or pharmaceutically acceptable salt thereof, R¹⁵ is C₁₋₆ haloalkyl. In some further embodiments, R¹⁵ is C₁₋₃ haloalkyl. In still further embodiments, R¹⁵ is C₁ haloalkyl. In yet further embodiments, R¹⁵ is CF₃.

In some embodiments of the compound of Formula VIb or pharmaceutically acceptable salt thereof, R¹⁴ and R¹⁵ are each independently halo or C₁₋₃ haloalkyl (In some further embodiments, each of R¹², R¹³, and R¹⁶ is H.). In some further embodiments, R¹⁴ and R¹⁵ are each independently halo or C₁ haloalkyl. In yet further embodiments, R¹⁴ and R¹⁵ are each independently halo.

In some embodiments, the compound of Formula V is a compound of Formula VII:

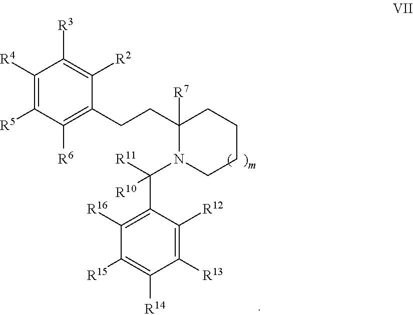

In some embodiments of compounds of Formula VII or pharmaceutically acceptable salt thereof, m is 1.

In some embodiments of compounds of Formula VII or pharmaceutically acceptable salt thereof, m is 0.

In some embodiments of compounds of Formula VII or pharmaceutically acceptable salt thereof, at least one of R², R³, R⁴, R⁵, and R⁶ is selected from OH, C₁₋₆ alkoxy, and C₁₋₆ haloalkoxy.

In some embodiments of compounds of Formula VII or pharmaceutically acceptable salt thereof, at least two of R², R³, R⁴, R⁵, and R⁶ are independently selected from OH, C₁₋₆ alkoxy, and C₁₋₆ haloalkoxy.

In some embodiments of compounds of Formula VII or pharmaceutically acceptable salt thereof, at least one of R¹², R¹³, R¹⁴, R¹⁵, and R¹⁶ is other than H.

In some embodiments of the compound of Formula VII or pharmaceutically acceptable salt thereof, R¹⁴ and R¹⁵ are each independently halo or C₁₋₃ haloalkyl. In some further embodiments, R¹⁴ and R¹⁵ are each independently halo or C₁ haloalkyl.

In some embodiments, the sigma-2 ligand is a a compound of Formula VIII:

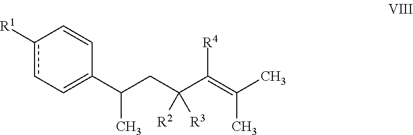

or pharmaceutically acceptable salt thereof, wherein: [symbol]
is a single bond or a double bond;
R₁ is H, CH₃, CF₃, F, Cl, Br, or —OCF₃;
R₂ is H, C₁₋₆ alkyl, C₁₋₆ haloalkyl, C₃₋₇ cycloalkyl, or C₆₋₁₀ aryl, wherein each of the C₁₋₆ alkyl, C₁₋₆ haloalkyl, C₃₋₇ cycloalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy;
R³ is OH or NR₃ₐNR_{3b};
R₃ₐ is H, C₁₋₆ alkyl, C₁₋₆ haloalkyl, cycloalkylalkyl, C₃₋₇ cycloalkyl, arylalkyl, or C₆₋₁₀ aryl, wherein each of the C₁₋₆ alkyl, C₁₋₆ haloalkyl, C₃₋₇ cycloalkyl, arylalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy;
R^{3b} is H, C₁₋₆ alkyl, C₁₋₆ haloalkyl, cycloalkylalkyl, C₃₋₇ cycloalkyl, arylalkyl, or C₆₋₁₀ aryl, wherein each of the C₁₋₆ alkyl, C₁₋₆ haloalkyl, C₃₋₇ cycloalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy;
or R^{3a} and R^{3b} together with the N atom to which they are attached form a 4-, 5-, 6- or 7-membered heterocycloalkyl group that is substituted with 0, 1, 2, 3, 4, or 5 substituents each independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl; and
R⁴ is H, C₁₋₆ alkyl, C₁₋₆ haloalkyl, C₃₋₇ cycloalkyl, or C₆₋₁₀ aryl, wherein each of the C₁₋₆ alkyl, C₁₋₆ haloalkyl, C₃₋₇ cycloalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy.

In some embodiments of compound VIII, when [symbol] is a double bond and R³ is OH, then at least one of R¹, R², and R⁴ is other than H. In some embodiments, the compound of Formula VIII is other than 2-methyl-6-p-tolylhept-2-en-4-ol.

In some embodiments, the compound of the present invention or pharmaceutically acceptable salt thereof is a compound of Formula VIIIa:

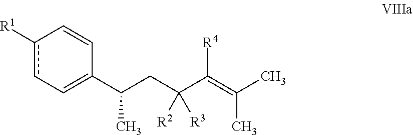

or pharmaceutically acceptable salt thereof.

In some embodiments the species of VIII are selected from one or more of the compounds:

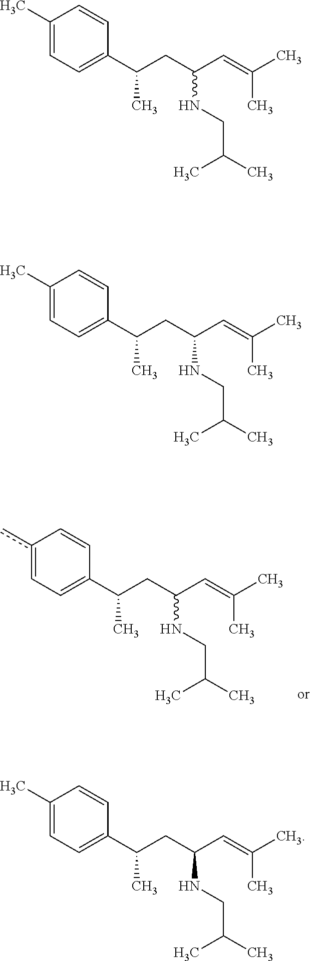

In some embodiments, the compound of Formula VIIIa is other than (6S)-2-methyl-6-p-tolylhept-2-en-4-ol.

In some embodiments, the compound of the present invention or pharmaceutically acceptable salt thereof is a compound of Formula VIIIb:

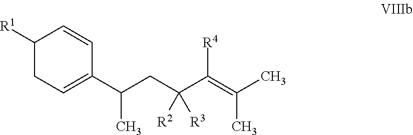

or pharmaceutically acceptable salt thereof.

In some embodiments, the sigma-2 ligand of the present invention or pharmaceutically acceptable salt thereof is a compound of Formula VIIIc:

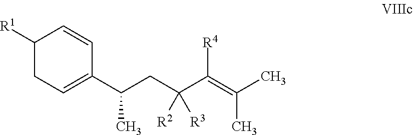

or pharmaceutically acceptable salt thereof.

In some embodiments, the sigma-2 ligand of the present invention or pharmaceutically acceptable salt thereof is a compound of Formula VIIId:

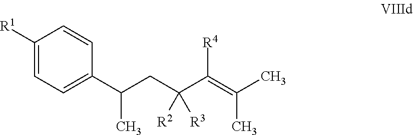

or pharmaceutically acceptable salt thereof.

In some embodiments, the compound of the present invention or pharmaceutically acceptable salt thereof is a compound of Formula VIIIe:

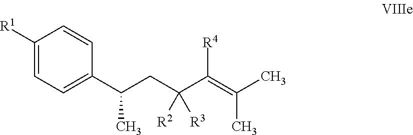

or pharmaceutically acceptable salt thereof.

In some embodiments, the sigma-2 ligand contemplated in the present invention or pharmaceutically acceptable salt thereof is a compound of Formula VIIIf:

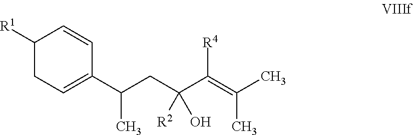

or pharmaceutically acceptable salt thereof.

In some embodiments, the compound of the present invention or pharmaceutically acceptable salt thereof is a compound of Formula VIIIg:

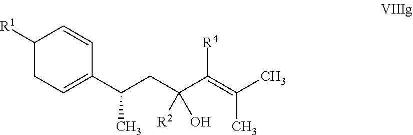

or pharmaceutically acceptable salt thereof.

In some embodiments, the sigma-2 ligand contemplated by the present invention or pharmaceutically acceptable salt thereof is a compound of Formula VIIIh:

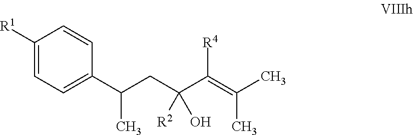

or pharmaceutically acceptable salt thereof.
In some embodiments, the compound of the present invention or pharmaceutically acceptable salt thereof is a compound of Formula VIIIi:

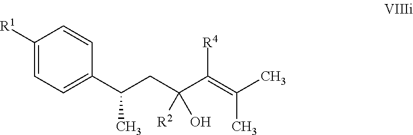

or pharmaceutically acceptable salt thereof.

In some embodiments, the compound of the present invention or pharmaceutically acceptable salt thereof is a compound of Formula VIIIj:

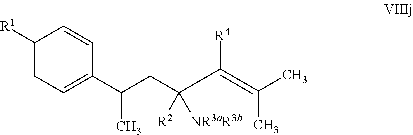

or pharmaceutically acceptable salt thereof.

In some embodiments, a sigma-2 ligand contemplated by the present invention or pharmaceutically acceptable salt thereof is a compound of Formula VIIIk:

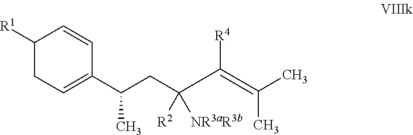

or pharmaceutically acceptable salt thereof.

In some embodiments, the compound of the present invention or pharmaceutically acceptable salt thereof is a compound of Formula VIIIm:

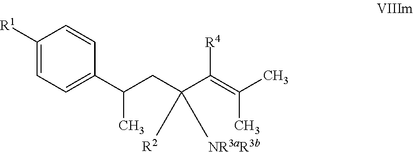

or pharmaceutically acceptable salt thereof.

In some embodiments, the compound of the present invention or pharmaceutically acceptable salt thereof is a compound of Formula VIIIn:

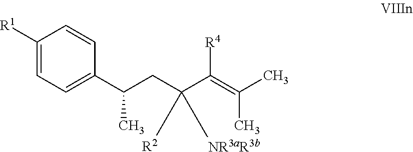

or pharmaceutically acceptable salt thereof.

In some embodiments of the compound of VIII and subset formulae thereof, R¹ is H, CH₃, or CF₃. In some further embodiments, R¹ is CH₃ or CF₃. In yet further embodiments, R¹ is CH₃.

In some embodiments of the compound of VIII and subset formulae thereof, R¹ is H or CH₃.

In some embodiments of the compound of VIII and subset formulae thereof, R¹ is F, Cl, or Br. In other embodiments, R¹ is OCF₃.

In some embodiments of the compound of VIII and subset formulae thereof, R² is H, C₁₋₆ alkyl, C₁₋₆ haloalkyl, C₃₋₇ cycloalkyl, or C₆₋₁₀ aryl. In some further embodiments, R² is H, C₁₋₆ alkyl, or C₃₋₇ cycloalkyl.

In some embodiments of the compound of VIII and subset formulae thereof, R² is H or C₁₋₆ alkyl. In some further embodiments, R² is H or methyl. In yet further embodiments, R² is H.

In some embodiments of the compound of VIII and subset formulae thereof, R¹ is H, CH₃, or CF₃; and R² is H or C₁₋₆ alkyl. In some further embodiments, R¹ is CH₃ or CF₃; and R² is H. In yet further embodiments, R¹ is CH₃; and R² is H.

In some embodiments of the compound of VIII and subset formulae thereof, R^{3a} is H, C₁₋₆ alkyl, C₁₋₆ haloalkyl, cycloalkylalkyl, C₃₋₇ cycloalkyl, arylalkyl, or C₆₋₁₀ aryl, wherein each of the C₁₋₆ alkyl, C₁₋₆ haloalkyl, cycloalkylalkyl, C₃₋₇ cycloalkyl, arylalkyl, or C₆₋₁₀ aryl is substituted by 0, 1, 2, 3, 4, or 5 substituents each independently selected from halo, C₁₋₆ alkyl, C₁₋₆ haloalkyl, C₁₋₆ alkoxy, and C₁₋₆ haloalkoxy. In some further embodiments, R^{3a} is H, C₁₋₆ alkyl, C₁₋₆ haloalkyl, cycloalkylalkyl, C₃₋₇ cycloalkyl, arylalkyl, or C₆₋₁₀ aryl.

In some embodiments of the compound of VIII and subset formulae thereof, R^{3b} is H, C₁₋₆ alkyl, C₁₋₆ haloalkyl, cycloalkylalkyl, C₃₋₇ cycloalkyl, arylalkyl, or C₆₋₁₀ aryl, wherein each of the C₁₋₆ alkyl, C₁₋₆ haloalkyl, cycloalkylalkyl, C₃₋₇ cycloalkyl, arylalkyl, or C₆₋₁₀ aryl is substituted by 0, 1, 2, 3, 4, or 5 substituents each independently selected from halo, C₁₋₆ alkyl, C₁₋₆ haloalkyl, C₁₋₆ alkoxy, and C₁₋₆ haloalkoxy. In some further embodiments, R^{3b} is H, C₁₋₆ alkyl, C₁₋₆ haloalkyl, cycloalkylalkyl, C₃₋₇ cycloalkyl, arylalkyl, or C₆₋₁₀ aryl.

In some embodiments of the compound VIII and subset formulae thereof, R^{3a} is H, C₁₋₆ alkyl, C₁₋₆ haloalkyl, C₃₋₇ cycloalkyl, or C₆₋₁₀ aryl, wherein each of the C₁₋₆ alkyl, C₁₋₆ haloalkyl, C₃₋₇ cycloalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy.

In some embodiments of the compound of VIII and subset formulae thereof, R^{3b} is H, C₁₋₆ alkyl, C₁₋₆ haloalkyl, C₃₋₇ cycloalkyl, or C₆₋₁₀ aryl, wherein each of the C₁₋₆ alkyl, C₁₋₆ haloalkyl, C₃₋₇ cycloalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy.

In some embodiments of the compound of VIII and subset formulae thereof, R^{3a} is H; and R^{3b} is H, C₁₋₆ alkyl, C₁₋₆ haloalkyl, cycloalkylalkyl, C₃₋₇ cycloalkyl, arylalkyl, or C₆₋₁₀ aryl, wherein each of the C₁₋₆ alkyl, C₁₋₆ haloalkyl, cycloalkylalkyl, C₃₋₇ cycloalkyl, arylalkyl, or C₆₋₁₀ aryl is substituted by 0, 1, 2, 3, 4, or 5 substituents each independently selected from OH, amino, halo, C₁₋₆ alkyl, C₁₋₆ haloalkyl, C₁₋₆ alkoxy, and C₁₋₆ haloalkoxy. In some further embodiments, R^{3a} is H; and R^{3b} is H, C₁₋₆ alkyl, C₁₋₆ haloalkyl, cycloalkylalkyl, C₃₋₇ cycloalkyl, arylalkyl, or C₆₋₁₀ aryl, wherein each of the C₁₋₆ alkyl, C₁₋₆ haloalkyl, cycloalkylalkyl, C₃₋₇ cycloalkyl, arylalkyl, or C₆₋₁₀ aryl is substituted by 0, 1, 2, 3, 4, or 5 substituents each independently selected from halo, C₁₋₆ alkyl, C₁₋₆ haloalkyl, C₁₋₆ alkoxy, and C₁₋₆ haloalkoxy.

In some embodiments of the compound of VIII and subset formulae thereof, R^{3a} is H; and R^{3b} is H, C₁₋₆ alkyl, C₁₋₆ haloalkyl, cycloalkylalkyl, C₃₋₇ cycloalkyl, arylalkyl, or C₆₋₁₀ aryl.

In some embodiments of the compound of VIII and subset formulae thereof, R^{3a} is H, C₁₋₆ alkyl, C₁₋₆ haloalkyl, C₃₋₇ cycloalkyl, or C₆₋₁₀ aryl; and R^{3b} is H, C₁₋₆ alkyl, C₁₋₆ haloalkyl, C₃₋₇ cycloalkyl, or C₆₋₁₀ aryl.

In some embodiments of the compound of VIII and subset formulae thereof, R^{3a} is H, C₁₋₆ alkyl, C₁₋₆ haloalkyl, C₃₋₇ cycloalkyl, or C₆₋₁₀ aryl; and R^{3b} is H, C₁₋₆ alkyl, C₁₋₆ haloalkyl, C₃₋₇ cycloalkyl, or C₆₋₁₀ aryl;

In some embodiments of the compound of VIII and subset formulae thereof, R^{3a} is H; and R^{3b} is H, C₁₋₆ alkyl, C₁₋₆ haloalkyl, or C₃₋₇ cycloalkyl.

In some embodiments of the compound of VIII and subset formulae thereof, R^{3a} is H; and R^{3b} is C₁₋₆ alkyl or C₁₋₆ haloalkyl.

In some embodiments of the compound of VIII and subset formulae thereof, R^{3a} is H; and R^{3b} is C₁₋₆ alkyl.

In some embodiments of the compound of VIII and subset formulae thereof, R^{3a} and R^{3b} together with the N atom to which they are attached form pyrrolidinyl, piperidinyl, piperazinyl, or morpholinyl, each substituted with 0, 1, 2, 3, 4, or 5 substituents each independently selected from OH, amino, halo, C₁₋₆ alkyl, C₁₋₆ haloalkyl, C₁₋₆ alkoxy, C₁₋₆ haloalkoxy, aryl, arylalkyl, heteroaryl, heteroarylalkyl, cycloalkyl, and heterocycloalkyl.

In some embodiments of the compound of VIII and subset formulae thereof, R^{3a} and R^{3b} together with the N atom to which they are attached form pyrrolidinyl, piperidinyl, piperazinyl, or morpholinyl, each substituted with 0, 1, 2, 3, 4, or 5 substituents each independently selected from OH, amino, halo, C₁₋₆ alkyl, C₁₋₆ haloalkyl, C₁₋₆ alkoxy, C₁₋₆ haloalkoxy, phenyl, and benzyl.

In some embodiments of the compound of VIII and subset formulae thereof, R⁴ is H, C₁₋₆ alkyl, C₁₋₆ haloalkyl, C₃₋₇ cycloalkyl, or C₆₋₁₀ aryl. In some further embodiments, R⁴ is H, C₁₋₆ alkyl, or C₃₋₇ cycloalkyl.

In some embodiments, R⁴ is H or C₁₋₆ alkyl. In some further embodiments, R⁴ is H or methyl. In yet further embodiments, R⁴ is H.

In some embodiments of the compound of VIII and subset formulae thereof, R² is H or C₁₋₆ alkyl, and R⁴ is H or C₁₋₆ alkyl.

In some embodiments of the compound of VIII and subset formulae thereof, R² is H or methyl, and R⁴ is H or methyl.

In some embodiments of the compound of Formula VIII and subset formulae thereof, R¹ is H, CH₃, or CF₃; R² is H or C₁₋₆ alkyl, and R⁴ is H or C₁₋₆ alkyl. In some further embodiments, R¹ is CH₃ or CF₃; R² is H; and R⁴ is H. In yet further embodiments, R¹ is CH₃; R² is H; and R⁴ is H.

In another embodiment, the sigma-2 ligands of the present invention are those of Formula VIIIo

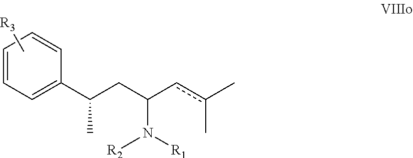

wherein: [symbol]
is a single bond or a double bond;
R₁ is C₁₋₆ alkyl, C₁₋₆ haloalkyl, unsubstituted benzyl or benzyl substituted with halo, C₁₋₆ alkyl, or C₁₋₆ haloalkyl;

### R₂ is H, or

R₁ and R₂ together with nitrogen form the ring

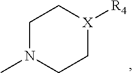

wherein

X is CH, N, or O, and

R₄ is absent, or is H, C₁₋₆ alkyl, or unsubstituted phenyl or phenyl substituted with halo, C₁₋₆ alkyl, or C₁₋₆ haloalkyl; and

R₃ is C₁₋₄ alkyl, halo, or C₁₋₆ haloalkoxy, or pharmaceutically acceptable salts thereof.

In some embodiments, the sigma-2 ligands of the present invention are those of Formula VIIIo

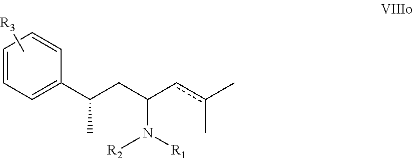

wherein: [symbol]
is a single bond or a double bond;
R₁ is isobutyl, benzyl or benzyl substituted with chloro, methyl, or CF₃;

### R₂ is H, or

R₁ and R₂ together with nitrogen form the ring

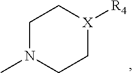

wherein

X is CH, N, or O, and

R₄ is absent, or is H, isopropyl, or unsubstituted phenyl; and

R₃ is ortho-Me, meta-Me, para-Me, para-F, or para-OCF₃, or pharmaceutically acceptable salts thereof.

In some more specific embodiments, the sigma-2 ligands of the present invention are those of Formula VIIIp

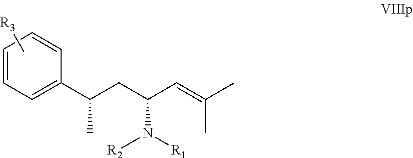

wherein R₁-R₃ are as defined above for Formula VIIIa, or pharmaceutically acceptable salts thereof.

In some more specific embodiments, the sigma-2 ligands of the present invention are those of Formula VIIIq

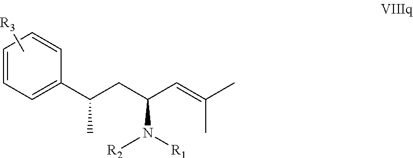

wherein R₁-R₃ are as defined above for Formula VIIIa, or pharmaceutically acceptable salts thereof.

TABLE 1C
Compounds of Formula VIIIo-q SPecific exemplary
compounds of the invention are set forth in the table below:
Compounds of Formulae VIIIo-q

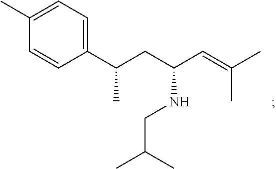 [SMILES: CC(C)=C[C@@H](C[C@H](C)c1ccc(C)cc1)NCC(C)C]

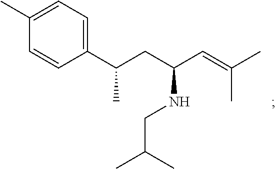 [SMILES: CC(C)=C[C@H](C[C@H](C)c1ccc(C)cc1)NCC(C)C]

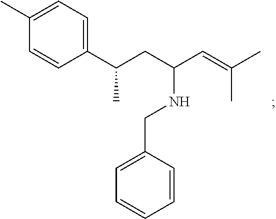 [SMILES: CC(C)=CC(C[C@H](C)c1ccc(C)cc1)NCc1ccccc1]

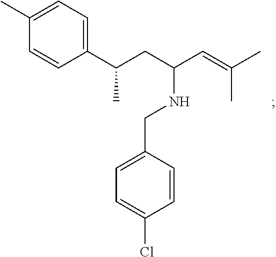 [SMILES: CC(C)=CC(C[C@H](C)c1ccc(C)cc1)NCc1ccc(Cl)cc1]

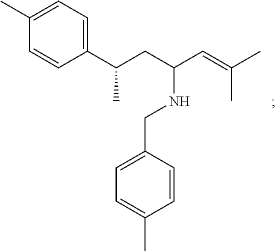 [SMILES: CC(C)=CC(C[C@H](C)c1ccc(C)cc1)NCc1ccc(C)cc1]

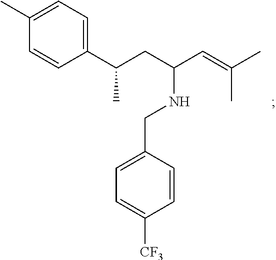 [SMILES: CC(C)=CC(C[C@H](C)c1ccc(C)cc1)NCc1ccc(C(F)(F)F)cc1]

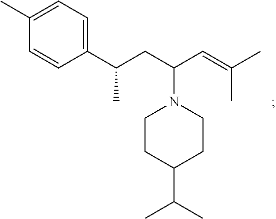 [SMILES: CC(C)=CC(C[C@H](C)c1ccc(C)cc1)N1CCC(C(C)C)CC1]

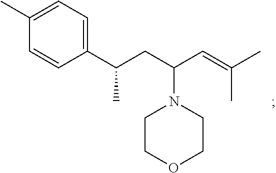 [SMILES: CC(C)=CC(C[C@H](C)c1ccc(C)cc1)N1CCOCC1]

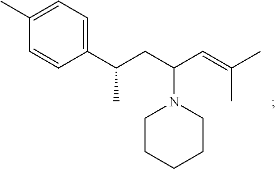 [SMILES: CC(C)=CC(C[C@H](C)c1ccc(C)cc1)N1CCCCC1]

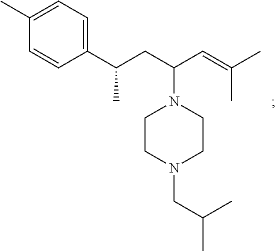 [SMILES: CC(C)=CC(C[C@H](C)c1ccc(C)cc1)N1CCN(CC(C)C)CC1]

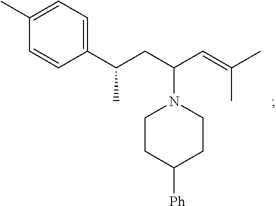 [SMILES: CC(C)=CC(C[C@H](C)c1ccc(C)cc1)N1CCC(c2ccccc2)CC1]

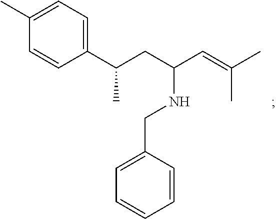 [SMILES: CC(C)=CC(C[C@H](C)c1ccc(C)cc1)NCc1ccccc1]

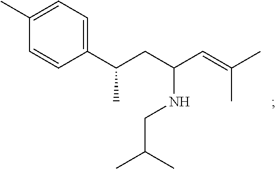 [SMILES: CC(C)=CC(C[C@H](C)c1ccc(C)cc1)NCC(C)C]

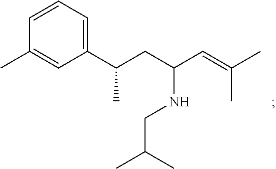 [SMILES: CC(C)=CC(C[C@H](C)c1cccc(C)c1)NCC(C)C]

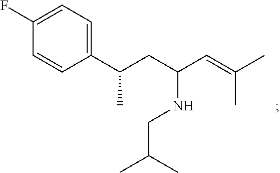 [SMILES: CC(C)=CC(C[C@H](C)c1ccc(F)cc1)NCC(C)C]

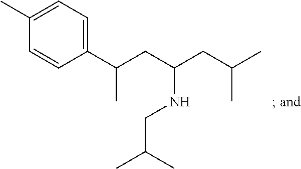 [SMILES: Cc1ccc(C(C)CC(CC(C)C)NCC(C)C)cc1]

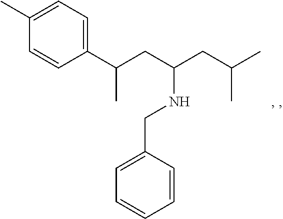 [SMILES: Cc1ccc(C(C)CC(CC(C)C)NCc2ccccc2)cc1]

or pharmaceutically acceptable salts thereof.
Preferred salts for use in the present invention include the hydrochloride salts of the above compounds, including the following:

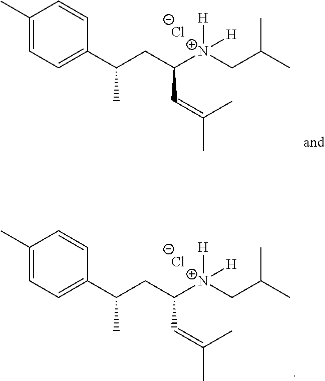

In some embodiments, each of the general formulae above may contain a proviso to remove one or more of the following compound:

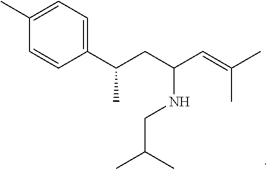

In a further embodiment, the sigma-2 ligand is a compound of Formula IX:

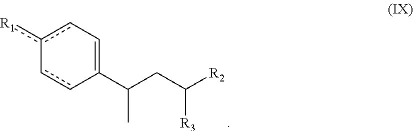

or pharmaceutically acceptable salt thereof, wherein:

[symbol] is a single bond or a double bond;

R₁ is selected from CH₃, CH₂, F, Cl, Br, CF₃, O-alkyl and OCF₃;

R₂ is selected from CH₂C(CH₃)₂OH, and CH═C(CH₃)₂;

R₃ is selected from OH, or NHCH₂CH(CH₃)₂, or mixtures thereof.

In some embodiments, the compounds of Formula IX can be prepared by reductive amination, for example, the route shown in Scheme 8.

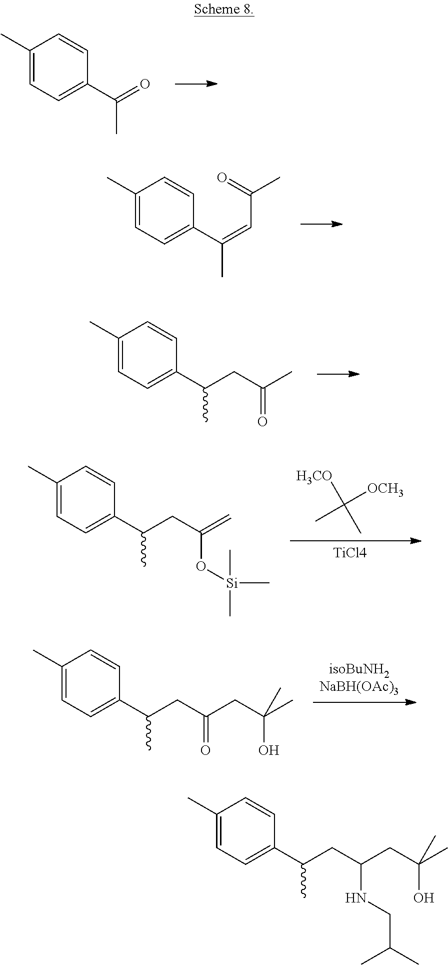

In some embodiments, the species of formula IX can be selected from:

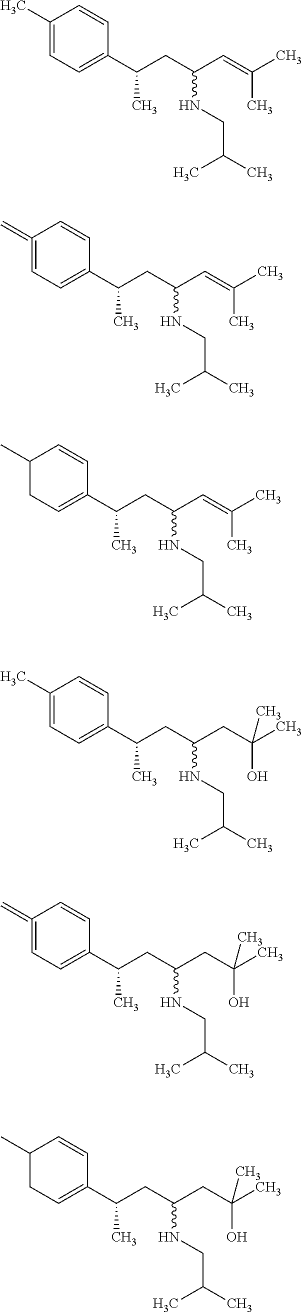

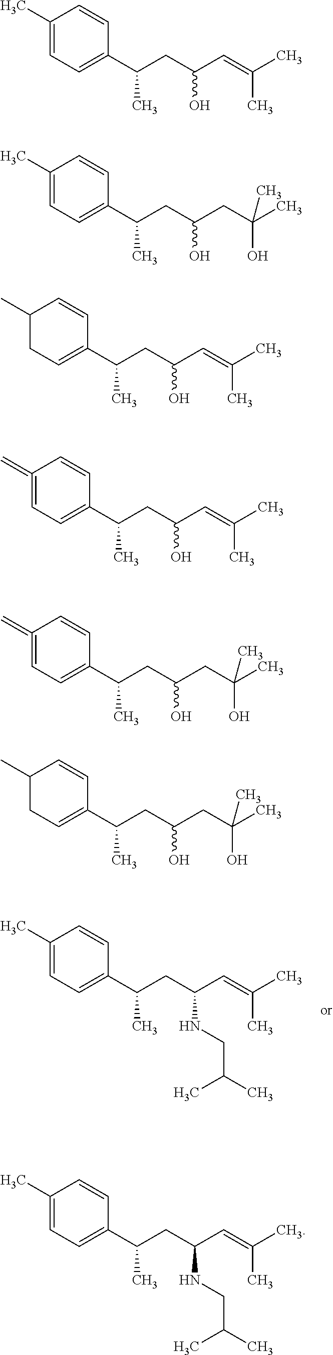

Examples of compounds of Formula IX include compounds below, which are mixtures of diastereomers, and including active aromatic amine alkenes and amino alcohol components IXa and IXb.

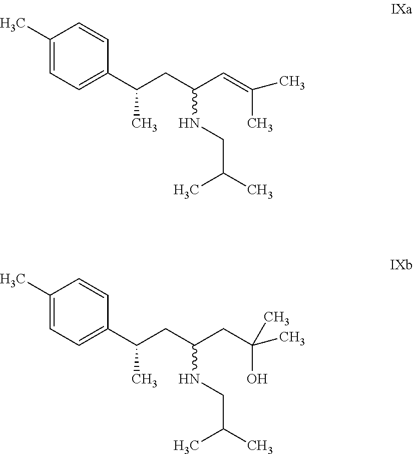

In specific embodiments, the sigma-2 receptor antagonists are selected from compounds IXa and IXb, as well as enantiomers and pharmaceutically acceptable salts.

In specific embodiments, the selective, high affinity sigma-2 receptor antagonists are selected from IXa-1 and IXa-2.

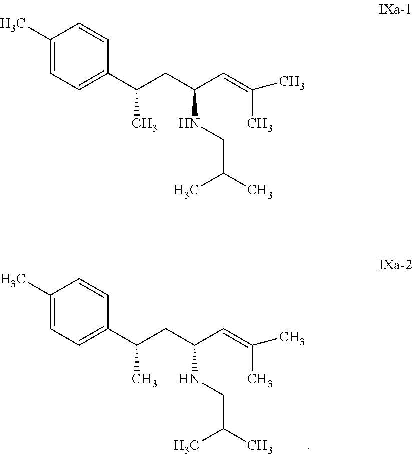

### Salts, Solvates, Stereoisomers, Derivatives, Prodrugs and Active Metabolites of the Novel Compounds of the Invention.

The present invention further encompasses salts, solvates, stereoisomers, prodrugs and active metabolites of the compounds of any of the formulae above.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

In some embodiments, the sigma-2 receptor ligand compound is selected from the compounds in the Table 1D below.

TABLE 1D
Sigma-2 Receptor Ligands Hydrochloride Salt Compounds.

| HCl Salt | Sigma-1 | Sigma-2 | MTTX |
|---|---|---|---|
| Formulation | (Ki, nM) | (Ki, nM) | (EC50, uM) |
| W | 270 | 120 | 1.2 |
| CF | 180 | 50 | 12 |
| CB | 19 | 48 | 6.5 |
| CU | 5.8 | 1.3 | 3.9 |
| DC | 330 | 3200 | 10 |
| DB | 530 | 4000 | 5 |

The acid addition salts of the compounds of any of the formulae above may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Also included are both total and partial salts, that is to say salts with 1, 2 or 3, preferably 2, equivalents of base per mole of acid of a, e.g., formula I compound or salt, with 1, 2 or 3 equivalents, preferably 1 equivalent, of acid per mole of base of a any of the formulae above compound.

For the purposes of isolation or purification it is also possible to use pharmaceutically unacceptable salts. However, only the pharmaceutically acceptable, non-toxic salts are used therapeutically and they are therefore preferred.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the invention may have both a basic and an acidic center and may therefore be in the form of zwitterions or internal salts.

Typically, a pharmaceutically acceptable salt of a compound of any of the formulae above may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of any of the formulae above and the resulting mixture evaporated to dryness (lyophilized) to obtain the acid addition salt as a solid. Alternatively, a compound of any of the formulae above may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of any of the formulae above may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.).

The present invention also encompasses N-oxides of the compounds of formulas I. The term “N-oxide” means that for heterocycles containing an otherwise unsubstituted sp² N atom, the N atom may bear a covalently bound O atom, i.e., —N→O. Examples of such N-oxide substituted heterocycles include pyridyl N-oxides, pyrimidyl N-oxides, pyrazinyl N-oxides and pyrazolyl N-oxides.

Compounds of any of the formulae above may have one or more chiral centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. A mixture containing unequal portions of the enantiomers is described as having an “enantiomeric excess” (ee) of either the R or S compound. The excess of one enantiomer in a mixture is often described with a % enantiomeric excess (% ee) value determined by the formula:

% ee=(R)−(S)/(R)+(S)

The ratio of enantiomers can also be defined by “optical purity” wherein the degree at which the mixture of enantiomers rotates plane polarized light is compared to the individual optically pure R and S compounds. Optical purity can be determined using the following formula:

Optical purity=enant.ₘₐⱼₒᵣ/(enant.ₘₐⱼₒᵣ+enant.ₘᵢₙₒᵣ)

The compounds can also be a substantially pure (+) or (−) enantiomer of the compounds described herein. In some embodiments, a composition comprising a substantially pure enantiomer comprises at least 90, 91, 92, 93, 94, 95, 96, 97, 98, or 99% of one enantiomer. In some embodiments, a composition comprising a substantially pure enantiomer is at least 99.5% one enantiomer. In some embodiments, the composition comprises only one enantiomer of a compound described herein.

The present invention encompasses all individual isomers of the compounds of any of the formulae above. The description or naming of a particular compound in the specification and claims is intended to include both individual enantiomers and mixtures, racemic or otherwise, thereof. Methods for the determination of stereochemistry and the resolution or stereotactic synthesis of stereoisomers are well-known in the art. Specifically, there is a chiral center shown in the compounds of any of the formulae above which gives rise to one set of enantiomers. Additional chiral centers may be present depending on the substituents.

For many applications, it is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions. Those of skill in the art will further recognize that invention compounds may exist in polymorphic forms wherein a compound is capable of crystallizing in different forms. Suitable methods for identifying and separating polymorphisms are known in the art.

Diastereomers differ in both physical properties and chemical reactivity. A mixture of diastereomers can be separated into enantiomeric pairs based on solubility, fractional crystallization or chromatographic properties, e.g., thin layer chromatography, column chromatography or HPLC.

Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into compositions comprising pure enantiomers. Resolution of enantiomers typically requires reaction or molecular interaction with a chiral agent, e.g., solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diastereomeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral adsorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g., column, at different rates, allowing for their separation.

Chiral resolving columns are well known in the art and are commercially available (e.g., from MetaChem Technologies Inc., a division of ANSYS Technologies, Inc., Lake Forest, Calif.). Enantiomers can be analyzed and purified using, for example, chiral stationary phases (CSPs) for HPLC. Chiral HPLC columns typically contain one form of an enantiomeric compound immobilized to the surface of a silica packing material.

D-phenylglycine and L-leucine are examples of Type I CSPs and use combinations of π-π interactions, hydrogen bonds, dipole-dipole interactions, and steric interactions to achieve chiral recognition. To be resolved on a Type I column, analyte enantiomers must contain functionality complementary to that of the CSP so that the analyte undergoes essential interactions with the CSP. The sample should preferably contain one of the following functional groups: π-acid or π-base, hydrogen bond donor and/or acceptor, or an amide dipole. Derivatization is sometimes used to add the interactive sites to those compounds lacking them. The most common derivatives involve the formation of amides from amines and carboxylic acids.

The MetaChiral ODM™ is an example of a type II CSP. The primary mechanisms for the formation of solute-CSP complexes is through attractive interactions, but inclusion complexes also play an important role. Hydrogen bonding, π-π interactions, and dipole stacking are important for chiral resolution on the MetaChiral™ ODM. Derivatization maybe necessary when the solute molecule does not contain the groups required for solute-column interactions. Derivatization, usually to benzylamides, may be required for some strongly polar molecules like amines and carboxylic acids, which would otherwise interact strongly with the stationary phase through non-specific-stereo interactions.

Where applicable, compounds of any of the formulae above can be separated into diastereomeric pairs by, for example, separation by column chromatography or TLC on silica gel. These diastereomeric pairs are referred to herein as diastereomer with upper TLC Rf; and diastereomer with lower TLC Rf. The diastereomers can further be enriched for a particular enantiomer or resolved into a single enantiomer using methods well known in the art, such as those described herein.

The relative configuration of the diastereomeric pairs can be deduced by the application of theoretical models or rules (e.g. Cram's rule, the Felkin-Ahn model) or using more reliable three-dimensional models generated by computational chemistry programs. In many instances, these methods are able to predict which diastereomer is the energetically favored product of a chemical transformation. As an alternative, the relative configuration of the diastereomeric pairs can be indirectly determined by discovering the absolute configurations of a single enantiomer in one (or both) of the diastereomeric pair(s).

The absolute configuration of the stereocenters can be determined by very well known method to those skilled in the art (e.g. X-Ray diffraction, circular dichroism). Determination of the absolute configuration can be useful also to confirm the predictability of theoretical models and can be helpful to extend the use of these models to similar molecules prepared by reactions with analogous mechanisms (e.g. ketone reductions and reductive amination of ketones by hydrides).

The present invention may also encompass stereoisomers of the Z-E type, and mixtures thereof due to R₂-R₃ substituents to the double bond not directly linked to the ring. Additional Z-E stereoisomers are encountered when m is not 1 and m and n are different. The Cahn-Ingold-Prelog priority rules are applied to determine whether the stereoisomers due to the respective position in the plane of the double bond of the doubly bonded substituents are Z or E. The stereoisomer is designated as Z (zusammen=together) if the 2 groups of highest priority lie on the same side of a reference plane passing through the C═C bond. The other stereoisomer is designated as E (entgegen=opposite).

Mixture of stereoisomers of E-Z type can be separated (and/or characterized) in their components using classical method of purification that are based on the different chemico-physical properties of these compounds. Included in these method are fractional crystallization, chromatography carried out by low, medium or high pressure techniques, fractional distillation and any other method very well known to those skilled in the art.

The present invention also encompasses prodrugs of the compounds of any of the formulae above, i.e., compounds which release an active drug according to any of the formulae above in vivo when administered to a mammalian subject. A prodrug is a pharmacologically active or more typically an inactive compound that is converted into a pharmacologically active agent by a metabolic transformation. Prodrugs of a compound of any of the formulae above are prepared by modifying functional groups present in the compound of any of the formulae above in such a way that the modifications may be cleaved in vivo to release the parent compound. In vivo, a prodrug readily undergoes chemical changes under physiological conditions (e.g., are hydrolyzed or acted on by naturally occurring enzyme(s)) resulting in liberation of the pharmacologically active agent. Prodrugs include compounds of any of the formulae above wherein a hydroxy, amino, or carboxy group is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of any of the formulae above or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

Unless specifically indicated, the term “active ingredient” is to be understood as referring to a compound of any of the formulae above as defined herein.

The present invention also encompasses metabolites. “Metabolite” of a compound disclosed herein is a derivative of a compound which is formed when the compound is metabolized. The term “active metabolite” refers to a biologically active derivative of a compound which is formed when the compound is metabolized. The term “metabolized” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17. Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

### Use of the Sigma-2 Receptor Antagonists

In some embodiments, the present invention provides methods of inhibiting synapse number decline or membrane trafficking abnormalities associated with exposure of a neuronal cell to Abeta species by administration of a sigm-2 receptor antagonist. The present invention also provides methods for treating cognitive decline and/or a neurodegenerative disease, e.g. Alzheimer's disease or mild cognitive impairment (MCI) in a patient comprising administering to the patient a sigma-2 antagonist described herein, e.g., those encompassed by any of the formulae described herein, or a pharmaceutically acceptable salt thereof. In some embodiments, the method of inhibiting, or treating, cognitive decline and/or a neurodegenerative disease, e.g. Alzheimer's disease comprises inhibiting, or treating one or more symptoms of cognitive decline selected from the group consisting of memory loss, confusion, impaired judgment, personality changes, disorientation, and loss of language skills. In some embodiments, the method comprises inhibiting, or treating, diseases or disorders or conditions mediated by or associated with Abeta oligomers (see paragraph 002). In some embodiments, the method of inhibiting, or treating, cognitive decline and/or a neurodegenerative disease, e.g. Alzheimer's disease, comprises one or more of: (i) restoration of long term potentiation (LTP), long term depression (LTD) or synaptic plasticity detectable by electrophysiological measurements or any of the other negative changes in cognitive function as mentioned in the definition of the term above; and/or (ii) inhibiting, or treating, neurodegeneration; and/or (iii) inhibiting, or treating, general amyloidosis; and/or (iv) inhibiting, or treating, one or more of amyloid production, amyloid assembly, amyloid aggregation, and amyloid oligomer binding, and amyloid deposition; and/or (v) inhibiting, treating, and/or abating an effect, notably a nonlethal effect, of one or more of Abeta oligomers on a neuron cell. In some embodiments, the method of inhibiting, treating, and/or abating cognitive decline and/or a neurodegenerative disease, e.g. Alzheimer's disease comprises inhibiting, treating, and/or abating one or more of amyloid production, amyloid assembly, the activity/effect of one or more of Abeta oligomers on a neuron cell, amyloid aggregation, amyloid binding, and amyloid deposition. In some embodiments, the method of inhibiting, treating, and/or abating cognitive decline and/or a neurodegenerative disease, e.g. Alzheimer's disease comprises inhibiting, treating, and/or abating one or more of the activity/effect of one or more of Abeta oligomers on a neuron cell.

In some embodiments, the activity/effect of one or more of Abeta oligomers on a neuron cell, amyloid aggregation and amyloid binding is the effect of Abeta oligomers on membrane trafficking or synapse number. In some embodiments, the sigma-2 antagonist inhibits the Abeta oligomer effect on membrane trafficking or synapse number or Abeta oligomer binding.

In some embodiments, the present invention provides methods of treating a proteopathic disease associated with Abeta oligomer toxicity, specifically nomlethat Abeta oligomer effects. In some embodiments, the method comprises contacting a subject with such a proteopathic disease with a sigma-2 antagonist of the present invention or a composition containing the same that binds the sigma-2 receptor.

In some embodiments, the proteopathic disease is a CNS proteopathy, characterized by an increase in Abeta protein, such as MCI, Down's Syndrome, macular degeneration or Alzheimer's disease, and the like.

In some embodiments, the present invention provides methods of treating one or more mild cognitive impairment (MCI), or dementia by administering a sigma-2 antagonist in accordance with the invention. In some embodiments, the present invention provides methods of treating MCI, and dementia.

In some embodiments, the present invention provides methods of treating an individual with a sigma-2 antagonist according to the invention to restore, partially or totally, the subject's cells to a normal phenotype in terms of functions affected adversely by Abeta species, such as Abeta oligomers. Examples are synaptic number reduction and membrane trafficking abnormalities, which can be measured by various methods including assays described herein. The normal phenotype can be, for example, normal membrane trafficking. In some embodiments, the normal phenotype is normal cognitive ability. The “normal” phenotype can be determined by comparing a subject's results with a sample of normal subjects. The sample may be as small as 1 subject or 1 sample or may be more than 10 samples or subjects and the norm is an average that is calculated based upon a plurality of subjects.

In some embodiments, the method comprises administering to a subject afflicted with cognitive decline or with a neurodegenerative disease a compound or composition that binds a sigma-2 protein and inhibits a beta-amyloid pathology. In some embodiments, the beta-amyloid pathology is a membrane trafficking defect, a decrease in synapse number, a decrease in dendritic spine number, a change in dendritic spine morphology, a change in LTP, a change in LTD, a defect in measures of memory and learning in an animal, or any combination thereof, and the like. The foregoing uses result from evidence adduced by the inventors as follows:

Sigma-2 receptor ligands within the formulae above, have been shown to be selective high affinity sigma-2 receptor ligands. For example Compound II exhibits Kᵢ 9+/−4 nM at displacement of [³H]DTG/300 nM (+)-pentazocine, at sigma-2 receptors in rat neocortex homogenate and Ki of 500+/−200 nM at displacement of [3H]-((+)-pentazocine, at sigma-1 receptors in human Jurkat cell membranes. Compound IXa,IXb exhibits Ki of 54+/−22 nM at displacement of [³H]DTG/300 nM (+)-pentazocine, at sigma-2 receptors in rat neocortex homogenate and Ki of 31+/12 nM at displacement of [3H]-((+)-pentazocine, at sigma-1 receptors in human Jurkat cell membranes. Similarly, Compound II exhibits Kᵢ 59.7+/−10.4 nM at displacement of [³H]DTG/500 nM (+)-pentazocine, at sigma-2 receptors in rat liver homogenate and Ki of 108.1+/−19.9 nM at displacement of [3H]-((+)-pentazocine, at sigma-1 receptors in guinea pig brain membranes. Compound IXa,IXb exhibits Ki of 30.8+/−2.3 nM at displacement of [³H]DTG/500 nM (+)-pentazocine, at sigma-2 receptors in rat liver homogenate and Ki of 6.37+/0.81 nM at displacement of [3H]-((+)-pentazocine, at sigma-1 receptors in guinea pig brain membranes

Sigma-2 receptor ligands within the formulae above, have been shown to act as sigma-2 receptor functional neuronal antagonists; for example, Compounds II, and IXa and IXb have been shown herein to inhibit synapse reduction associated with soluble Abeta oligomers in neuronal cells and, when added before or after Abeta oligomer introduction, to inhibit abnormalities in membrane trafficking in neuronal cells (e.g., using the MTT assay described below) attending exposure of such cells to Abeta oligomers in synthetic preparations or in preparations isolated from Alzheimer's human brains (the latter being substantially more potent in mediating amyloid pathologies in vitro). Other compounds within the formulae above have also been shown to inhibit abnormalities in membrane trafficking. Compound II, and Compounds IXa and IXb have also been shown herein to inhibit cognitive deficits exhibited in transgenic and induced animal models of Alzheimer's disease as described herein, which correlate with cognitive decline and memory loss. Compound II as well as other compounds within the Formulae above, such as Compound B, have also been shown in pharmacokinetic studies to be systemically absorbed and to cross the blood brain barrier and to be bioavailable. As a result of these properties, and given the state of the art which ascribes a strong role [see this] to Abeta oligomers and other Abeta species such as assemblies in the development of amyloid pathology, such as that of early stages of Alzheimer's disease, it is anticipated that Compound II and other compounds disclosed herein will be active in treatment of and protection against mild cognitive impairment and in the treatment (as defined herein) of Alzheimer's disease. Furthermore, because of their structural similarity to Compound II and because there has been confirmation of the foregoing in vitro activities for Compound II, pharmacokinetic properties and sigma-2 ligand status for a representative number of other compounds within Formulae I, II, III, IV, V, VI and VII among those specifically disclosed above, all the compounds within Formulae I, II, III, IV, V, VI and VII are expected to be similarly active in vivo. Likewise, because of their structural similarity to compounds IXa and IXb and because there has been confirmation of the foregoing in vitro activities for compounds IXa and IXb, all the compounds within Formule VIII and IX, and especially IX, are expected to be similarly active in vivo as well.

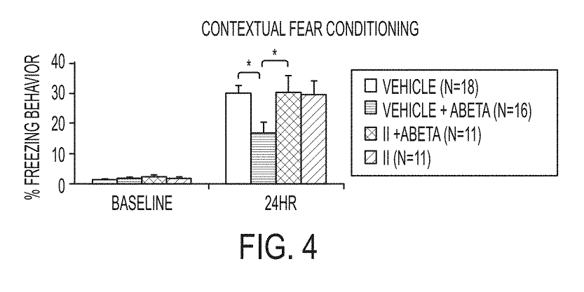

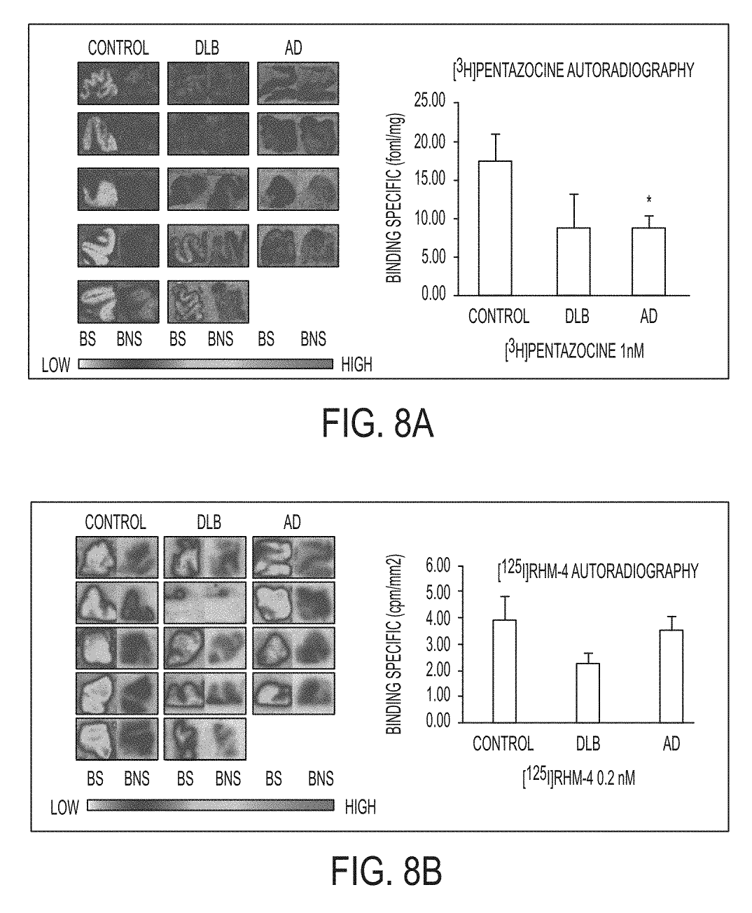

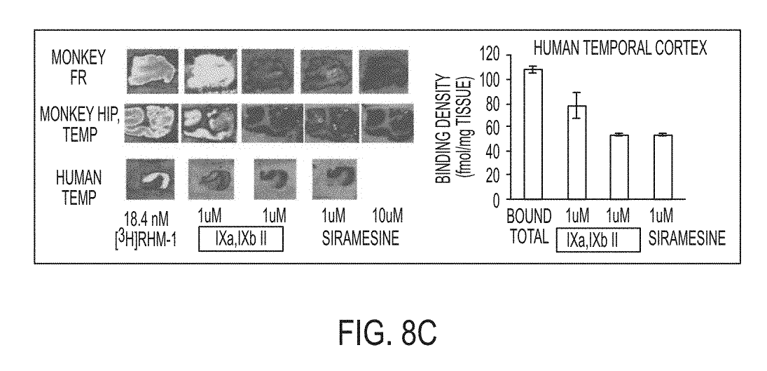

Compound II Behavioral Efficacy: Abeta oligomer-induced memory deficits in mouse fear conditioning is a model established in the laboratory of Dr. Ottavio Arancio of Columbia University (Puzzo '08). Several pharmaceutical companies use this same model in their discovery efforts. Contextual fear conditioning is an accepted model of associative memory formation which correlates to human cognitive function and specifically the creation of new memories (Delgado '06). Abeta oligomers are injected into the hippocampus of wild-type animals immediately before conditioning training and memory is assessed via freezing behavior after 24 hours. See, for example, FIGS. 4 and 8. Details are provided in Example 9. Therein, Compound II was able to completely eliminate memory deficits in the mice without inhibiting memory when dosed alone or causing any behavioral or motor toxicities. This model system was chosen because intrahippocampal administration of oligomers allows rapid comparative assessment of compound activity and off-target toxicity. The results are shown graphically in FIG. 4.

Compound II was also tested in vivo in two transgenic Alzheimer's models to show the compound's effect in reversing Abeta oligomer-associated memory loss. Specifically, compound II restored the ability of two different mutant mouse models which on aging progressively develop cognitive decline characterized by memory loss, to remember skills acquired prior to the onset of the memory loss. In addition, in the aforementioned fear conditioning assay, Compound II and Compound IXa, IXb significantly inhibited the effect of hippocampal Abeta oligomer exposure of wild-type mice, preserving the ability of the mice to acquire new memory.

These behavioral studies collectively demonstrated that Compound II causes improvement in learning and memory in two different behavioral tasks, with two different models of Alzheimer's disease, in both genders and following short or long-term administration and demonstrate that the in vitro assays correlate with in vivo activity. Despite its different structure, Compound IXa/IXb has similar activities in vitro and in vivo and is also a sigma-2 antagonist. Lastly, several sigma-2 antagonists also show activity in vitro despite different structures. Accordingly, combined, these results indicate that Compound II can be used to treat neurodegenerative diseases, such as Alzheimer's Disease. Other compounds within Formula I, II, III, IV, V, VI and VII have also been found to bind to sigma-2 receptor and to have similar in vitro activities as Compound II. Based on their similarity with Compound II they are expected to have similar activity in vitro and in vivo to Compound II. Indeed, to the extent these compounds have been tested in vitro, they have the same type of activity as Compound II and are therefore expected to have the similar activities in vivo and therefore be useful for the same therapeutic indications. A number of other sigma-2 antagonist compounds within I, II, III, IV, V, VI and VII were or will be tested in the synapse reduction and/or membrane trafficking assay described herein and are expected to be active in inhibiting Abeta oligomer-associated synapse loss and in inhibiting Abeta oligomer-associated membrane trafficking abnormalities and to be similarly active in inhibiting, e.g. cognitive decline and treat Alzheimer's disease. Likewise, because compounds IXa and IXb were also shown to be active in in vitro and in vivo models, the compounds of Formula VIII and IX, and especially IX, which are structurally similar are also generally expected to have activity both in vitro and in vivo and, therefore, useful in the treatment methods of the present invention. These compounds, to the extent they have been tested are sigma-2 ligands and the remainder are expected to be sigma-2 ligands.

As discussed herein, evidence suggests that Abeta oligomer-mediated reduction in neuronal surface receptor expression mediated by membrane trafficking are the basis for oligomer inhibition of electrophysiological measures of synaptic plasticity (LTP) and thus learning and memory (See Kamenetz F, Tomita T, Hsieh H, Seabrook G, Borchelt D, Iwatsubo T, Sisodia S, Malinow R. APP processing and synaptic function. Neuron. 2003 Mar. 27; 37(6):925-37; and Hsieh H, Boehm J, Sato C, Iwatsubo T, Tomita T, Sisodia S, Malinow R. AMPAR removal underlies Abeta oligomer-induced synaptic depression and dendritic spine loss. Neuron. 2006 Dec. 7; 52(5):831-43). Measuring membrane trafficking rate changes induced by oligomers via formazan morphological shifts has been used in cell lines to discover Abeta oligomer-blocking drugs [Maezawa I, Hong H S, Wu H C, Battina S K, Rana S, Iwamoto T, Radke G A, Pettersson E, Martin G M, Hua D H, Jin L W. A novel tricyclic pyrone compound ameliorates cell death associated with intracellular amyloid-beta oligomeric complexes. J Neurochem. 2006 July; 98(1):57-67; Liu Y, Schubert D. Cytotoxic amyloid peptides inhibit cellular 3-(4,5-dimethylthiazol-2-yl)-2,5-diphenyltetrazolium bromide (MTT) reduction by enhancing MTT formazan exocytosis. J Neurochem. 1997 December; 69(6):2285-93; Liu Y, Dargusch R, Banh C, Miller C A, Schubert D. Detecting bioactive amyloid beta peptide species in Alzheimer's disease. J Neurochem. 2004 November; 91(3):648-56; Liu Y, Schubert D. Treating Alzheimer's disease by inactivating bioactive amyloid beta peptide. Curr Alzheimer Res. 2006 April; 3(2):129-35; Rana S, Hong H S, Barrigan L, Jin L W, Hua D H. Syntheses of tricyclic pyrones and pyridinones and protection of Abeta-peptide induced MC65 neuronal cell death. Bioorg Med Chem Lett. 2009 Feb. 1; 19(3):670-4. Epub 2008 Dec. 24; and Hong H S, Maezawa I, Budamagunta M, Rana S, Shi A, Vassar R, Liu R, Lam K S, Cheng R H, Hua D H, Voss J C, Jin L W. Candidate anti-Abeta fluorene compounds selected from analogs of amyloid imaging agents. Neurobiol Aging. 2008 Nov. 18. (Epub ahead of print)] that lower Abeta brain levels in rodents in vivo [Hong H S, Rana S, Barrigan L, Shi A, Zhang Y, Zhou F, Jin L W, Hua DH. Inhibition of Alzheimer's amyloid toxicity with a tricyclic pyrone molecule in vitro and in vivo. J Neurochem. 2009 February; 108(4):1097-1108]. Accordingly, the foregoing tests have established relevance in identifying compounds to treat early Alzheimer's disease and mild cognitive impairment.

In some embodiments, a compound of any of the formulae above has an IC₅₀ value of less than 100 μM, 50 μM, 20 μM, 15 μM, 10 μM, 5 μM, 1 μM, 500 nM, 100 nM, 50 nM, or 10 nM with respect to inhibition of one or more of the effect of Abeta oligomers on neurons (such as neurons in the brain), amyloid assembly or disruption thereof, and amyloid (including amyloid oligomer) binding, and amyloid deposition. In some embodiments, the compound has an IC₅₀ value of less than 100 μM, 50 μM, 20 μM, 15 μM, 10 μM, 5 μM, 1 μM, 500 nM, 100 nM, 50 nM, or 10 nM with respect to inhibition of the activity/effect of Abeta species such as oligomers on neurons (such as central nervous system neurons).

In some embodiments, percentage inhibition by the compound of the invention of one or more of the effects of Abeta species such as oligomers on neurons (such as neurons in the brain), such as amyloid (including amyloid oligomer) binding to synapses, and abnormalities in membrane trafficking mediated by Abeta oligomer was measured at a concentration of from 10 nM to 10 μM. In some embodiments, the percentage inhibition measured is about 1% to about 20%, about 20% to about 50%, about 1% to about 50%, or about 1% to about 80%. Inhibition can be assessed for example by quantifying synapse number of a neuron prior to and after exposure to an amyloid beta species or quantifying the number of synapses in the presence of both of a sigma-2 antagonist and the Abeta species wherein the sigma-2 antagonist is simultaneous with, or precedes or follows, Abeta species exposure. As another example, inhibition can be assessed by determining membrane trafficking and comparing one or more parameters that measure exocytosis rate and extent, endocytosis rate and extent, or other indicators of cell metabolism in the presence and absence of an Abeta species and in the presence and absence of a sigma-2 antagonist according to the invention. The present inventors have adduced biochemical assay evidence that compounds of the invention also inhibit amyloid aggregation (data not shown).

In some embodiments, the compounds described herein bind specifically to a sigma-2 receptor. A compound that binds specifically to a specific receptor refers to a compound that has a preference for one receptor over another. For example, although a compound may be capable of binding both sigma-1 and sigma-2 receptor, a compound can be said to be specific for a sigma-2 receptor when it binds with a binding affinity that is at least 10% greater than to the sigma-1 receptor. In some embodiments, the specificity is at least 10, 20, 30, 40, 50, 60, 70, 80, 90, 100, 200, 300, 400, 500, or 1000% greater for one binding partner (e.g. receptor) than a second binding partner.

In some embodiments, the present invention provides methods of measuring beta-amyloid-associated cognitive decline in an animal using a labeled sigma-2 ligand. In some embodiments, the method comprises contacting the animal with a labeled sigma-2 ligand according to the invention and measuring sigma-2 activity or expression. In some embodiments, the method comprises comparing the sigma-2 activity or expression in the animal with an animal known to have beta-amyloid induced cognitive decline. If the activity or expression is the same as the animal known to have beta-amyloid induced cognitive decline the animal is said to have the same level of cognitive decline. The animals can be ranked according the similarities in known activity or expression of various stages of beta amyloid induced cognitive decline. Any of the sigma-2 ligands described herein can be labeled so that the labeled sigma-2 ligand can be used in vivo.

In determining whether a compound of any of the formulae above and other compounds described as sigma-2 antagonists above is effective in treating the various conditions described herein, in vitro assays can be used. The in vitro assays have been correlated with an in vivo effect using Compound II For example, if a compound of formulae III-IV which bears structural similarity to compound II is active, for example, in the in vitro assays described herein, it can also be used in vivo to treat or ameliorate the conditions described herein including inhibiting or restoring synapse loss, modulating a membrane trafficking change in neuronal cells, protecting against or restoring memory loss, and treating cognitive decline conditions, diseases and disorders such as MCI and Alzheimer's disease. The assays are based, in part, on the amyloid beta oligomers and their function in binding to neurons at the synapses and the effect that amyloid beta oligomers have on neurons in vitro. In some embodiments, an Abeta oligomer receptor in neurons which the present inventors believe includes a sigma-2 protein is contacted with an amyloid beta assembly as described herein and a compound according to Formula I, III, IV, V, VI and VII that binds to the sigma-2 protein will inhibit the binding of the amyloid beta assembly to the receptor. In competitive radioligand binding assays the present inventors have shown that the present compounds are specific for the sigma-2 receptor. The inventors have also shown that the compounds of the invention inhibit binding of Abeta oligomers to their heretofore unidentified receptor on the surface of neurons. In some embodiments, methods are provided to determine a compound of any above formula's sigma-2 ligand efficacy in neuronal signaling. In some embodiments, the method comprises contacting a cell, such as but not limited to, a primary neuron, with a sigma-2 ligand and measuring neuronal function. In some embodiments, the cell is contacted in vitro. In some embodiments the cell is contacted in vivo. The neuronal activity can be signaling activity, electrical activity, the production or release of synaptic proteins, and the like. A sigma-2 antagonist that enhances or restores the signaling is identified as a compound that is effective in modulating neuronal activity. In some embodiments, the cell is derived from a pathological sample. In some embodiments, the cell is derived from a subject having a neurodegenerative disease. In some embodiments, the neurodegenerative disease is MCI or Alzheimer's Disease, especially mild Alzheimer's disease.

### Receptor Binding Assays and Compound Screening

The present invention also provides methods of identifying another compound that inhibits cognitive decline or treats a neurodegenerative disease. In some embodiments, the method comprises contacting a cell with a compound that binds a sigma-2 receptor. In some embodiments, the method comprises determining if the compound inhibits beta-amyloid pathology, wherein a compound that inhibits beta-amyloid pathology is identified as a compound that binds a sigma-2 receptor and that inhibits cognitive decline or treats a neurodegenerative disease. In some embodiments, the method also comprises identifying an additional compound that binds a sigma-2 receptor. In some embodiments, a method of identifying a compound that binds to a sigma-2 receptor comprises a competitive binding assay wherein a test compound is contacted with a sigma-2 receptor in the presence of a known sigma-2 ligand, such as the compounds of any formulae above and other compounds described as sigma-2 ligands above, wherein a test compound that competitively inhibits the binding of the known ligand is identified as a sigma-2 receptor ligand.

Methods of determining whether a compound can bind to a sigma-2 receptor are known and any method can be used. For example, testing was performed by a contract research organization. can be used to determine if a compound binds to Sigma-2. Various assays can be performed to determine if a compound binds to a Sigma-2 receptor. In some embodiments, cells, such as but not limited to, human embryonic kidney (HEK293), Jurkat cells, or Chinese hamster ovary (CHO) cells that stably express homogeneous populations of human receptors, including but not limited to sigma-2 receptor are used. In other cases, tissue sources of sigma-2 receptors such as rodent neocortical membranes are used. An example of this is described in the Examples section herein.

In some embodiments, a test compound is contacted with the cell or cell membrane to determine if the test compound can bind to the sigma-2 receptor. In some embodiments, the test compound is dissolved in a carrier or vehicle, such as but not limited to, dimethyl sulfoxide. In some embodiments, the cells are cultured until confluent. In some embodiments, upon confluence, the cells can be detached by gentle scraping. In some embodiments, the cells are detached by trypsinization, or any other suitable detachment means.

In some embodiments, the binding of the test compound to the sigma-2 receptor can be determined by, for example, a competitive radioligand binding assay. Radioligand binding assays can be carried out on intact cells stably expressing human receptors or a tissue source. The detached cells or tissue can, for example, be washed, centrifuged, and/or resuspended in a buffer. The test compound can be radiolabeled according to any method including, but not limited to, those described herein. The radioligand can be used at a fixed concentration of 0.1 μCi in the absence and presence of various concentrations (the range can be, for example, 10¹⁰-10³M OR 10¹¹-10⁴M of competing drugs. The drugs can be added to the tissue or cells (˜e.g., 50,000 cells) in a buffer and allowed to incubate. Nonspecific binding can be determined in the presence of broad spectrum activators or inhibitors or functional agonists or antagonists for each receptor subtype (for example, for sigma receptors, in the presence of e.g., 10 μM of an appropriate ligand for each receptor). Reactions can be terminated by rapid filtration, which can be followed by washes with ice-cold buffer twice. Radioactivity on the dried filter discs can be measured using any method, including but not limited to, a liquid scintillation analyzer. The displacement curves can be plotted and the Ki values of the test ligands for the receptor subtypes cam be determined using, for example, GraphPad Prism (GraphPad Software Inc., San Diego, Calif.). The percentage specific binding can be determined by dividing the difference between total bound (disintegrations per minute) and nonspecific bound (disintegrations per minute) by the total bound (disintegrations per minute).

In some embodiments, for binding studies in cell lines or tissues sources, varying concentrations of each drug were added in duplicate within each experiment, and the individual IC₅₀ values were determined using, for example, GraphPad Prism software. The Ki value of each ligand can be determined according to the equation described by Cheng and Prusoff (1973), and final data can presented as pKi+S.E.M., where in some embodiments, the number of tests is about 1-6.

In some embodiments, the method further comprises determining whether a compound that binds to a sigma-2 receptor acts as a functional antagonist at a sigma-2 receptor by inhibiting soluble Aβ oligomer induced neurotoxicity with respect to inhibiting soluble Aβ oligomer induced synapse loss, and inhibiting soluble Aβ oligomer induced deficits in a membrane trafficking assay. In some embodiments the method further determining that the sigma-2 receptor antagonist does not affect trafficking or synapse number in the absence of Abeta oligomer; does not induce caspase-3 activity in a neuronal cell; inhibits induction of caspase-3 activity by a sigma-2 receptor agonist; and/or decreases or protects against neuronal toxicity in a neuronal cell caused by a sigma-2 receptor agonist.

The testing can also include a functional assay to determine the effect of the test compound on the function of the binding partner, which can be, but is not limited to sigma-2 receptor. A variety of standard assay technologies can be used. For example, methods can be used to measure functional agonist-like or antagonist-like activity of compounds in living cells or tissues. Methods include, but are not limited to, TR-FRET to determine cAMP concentration and IP1 levels, real time fluorescence to monitor calcium flux, cellular dielectric spectroscopy to measure impedance modulation, ileum contraction, or tumor cell apoptosis. The specificity of the test compound can also be determined by, for example, determining if the compound binds to Sigma-1 receptor, Sigma-2 receptor, neither, or both. A method for determining if a test compound binds to a Sigma-1 receptor is described in Ganapathy, M. E et al. (1999) J. Pharmacol. Exp. Ther., 289: 251-260, which is hereby incorporated by reference in its entirety. A method for determining if a test compound binds to a Sigma-1 receptor is described in Bowen, W. D et al. (1993) Mol. Neuropharmacol., 3: 117-126, which is hereby incorporated by reference in its entirety, and also Xu, J. et al, Nature Communications, 2011, 2:380 DOI: 10.1038/ncomms 1386 which is also hereby incorporated by reference here in its entirety.

In various embodiments, the disclosure provides assay protocols for identification of a selective, high affinity sigma-2 receptor ligands that can act as a functional antagonist at a sigma-2 receptor by inhibiting soluble Aβ oligomer-induced neurotoxicity with respect to inhibiting soluble Aβ oligomer induced synapse loss, that inhibits soluble Aβ oligomer induced deficits in a membrane trafficking assay, that does not affect trafficking or synapse number in the absence of Abeta oligomer; and that exhibits good drug like properties as described herein such that the selective, high affinity sigma-2 receptor antagonist compound thus identified can be used to treat soluble Aβ oligomer-induced synaptic dysfunction in vivo.

In some embodiments, screening methods are provided for identifying compounds that will be active in abating or protecting against nonlethal Abeta oligomer toxicity would substantially benefit from incorporating as a screening criterion an ability of a test compound to bind to sigma-2 receptor, assessed for example by its ability to displace known ligands or by any other method. In addition, the test compound should be subjected to at least one in vitro test that can assess the ability of the compound to block or to abate nonlethal deleterious effects of Abeta oligomers on neurons, such as the membrane trafficking assay or the synapse number or oligomer binding assay described herein or an in vivo assay assessing treatment of cognitive decline, such as those described herein.

In some embodiments, the present invention provides methods of determining whether a subject should be treated with a sigma-2 antagonist, wherein the subject is suspected of having cognitive decline or a neurodegenerative disease or other condition, disease or disorder described herein. In some embodiments, the method comprises contacting a sample derived from the patient with a sigma-2 antagonist and determining whether the sigma-2 modulating compound inhibits or ameliorates a beta-amyloid pathology present in the sample, wherein a sample that shows inhibition or amelioration of the beta-amyloid pathology present in the sample indicates that the subject should be treated with a sigma-2 antagonist.

Additionally, the present invention includes methods to identify sigma-2 antagonists that inhibit an Aβ oligomer induced reduction in synapse number, and the like. In some embodiments, the methods can be used to identify sigma-2 antagonists for treating a beta-amyloid pathology. In some embodiments, the methods are used to determine the efficacy of a treatment to treat a beta-amyloid pathology. In some embodiments, the beta-amyloid pathology is a defect in membrane trafficking, synaptic dysfunction, memory and learning defect in an animal, reduction in synapse number, change in dendritic spine length or spine morphology, a defect in LTP, or an increase in the phosphorylation of Tau protein.

### Amyloid Beta as Used in the Present Disclosure

Human amyloid β is the cleavage product of an integral membrane protein, amyloid precursor protein (APP), found concentrated in the synapses of neurons. Amyloid β self-associates to form metastable, oligomeric assemblies. At higher concentrations, Abeta will polymerize and assemble into linear-shaped fibrils, facilitated by lower pH. It is not presently clear whether fibrils are formed from oligomers. Amyloid β oligomers have been demonstrated to cause Alzheimer's disease in animal models by inducing changes in neuronal synapses that block learning and memory, and amyloid β fibrils have long been associated with the advanced stages Alzheimer's disease in animals and humans. In fact, the modern working hypothesis for Alzheimer's disease, and one that has gained a lot of support, is that Abeta assemblies and notably Abeta oligomers are at the center of early pathology associated with Alzheimer's as well as of pathologies associated with less grave dementias, such as MCI and mild AD. Cleary, James P. et al. “Natural oligomers of the amyloid-β protein specifically disrupt cognitive function.” Nature Neuroscience Vol. 8 (2005): 79-84; Klyubin, I. et al. “Amyloid beta protein dimer-containing human CSF disrupts synaptic plasticity: prevention by systemic passive immunization.” J Neurosci. Vol. 28 (2008): 4231-4237. However, very little is known about how oligomers form and the structural state of the oligomer. For example, the number of amyloid β subunits that associate to form the oligomer is currently unknown, as is the structural form of the oligomers, or which residues are exposed. There is evidence to suggest that more than one structural state of oligomer is neuroactive. Reed, Jess D. et al. “MALDI-TOF mass spectrometry of oligomeric food polyphenols.” Phytochemistry 66:18 (September 2005): 2248-2263; Cleary, James P. et al. “Natural oligomers of the amyloid-β protein specifically disrupt cognitive function.” Nature Neuroscience Vol. 8 (2005): 79-84.

Amyloid β has affinity for many proteins found in the brain, including ApoE and ApoJ. However, it is unclear whether chaperones or other proteins form associations with the protein that can affect its final structural state and/or its neuroactivity.

Soluble Abeta peptide is likely to play a key role during early stages of AD by perturbing synaptic disfunction and cognitive processes. For example, Origlia et al. showed soluble Abeta (Abeta 42) impairs long term potentiation (LTP) in the entorhinal cortex through neuronal receptor for advanced glycation end products (RAGE)-mediated activation of p38MAPK. Origlia et al. 2008, Receptor for advanced glycation end product-dependent activation of p38 mitogen-activated protein kinase contributes to amyloid-beta-mediated cortical synaptic dysfunction. J. Neuroscience 28(13):3521-3530, incorporated herein by reference.

Synaptic dysfunction is involved in early stages of Alzheimer's disease. Amyloid beta peptides have been shown to alter synaptic function. Puzzo et al reported that a synthetic fibrillar form of Abeta impairs the late protein synthesis dependent phase of LTP without affecting the early protein synthesis phase. The report is consistent with earlier reports that Abeta oligomers are highly toxic to cells and involved in synaptic dysfunction. Puzzo et al., 2006, Curr Alzheimer's Res 3(3):179-183, which is incorporated herein by reference. Abeta has been found to markedly impair hippocampal long-term potentiation (LTP) by various second messenger cascades including a nitric oxide cascade. NO/cGMP/cGK/CREB. Puzzo et al., J Neurosci. 2005, In some embodiments, the disclosure provides compositions and methods comprising sigma-2 receptor antagonists for inhibiting amyloid beta oligomer-induced synaptic dysfunction of a neuronal cell; and inhibiting suppression of hippocampal long term potention caused by exposure of neurons to Abeta oligomers.

Any form of amyloid β may be used in the practice of the screening methods and of the assays according to the invention, including amyloid β monomers, oligomers, fibrils, as well as amyloid β associated with proteins (“protein complexes”) and more generally amyloid β assemblies. For example, screening methods can employ various forms of soluble amyloid β oligomers as disclosed, for example, in U.S. patent application Ser. No. 13/021,872; U.S. Patent Publication 2010/0240868; International Patent Application WO/2004/067561; International Patent Application WO/2010/011947; U.S. Patent Publication 20070098721; U.S. Patent Publication 20100209346; International Patent Application WO/2007/005359; U.S. Patent Publication 20080044356; U.S. Patent Publication 20070218491; WO/2007/126473; U.S. Patent Publication 20050074763; International Patent Application WO/2007/126473, International Patent Application WO/2009/048631, and U.S. Patent Publication 20080044406,U.S. Pat. No. 7,902,328 and U.S. Pat. No. 6,218,506, each of which is incorporated herein by reference.

Amyloid β forms, including monomers or oligomers of amyloid β may be obtained from any source. For example, in some embodiments, commercially available amyloid β monomers and/or amyloid β oligomers may be used in the aqueous solution, and in other embodiments, amyloid β monomers and/or amyloid β oligomers that are used in the aqueous protein solution can be isolated and purified by the skilled artisan using any number of known techniques. In general, the amyloid β monomers and/or amyloid β oligomers used in the preparation of the aqueous solution of proteins and amyloid β of various embodiments may be soluble in the aqueous solution. Therefore, both the proteins of the aqueous solution and the amyloid β may be soluble.

The amyloid β added may be of any isoform. For example, in some embodiments, the amyloid β monomers may be amyloid β 1-42, and in other embodiments the amyloid β monomers may be amyloid β 1-40. In still other embodiments, the amyloid β may be amyloid β 1-39 or amyloid β 1-41. Hence, the amyloid β of various embodiments may encompass any C-terminal isoform of amyloid β. Yet other embodiments include amyloid β in which the N-terminus has been frayed, and in some embodiments, the N-terminus of any of amyloid β C-terminal isomers described above may be amino acid 2, 3, 4, 5, or 6. For example, amyloid β 1-42 may encompass amyloid β 2-42, amyloid β 3-42, amyloid β 4-42, or amyloid β 5-42 and mixtures thereof, and similarly, amyloid β 1-40 may encompass amyloid β 2-40, amyloid β 3-40, amyloid β 4-40, or amyloid β 5-40.

The amyloid β forms used in various embodiments may be wild type, i.e. having an amino acid sequence that is identical to the amino acid sequence of amyloid β synthesized in vivo by the majority of the population, or in some embodiments, the amyloid β may be a mutant amyloid β. Embodiments are not limited to any particular variety of mutant amyloid β. For example, in some embodiments, the amyloid β introduced into the aqueous solution may include a known mutation, such as, for example, amyloid β having the “Dutch” (E22Q) mutation or the “Arctic” (E22G) mutation. Such mutated monomers may include naturally occurring mutations such as, for example, forms of amyloid β isolated from populations of individuals that are predisposed to, for example, Alzheimer's disease, familial forms of amyloid β. In other embodiments, mutant amyloid β monomers may be synthetically produced by using molecular techniques to produce an amyloid β mutant with a specific mutation. In still other embodiments, mutant amyloid β monomers may include previously unidentified mutations such as, for example, those mutants found in randomly generated amyloid β mutants. The term “amyloid β” as used herein is meant to encompass both wild type forms of amyloid β as well as any of the mutant forms of amyloid β.

In some embodiments, the amyloid β in the aqueous protein solution may be of a single isoform. In other embodiments, various C-terminal isoforms of amyloid β and/or various N-terminal isoforms of amyloid β may be combined to form amyloid β mixtures that can be provided in the aqueous protein solution. In yet other embodiments, the amyloid β may be derived from amyloid precursor protein (APP) that is added to the protein containing aqueous solution and is cleaved in situ, and such embodiments, various isoforms of amyloid β may be contained within the solution. Fraying of the N-terminus and/or removal of C-terminal amino acids may occur within the aqueous solution after amyloid β has been added. Therefore, aqueous solutions prepared as described herein may include a variety of amyloid β isoforms even when a single isoform is initially added to the solution.

The amyloid β monomers added to the aqueous solution may be isolated from a natural source such as living tissue, and in other embodiments, the amyloid β may be derived from a synthetic source such as transgenic mice or cultured cells. In some embodiments, the amyloid β forms, including monomers, oligomers, or combinations thereof are isolated from normal subjects and/or patients that have been diagnosed with cognitive decline or diseases associated therewith, such as, but not limited to, Alzheimer's disease. In some embodiments, the amyloid β monomers, oligomers, or combinations thereof are Abeta assemblies that have been isolated from normal subjects or diseased patients. In some embodiments, the Abeta assemblies are high molecular weight, e.g. greater than 100 KDa. In some embodiments, the Abeta assemblies are intermediate molecular weight, e.g. 10 to 100 KDa. In some embodiments, the Abeta assemblies are less than 10 kDa.

The amyloid β oligomers of some embodiments may be composed of any number of amyloid β monomers consistent with the commonly used definition of “oligomer.” For example, in some embodiments, amyloid β oligomers may include from about 2 to about 300, about 2 to about 250, about 2 to about 200 amyloid β monomers, and in other embodiments, amyloid β oligomers may be composed from about 2 to about 150, about 2 to about 100, about 2 to about 50, or about 2 to about 25, amyloid β monomers. In some embodiments, the amyloid β oligomers may include 2 or more monomers. The amyloid β oligomers of various embodiments may be distinguished from amyloid β fibrils and amyloid β protofibrils based on the confirmation of the monomers. In particular, the amyloid β monomers of amyloid β oligomers are generally globular consisting of β-pleated sheets whereas secondary structure of the amyloid β monomers of fibrils and protofibrils is parallel β-sheets.

### Identification of Subjects Having or at Risk of Having Alzheimer's Disease

Alzheimer's disease (AD) is defined histologically by the presence of extracellular β-amyloid (Aβ) plaques and intraneuronal neurofibrillary tangles in the cerebral cortex. Various diagnostic and prognostic biomarkers are known in the art, such as magnetic resonance imaging, single photon emission tomography, FDG PET, PiB PET, CSF tau and Abeta analysis, as well as available data on their diagnostic accuracy are discussed in Alves et al., 2012, Alzheimer's disease: a clinical practice-oriented review, Frontiers in Neurology, April, 2012, vol 3, Article 63, 1-20, which is incorporated herein by reference.

The diagnosis of dementia, along with the prediction of who will develop dementia, has been assisted by magnetic resonance imaging and positron emission tomography (PET) by using [(18)F]fluorodeoxyglucose (FDG). These techniques are not specific for AD. See, e.g., Vallabhajosula S. Positron emission tomography radiopharmaceuticals for imaging brain Beta-amyloid. Semin Nucl Med. 2011 July; 41(4):283-99. Another PET ligand recently FDA approved for imaging moderate to frequent amyloid neuritic plaques in patients with cognitive impairment is Florbetapir F 18 injection, (4-((1E)-2-(6-{2-(2-(2-(18F)fluoroethoxy)ethoxy)ethoxy}pyridin-3-yl)ethenyl)-N-methylbenzenamine, AMYVID®, Lilly). Florbetapir binds specifically to fibrillar Abeta, but not to neurofibrillary tangles. See, e.g., Choi S R, et al., Correlation of amyloid PET ligand florbetapir F 18 binding with Aβ aggregation and neuritic plaque deposition in postmortem brain tissue. Alzheimer Dis Assoc Disord. 2012 January; 26(1):8-16. The PET ligand florbetapir suffers from low specificity with respect to qualitative visual assessment of the PET scans. Camus et al., 2012, Eur J Nucl Med Mol Imaging 39:621-631. However, many people with neuritic plaques seem cognitively normal.

CSF markers for Alzheimer's disease include total tau, phosphor-tau and Abeta42. See, for example, Andreasen, Sjogren and Blennow, World J Biol Psyciatry, 2003, 4(4): 147-155, which is incorporated herein by reference. Reduced CSF levels of the 42 amino acid form of Abeta (Abeta42) and increased CSF levels of total tau in AD have been found in numerous studies. In addition, there are known genetic markers for mutations in the APP gene useful in the identification of subjects at risk for developing AD. See, for example, Goate et al., Segregation of a missense mutation in the amyloid precursor protein gene with familial Alzheimer's disease, Nature, 349, 704-706, 1991, which is incorporated herein by reference. In embodiments, any known diagnostic or prognostic method can be employed to identify a subject having or at risk of having Alzheimer's disease. Pharmaceutical Compositions Comprising a Sigma-2 Receptor Antagonist

The sigma-2 receptor antagonist compounds, antibodies, or fragments, identified by means of the present invention can be administered in the form of pharmaceutical compositions. These compositions can be prepared in a manner well known in the pharmaceutical art, and can be administered by a variety of routes, depending upon whether local or systemic treatment is desired and upon the area to be treated.

Thus, another embodiment of the present invention comprises pharmaceutical compositions comprising a pharmaceutically acceptable excipient or diluent and a therapeutically effective amount of a sigma-2 receptor antagonist compound of the invention, including an enantiomer, diastereomer, N-oxide or pharmaceutically acceptable salt thereof.

While it is possible that a compound may be administered as the bulk substance, it is preferable to present the active ingredient in a pharmaceutical formulation, e.g., wherein the active agent is in admixture with a pharmaceutically acceptable carrier selected with regard to the intended route of administration and standard pharmaceutical practice.

Accordingly, in one aspect, the present invention provides a pharmaceutical composition comprising at least one compound, antibody or fragment, of any of the formulae above and other compounds described as sigma-2 receptor antagonists above described above or a pharmaceutically acceptable derivative (e.g., a salt or solvate) thereof, and, optionally, a pharmaceutically acceptable carrier. In particular, the invention provides a pharmaceutical composition comprising a therapeutically effective amount of at least one compound of any of the formulae above or a pharmaceutically acceptable derivative thereof, and, optionally, a pharmaceutically acceptable carrier.

### Combinations

For the compositions and methods of the invention, a compound of any of the formulae above and other compounds described as sigma-2 receptor antagonists above described above may be used in combination with other therapies and/or active agents.

In some embodiments, the sigma-2 antagonist compound can be combined with one or more of a cholinesterase inhibitor, an N-methyl-D-aspartate (NMDA) glutamate receptor antagonist, a beta-amyloid specific antibody, a beta-secretase 1 (BACE1, beta-site amyloid precursor protein cleaving enzyme 1) inhibitor, a tumor necrosis factor alpha (TNF alpha) modulator, an intravenous immunoglobulin (IVIG), or a prion protein antagonist. In some embodiments the sigma-2 receptor antagonist is combined with a cholinesterase inhibitor selected from tacrine (COGNEX®; Sciele), donepezil (ARICEPT®; Pfizer), rivastigmine (EXELON®; Novartis), or galantamine (RAZADYNE®; Ortho-McNeil-Janssen). In some embodiments, the sigma-2 receptor antagonist is combined with a TNFalpha modulator that is perispinal etanercept (ENBREL®, Amgen/Pfizer). In some embodiments, the sigma-2 receptor antagonist is combined with a beta-amyloid specific antibody selected from bapineuzumab (Pfizer), solanezumab (Lilly), PF-04360365 (Pfizer), GSK933776 (GlaxoSmithKline), Gammagard (Baxter) or Octagam (Octapharma). In some embodiments, the sigma-2 receptor antagonist is combined with an NMDA receptor antagonist that is memantine (NAMENDA®; Forest). In some embodiments, the BACE1 inhibitor is MK-8931 (Merck). In some embodiments, the sigma-2 receptor antagonist is combined with IVIG as described in Magga et al., J Neuroinflam 2010, 7:90, Human intravenous immunoglobulin provides protection against Ab toxicity by multiple mechanisms in a mouse model of Alzheimer's disease, and Whaley et al., 2011, Human Vaccines 7:3, 349-356, Emerging antibody products and Nicotiana manufacturing; each of which is incorporated herein by reference. In some embodiments, the sigma-2 receptor antagonist is combined with a prion protein antagonist as disclosed in Strittmatter et al., US 2010/0291090, which is incorporated herein by reference.

Accordingly, the present invention provides, in a further aspect, a pharmaceutical composition comprising at least one compound of any of the formulae above or a pharmaceutically acceptable derivative thereof, a second active agent, and, optionally a pharmaceutically acceptable carrier.

When combined in the same formulation it will be appreciated that the two compounds, antibodies or fragments must be stable and compatible with each other and the other components of the formulation. When formulated separately they may be provided in any convenient formulation, conveniently in such manner as are known for such compounds in the art.

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

With respect to biologics such as monoclonal antibodies or fragments, suitable excipients will be employed to prevent aggregation and stabilize the antibody or fragment in solution with low endotoxin, generally for parenteral, for example, intravenous, administration. For example, see Formulation and Delivery Issues for Monoclonal Antibody Therapeutics, Daugherty et al., in Current Trends in Monoclonal Antibody Development and Manufacturing, Part 4, 2010, Springer, New York pp 103-129.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see WO 02/00196 (SmithKline Beecham).

### Routes of Administration and Unit Dosage Forms

The routes for administration (delivery) include, but are not limited to, one or more of: oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration etc. Administration of an antibody or fragment will generally be by parenteral means.

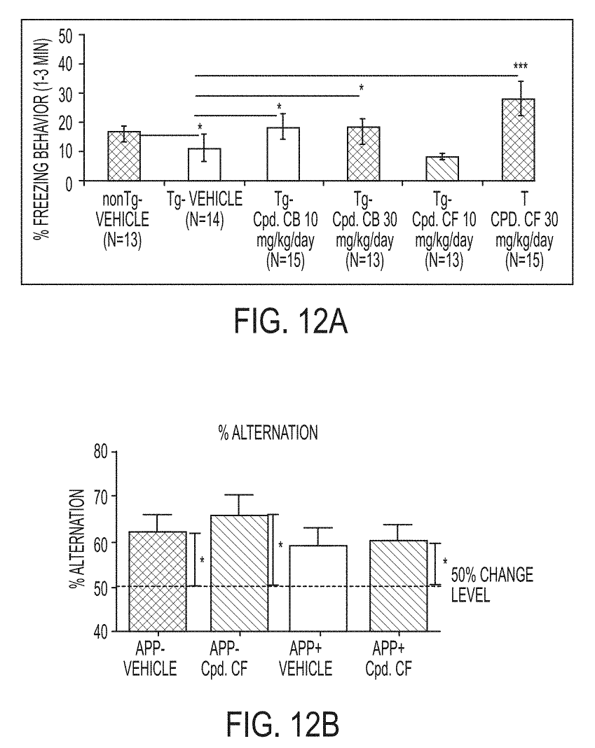

Therefore, the compositions of the invention include those in a form especially formulated for, the mode of administration. In certain embodiments, the pharmaceutical compositions of the invention are formulated in a form that is suitable for oral delivery. For example compound CB and compound CF are sigma-2 receptor antagonist compounds that are orally bioavailable in animal models and have been administered orally once per day and shown efficacy in a fear conditioning model, see for example FIG. 12B Orally bioavailable compounds as described herein can be prepared in an oral formulation. In some embodiments, the sigma-2 antagonist compound is an orally bioavailable compound, suitable for oral delivery. In other embodiments, the pharmaceutical compositions of the invention are formulated in a form that is suitable for parenteral delivery In some embodiments, the sigma-2 receptor antagonist compound is an antibody or fragment thereof, wherein the antibody or fragment is formulated in a parenteral composition. For example, an anti-sigma-2 receptor antibody such as an anti-PGRMC1 antibody that blocks binding of Abeta oligomers to the sigma-2 receptor can be formulated for parenteral delivery.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The antibody or antibody fragment molecules of the present invention can be formulated and administered by any of a number of routes and are administered at a concentration that is therapeutically effective in the indication or for the purpose sought. To accomplish this goal, the antibodies may be formulated using a variety of acceptable excipients known in the art. Typically, the antibodies are administered by injection, for example, intravenous injection. Methods to accomplish this administration are known to those of ordinary skill in the art. For example, Gokarn et al., 2008, J Pharm Sci 97(8):3051-3066, incorporated herein by reference, describe various high concentration antibody self buffered formulations. For example, monoclonal antibodies in self buffered formulation at e.g., 50 mg/mL mAb in 5.25% sorbitol, pH 5.0 or 60 mg/mL mAb in 5% sorbitol, 0.01% polysorbate 20, pH 5.2; or conventional buffered formulations, for example, 50 mg/mL mAb1 in 5.25% sorbitol, 25 or 50 mM acetate, glutamate or succinate, at pH 5.0; or 60 mg/mL in 10 mM acetate or glutamate, 5.25% sorbitol, 0.01% polysorbate 20, pH 5.2; other lower concentration formulations can be employed as known in the art.

Because the preferred sigma-2 receptor antagonist compounds of the invention cross the blood brain barrier they can be administered in a variety of methods including for example systemic (e.g., by iv, SC, oral, mucosal, transdermal route) or localized methods (e.g., intracranially). Where the compound of the invention is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the sigma-2 antagonist compounds selected from the sigma-2 ligands and prepared for oral administration described above may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

Where appropriate, the pharmaceutical compositions can be administered by inhalation, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

Where the composition of the invention is to be administered parenterally, such administration includes without limitation: intravenously, intraarterially, intrathecally, intraventricularly, intracranially, intramuscularly or subcutaneously administering the compound of the invention; and/or by using infusion techniques. Antibodies or fragments are typically administered parenterally, for example, intravenously.

Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or acsorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminum monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of formulas I, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the sigma-2 receptor antagonists and desired excipients for subsequent preparation of sterile solutions.

The compounds according to the invention may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds of the invention can be administered in the form of tablets, capsules, ovules, elixirs, solutions or suspensions, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, or suspensions, or a dry powder for reconstitution with water or other suitable vehicle before use. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthine resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable-lubricants useful in the compositions of the invention include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

As indicated, the compounds of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

The compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage form of the present invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the compounds of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

The amount of the compound to be administered can range between about 0.01 and about 25 mg/kg/day, usually between about 0.1 and about 10 mg/kg/day and most often between 0.2 and about 5 mg/kg/day. It will be understood that the pharmaceutical formulations of the present invention need not necessarily contain the entire amount of the compound that is effective in treating the disorder, as such effective amounts can be reached by administration of a plurality of divided doses of such pharmaceutical formulations.

In a preferred embodiment of the present invention, the compounds I are formulated in capsules or tablets, usually containing 10 to 200 mg of the compounds of the invention, and are preferably administered to a patient at a total daily dose of 10 to 300 mg, preferably 20 to 150 mg and most preferably about 50 mg.

A pharmaceutical composition for parenteral administration contains from about 0.01% to about 100% by weight of the active compound of the present invention, based upon 100% weight of total pharmaceutical composition.

Generally, transdermal dosage forms contain from about 0.01% to about 100% by weight of the active compound versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. To this purpose, the combined active principles are formulated into a simple dosage unit.

Synthesis of the Compounds of the Invention

Compounds of formulas I and II and enantiomers, diastereomers, N-oxides, and pharmaceutically acceptable salts thereof may be prepared by the general methods outlined hereinafter, said methods constituting a further aspect of the invention. In the following description, the groups R₁-6, have the meaning defined for the compounds of any of the formulae above unless otherwise stated.

It will be appreciated by those skilled in the art that it may be desirable to use protected derivatives of intermediates used in the preparation of the compounds I. Protection and deprotection of functional groups may be performed by methods known in the art (see, for example, Green and Wuts Protective Groups in Organic Synthesis. John Wiley and Sons, New York, 1999.). Hydroxy or amino groups may be protected with any hydroxy or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

The synthesis of the target compounds is completed by removing any protecting groups which may be present in the penultimate intermediates using standard techniques, which are well-known to those skilled in the art. The deprotected final products are then purified, as necessary, using standard techniques such as silica gel chromatography, HPLC on silica gel and the like, or by recrystallization. The compounds above can be synthesized via any synthetic route. For example, the compounds can be prepared according to the following scheme (Scheme 1).

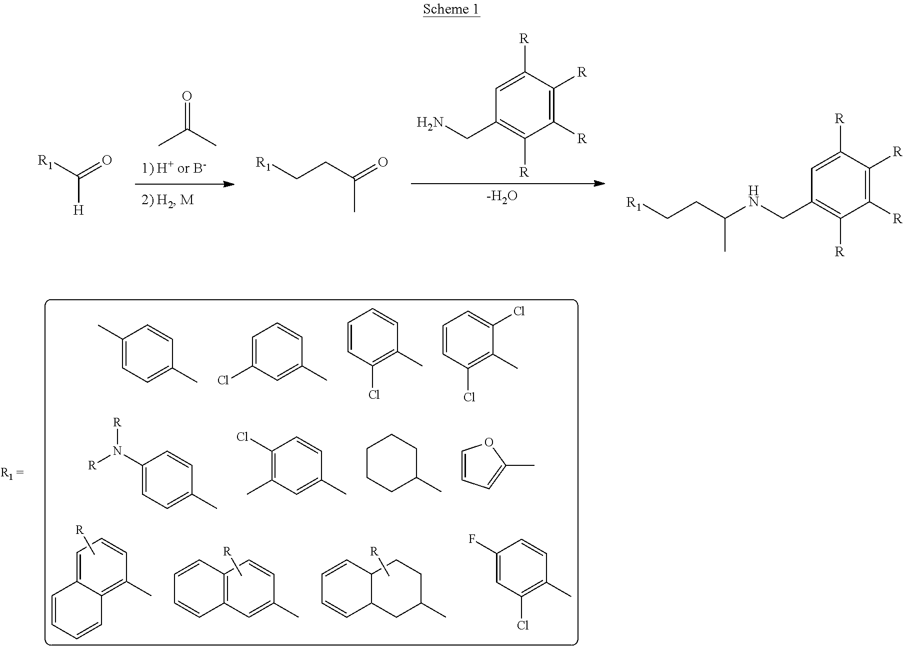

This scheme can produce a racemic mixture of the analogues described herein. Additional R1 groups can also be used to generate other analogues.

In some embodiments, the synthesis is performed asymmetrically in order to produce a substantially pure or pure enantiomer of one of an analogue. In some embodiments, the asymmetric synthesis of a compound described herein is prepared according to Scheme 2 (* indicates chiral center):

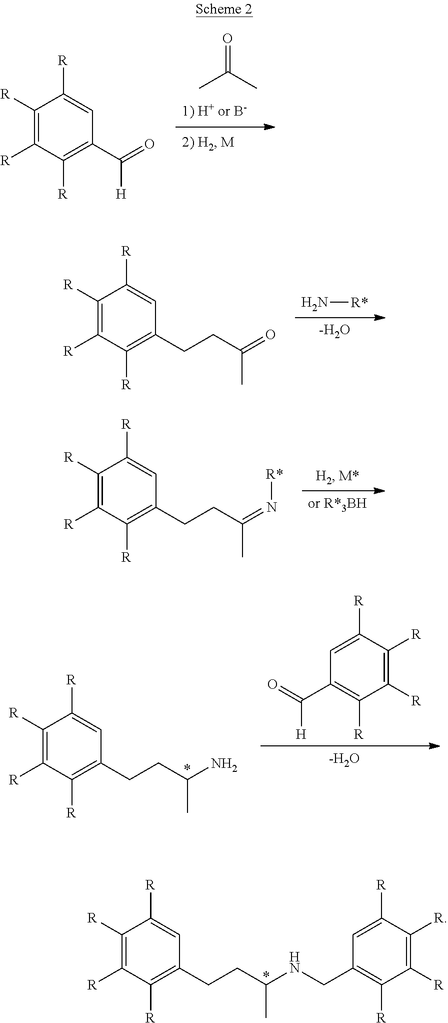

In some embodiments, the asymmetric synthesis of a compound described herein is prepared according to Scheme 3 (* indicates chiral center):

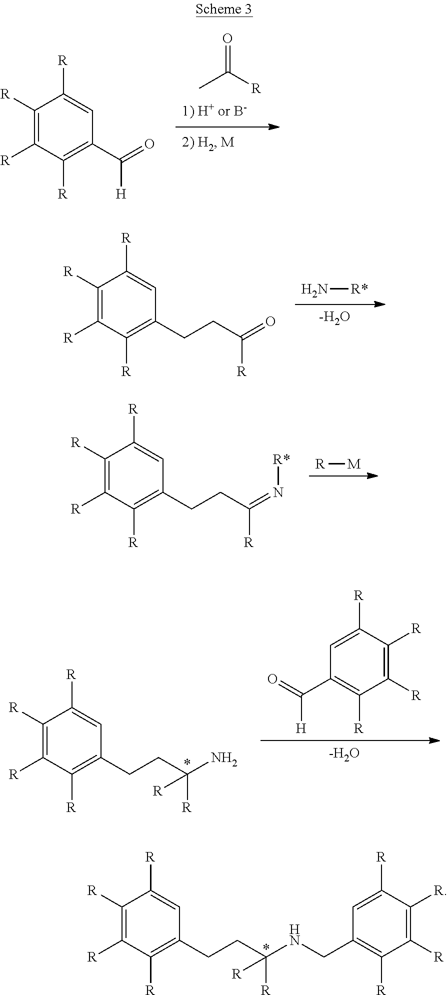

The synthetic scheme can be altered depending upon the end-product desired. The “R” groups are exemplary and can be substituted with any substituent described herein.

The following is a general method for preparing the compounds of Formula IX. As shown in Scheme 4, ketone 4-1 can be reacted with Wittig reagents such as 4-2, followed by hydrolysis (for example under acidic condition) to afford ketone 4-3. The enolate of ketone 4-3 with a reagent such as LDA, and condensed with acetone followed by conjugate reduction of the alkene toto form ketone 4-4. Reductive amination of ketone 4-5 with a suitable amine R^{3b}NH₂ in the presence of a suitable hydride such as sodium borohydride can afford amine 4-6. Different diastereomers of amine 4-6 can be separated by methods known to those skilled in the art such as column chromatography.

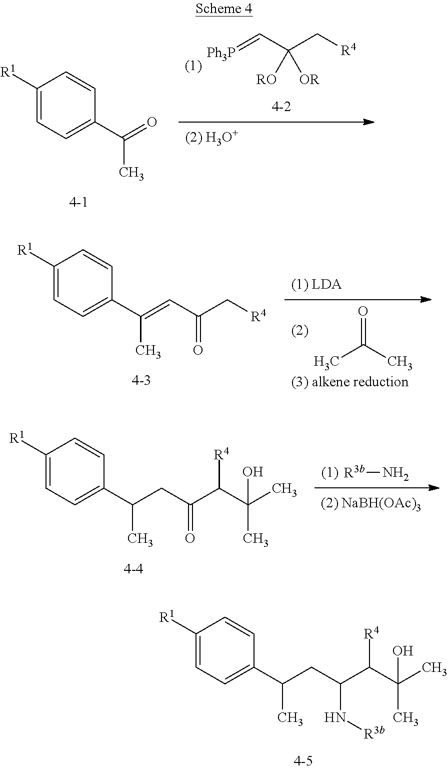

As shown in Scheme 4a, aromatic compound 4a-0-1 can be reduced to cyclohexa-1,4-diene 4a-02 under Birch reduction conditions. See e.g. Rabideau, P. W., “The metal-ammonia reduction of aromatic compounds”, Tetrahedron, Volume 45, Issue 6, 1989, pages 1579-1603. Under acidic conditions (such as in the presence of catalytic amount of HCl or acetic acid), cyclohexa-1,4-diene 4a-02 can rearrange to the thermodynamically more stable cyclohexa-1,3-diene 4a-1. Cyclohexa-1,3-diene 4a-1. can be converted to alcohol 4a-6 or amine 4a-8 according to methods similar to those described in Scheme 4.

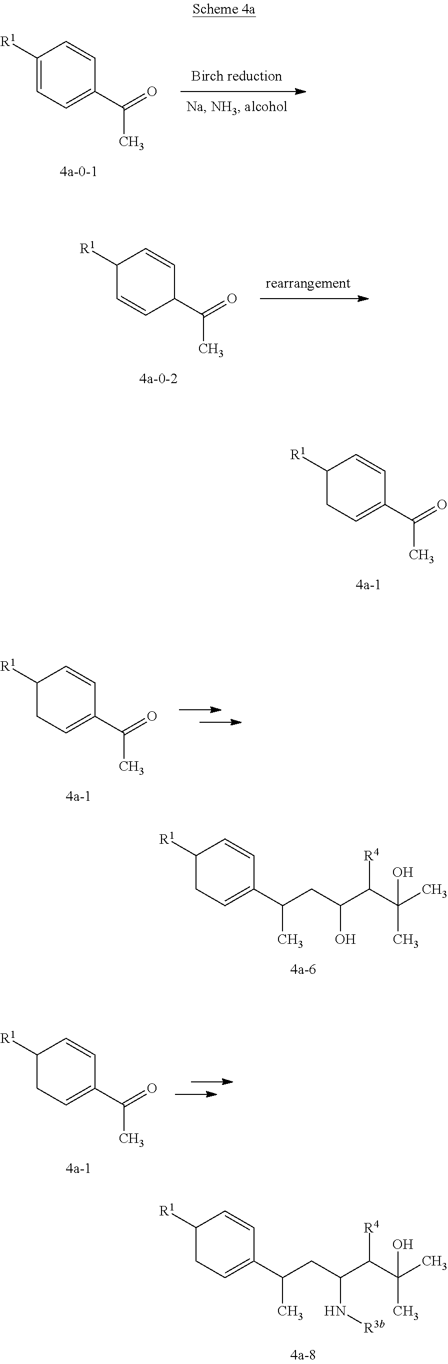

As shown Scheme 5, treatment of styrene derivative 5-1 with AD-mix-α (See e.g. Sharpless, K. B.; Amberg, W.; Bennani, Y. L.; Crispino, G. A.; et al. J. Org. Chem. 1992, 57, 2771) affords diol 5-2. See A. Li, et. al, “Total asymmetric synthesis of (7S,9R)-(+)-bisacumol”, Tetrahedron: Asymmetry (2003), 14(1), 75-78. Stereo-selective reduction of the benzylic OH of diol 5-2 with Raney nickel gives alcohol 5-3. See id. Both the isomer of 5-2 can be obtained based on selection of the Sharpless catalyst. Treatment of alcohol 5-3 with PPh₃ and CBr₄ in a suitable solvent such as methylene chloride affords bromide 5-4. Conversion of bromide 5-4 to the corresponding Grignard reagent in the presence of magnesium powder and CH₃I (by metal-halogen exchange), followed by reaction with acetaldehyde, provides alcohol 5-5. Different diastereomers of alcohol 5-5 can be separated by methods known to those skilled in the art such as column chromatography. See id. Alcohol 5-5 can be transformed into its corresponding amine compound 5-6 using similar methods to those outlined in Scheme 4. The isomers of the amine compound 5-8 can be obtained by stereoselective imine reduction.

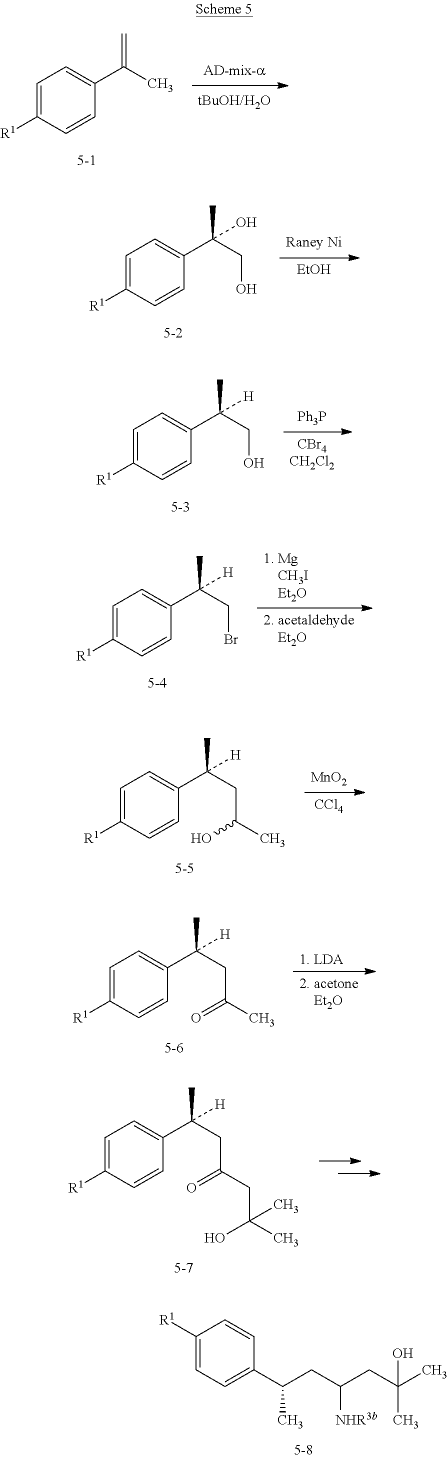

Those skilled in the art can recognize that in all of the schemes described herein, if there are functional (reactive) groups present on a substituent group such as R¹, R², R³, and R⁴, etc., further modification can be made if appropriate and/or desired. For example, an OH group can be converted into a better leaving group such as mesylate, which in turn is suitable for nucleophilic substitution, such as by Br. Thus, a compound of Formula I (such as compound 4-8 of Scheme 4) having a substituent which contains a functional group can be converted to another compound of Formula I having a different substituent group.

In some embodiments certain compounds of formulas I-VI are prepared, for example, by the enantioselective route shown in Scheme 6.

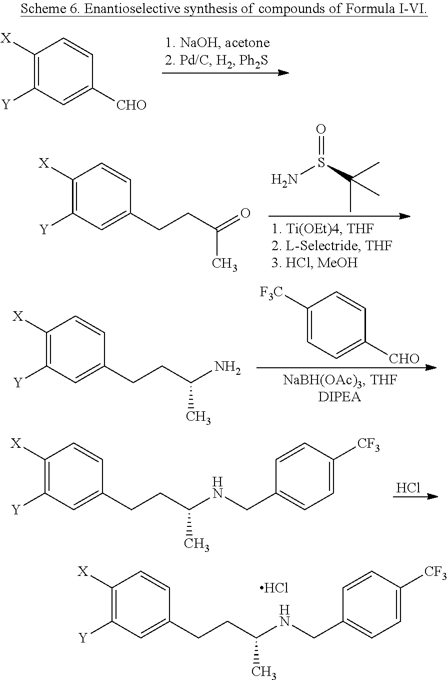

In some embodiments, the sigma-2 antagonist is a compound of formula VIIIa. Certain compounds of various Formulas VIII can be prepared by reductive amination of corresponding ketone intermediates, for example, by the representative route shown in Scheme 7.

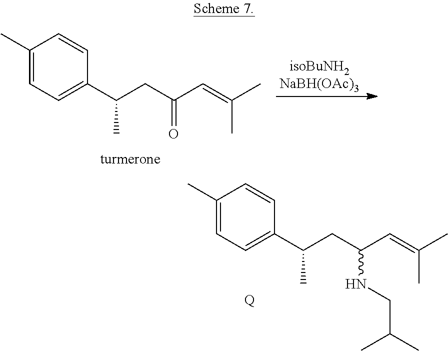

### WORKING AND SYNTHESIS EXAMPLES

Examples 1 and 2 describe Abeta oligomer preparations that could be used for experiments such as those described herein. The particular preparations used in the membrane trafficking and oligomer bindin/synapse reduction assays as well as those used in the in vivo assays described below are each described in the example to which they pertain.

### Example 1: Preparation of Amyloid β Oligomers

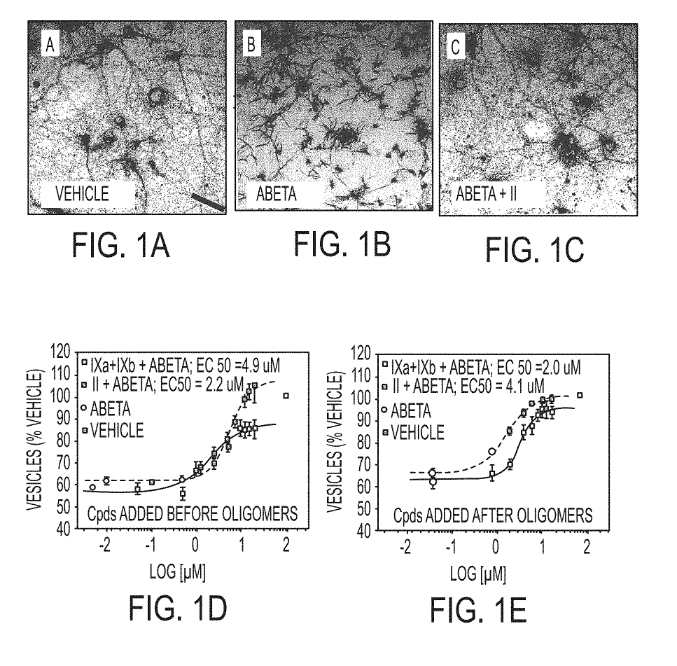

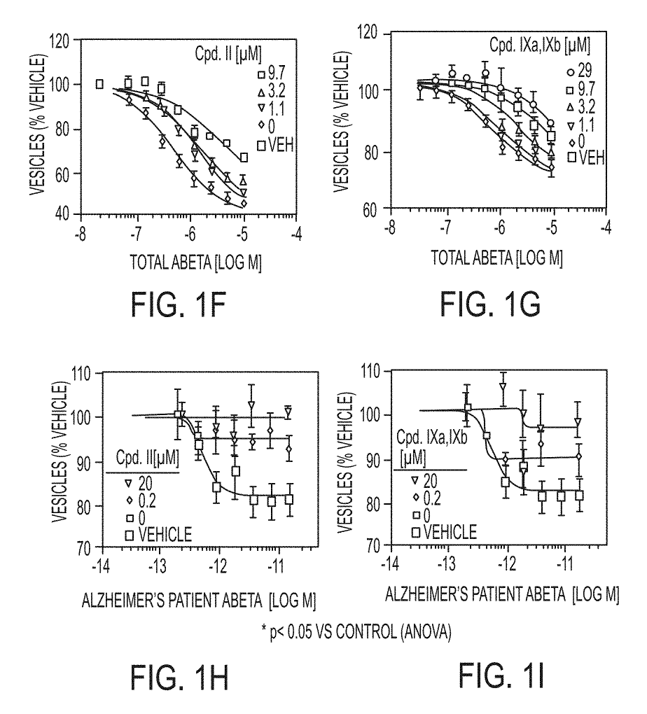

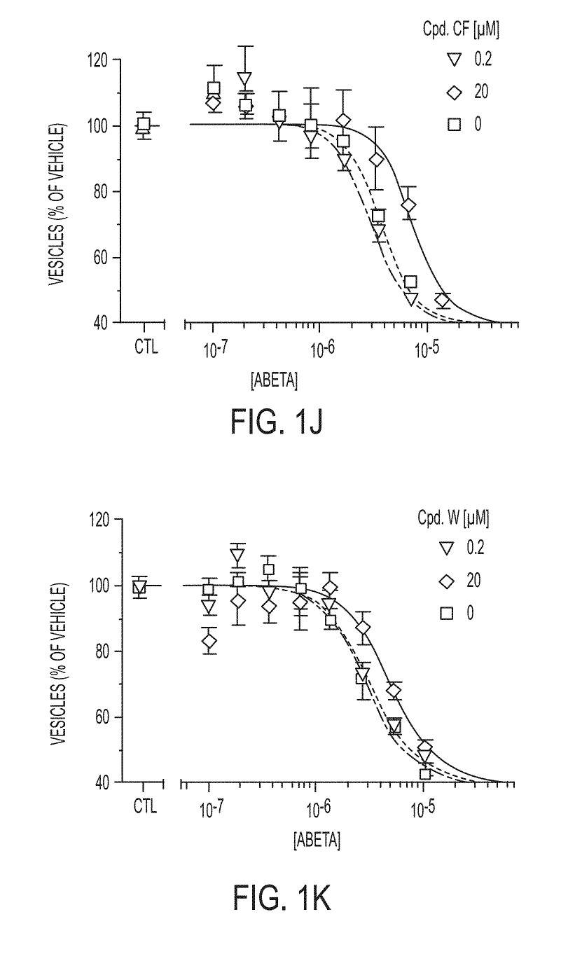

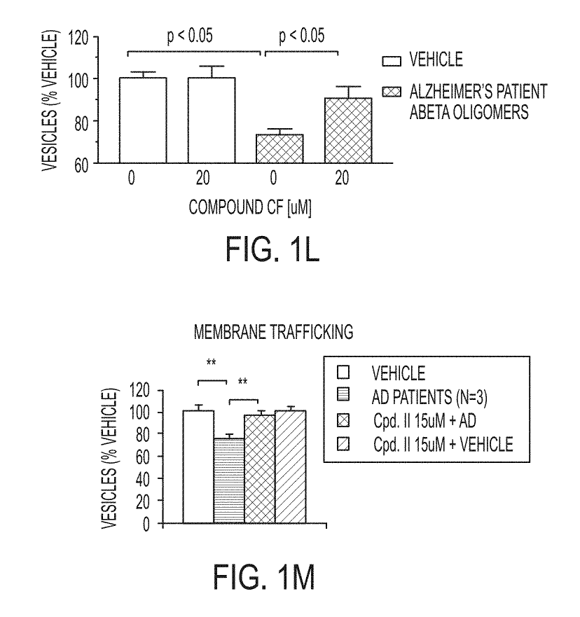

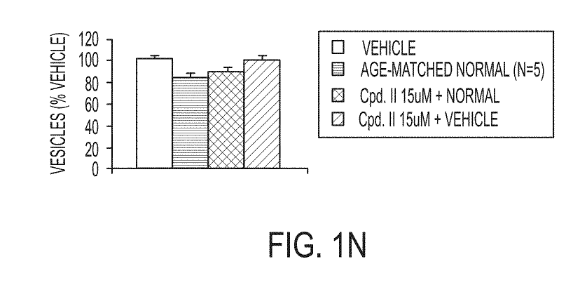

The conditions in which amyloid β may oligomerize in nervous tissue, a milieu of aqueous-soluble proteins with which it may associate, were re-created to identify the more disease-relevant structural state of amyloid β oligomers and fibrils. Aqueous soluble proteins were prepared from rat brain by ultracentrifugation. Specifically, 5 volumes of TBS buffer (20 mM Tris-HCL, pH 7.5, 34 mM NaCl and a complete protease inhibitor cocktail (Santa Cruz) per gram of brain tissue was added to the rat brain tissue on ice. Dounce homogenization was then carried out with a tight-fitting pestle. The homogenized brain tissues were then centrifuged at 150,000×g for 1 hour at 4° C. (40,000 rpm Ty65). The infranatant (between floating myelin and a half cm above the pellet) was then removed and aliquots were frozen at −75° C. The pellets were then resuspended in TBS to the original volume and frozen in aliquots at −75° C. Synthetic, monomeric human amyloid β 1-42 was added to this mixture to provide a final concentration of 1.5 uM amyloid β, and the solution was incubated for 24 hours at 4° C. Centrifugation of the mixture at 5,800 g for 10 minutes was performed to remove fibrillar assemblies and then Immunoprecipitation was performed using 6E10 conjugated agarose spin columns (Pierce Chemical Company) for 24 hours at 4° C. The eluted amyloid 3 oligomers were then subject to MALDI-Tof mass spectroscopic analysis to identify the contents of the sample, FIG. 1.

The amyloid β self-associated in the protein containing solution to form subunit assemblies of 22,599 Da, 5 subunit pentamers and 31,950 Da, 7 subunit, 7mers. Another peak at 49,291 Da may represent 12 subunit, 12mers, although this would not appear to be an accurate molecular weight for amyloid β 12mers. Notably, no peaks are observed at either 4518 Da or 9036 Da which would represent amyloid β monomers and dimers. However, peaks at 9,882 Da and 14,731 Da could represent amyloid β dimers associated with a 786 Da (or 2×393 Da) lipids or proteins and amyloid β trimers associated with 3×393 Da lipids or proteins, respectively. In addition, the presence of a peak at 19,686 Da is indicative of an assembly state possibly involving a trimer complex and a rat amyloid β fragment of 4954 Da. Accordingly these data may reflect the association of small lipids or proteins with dimers and trimers of amyloid β which may direct the assembly of conformational states unique to physiological systems.

### Example 2: Preparation of Beta-Amyloid Oligomers

A solution of 1.5 uM monomeric human amyloid β 1-42 in a mixture of rat brain soluble proteins was incubated for 24 hours at 4° C. as described in Example 1. This solution was then treated with tri-fluoro ethanol (TFE) prior to taking the spectra. In TFE, assembled protein structures and non-covalently bound protein complexes dissociate into denatured proteins, and the peaks associated with assembled oligomers are expected to disappear. The majority of protein peaks observed in Example 1 disappeared including the 9822 Da, 14,731 Da, 31,950 Da, and 49,291 Da peaks identified above. However, an abundant peak is observed at 4518 Da which represents amyloid β monomer peak. A peak at 4954.7 is apparent which may represent a longer abeta fragment similar to amyloid β 1-46. An additional peak is observed at 7086 Da which was not present in the preparation described in Example 1, which may represent amyloid β monomers associated with a 2550 Da covalently bound protein.

### Example 3: Isolation of Beta-Amyloid Oligomers from Human AD Brain Tissue

TBS Soluble Extracts:

Samples of post-mortem brain tissue from human patients characterized via histopathological analysis as Braak Stage V/VI Alzheimer's disease (AD) were obtained from a hospital brain tissue bank. Age and gender matched AD and normal tissue specimens were diluted to 0.15 gm tissue/ml in 20 mM Tris-HCL, 137 mM NaCl, pH 7.6 containing 1 mM EDTA and 1 mg/ml complete protease inhibitor cocktail (Sigma P8340) and homogenized. Ultracentrifugation of the tissue homogenates was performed at 105,000 g for 1 hour in a Beckman Optima XL-80K Ultracentrifuge. The resulting TBS soluble fractions were immunodepleted using protein-A and protein-G agarose columns (Pierce Chemical) and then size fractionated with Amicon Ultra 3, 10 & 100 kDa NMWCO filters (Millipore Corporation).

Immunoprecipitation:

Size fractionated and immunodepleted TBS soluble extracts were concentrated to approximately 200 ul in the appropriate NMWCO Amicon Ultra filters. The concentrated TBS soluble extracts were diluted up to 400 ul with TBS sample buffer (Pierce Chemical) and centrifuged for 10 minutes at 5,800 g to remove fibrils. The resulting supernatant was then immunoprecipitated with 6E10-conjugated agarose beads overnight at 4° C. followed by antigen elution using high osmotic strength Gentle elution buffers (Pierce Chemical) to isolate Abeta containing protein species.

MALDI-Mass Spectrometry:

Immunoisolated beta amyloid was subjected to mass spectroscopic analysis using an Applied Biosystems (ABI) Voyager DE-Pro MALDI-Tof instrument. Samples were analyzed using various matrix types such as α-Cyano-4-hydroxycinnamic acid (CHCA), Sinapic acid (SA), or 6-Aza-2-thiothymine (ATT) depending on the target molecular weight range of the analysis. The instrument was run in a linear-positive ion mode along with a variable extraction delay. Non-accumulated spectra represented 100 shots of a “hot spot” per acquisition while accumulated spectra were represented by 12 separate areas of each spot with 200 laser shots per acquisition.

Data analysis: Data acquisition and analysis was performed using Voyager's Data Explorer software package. Standard processing of the mass spectra included smoothing of the spectrum and baseline subtraction functions in addition to variations in the signal to noise ratio.

ELISA for Ab quantification: Immunoprecipitated TBS soluble fractions were analyzed for both “total” Abeta and Abeta oligomer concentration using a modified sandwich ELISA technique. Briefly, 6E10 and 4G8 coated Nunc MaxiSorp 96-well plates were incubated with Abeta containing samples and then probed with a Biotinylated 4G8 detection antibody. Incubation with Streptavidin-HRP (Rockland) followed by development of a Tetramethyl benzidine (TMB) substrate allowed for colorimetric detection (OD 450) of abeta on a BioTEk Synergy HT plate reader. Monomeric Abeta 1-42 was used for generation of a standard curve and along with GEN 5 software allowed for quantification of Abeta levels in the immuno-precipitated samples.

### Example 4A: Receptor Binding Assays

Compound II interacted with several receptors by blocking the binding or action of their agonists or antagonists. Compound II was tested to see whether it interacted directly with known cellular receptor or signaling proteins. Compound II (1 μM) was tested for its ability to displace binding of known agonists or antagonists of a given human receptor that was overexpressed in cell lines or isolated from tissue. It was also tested for its ability to block downstream signaling induced by agonists or antagonists of a given human receptor. Compound II was tested for action at 100 known receptors, and Compound II showed activity >50% (assay window) at only 5 of these receptors (Table 1E). This indicates that Compound II is highly specific and active at only a small subset of CNS-relevant receptors. It binds the sigma-2 receptor with the highest affinity and is therefore a sigma-2 ligand.

TABLE 1E
Compound II (10 uM) inhibition of binding to known receptors.

| Compound II (10 uM) inhibition of | % Inhibition | |
|---|---|---|
| binding to known receptors | of Control | SEM % control |
| sigma 2 (agonist radioligand) | 89 | 0.6 |
| mu (MOP) (h) (agonist radioligand) | 60 | 1.4 |
| Na+ channel (site 2) (antagonist | 54 | 4.7 |
| radioligand) | | |
| D3 (h) (agonist effect) | 66 | 4.0 |
| alpha 1A (h) (antagonist effect) | 56 | 1.1 |

Using the same protocol, the compounds for which membrane trafficking data are given in Table 5 (below) were tested for recognition of sigma-2 receptor. The results confirmed that these compounds, structurally similar to Compound II, are sigma-2 receptor ligands, i.e., preferentially bind to the sigma-2 receptor. Lastly, compounds similar to Compounds IXa and IXb, such as the compounds of Formulae VIII and IX, also are sigma-2 receptor ligands.

Competitive Radioligand Binding Assay.

Radioligand binding assays for Sigma-1 receptors and Sigma-2 receptors were carried out, by a commercial contract research organization. For Sigma-1 binding, various concentrations of test compounds from 100 μM to 1 nM were used to displace 8 nM [³H](+)pentazocine from endogenous receptors on Jurkat cell membranes (Ganapathy M E et al. 1991, J Pharmacol. Exp. Ther. 289:251-260). 10 μM Haloperidol was used to define non-specific binding. For Sigma-2 receptors various concentrations of test compounds from 100 μM to 1 nM were used to displace 5 nM [³H] 1,3-Di-(2-tolyl)guanidine from endogenous receptors on membranes from rat cerebral cortex in the presence of 300 nM (+)pentazocine to mask Sigma-1 receptors. (Bowen W D, et al. 1993, Mol. Neuropharmcol 3:117-126). 10 μM Haloperidol was used to define non-specific binding. Reactions were terminated by rapid filtration through Whatman GF/C filters using a Brandel 12R cell harvester followed by two washes with ice-cold buffer. Radioactivity on the dried filter discs was measured using a liquid scintillation analyzer (Tri-Carb 2900TR; PerkinElmer Life and Analytical Sciences). The displacement curves were plotted and the Ki values of the test ligands for the receptor subtypes were determined using GraphPad Prism (GraphPad Software Inc., San Diego, Calif.). The percentage specific binding was determined by dividing the difference between total bound (disintegrations per minute) and nonspecific bound (disintegrations per minute) by the total bound (disintegrations per minute).

For known prior art compounds, affinities for Sigma-1 and Sigma-2 receptors were obtained from published studies using cerebral tissue homogenates with [³H](+)pentazocine to measure displacement from Sigma-1 receptors and [³H]1,3-Di-(2-tolyl)guanidine in the presence of 300 nM (+)pentazocine to measure displacement from Sigma-2 receptors. Results are shown in Table 2.

TABLE 2
Sigma-2 and Sigma-1 Receptor Affinity.

| | | Sigma 1 Binding Ki | | Sigma 2 Binding Ki |
|---|---|---|---|---|
| Compound | | (nM) | | (nM) |
| II (three different batches: | | 500 | | 9 |
| racemic mixture, (+) | | 100 | | 52 |
| isomer and (−) isomer) | | 46 | | 63 |
| Compound A | | 47 | | 16 |
| Compound B | | 47 | | 16 |
| Compound E | | 1890 (no substantial | | 16 |
| | | affinity to sigma-1 | | |
| | | receptor) | | |
| Compound P | | 320 | | 110 |
| Compound R′ | | 26 | | 27 |
| Compound S′ | | 31 | | 27 |
| Compound IXa | | 89 | | 21 |
| Compound IXb | | 190 | | 23 |
| Compound W | | 270 | | 120 |
| Compound AC | | 23 | | 240 |
| Compound AE | | 16 | | 35 |
| Compound AF | | 8 | | 110 |
| Compound AH | | 23 | | 50 |
| Compound AI | | 250 | | 130 |
| Compound AL | | 3100 | | 690 |
| Compound AX | | 620 | | 440 |
| Compound AY | | 5 | | 23 |
| Compound AZ | | 34 | | 340 |
| Compound BB | | 0.72 | | 5.2 |
| Compound BC | | 4.2 | | 13 |
| Compound BD | | 2.1 | | 19 |
| Compound BE | | 7.4 | | 14 |
| Compound BH | | 4 | | 7.4 |
| Compound BJ | | 6.2 | | 25 |
| Compound BP | | 53 | | 8.9 |
| Compound BT | | 1 | | 4 |
| Compound CB | | 19 | | 48 |
| Compound CC | | 12 | | 3.9 |
| Compound CD | | 56 | | 2.7 |
| Compound CE | | 33 | | 2.2 |
| Compound CF | | 180 | | 50 |
| Compound CG | | 360 | | 3200 |
| Compound CJ | | 44 | | 810 |
| Compound CL | | 190 | | 5,000 |
| Compound CO | | 130 | | 7,200 |
| Compound CR | | 3.5 | | 16 |
| Compound CS | | 78 | | 85 |
| Compound DH | | 23 | | 8.3 |
| Compound DR | | 330 | | 3,200 |
| | | Sigma 1 | | |
| | | Binding Ki (nM) | | Sigma 2 |
| | Other Compounds | (Type of Activity) | | Binding Ki (nM) |
| | BD1047 | 0.9 | (antagonist) | 47 |
| | DTG | 88 | (agonist) | 35 |
| | Haloperidol | 5 | (antagonist) | 110 |
| | Ifenprodil | 26 | | 4.9 |
| | Mach-14 | 12,900 | | 8 |
| | NE 100 | 1.1 | (antagonist) | 170 |
| | PB 28 | 15 | | 0.8 |
| | PRE-84 | 2.2 | (agonist) | 13,091 |
| | SM-21 | 1050 | (antagonist) | 145 |
| | threo-ifenprodil | 59 | | 0.9 (agonist)* |
| | Sertraline | 8.6 | (antagonist) | 170 |
| | PPBP | 0.8 | (agonist) | 1 |
| | BD1008 | 2.2 | (antagonist) | 8 |
| | Fluvoxamine | 13 | (agonist) | 710 |
| | BD1063 | 8.8 | (antagonist) | 625 |
| | SFK10047 | 597 | (agonist) | 39,740 |

| | | |
|---|---|---|
| siramesine | 19 | 0.19 (agonist) |

*Monassier et al., JPET, 322 (1): 341-350, 2007.

Competitive Radioligand Binding Assay 2.

The affinity of candidate sigma-2 ligand compounds at sigma-1 and sigma-2 receptors was also determined by displacement of different known labeled sigma-2 or sigma-1 ligands. Filtration assays were conducted according the previously published procedure (Xu, et al., 2005). Test compounds were dissolved in N,N-Dimethylformamide (DMF), dimethyl sulfoxide (DMSO) or ethanol and then diluted in 50 mM Tris-HCl pH 7.4 buffer containing 150 mM NaCl and 100 mM EDTA. Membrane homogenates were made from guinea pig brain for sigma-1 binding assay and rat liver for sigma-2 binding assay. Membrane homogenates were diluted with 50 mM Tris-HCl buffer, pH 8.0 and incubated at 25° C. in a total volume of 150 uL in 96 well plates with the radioligand and test compounds with concentrations ranging from 0.1 nM to 10 uM. After incubation was completed, the reactions were terminated by the addition of 150 uL of ice-cold wash buffer (10 mM Tris HCl, 150 mM NaCl, pH 7.4) using a 96 channel transfer pipette (Fisher Scientific, Pittsburgh, Pa.) and the samples harvested and filtered rapidly through 96 well fiber glass filter plate (Millipore, Billerica, Mass.) that had been presoaked with 100 uL of 50 mM Tris-HCl buffer. Each filter was washed four times with 200 uL of ice-cold wash buffer (10 mM Tris-HCl, 150 mM NaCl, pH 7.4). A Wallac 1450 MicroBeta liquid scintillation counter (Perkin Elmer, Boston, Mass.) was used to quantitate the bound radioactivity.

The sigma-1 receptor binding assays were conducted using guinea pig brain membrane homogenates (˜300 ug protein) and ˜5 nM [³H](+)-pentazocine (34.9 Ci/mmol, Perkin Elmer, Boston, Mass.), incubation time was 90 min at room temperature. Nonspecific binding was determined from samples that contained 10 μM of cold haloperidol.

The sigma-2 receptor binding assays were conducted using rat liver membrane homogenates (˜300 ug protein) and ˜2 nM sigma-2 highly selective radioligand [³H]RHM-1 only (no other blockers) (America Radiolabeled Chemicals Inc. St. Louis, Mo.), ˜10 nM [³H]DTG (58.1 Ci/mmol, Perkin Elmer, Boston, Mass.) or ˜10 nM [³H]Haloperidol (America Radiolabeled Chemicals Inc., St. Louis, Mo.) in the presence of 1 uM (+)-pentazocine to block sigma-1 sites, incubation times were 6 minutes for [³H]RHM-1, 120 min for [³H]DTG and [³H]haloperidol at room temperature. Nonspecific binding was determined from samples that contained 10 uM of cold haloperidol.

Data from the competitive inhibition experiments were modeled using nonlinear regression analysis to determine the concentration of inhibitor that inhibits 50% of the specific binding of the radioligand (IC₅₀ value). The binding affinity, Ki values was calculated using the method of Cheng and Prusoff. The Kd value used for [³H](+)-pentazocine in guinea pig brain was 7.89 nM, for [³H]RHM-1 and [³H]DTG in rat liver were 0.66 nM and 30.73 nM respectively. The standard compound haloperidol was used for quality assurance. Affinity data at sigma-1 and sigma-2 receptor for compound IXa,IXb and compound II are shown in Table 3. Therefore, any sigma-2 receptor binding assay known in the art can be employed to determine the Ki or IC50 of a candidate compound.

TABLE 3
Sigma-2 and Sigma-1 Receptor Affinity for Candidate
Sigma-2 Ligands in Competitive Radioligand Binding Assay 2.

| | | Sigma-1 | Sigma-2 |
|---|---|---|---|
| | | (Ki, nM) ± mean | (Ki, nM) ± mean |
| No | Compound | SEM | SEM |
| 1 | IXa, IXb | 6.37 ± 0.81 | 30.8 ± 2.3 |
| 2 | II | 108.1 ± 19.9 | 59.7 ± 10.4 |

### Example 4B. Anti-Receptor Antibody-Mediated Reduction of Oligomer Binding to Receptor

As described herein, progesterone receptor membrane component 1 (PGRMC1) was recently identified as the critical 25 kDa component of sigma-2 receptor activity by Xu et al. 2011. Specifically, PGRMC1 was identified in rat liver by use of a photoaffinity probe WC-21, which irreversibly labels sigma-2 receptors in rat liver. Xu et al. Identification of the PGRMC1 protein complex as the putative sigma-2 receptor binding site. Nature Communications 2, article number 380, Jul. 5, 2011, incorporated herein by reference. Therefore, monoclonal antibodies specific for various C-terminal or N-terminal amino acid sequences of human PGRMC1 were employed in these experiments.

The ability of receptor antibodies to affect Abeta oligomer binding are tested using the following general assay procedure. Positive control: 6E10 antibody (Covance) (recognizes the N-terminus of all Abeta species, and will reduce Abeta binding to neurons by virtue of high affinity binding to oligomer in solution prior to receptor binding). Neurons in culture were prepared as for the membrane trafficking assay. Negative control: non-immune IgG. Methods:
1. Aspirate plate to 10 uL volume
2. Add 30 ul of specified concentration of receptor monoclonal antibody (mAb), 6E10 or non-immune IgG for 30 minutes at 37° C.
3. Add 10 uL of Abeta oligomers at specified concentration; incubate for 1 hour at 37° C.
4. Fix and wash plate 3 times with PBS
5. Block plate for one hour using blocking compound (1 L PBS, 50 mL normal goat serum, 50 mL 10% TritonX)
6. Aspirate plate to 10 uL
7. Add 25 uL primary detection antibodies 4G8 and MAP2; seal and refrigerated over night.
8. Wash plate 3 times with blocking compound (aspirate to 10 uL, wash with 60 uL block)
9. Add 25 uL of secondary antibodies (Alexa fluor 647 goat anti mouse, Alexa flour 488 goat anti rabbit, Alexa flour 646 goat anti chicken); let sit for an hour at room temperature.
10. Aspirate plate to 10 uL, wash with 60 uL PBS, aspirate plate to 10 uL add 60 uL Dapi, aspirate plate to 10 uL wash with 60 uL PBS.
11. Cover with aluminum seal
12. Scan and analyze images with proprietary Cellomics Arrayscan protocol.

Specific Antibody Blocking Experiment.

Antibodies recognizing the synthetic peptide: C-EPKDESARKND (SEQ ID NO: 7), corresponding to C terminal amino acids 185-195 of human PGRMC1 (#EB07207, Everest Biosciences), or residues 1-46 at the N-terminus of human PGRMC1 protein (MAAEDVVATGADPSDLESGGLLHEIFTSPLNLLLLGLCIFLLYKI (SEQ ID NO: 9), #sc-98680, Santa Cruz), or nonimmune control IgG were employed in these experiments. Each antibody was applied to neurons for 30 minutes at 38 nM (5×), 58 nM (7.5×) or 77 nM (10×) Final Assay Concentrations.

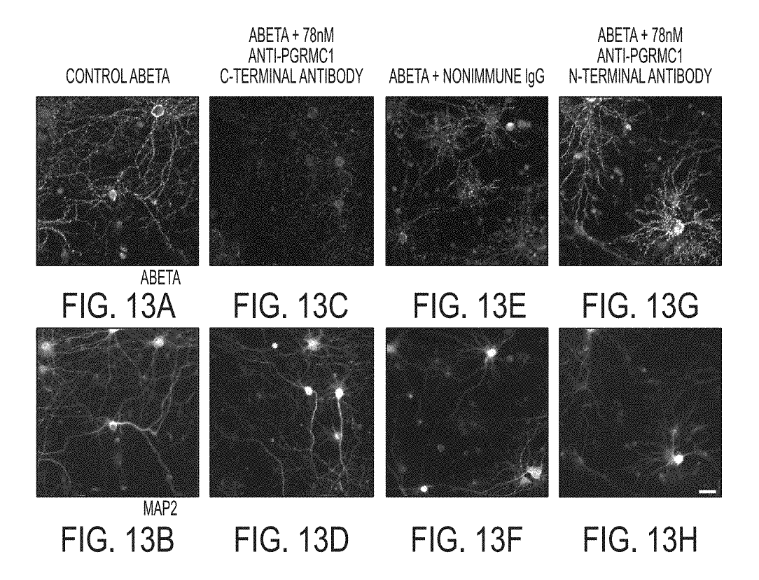

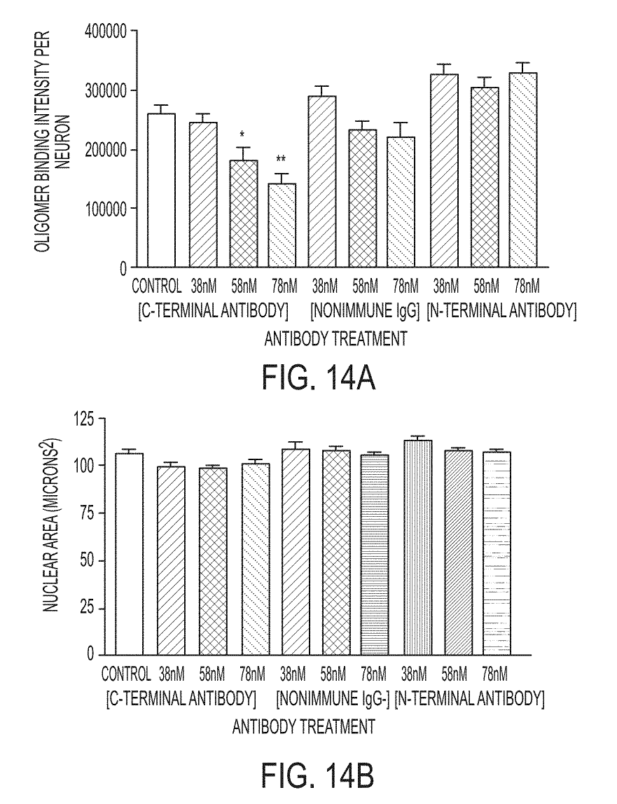

Abeta 1-42 oligomers were then added at 500 nM total Abeta concentration and allowed to bind to neurons for an additional 15 minutes. Cultures were then fixed and immunolabeled for bound Abeta species using 6E10 antibody. Oligomers bound to postsynaptic membranes in a characteristic punctuate pattern were quantified via automated image processing. Neurons were identified via MAP2 immunolabeling. Quantitative measures of neuron health such as the average nuclear area were quantified via image processing and results are shown in FIGS. 13A to 13H.

There was a dose-dependent reduction of the intensity of Abeta oligomer binding with neurons incubated with C-terminal antibody (FIG. 13C), compared to control Abeta (FIG. 13A), but not with N-terminal antibody (FIG. 13G) or nonimmune IgG (FIG. 13E). This reduction in intensity appears to consist of a reduction in the number and area of oligomer-positive puncta. Whether this reduction in oligomer binding intensity is due to competition for binding epitopes between oligomer and antibody, or an antibody-mediated reduction in surface PGRMC1 protein expression is not known. In some embodiments, anti-sigma-2 receptor antibodies and anti-PGRMC1 antibodies that block binding between soluble Abeta oligomers and a sigma-2 receptor are considered to be sigma-2 receptor antagonist compounds.

### Example 5: Memory Loss in Transgenic Mice: Morris Swim Test

Compound II was tested to determine if it could reverse memory loss seen in older transgenic mouse models of Alzheimer's disease, where oligomers build up with age. For this study hAPP mice expressing human APP751 Swedish (670/671) and London (717) mutations under the control of the murine Thy-1 promoter were chosen. These mice exhibit an age-dependent increase in the amount of Abeta, with plaques developing beginning at 3-6 months and exhibit established cognitive deficits by 8 month of age. In this study, rather than preventing deficits from occurring, deficits that were already established were treated. These studies were performed pursuant to a service contract by scientists who were blind to the experimental conditions. The compound was infused at 0.5 and 0.1 mg/kg/day for one month in 8 month old female mice via subcutaneous minipump and cognitive performance was tested in the Morris water maze, a test of hippocampal-based spatial learning and memory. This mouse model does not exhibit neuronal loss so the restoration of memory cannot be attributed to aversion of apoptosis.

The swim speed was analyzed as part of the Morris measurements to determine if there were any motor or motivational deficits. Our vehicle is a 5% DMSO/5% Solutol, 90% saline mixture. The transgenic animals treated were with a low dose (0.1 mg/kg/day) and a high dose (0.5 mg/kg/day) of compound II. The average of three daily trials on each of four consecutive days were determined. We could detect no significant motor deficits or abnormal behaviors of any kind, and lost only one animal from the transgenic vehicle group during the course of the study, below expected mortality levels at this age. In addition we maintained a sentinel group of animals that had periodic blood draws to monitor plasma levels of compound, and these showed very little change from the plasma levels seen in the preliminary PK study.

Escape latency measurements from the Morris water test were taken. On the second day of testing a significant difference between wild-type and transgenic animals was observed, with the wild-type learning faster than transgenics. On this day a significant improvement in transgenic performance at the higher compound dose vs. vehicle was also observed. Therefore, it is concluded that Compound II administered at 0.5 mg/kg/day is capable of improving cognitive performance in transgenic models of AD.

Abeta 42 oligomers caused an 18% decrease in synapse number; 100% of this loss is eliminated by Compound II and its enantiomer. Similar to compound II, several other sigma-2 receptor antagonists also block synapse loss. Known prior art Sigma-2 receptor ligands NE-100 and haloperidol completely eliminated synapse loss, while SM-21, a selective Sigma 1 ligand was only weakly active in eliminating synapse loss (20% recovery).

A mixture of Compounds IXa and IXb was also tested using a similar assay. The mixture of compounds IXa and IXb (1 mg/kg/day, N=8 or 10 mg/kg/day, N=8) or vehicle (5% DMSO/5% Solutol/90% saline, N=15) was systemically administered via subcutaneous dosing (Alzet minipump) to 9 month old male hAPPSL transgenic mice (N=8) or nontransgenic littermates (N=6) for 20 days and spatial learning and memory of these mice were evaluated in the Morris water maze. During the final four days of treatment, mice were tested to find the hidden platform in three trials/day. A computerized tracking system automatically quantified escape latency, or swim length.

There was no significant difference in the performance of transgenic animals vs. nontransgenic animals on any day of the test (analysis restricted to these 2 groups; two-way (genotype and time) ANOVA with repeated measures followed by Bonferroni's post-hoc test). A similar analysis, restricted to the transgenic groups (treatment and time), showed that transgenic animals treated with 10 mg/kg/day of a mixture of Compounds IXa and IXb performed significantly better than vehicle-treated transgenic animals on the second and fourth day of testing (p<0.05, analyzed by Student's t-test). Nontransgenic vehicle-treated animals performed significantly better than transgenic vehicle-treated animals on the first and second day of testing. Treatment with the mixture of compounds IXa and IXb significantly improved transgenic animal performance compared to vehicle treatment on the first (both doses) second (10 mg/kg/day dose) and fourth (10 mg/kg/day dose) days of testing (p<0.05; swim length).

This demonstrates that a mixture of compounds IXa and IXb is capable of reversing established behavioral deficits in learning and memory in aged transgenic animals in a dose-dependent manner.

### Example 6: Inhibition of Abeta Oligomer Effect on Neuronal Cells in Membrane Trafficking Assay

Sigma-2 ligands selected from Table 2 above were tested for their ability to inhibit an amyloid beta effect on the cells. The sigma-2 ligands were able to inhibit the amyloid beta effect as measured by a membrane trafficking/exocytosis assay (MTT assay). The results are indicated in Table 5 below. The rationale for this assay was as follows:

Since synaptic and memory deficits, and not widespread cell death, predominate at the earliest stages of Alzheimer's disease, assays that measure these changes are particularly well suited to discovering small molecule inhibitors of oligomer activity. The MTT assay is frequently used as a measure of toxicity in cultures. Yellow tetrazolium salts are endocytosed by cells and reduced to insoluble purple formazan in the endosomal pathway. The level of purple formazan is a reflection of the number of actively metabolizing cells in culture, and reduction in the amount of formazan is taken as a measure of cell death or metabolic toxicity in culture. When observed through a microscope, the purple formazan is first visible in intracellular vesicles that fill the cell. Over time, the vesicles are exocytosed and the formazan precipitates as needle-shaped crystals on the outer surface of the plasma membrane as the insoluble formazan is exposed to the aqueous media environment. Liu and Schubert ('97) discovered that cells respond to sublethal levels of Abeta oligomers by selectively accelerating the exocytosis rate of reduced formazan, while leaving endocytosis rate unaffected. The inventors have replicated these observations in mature primary neurons in culture and quantified these morphological shifts via automated microscopy and image processing. Under these circumstances, there is no overall change in the total amount of reduced formazan, simply a shift in its morphology reflective of changes in rate of its formation and/or expulsion from the cell. The inventors have confirmed previous findings that this assay is sensitive to low levels of oligomers that do not cause cell death (Liu and Schubert '04, Hong et al., '07). Indeed, low amounts of oligomers that lead to inhibition of LTP do not lead to cell death (Tong et al., '04) and are not expected to change total amounts of formazan in culture (or in brain slices).

Evidence adduced by other investigators suggests that Abeta oligomer-mediated reduction in neuronal surface receptor expression mediated by membrane trafficking is the basis for oligomer inhibition of electrophysiological measures of synaptic plasticity (LTP) and thus learning and memory (Kamenetz et al., '03, Hseih et al., '06). Measuring membrane trafficking rate changes induced by oligomers via formazan morphological shifts has been used in cell lines to discover Abeta oligomer-blocking drugs (Maezawa et al., '06, Liu and Schubert '97, '04, '06, Rana et al., '09, Hong et al., '08) that lower Abeta brain levels in rodents in vivo (Hong et al., '09). Similar procedures for exocytosis assays/MTT assays can be found in the literature. See e.g., Liu Y, et. al., Detecting bioactive amyloid beta peptide species in Alzheimer's disease. J Neurochem. 2004 November; 91(3):648-56; Liu Y, and Schubert D. “Cytotoxic amyloid peptides inhibit cellular 3-(4,5-dimethylthiazol-2-yl)-2,5-diphenyltetrazolium bromide (MTT) reduction by enhancing MTT formazan exocytosis.” J Neurochem. 1997 December; 69(6):2285-93; and Liu Y, and Schubert D. “Treating Alzheimer's disease by inactivating bioactive amyloid beta peptide” Curr. Alzheimer Res. 2006 April; 3(2):129-35. Therefore the approach is valid.

The present exocytosis assay was adapted for use with mature primary neuronal cultures grown for 3 weeks in vitro. See WO/2011/106785, incorporated by reference in its entirety. Abeta oligomers cause a dose-dependent decrease in the amount of intracellular vesicles (puncta) filled with reduced purple formazan as measured via image processing using a Cellomics VTI automated microscopy system. Compare for example FIG. 1A (a photomicrograph for a cultured neuronal cell exposed to vehicle alone showing vesicles filled with formazan) with FIG. 1B (a photomicrograph of a neuronal cell exposed to vehicle plus Abeta oligomer showing considerably fewer vesicles filled with formazan and instead exocytosed formazan which when encountering the extracellular environment precipitates into crystals). Increasing the amount of Abeta oligomers eventually results in overt toxicity. Thus, the concentration of neuroactive Abeta oligomers used in the assay is much lower than that causing cell death. The inventors confirmed that the assay is operative by showing that the effects of Abeta oligomer are blocked upon addition of anti-Abeta antibody but antibody alone has no effect on its own (data not shown). When configured in this manner, the assay is able to detect compounds that inhibit nonlethal effects of Abeta oligomer whether these compounds act via disruption of oligomers, inhibition of oligomer binding to neurons, or counteraction of signal transduction mechanisms of action initiated by oligomer binding.

The methods used to generate the results were as follows in the Membrane Trafficking/Exocytosis (MTT) assay.

Primary hippocampal neurons from E18 Sprague-Dawley rat embryos were plated at optimized concentrations in 384 well plates in NB media (Invitrogen). Neurons were maintained in cultures for 3 weeks, with twice weekly feeding of NB media with N₂ supplement (Invitrogen). These neurons express the full complement of synaptic proteins characteristic of neurons in the mature brain, and exhibit a complex network of activity-dependent electrical signaling. Neurons and glia in such cultures have molecular signaling networks exhibiting excellent registration with intact brain circuitry, and for this reason have been used for over two decades as a model system for learning and memory (See e.g. Kaech S, Banker G. Culturing hippocampal neurons. Nat Protoc. 2006; 1(5):2406-15. Epub 2007 Jan. 11; See also Craig A M, Graf E R, Linhoff M W. How to build a central synapse: clues from cell culture. Trends Neurosci. 2006 January; 29(1):8-20. Epub 2005 Dec. 7. Review).

A test compound was added to cells at concentrations ranging from 100 uM to 0.001 nM followed by addition of vehicle or Abeta oligomer preparations (3 μM total Abeta protein concentration), and incubated for 1 to 24 hr at 37° C. in 5% CO₂. MTT reagent (3-(4,5-dimethylthizaol-2yl)-2,5diphenyl tetrazolium bromide) (Roche Molecular Biochemicals) was reconstituted in phosphate buffered saline to 5 mg/mL. 10 μL of MTT labeling reagent is added to each well and incubated at 37° C. for 1 h, then imaged. Exocytosis was assessed by automated microscopy and image processing to quantify the amount of endocytosed and exocytosed formazan.

Each assay plate was formatted so that compounds are tested with and without Abeta oligomer on each plate. This design eliminates toxic or metabolically active compounds early on in the screening cascade (at the level of the primary screen). Reduced formazan was first visible in intracellular vesicles. Eventual formazan exocytosis was accelerated via Abeta oligomers. FIGS. 1A and 1B are examples of photomicrographs of neurons, the first of intracellular vesicles where formazan is first seen and the second of a neuron covered with insoluble purple dye that has been extruded via exocytosis. The dye precipitated in the aqueous environment of the culture and formed needle-shaped crystals on the surface of the neuron.

In the presence of 15 micromolar Compound II, the membrane traffic changes captured in FIG. 1B are blocked (see FIG. 1C) and the cell in FIG. 1C is indistinguishable from a vehicle-treated neuron. Furthermore, this effect of Compound II appears to be independent of whether Compound II is added before or after exposure of the cells to Abeta oligomer, which indicates a therapeutic as well as a prophylactic effect. See FIG. 1D, a plot (dose response curve) of membrane trafficking changes expressed as percent vesicles seen on image processing versus the log of Compound II concentration in the presence of various amounts of Abeta oligomer added before (FIG. 1D) or after (FIG. 1E) addition of various amounts of Compounds II or a mixture of IXa,IXb. Abeta oligomer alone is indicated by the circle at bottom left of FIGS. 1D and 1E. Vehicle alone is indicated by filled squares. When added before oligomers (prevention mode) compound II blocks oligomer effects with EC₅₀=2.2 uM and compound IXa,IXb blocks oligomer effects with EC₅₀=4.9 uM. When added after oligomers (treatment mode), compound II blocks oligomer effects with EC₅₀=4.1 uM and compound IXa,IXb blocks oligomer effects with EC₅₀=2.0 uM. In either case, Compound II or a mixture of IXa,IXb each blocks membrane trafficking effects of Abeta oligomer seen in this assay. Ascending doses of selective, high affinity sigma-2 receptor antagonist compounds from two structurally distinct series (II and IXa,IXb) stop oligomer effects and make the cultures look more like vehicle-treated cultures.

Based on these results, selective, high affinity sigma-2 receptor antagonist compounds as disclosed herein are that effective for inhibiting Abeta oligomer toxicity are promising as therapeutic and (in very early stages) prophylactic modalities for amyloid beta oligomer toxicity related cognitive decline such as that seen in Alzheimer's disease. Saturable competitive binding to Abeta oligomers could not be demonstrated in these experiments because toxicity limits the upper concentrations.

Synthetic Abeta oligomers were dosed in the membrane trafficking assay as seen in the FIGS. 1F and 1G, where it exhibited an EC50 of 820 nM. Each concentration of Abeta was tested against several concentrations of each selective high affinity sigma-2 receptor antagonist compound drug candidates II and IXa,IX, which each caused a rightward shift in the EC₅₀ by almost two orders of magnitude. When the data were fitted to classical linear and non linear models, the data were linear with a Schild analysis (Hill slope nH of 1), which indicates that the sigma-2 receptor compound compounds exhibit true pharmacological competition between oligomers and compound for targets that mediate membrane trafficking. Abeta oligomers derived from Alzheimer's patient's brains were dosed against these compounds as shown in FIGS. 1J and 1K, and also a rightward shift was also exhibited by compound exposure. Specifically, at effective doses, compound II and IXa,IXb exhibit pharmacological competition with both synthetic (FIG. 1F,G, Schild slope=−0.75, −0.51) and human Alzheimer's patient-derived (FIG. 1J, 1K) oligomers. The net effect of this is that these two selective high affinity sigma-2 receptor antagonist compound candidate drugs effectively make Abeta oligomers less synaptotoxic, and these are the only therapeutics to date we're aware of that have demonstrated this property. Without being bound by theory, the simplest possible mechanism of action is that the sigma-2 receptor compounds act as competitive receptor antagonists.

In a related experiment, a rightward shift in dose response curves (% vesicles against Abeta oligomer concentration) was observed based on the effect of 0 or 20 μM of Compound II enantiomers: see Table 4 below. The (+) enantiomer was shown to be more effective at higher concentrations of Abeta oligomer.

TABLE 4

| | | | | EC 50 in |
|---|---|---|---|---|
| | EC50 against | | | screening assay |
| | Abeta at | EC50 against | | using single |
| | Concen- | Abeta at | Curve | concentration |
| | tration | Concentration | shift | of Abeta |
| Compound | 0 uM | 20 μM | (fold) | oligomer |
| (+) enantiomer | 1.19 | 1.46 | 2.86 | 5.6 uM |
| of Cpd II | | | | |
| (−) enantiomer | 1.05 | 2.82 | 1.22 | 10.9 uM |
| of Cpd II | | | | |

As shown in Table 4 above, the rightward shift in the dose response curve of % vesicles against Abeta oligomer concentration for 20 μM of enantiomer versus 0 μM of enantiomer (i.e., Abeta oligomer alone) is significantly more pronounced for the (+) enantiomer at higher concentrations of Abeta oligomer.

### Experimental Controls:

Abeta 1-42 oligomers made according to published methods were used as positive controls. [See e.g. Dahlgren et al., “Oligomeric and fibrillar species of amyloid-beta peptides differentially affect neuronal viability” J Biol Chem. 2002 Aug. 30; 277(35):32046-53. Epub 2002 Jun. 10.; LeVine H 3rd. “Alzheimer's beta-peptide oligomer formation at physiologic concentrations” Anal Biochem. 2004 Dec. 1; 335(1):81-90; Shrestha et. al, “Amyloid beta peptide adversely affects spine number and motility in hippocampal neurons” Mol Cell Neurosci. 2006 November; 33(3):274-82. Epub 2006 Sep. 8; Puzzo et al., “Amyloid-beta peptide inhibits activation of the nitric oxide/cGMP/cAMP-responsive element-binding protein pathway during hippocampal synaptic plasticity” J Neurosci. 2005 Jul. 20; 25(29):6887-97; Barghorn et al., “Globular amyloid beta-peptide oligomer—a homogenous and stable neuropathological protein in Alzheimer's disease” J Neurochem. 2005 November; 95(3):834-47. Epub 2005 Aug. 31; Johansson et al., Physiochemical characterization of the Alzheimer's disease-related peptides A beta 1-42 Arctic and A beta 1-42 wt. FEBS J. 2006 June; 2 73(12):2618-30] as well as brain-derived Abeta oligomers (See e.g. Walsh et al., Naturally secreted oligomers of amyloid beta protein potently inhibit hippocampal long-term potentiation in vivo. Nature (2002). 416, 535-539; Lesne et al., A specific amyloid-beta protein assembly in the brain impairs memory. Nature. 2006 Mar. 16; 440(7082):352-7; Shankar et al, Amyloid-beta protein dimers isolated directly from Alzheimer's brains impair synaptic plasticity and memory. Nat Med. 2008 August; 14(8):837-42. Epub 2008 Jun. 22). It should be noted that any Abeta oligomer preparation can be used in this assay or as a control, including preparations described in the patent literature, cited above and incorporated by reference in their entirety.

Various different Abeta oligomer preparations were demonstrated to cause an Abeta effect in the membrane trafficking assay, including notably oligomer preparations isolated from the brain of Alzheimer's disease patients.

Oligomers were isolated from postmortem human hippocampus or prefrontal cortex without the use of detergents and inhibited membrane trafficking in a dose-dependent manner with a Kd of 6 pMolar. Human Alzheimer's disease patient-derived Abeta oligomers (137 pM, second bar FIG. 1J) produce a statistically significant inhibition of membrane trafficking compared to vehicle (first bar, FIG. 1J). Compound II (third bar) eliminates the membrane trafficking deficits induced by AD brain-derived Abeta oligomers, but does not affect trafficking when dosed in the absence of Abeta (fourth, hatched, bar). The data are averaged from 3 experiments (n=3).

Although potencies of various Abeta oligomer preparations differ (for example native Alzheimer's isolates are more potent than any of the synthetic preparations tested—data not shown), the results are qualitatively the same: pathologies mediated by oligomers are countered by compositions of the invention comprising a sigma-2 receptor antagonist compound.

In the presence of Compound II at an excess (15 uM, third bar FIG. 1J) shown in the black bar, oligomer-induced membrane trafficking deficits are completely eliminated. Compound II has no significant effect on membrane trafficking when dosed on its own (black diagonal bar, FIG. 1J).

In contrast, oligomers isolated from the same postmortem brain areas taken from cognitively normal age-matched individuals are generally present at lower concentrations per gram weight of tissue, 90 pM as opposed to 137 pM, (FIG. 1K, second bar), and do not produce significant deficits in membrane trafficking vs. vehicle (FIG. 1K, first bar). Under these conditions, Compound II has no effect when dosed with oligomers or alone (FIG. 1K, third and 4^{th} bar respectively. Again, data are averaged (n=3 except for second bar, wherein n=5).

Negative controls include vehicle-treated neurons as well as neurons treated with supraphysiological, 28 μM, concentrations of memantine. Memantine produces 50% inhibition of oligomer effects at this dose. These controls, on each plate, serve as normalization tools to calibrate assay performance on a plate-by-plate basis.

### Primary Neuronal Cultures

Optimal cell density is determined based on cellular response to Abeta oligomers using the exocytosis assay as a readout, and immunohistochemical analysis of the relative proportion of glia to neurons in the cultures. Cultures are monitored on a weekly basis with immunohistochemistry and image processing-based quantification to monitor the percentage of the cultures that are neurons vs. glia (Glial cells). Cultures containing more than 20% glia (positive for GFAP) vs. neurons (staining positively with (chicken polyclonal) antibodies (Millipore) directed against MAP2 at 1:5000 (concentration variable)) at the screening age of 21 days in vitro (21 DIV) are rejected.

### Abeta Oligomer Preparations

Human amyloid peptide 1-42 was obtained from a number of commercial vendors such as California Peptide, with lot-choice contingent upon quality control analysis. Quality controls of oligomer preparations consist of Westerns to determine oligomer size ranges and relative concentrations, and the MTT assay to confirm exocytosis acceleration without toxicity. Toxicity was monitored in each image-based assay via quantification of nuclear morphology visualized with the DNA binding blue dye DAPI (Invitrogen). Nuclei that are fragmented are considered to be in late stage apoptosis (Majno and Joris '95) and the test would be rejected. Peptide lots producing unusual peptide size ranges or significant toxicity at a standard 1.5 μM concentration on neurons would also be rejected.

Plate-based controls—The assay optimization was considered complete when reformatted plates achieve a minimum of statistically significant two-fold separation between vehicle and Abeta oligomer-treated neurons (p<0.01, Student's t-test, unequal variance) on a routine basis, with no more than 10% CV between plates.

### Statistical Software and Analysis:

Data handling and analysis were accomplished by Cellomics VTI image analysis software and STORE automated database software. Because of the low dynamic range and neuronal well-to-well variability after three weeks in culture, statistical comparisons are made via pairwise Tukey-Kramer analysis to determine the significance of the separation between compound+Abeta oligomers from Abeta alone, and between compound alone from vehicle. The ability of mature primary neurons to more closely approximate the electrophysiologically mediated signal transduction network of the adult brain justifies this screening strategy. Power analysis was set for a number of replicate screening wells that minimized false negatives (e.g. N=4). Test compounds of the invention significantly reverse the effects of Abeta oligomers on membrane trafficking but do not affect neuronal metabolism themselves.

Selected compounds within the Formulas of the invention, including Formulas III-IV, VIIIq and VIIIo as indicated in the Table 5 below were dosed in the MTT assay described herein prior to Abeta oligomer addition and were shown to block the Abeta oligomer-induced membrane trafficking deficits with the indicated EC₅₀. Specifically, these results indicate that compounds block/abate the activity/effect of Abeta oligomer on membrane trafficking of neuron cells at micromolar concentrations.

TABLE 5
Sigma-2 Receptor Ligands and Ability to inhibit amyloid
oligomer effects on membrane trafficking:

| | EC₅₀ in inhibiting | | |
|---|---|---|---|
| | amyloid beta effect in | | |
| | Cell Measured by | | Max Inhibition |
| | Membrane Trafficking | | of Abeta |
| | Assay | | (%) |
| Sigma-2 Receptor Ligand | | | |
| Compound II | 2.2 | uM | 68 |
| (three different batches: | 5.6 | uM | (78) |
| racemic mixture, (+) | 10.9 | uM | (64) |
| isomer and (−) isomer) | | | |
| Compound A | 3.4 | uM | 78 |
| Compound B | 5.5 | uM | 84 |
| Compound C | 5.4 | uM | 93 |
| Compound D | 8.9 | uM | 58 |
| Compound E | 8.2 | uM | 68 |
| Compound F | 2.6 | uM | 69 |
| Compound G | 5.8 | uM | 92 |
| Compound H | 2.2 | uM | — |
| Compound I | 3.4 | uM | 100 |
| Compound J | 3.9 | uM | 97 |
| Compound K | 14 | uM | 13 |
| Compound L | 2.4 | uM | 34 |
| Compound M | 0.6 | uM | 60 |
| Compound N | 5.2 | uM | 46 |
| Compound O | 2.7 | uM | 27 |
| Compound P | 20.0 | uM | 43 |
| | (19.5 | uM) | (73) |
| Compound Q | 0.5 | uM | 82 |
| Compound R | 6.7 | μM | 55 |
| Compound R′ | 39 | μM (inactive) | 38 |
| Compound S | 5.4 | μM | 100 |
| Compound S′ | >30 | μM (inactive) | 0 |
| Compound T | 7.7 | μM | 45 |
| Compound IXa | 4.9 | μM | 76 |
| Compound IXb | 6.9 | μM | 97 |
| Compound AC | 2.4 | μM | — |
| Compound AD | 0.7 | μM | — |
| Compound AG | 6.1 | μM | — |
| Compound BA | <1.0 | μM | — |
| Compound BT | 0.4 | μM | — |
| Compound BY | 0.8 | μM | — |
| Compound CA | 1.9 | μM | — |
| Compound CB | 18.2 | μM | — |
| Compound CR | 1 | μM | — |
| Compound CS | 6.9 | μM | — |
| Compound CT | 3 | μM | — |
| Compound CW | 2.5 | μM | — |
| Compound CX | 1.3 | μM | — |
| Compound CY | 14 | μM | — |
| Compound DE | >20.0 | μM | — |

| | | |
|---|---|---|
| Compound | | |
| BD1047 | 11 | 100 |
| DTG | N/A | 23 |
| Haloperidol | 6.2 | 100 |
| Ifenprodil | 1.3 | 38 |
| Mach-14 | 1 | 80 |
| NE 100 | 9.1 | 98 |
| PB 28 | 2.2 | 84 |
| PRE-84 | N/A | 38 |
| SM-21 | 11 | 65 |
| threo-ifenprodil | N/A | 39 |

The compounds in Table 5 were shown to block the Abeta oligomer-induced acceleration of exocytosis with the indicated EC₅₀. Accordingly, the compounds in Table 5 significantly blocked Abeta oligomer-mediated changes in membrane trafficking. These results indicate that compounds block/abate the activity/effect of Abeta oligomer on neuron cells and that sigma-2 ligands can be used to block the Abeta oligomer induced membrane trafficking abnormalities.

Table 6A shows membrane trafficking EC₅₀ data for certain additional compounds.

TABLE 6A
Additional Membrane Trafficking Data.

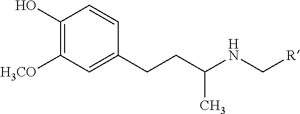

| | | Membrane |
|---|---|---|
| | | Trafficking |
| Entry | R′ | EC₅₀ (μM) |
| 1 | 4-CF3—Ph— | 2.2 |
| 2 | 4-Cl—Ph— | 12 |
| 3 | Ph— | 20 |
| 4 | isoBu— | >30 |
| 5 | H— | 30 |

### Correlation Between Trafficking Assay and Sigma Receptor Binding Data

Binding affinity of compounds to Sigma-1 or Sigma-2 receptors (from Table 2) and their EC₅₀ and maximum effect in the membrane trafficking assay (from Table 5) were analyzed using Spotfire software to discover correlations between receptor binding and assay activity. The goodness of fit between the log of the EC₅₀ in the MTTX was calculated vs Log sigma-1 and sigma-2 binding Ki, between the max inhibition of Abeta vs Log sigma-1 and sigma-2 binding Ki and for Log EC₅₀ in the trafficking assay vs the ratio of sigma-1 binding Ki to sigma-2 binding Ki. All calculations were performed for 4 groupings of compounds: A) bolded compound plus known prior art compounds listed in Table 5, B) only bolded compounds from Table 5, C) only reference compounds from Table 5, and D) All compounds from Group A except sigma-1 antagonists (NE-100, Haloperidol, BD1047, SM21).

TABLE 6B
Correlation between sigma binding affinity and trafficking assay activity.

| | | | | | R² (P |
|---|---|---|---|---|---|
| | | | R² (P Value) | R² (P | Value) |
| | | R² (P value) | Table 5 | Value) | W/O |
| | | All 19 | Bolded | 10 Ref | sigma-1 |
| Y Axis | X Axis | compounds | Compounds | Compounds | antagonists |
| MTTX | Vs Log | 0.06 (ns) | 0.01 (ns) | 0.02 (ns) | 0.02 (ns) |
| log (EC50) | S1 Ki | | | | |
| | Vs Log | 0.15 (ns) | 0.70 (<0.001) | 0.27 (ns) | 0.78 (<0.001) |
| | S2 Ki | | | | |
| Max | Vs Log | 0.00 (ns) | 0.14 (ns) | 0.06 (ns) | 0.00 (ns) |
| inhibition | S1 Ki | | | | |
| of | Vs Log | 0.00 (ns) | 0.00 (ns) | 0.00 (ns) | 0.00 (ns) |
| Abeta | S2 Ki | | | | |
| MTTX | S1/S2 | 0.01 (ns) | 0.11 (ns) | 0.04 (ns) | 0.01 (ns) |
| log (EC50) | | | | | |

As can be seen in Table 6B, the highest correlation for the bolded compounds alone, was between their Log (EC₅₀) in the MTTX assay and the Log (Kᵢ) for Sigma-2 binding (R²=0.70, P<0.001). No other comparisons were statistically significant. This same correlation was not statistically significant when the reference compounds were added into the analysis (R²=0.15, P>0.05) and the reference compounds alone did not show a statistically significant correlation between these parameters ((R²=0.27, P>0.05).

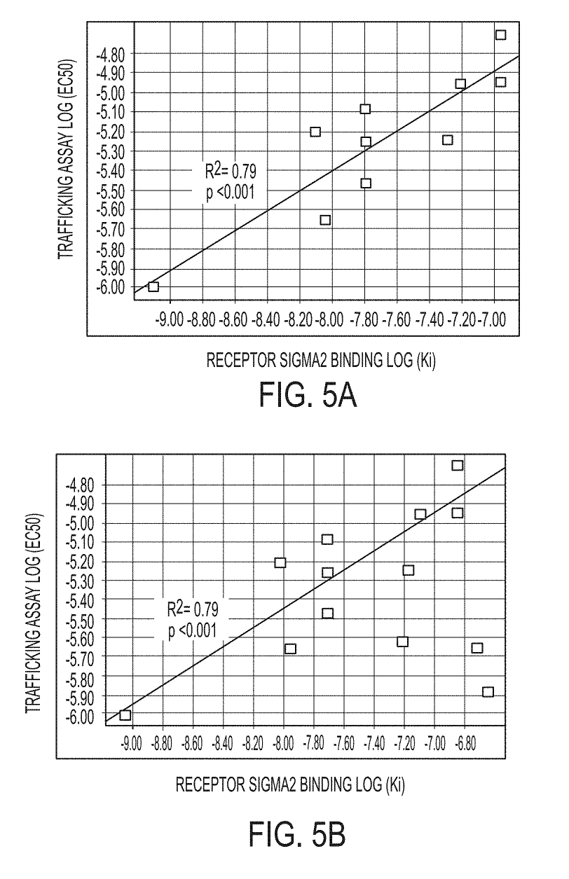

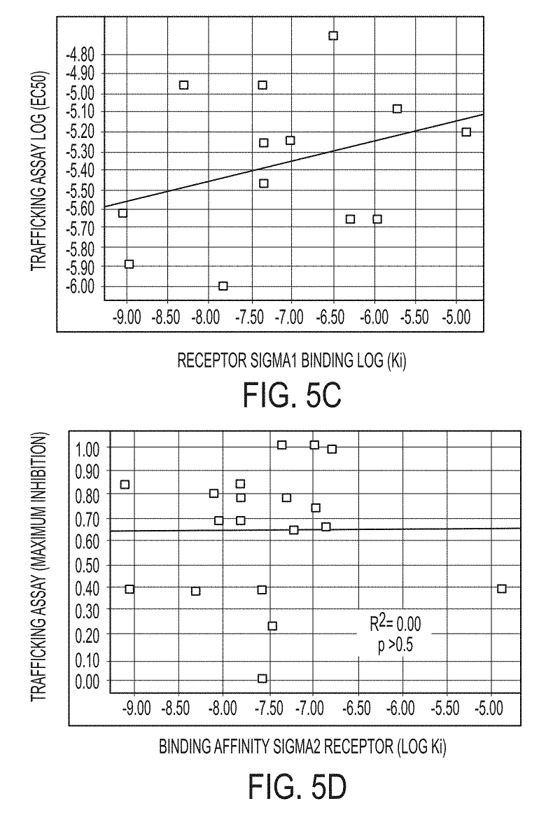

Graphs of a different representation of this correlation are also shown in FIGS. 5A-5D.

Of the known prior art compounds, Mach-14 is highly selective for Sigma-2 receptors (Sigma-1 Ki=12,900 nM, Sigma-2 Ki=9 nM) and it inhibited the Abeta effect in the trafficking assay by 80%. In contrast, PRE-84 is highly specific for Sigma-1 receptors (Sigma-1 Ki=2.2 nM, Sigma-2 Ki=13,091 nM) and was a poor inhibitor in the trafficking assay (max inhibition 38%). This result is consistent with the theory that Sigma-2 receptor binding, rather than sigma-1 receptor binding, is associated with reversing the effects of Abeta in the trafficking assay. Results of this assay provide further support for the development of the therapeutic phenotype.

When the bolded compounds from Table 5, along with known prior art compounds PB 28, Haloperidol and Mach 14 are graphed (FIG. 5A) there is a strong correlation between Sigma-2 binding affinity and potency in the trafficking assay (R2=0.79, P<0.001). In comparison, there is no significant correlation between binding to Sigma-1 receptors and potency in the MTTX assay (R²=0.02, P=0.18) (FIG. 5C). This result shows a strong relationship between binding to Sigma-2 receptors and inhibition of Abeta effects in trafficking and appears to indicate a poor relationship between binding to Sigma-1 receptors and inhibition of Abeta.

Three other known prior art compounds (BD1047, NE100 and SM-21) all were more potent in the membrane trafficking MTTX assay than could be accounted for by their Sigma-2 binding affinity alone. Haloperidol, PB 28 and Mach-14 demonstrated a close correlation between Sigma-2 binding and potency in the trafficking assay.

PRE-084 is inactive in the trafficking assay and this is consistent with the observation that it is a potent Sigma-1 agonist and is not potent at the sigma-2 receptor.

Two bolded compounds, R′ and S′, were inactive in the trafficking assay despite their substantial affinity for the sigma-2 receptor. In some embodiments, compounds R′ and S′ do not meet the therapeutic profile.

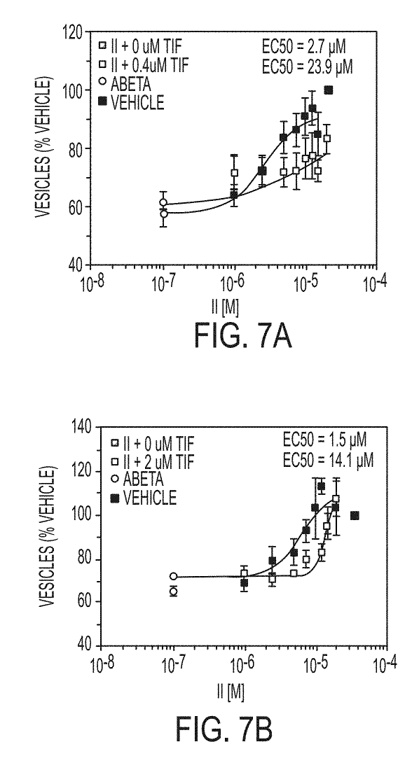

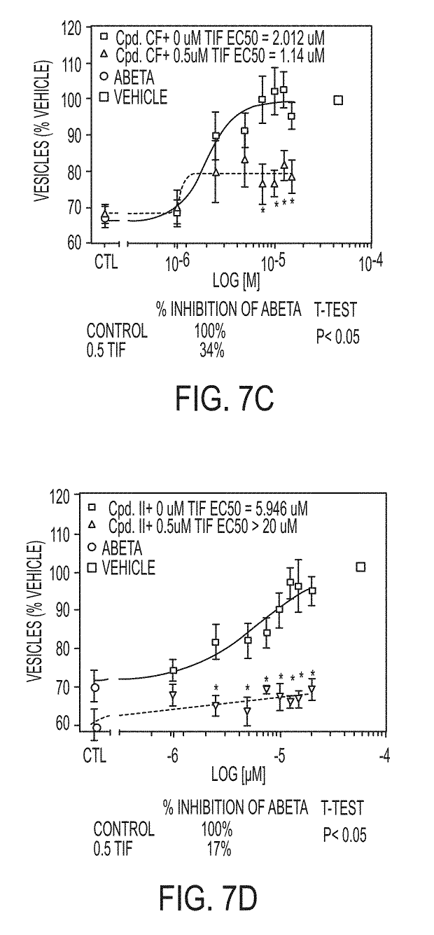

Additionally, a mixture of compounds IXa and IXb synergistically inhibit 100% of oligomer effects on membrane trafficking with a reproducible EC₅₀ of 5.2 μM+/−1.1 (FIG. 7). Similarly, additional compounds will be tested in the assay reported in this Example. These will also be selected from Formula I, II, III-VII, and IX as well as compounds encompassed by the other formulae (e.g., Formula XIII) and other compounds described as sigma-2 ligands described above.

Selected compounds in Table 2 were dosed in the membrane trafficking assay and were shown to block the Abeta oligomer-induced membrane trafficking abnormalities with the indicated EC₅₀. Accordingly, the compounds in Table 2 significantly blocked Abeta oligomer-mediated changes in membrane trafficking. These results indicate that compounds block/abate the activity/effect of Abeta oligomer on neuron cells and that sigma-2receptor ligands can be used as candidate compounds to block the Abeta oligomer induced membrane trafficking abnormalities. As the compounds embraced by the above formulae are expected to also be sigma-2 ligands, and will therefore also be useful in blocking the Abeta oligomer induced acceleration of exocytosis.

### Example 7. Pharmacokinetic and Metabolic Stability Studies

A first pharmacokinetic study was performed in microsomes of mice by a commercial contract research organization. The studies were performed according to Obach, R. S et al. (1997) J. Pharmacol. Exp. Ther., 283: 46-58, which is hereby incorporated by reference. The half-life of the compounds in Table 7A that were tested ranged from 2-72 minutes and the half-life of the remaining compounds is expected to be in about the same range.

The results for half-life in microsomes are shown in Table 7A and 7B.

TABLE 7A
Compound Mouse Microsome Stability.

| Compound | t_{1/2} in microsomes of mice |
|---|---|
| II | 16 |
| A | 33 |
| B | 55 |
| C | 10 |
| D | 2 |
| E | 46 |
| F | 72 |
| G | 42 |
| H | 24 |
| I | 33 |
| J | 47 |

The results indicate that several of the compounds tested had a substantially longer half-life in mouse liver microsomes than Compound II. This result portends greater bioavailability after oral administration for these compounds. The same compounds have been tested by the membrane trafficking assay described above and their activity as referred to herein.

The rate of intrinsic clearance of Compound II was rapid, suggesting substantial first pass metabolism. In order to improve pharmacokinetic properties, additional compounds were designed to enhance metabolic stability and improve drug-like properties. Microsomal stability experiments and plasma stability experiments were performed to determine metabolic and hepatic stability of candidate compounds.

A second PK study was conducted in vivo and involved measuring plasma levels and brain levels for test compounds administered by various routes and in an acute or chronic manner, as follows:

### HPLC-MS Optimization

A solution of each test compound was prepared and infused into the TSQ Quantum spectrometer (Fisher Thermo Scientific) source via syringe pump at a constant rate. Full scan MS (mass spectroscopy) analysis was conducted and total ion current chromatograms and corresponding mass spectra were generated for each test compound in both positive and negative ionization modes. The precursor ions for MS/MS were selected from either the positive or the negative mass spectrum, as a function of the respective ion abundance. In addition, product ion MS/MS analysis was performed in order to determine the appropriate selected fragmentation reaction for use in quantitative analysis. The final reaction monitoring parameters were chosen to maximize the ability to quantify the test compound when present within a complex mixture of components. Following identification of the specific SRM transition to be used for each test compound, the detection parameters were optimized using the automated protocol in the TSQ Quantum Compound Optimization workspace. Finally, the chromatographic conditions to be used for LC-MS analysis were identified by injection and separation of the analyte on a suitable LC column and adjustment of the gradient conditions as necessary.

### Formulation for IV Dosing:

The solubility of the test compound in phosphate-buffered saline, pH 7.4 (PBS) was first evaluated by visual inspection. PBS was used as the vehicle if the compound was soluble at the target concentration. (Other vehicles that are compatible with IV dosing may be evaluated if the compound is not completely soluble in PBS. Such vehicles include DMSO, polyethylene glycol (PEG 400), Solutol HS 15, and Cremophor EL among others.) In the experiments reported here a single bolus, 10 mg/kg, of Compound II was administered IV.

Formulation for PO dosing: The solubility of the test compound in PBS was first evaluated. PBS was used as the vehicle if the compound is soluble at the target concentration. (DMSO/Solutol HS 15/PBS (5/5/90, v/v/v), or DMSO/1% methylcellulose (5/95, v/v) may be used if the test compound is not completely soluble in PBS at the respective concentration.)

### Linearity in Plasma

Aliquots of plasma were spiked with the test compounds at the specified concentrations. The spiked samples were processed using acetonitrile precipitation and analyzed by HPLC-MS or HPLC-MS/MS. A calibration curve of peak area versus concentration was constructed. The reportable linear range of the assay was determined, along with the lower limit of quantitation (LLQ).

### Quantitative Bioanalysis of Plasma Samples

The plasma samples were processed using acetonitrile precipitation and analyzed by HPLC-MS or HPLC-MS/MS. A plasma calibration curve was generated. Aliquots of drug-free plasma were spiked with the test compound at the specified concentration levels. The spiked plasma samples were processed together with the unknown plasma samples using the same procedure. The processed plasma samples (dried extracts) were typically stored frozen (−20° C.) until the HPLC-MS or HPLC-MS/MS analysis. The dried extracts were reconstituted into a suitable solvent and after centrifugation were analyzed by HPLC-MS or HPLC-MS/MS. Peak areas were recorded, and the concentrations of the test compound in the unknown plasma samples were determined using the respective calibration curve. The reportable linear range of the assay was determined, along with the lower limit of quantitation (LLQ).

Animals used in the study were male C57BL/6 mice weighing 20-30 g each or male Sprague-Dawley rats weighing 180-250 g. Three animals were treated for each administration condition and each time point, so that each animal was subjected to only one blood draw. Subcutaneous compound administration was accomplished by intraperitoneal injection. Per oral administration was accomplished by gastric gavage. Intravenous administration was accomplished via jugular catheter.

Following compound administration at various concentrations, plasma samples were collected at 10, 30, 60, 120, 240, 360, 480 and 1440 min.

Plasma Sample Collection from Mice and Rats

Animals were sedated under general inhalant anesthesia (3% isoflurane) for blood collection by cardiac puncture (mice) or jugular catheter (rats). Blood aliquots (300-400 μL) were collected in tubes coated with lithium heparin, mixed gently, then kept on ice and centrifuged at 2,500×g for 15 minutes at 4° C., within 1 hour of collection. The plasma was then harvested and kept frozen at −20° C. until further processing.

### Animal Dosing Design—In Vivo PK—Non Cannulated, Nonfasted Animals

Group 1: SC, n=3 animals per time point (24 animals total) or IV, n=3 animals per time point (24 animals total)
Group 2: PO, n=3 animals per time point (24 animals total)
Group 3: Control animals (for drug-free blood), n=5 mice
Each animal was subject to one blood draw and one brain collection.

Brain Sample Collection from Animals

Immediately after blood sampling, animals were decapitated and the whole brains were quickly removed, rinsed with cold saline (0.9% NaCl, g/mL), surface vasculature ruptured, blotted dry with gauze, weighted, kept on ice until further processing within one hour of collection. Each brain was homogenized in 1.5 mL cold phosphate buffered saline, pH 7.4 (mice=1.5 mL, rats=), for 10 seconds on ice using the Power Gen 125. The brain homogenate from each brain was then stored at −20° C. until further processing.

Linearity in Brain Samples

Aliquots of brain homogenate were spiked with the test compound at the specified concentrations. To each brain aliquot an equal volume of chilled 26% (g/mL) neutral Dextran (average molecular Weight 65,000-85,000 from Sigma, catalog number D-1390) solution was added to obtain a final Dextran concentration of 13%. The homogenate was centrifuged at 54000×g for 15 minutes at 4° C. The supernatants were subsequently processed using acetonitrile precipitation and analyzed by HPLC-MS/MS. A calibration curve of peak are versus concentration was constructed. The reportable linear range of the assay was determined, along with the lower limit of quantitation (LLQ).

Quantitative Analysis of Brain Samples

To each brain homogenate aliquot an equal volume of chilled 26% (g/mL) neutral Dextran (average molecular Weight 65,000-85,000 from Sigma, catalog number D-1390) solution was added to obtain a final Dextran concentration of 13%. The homogenate was centrifuged at 54000×g for 15 minutes at 4° C. The supernatants were subsequently processed using acetonitrile precipitation and analyzed by HPLC-MS/MS. A brain calibration curve was generated. Aliquots of drug-free brain homogenate were spiked with the test compound at specified concentration levels. The spiked brain homogenate samples were processed together with the unknown brain homogenate samples using the same procedure. The processed brain samples were stored at −20° C. until the LC-MS/MS analysis, at which time peak areas were recorded, and the concentrations of test compound in the unknown brain samples were determined using the respective calibration curve. The reportable linear range of the assay was determined along with the lower limit of quantitation (LLQ).

### Brain Penetratrability

The concentrations of the test compound in brain (ng/g tissue) and in plasma (ng/mL) as well as the ratio of the brain concentration and the plasma concentration at each time point were determined by LC-MS/MS and reported as described above.

### Pharmacokinetics

Plots of plasma concentration of compound versus time were constructed. The fundamental pharmacokinetic parameters of compound after oral and SC dosing (AUClast, AUCINF, T1/2, Tmax, and Cmax) were obtained from the non-compartmental analysis (NCA) of the plasma data using WinNonlin (Pharsight). Noncompartmental analysis does not require the assumption of a specific compartmental model for either drug or metabolite. NCA allows the application of the trapezoidal rule for measurements of the area under a plasma concentration-time curve (Gabrielsson, J. and Weiner, D. Pharmacokinetic and Pharmacodynamic Data Analysis: Concepts and Applications. Swedish Pharmaceutical Press. 1997).

### Definitions of Terms Reported

Area Under the Curve (AUC)—Measure of the total amount of unchanged drug that reaches the systemic circulation. The area under the curve was a geometric measurement that was calculated by plotting concentration versus time and summing the incremental areas of each trapezoid.

WinNonlin has two computational methods for calculation of the area: the linear trapezoidal method and the linear-log trapezoidal method. Because the linear trapezoidal method may give biased results on the descending part of the concentration-time curve and overestimate the AUC, WinNonlin provides the linear-log option for calculation of AUC. By default, the log-linear trapezoidal method was used to measure the post-Tmax area for the remainder of the plasma concentration-time curve.

AUCₗₐₛₜ: area under the curve from the time of dosing to the time of last observation that was greater than the limit of quantitation.

AUC_{NF}: Area under the curve from the time of dosing extrapolated to infinity.

Cₘₐₓ—Maximum plasma drug concentration obtained after oral or non-IV administration of a drug between the time of doing and the final observed time point.

Tₘₐₓ—Time at maximum observed plasma concentration (Cmax) noted in minutes after administration of drug.

T_{1/2}—Terminal elimination half-life from both IV and non-IV dosing.

where lambda Z (z) is the first order rate constant associated with the terminal (log-linear) portion of the plasma concentration-time curve. z was estimated by linear regression of time versus log concentration.

The results showed that the tested compounds II and B were highly bioavailable and highly brain penetrability when they are administered at doses ranging from 0.1 to 0.5 mg/kg acutely or chronically (daily over 5 days). The results for acute administration of Compound II are shown in FIG. 2A. FIG. 2A is a graph wherein plasma levels of compound are shown on the left y-axis in units of ng/mL. Brain levels are shown on the right y-axis in green in units of ng/g. The x axis shows the time following bolus IV or SC administration at time zero. Following acute IV administration at 10 mg/kg i.v., Compound II reached a high brain concentration and at 180 minutes post-dosing still had a concentration of 171 ng/g (57× the efficacious brain dose in vivo, shown by the open diamond). A similar pattern followed acute SC administration. Compound B showed the same level of bioavailability on parenteral administration but was substantially more bioavailable by oral route. Both compounds tested were found to be clearly highly BBB-penetrant, and Compound II had a brain/plasma ratio of 8 at 3 hours.

The results for compound B and those for Compound II acute administration are shown in Table 8.

TABLE 8
Pharmacokinetics for Compounds B and II in Mice.

| | |
|---|---|
| Compound | Compound |
| B - 10 mg/kg | II - 10 mg/kg |

| | PO | SC | PO | SC |
|---|---|---|---|---|
| Parameter | Plasma | Plasma | Plasma | Plasma |
| Tₘₐₓ (min) | 30 | 120 | NC | 30 |
| Cₘₐₓ (ng/mL) | 599.3 | 607.7 | NC | 279 |
| t_{1/2} (min) | 210 | 218 | NC | 100 |
| AUCₐₗₗ | 2,851.1 | 5,242.3 | NC | 384.7 |
| ((ng * hr)/mL) | | | | |
| Fᵣₑₗ (%) | 54.4 | | NC | |

As shown in this table, the oral bioavailability of Compound B and its other PK parameters are improved over Compound II.

Dosing over a range of 0.1, 0.35 and 0.5 mg/kg gave relatively stable plasma levels of Compound II in chronic administration over the course of 5 days, with good brain exposure and similar brain/plasma ratios as the acute setting. The results are shown in FIG. 2B. FIG. 2B is a plot of pharmacokinetic data obtained in plasma (left ordinate) upon once daily subcutaneous administration of different amounts of Compound II (0.5 mg/kg/day: downward pointing filled triangles; 0.35 mg/kg/day: upward pointing filled triangles; and 0.1 mg/day filled squares) and in brain (right ordinate) upon SC administration of the same amounts (respectively downward pointing open triangle, upward pointing open triangle and open square) of Compound II.

Brain level measurements for Compound B showed a lower peak concentration than Compound II but at a higher sustained level. At the three-hour point, the amount of Compound II in the brain is almost 40× higher for PO and 60× higher for SC than the 50 ng/mL efficacious dose determined for Compound II. However, the three-hour time point for Compound II is still >3× higher for SC than the efficacious dose for that compound (data not shown).

A mixture of Compounds IXa and IXb, when dosed at 1 mg/kg intravenously in mice, displayed a half life of 2.7 hours. However, when the compound was dosed at 5 mg/kg orally, it displayed negligible amounts of drug in the plasma. A subsequent study (Table 6) of the mixture of Compounds IXa and IXb plus drug standards in mouse hepatic microsomes measured a half life for the mixture of 8.7 minutes and an intrinsic clearance of 267 microL/min/mg indicating that the mixture is susceptible to first pass metabolism. In general, CNS active compounds tend to have a low to moderate intrinsic clearance rate (CLᵢₙₜ<100 microL/min/mg) (Wager et al., '10). Human hepatic microsomal stability data of 13 CNS active drugs gave an average half life of 51±29 minutes (Orbach '99). Thus, reasonable goals for the improvement of Compound II to first pass metabolism would be a T_{1/2}>30 minutes and a CLᵢₙₜ<100 microL/min/mg. Compounds IXa, IXb exhibited comparable hepatic stability compared to certain known drugs, as shown in Table 9.

TABLE 9
Mouse hepatic microsome data for Compounds IXa and IXb and select
CNS drug standards. CL'int = intrinsic clearance. Experiments were
performed by a contract research organization and run as standards
with Compound II.

| | | Microsomal | | CL' INT |
|---|---|---|---|---|
| | | concentration | | (uL/min/ |
| Drug | Mode of action | (mg/mL) | T½ (min) | mg) |
| Mixture of | Candidate | 0.3 | 8.7 +/− 0.1 | 267 |
| Compounds | compound | | | |
| IXa and IXb | | | | |
| Imipramine | antidepressant | 0.3 | 11.5 | 200 |
| Propranolol | Beta blocker | 0.3 | 16.4 +/− 0.5 | 141 |
| Terfenadine | antihistamine | 14.6 | 8.7 +/− 1.1 | 159 |
| Verapamil | Ca++ channel | 0.3 | 11.4 +/− 0.5 | 204 |
| | blocker | | | |

### Example 8: Abeta 1-42 Oligomer Binding and Synapse Loss Assay

In this assay, Abeta oligomers were brought in contact with mature primary neurons in culture and their binding was determined by immunohistochemistry (anti-Abeta antibody) and quantified by image processing. The amount of Abeta in neuronal dendrites is assessed by counting the number of labeled puncta on the neuritis. Abeta oligomers are known to bind, saturably (Kd approximately 400 nM; Lauren 2009) and with high affinity to a subset of postsynaptic neurons present on a significant percentage (30 to 50%) of hippocampal neurons in primary cultures (Lacor et al, 2004; Lambert et al, 2007) and this correlates well with observations of Abeta binding in brains from Alzheimer's patients (Lambert et al, 2007). This labeling is associated with synapses, co-localizing with the post-synaptic scaffold protein PSD-95 (Lacor et al., '04). Abeta oligomers are also known to mediate synapse loss, reported as 18% in human hippocampal neurons in brain slices (Schef et al, 2007) and to inhibit long term potentiation (LTP). The number of synapses can also be quantified in this assay by immunofluorochemistry. Similar procedures for binding assays can be found in the literature. See e.g., Look G C, et. al. Discovery of ADDL—targeting small molecule drugs for Alzheimer's disease. Curr Alzheimer Res. 2007 December; 4(5):562-7. Review.

Measurement of the amount of Abeta bound to the surface of neurons can be used as a secondary screen to identify compounds acting via one or more of the following mechanisms: blocking Abeta effects by interference with Abeta oligomer binding to neuronal surface or by effecting alterations to the oligomers themselves (inverse agonism or oligomer dissociation) or alteration of the surface receptors that the oligomers bind to (allosteric modulation or classical receptor antagonism) It can also distinguish these compounds from compounds acting on downstream signaling events. Accordingly, this assay is relevant to disease states characterized by Abeta oligomer nonlethal effects on neurons and forms part of a screening cascade employed by the present inventors to identify clinically relevant compounds. Importantly, one of the compounds disclosed here, Compound II, has been active in membrane trafficking assay and in this binding/synapse loss assay and has been proved also active in two different transgenic models for Alzheimer's disease and in an induced model as well. Accordingly, this as well as the membrane trafficking assay is useful in identifying clinically relevant compounds and appears to have predictive value for in vivo results. The predictive validity of this assay is being confirmed by demonstrating its ability to predict compound properties using compounds outside of the scope of the present invention.

Primary hippocampal neuronal culture was established as in the membrane trafficking assay above. Compound II (at concentrations of 10⁻⁸ to 30 micromolar) was added and any other compound to be tested in the future (at concentrations of 10⁻⁸ to 30 micromolar) were added to a plate followed by an addition of Abeta 1-42 oligomer containing preparation at a concentration to reach saturation binding. Pretreatment with compounds to be tested lasted for 1 hr and addition of Abeta oligomers or no oligomer (vehicle alone) in a final concentration of 70 ul was followed by incubation for an additional 23 hrs.

The plates were fixed with 3.7% paraformaldehyde in phosphate buffered saline for 15 min. The plates were then washed 3× with PBS for 5 min each. The plates were blocked at RT for 1 hr in 5% goat serum and 0.5% Triton X-100 in PBS. Primary antibodies (anti-MAP 2 polyclonal, Millipore #AB5622 and anti-Beta Amyloid 6E10 monoclonal, Covance #SIG-39300, at 1 microgram/ml, and rabbit polyclonal anti-synaptophysin, Anaspec, at 0.2 microgram/ml) were diluted 1:1000 in 5% goat serum with PBS. Primary antibodies were incubated overnight at 4° C. The plates were then washed 3× with PBS for 5 min each. Secondary antibodies (Alex Flor 488 polyclonal, Invitrogen #A11008 and Alexa Flor 647 monoclonal, Invitrogen #A21235) were diluted 1:1000 in 5% goat serum with PBS. Secondary antibodies were incubated at RT for 1 hr. The plates were washed once with PBS. DAPI (4′,6-diamidino-2-phenylindole, Invitrogen) was then applied at 0.03 ug/ul and incubated at RT for 5 min, then washed with PBS. The results show that, as expected, Abeta oligomer, prepared as detailed below and dosed at 3 or 1 μM depending on the preparation used, bound to neurons at synapses, as was revealed by a red dye. In humans with early Alzheimer's disease, the number of synapses in the hippocampus has been shown to be reduced by 18% compared to age-matched cognitively normal individuals (Scheff et al., '07) and this result could also be visualized on this assay by 20% regression of fluorescent puncta and therefore of the number of synapses. In the co-presence of Compound II (15 uM), however, the Abeta binding was reduced to essentially control levels, and the green fluorescence was unaffected indicating an undiminished synapse number. See FIGS. 3A, 3B, 3C and 3D. In FIG. 3A-panel A, Abeta 42 oligomers bind to postsynaptic spines; FIG. 3A-panel B shows presynaptic spines are labeled with synaptophysin in primary neurons (21 DIV). FIG. 3A-Panels C and D shows the post-synaptic spines and synapses, respectively, at essentially control levels when IXa,IXb have been added to the culture. As shown quantitatively in the bar diagram of FIG. 3C, Abeta 42 oligomers added alone caused a 20% decrease in the density of synaptophysin puncta (as calculated) after 24 hrs (fourth bar) compared to vehicle alone (first bar). This loss was reversed by either Compound II or IXa,IXb (fifth or sixth bars) and this result was statistically significant. In the absence of Abeta oligomer, neither Compound IXa,IXb nor Compound II affects synaptic number (hatched bar) and it remains at levels comparable to control (vehicle alone). Scale bar=20 um. p<0.001 ANOVA. FIG. 3D (p<0.001 ANOVA) is also a bar diagram and shows that the Abeta binding intensity as calculated by the Abeta puncta is reduced by 18% in the presence of Compound II or IXa,IXb, yet this decrease is sufficient to permit synapse count to reach control levels in the presence of this compound.

Additionally, punctuate synaptic Abeta oligomer binding is reduced by 38% in the presence of a mixture of Compounds IXa and IXb in a concentration-dependent manner, with an IC₅₀ of 1.2 μM (data not shown). A histogram of puncta intensity reveals that the normal bimodal binding population (neurons with bright puncta and a population with less bright puncta) is left-shifted in the presence of drug (data not shown). Partial inhibition of Abeta oligomer binding has been reported to restore 100% of LTP function (Strittmatter S M et al., Cellular Prion Protein Mediates Impairment of Synaptic Plasticity by Amyloid-Beta Oligomers Nature (2009) 457 (7233:1128-32)). Further, as shown in FIG. 3C, Abeta oligomer (fourth bar) caused a 20% decrease in the density of synaptophysin puncta after 24 hrs compared to vehicle-treated (first bar), which was reversed by 5 μM of the mixture of Compounds IXa and IXb (fifth bar). In the absence of Abeta (second bar), the mixture of Compounds IXa and IXb do not affect synaptic number. Abeta oligomers cause an 18.2% decrease in synapse number; 100% of this loss is eliminated by 5 μM of compound IXa,IXb or II (FIG. 3C). The mixture of Compounds IXa and IXb cause a 17.7% decrease in the intensity of Abeta labeled puncta (FIG. 3D) with an IC₅₀ of 1.21 uM.

Nuclei, visualized with DAPI, exhibited a normal morphology, indicating an absence of neurodegeneration. The procedure will be repeated with additional test compounds selected from among those encompassed by Formula I-IX, as well as other compounds described as sigma-2 ligands above.

### Abeta Oligomer Preparations:

Human amyloid peptide 1-42 was obtained from California Peptide, with lot-choice contingent upon quality control analysis. Abeta 1-42 oligomers were made according to published methods as described above. [See e.g. Dahlgren et al., “Oligomeric and fibrillar species of amyloid-beta peptides differentially affect neuronal viability” J Biol Chem. 2002 Aug. 30; 277(35):32046-53. Epub 2002 Jun. 10; LeVine H 3rd. “Alzheimer's beta-peptide oligomer formation at physiologic concentrations” Anal Biochem. 2004 Dec. 1; 335(1):81-90; Shrestha et. al, “Amyloid beta peptide adversely affects spine number and motility in hippocampal neurons” Mol Cell Neurosci. 2006 November; 33(3):274-82. Epub 2006 Sep. 8; Puzzo et al., “Amyloid-beta peptide inhibits activation of the nitric oxide/cGMP/cAMP-responsive element-binding protein pathway during hippocampal synaptic plasticity” J Neurosci. 2005 Jul. 20; 25(29):6887-97; Barghorn et al., “Globular amyloid beta-peptide oligomer—a homogenous and stable neuropathological protein in Alzheimer's disease” J Neurochem. 2005 November; 95(3):834-47. Epub 2005 Aug. 31; Johansson et al., Physiochemical characterization of the Alzheimer's disease-related peptides A beta 1-42 Arctic and A beta 1-42 wt. FEBS J. 2006 June; 2 73(12):2618-30] as well as brain-derived Abeta oligomers (See e.g. Walsh et al., Naturally secreted oligomers of amyloid beta protein potently inhibit hippocampal long-term potentiation in vivo. Nature (2002). 416, 535-539; Lesne et al., A specific amyloid-beta protein assembly in the brain impairs memory. Nature. 2006 Mar. 16; 440(7082):352-7; Shankar et al, Amyloid-beta protein dimers isolated directly from Alzheimer's brains impair synaptic plasticity and memory. Nat Med. 2008 August; 14(8):837-42. Epub 2008 Jun. 22). Quality controls of oligomer preparations consist of Westerns to determine oligomer size ranges and relative concentrations, and the MTT assay to confirm exocytosis acceleration without toxicity. Toxicity was monitored in each image-based assay via quantification of nuclear morphology visualized with the DNA binding dye DAPI (Invitrogen). Nuclei that were fragmented are considered to be in late stage apoptosis and the test rejected (Majno and Joris Apoptosis, oncosis, and necrosis. An overview of cell death. Am J Pathol 1995; 146:3-16). Peptide lots producing unusual peptide size ranges or significant toxicity at standard concentrations on neurons would be rejected.

### Controls

Pre-adsorption of anti-Abeta antibody 6E10 with oligomer preparation inhibits synapse binding in a dose dependent manner (at 7.84×10⁻⁶) and is used as a positive control. The antibody was used at 1:1000 (1 microgram/ml). For the synapse loss assay, the NMDA antagonist dizocilpine (MK-801) is used as the positive control at 80 uM.

### Image Processing

Images were captured and analyzed with the Cellomics VTI automated microscope platform, using the Neuronal Profiling algorithm. For statistical analysis, a Tukey-Kramer pair-wise comparison with unequal variance was used.

### Western Blots

Samples containing Abeta 1-42 were diluted (1:5) in non-reducing lane marker sample buffer (Pierce #1859594). A 30 microliter (μL) sample was loaded onto an eighteen well precast 4-15% Tris-HCl gel (BIORAD #345-0028). Electrophoresis was performed in a BIO-RAD Criterian precast gel system using Tris-Glycine buffer at 125 volt (V) for 90 minutes. The gels were blotted onto 0.2 μM nitrocellulose membranes in Tris-Glycine/10% methanol buffer at 30V for 120 minutes. The membranes were boiled for 5 minutes in a PBS solution and blocked over night with TBS/5% milk solution at 4° C. The membrane was probed with 6E10-HRP (Covance #SIG-39345) diluted to 10 μg/mL in TBS/1% milk solution for one hour at room temperature. Membrane was washed three times for 40 minutes each with a solution of TBS/0.05% tween-20 and developed with ECL reagent (BIO-RAD #162-0112) for 5 minutes. Image acquisition was performed on an Alpha Innotech FluorChem Q quantitative imaging system and analyzed with AlphaView Q software.

### Activity

Compound II was shown and compounds selected from those specifically disclosed herein are expected to be shown to partially block binding of the Abeta oligomer ligand to neurons by about 25% according to the binding assay (using imaging processing algorithm).

### Example 9: Fear Conditioning Assay

Compound II was tested in an animal model of a memory-dependent behavioral task known as fear conditioning. The study protocol was designed based on published protocols (See e.g. Puzzo D, Privitera L, Leznik E, Fà M, Staniszewski A, Palmeri A, Arancio O. Picomolar amyloid-beta positively modulates synaptic plasticity and memory in hippocampus. J Neurosci. 2008 December 31; 28(53):14537-45.). The formation of contextual memories is dependent upon the integrity of medial temporal lobe structures such as the hippocampus. In this assay mice were trained to remember that a particular salient context (conditioned stimulus; CS) is associated with an aversive event, in this case a mild foot shock (the unconditioned stimulus, US). Animals that show good learning will express an increase in freezing behavior when placed back into the same context. This freezing is absent in a novel context. Increased freezing in the context indicates strong hippocampal-dependent memory formation in animals. Memory tested in Fear Conditioning is sensitive to elevations of soluble Aβ. Compound II was effective at stopping Abeta oligomer mediated effects on membrane trafficking. When administered to animals prior to Abeta oligomer administration, Compound II blocked oligomer effects on memory in a dose-dependent manner. The compound completely blocked oligomer-mediated memory deficits at the 2 pmol dose.

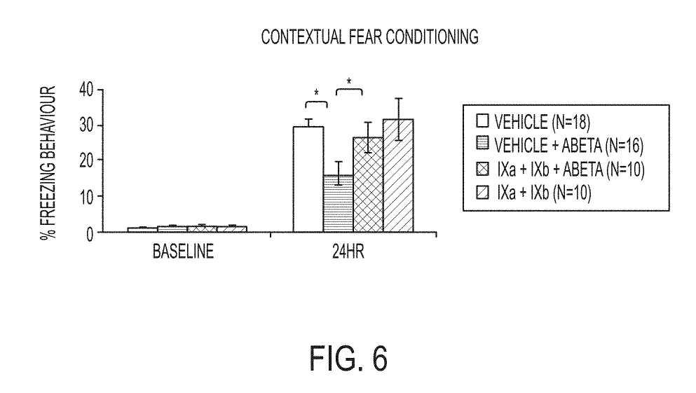

Indeed, as shown in FIG. 4, Compound II completely eliminated Abeta oligomer-induced deficits in memory (black bar) but did not affect memory when dosed alone (hatched bar). The effect of Abeta oligomer alone is shown by the red bar. Additionally, as shown in FIG. 6, a mixture of Compounds IXa and IXb provided a similar result. This behavioral efficacy demonstrates that the membrane trafficking assay is able to predict which compounds will be efficacious in treating the behavioral memory loss caused by oligomers. The fear condition model for memory was performed as described herein. No adverse behavioral changes were observed at any dose. Accordingly, there is a correlation between the performance of this compound in the membrane trafficking assay and its performance in the fear conditioning assay, the latter being an indicator of memory loss. It is anticipated that the compounds listed in Table 2 will be active in the fear conditioning assay and therefore will be shown to be efficacious in treating memory loss. The correlation between the performance of a compound in the fear condition model and its usefulness in treating memory loss has been established in the literature. (Delgado M R, Olsson A, Phelps E A. “Extending animal models of fear conditioning to humans” Biol. Psychol. 2006 July; 73(1):39-48).

### Example 10. Autoradiography Studies with Rat, Rhesus Monkey and Human Postmortem Brain Samples

Autoradiography imaging studies for the neurological and pharmacological profiling of the sigma-2 and sigma-1 receptor ligands were conducted by a modification of the protocol previously reported by Xu et al., 2010. Xu, J., Hassanzadeh B, Chu W, Tu Z, Vangveravong S, Tones L A, Leudtke R R, Perlmutter J S, Mintun M A, Mach R H. [³H]4-(Dimethylamino)-N-[4-(4-(2-methoxyphenyl)piperazin-1-yl)butyl]benzamide, a selective radioligand for dopamine D(3) receptors. II. Quantitative analysis of dopamine D3 and D2 receptor density ratio in the caudate-putamen. Synapse 64: 449-459(2010), which is incorporated herein by reference. Labeled RHM-1 was obtained by the method of Xu J, Tu Z, Jones L A, Wheeler K T, Mach R H. [³H]N-[4-(3,4-dihydro-6,7-dimethoxyisoquinolin-2(1H)-yl)butyl]-2-methoxy-5-methylbenzamide: a Novel Sigma-2 Receptor Probe. Eur. J. Pharmacol. 525: 8-17 (2005), which is incorporated herein by reference.

Brain sections in 20 μM thickness from rats, rhesus monkeys and postmortem human brains were cut using with a Microm cryotome and mounted on superfrost plus glass slides (Fisher Scientific, Pittsburgh, Pa.)., and serial sections through the brain regions of cerebral cortex and hippocampus were used in this study. Brain section were incubated with 5 nM [³H](+)-Pentazocine for sigma-1 receptor profiling, 4 nM [³H]RHM-1 only for sigma-2 receptor characterization, 10 nM [³H]DTG and [³H]Haloperidol in the presence of sigma-1 receptor block (+)-Pentazocine to image the sigma-2 receptor distribution; after incubation with the radioligands for 30 minutes, the brain sections containing glass slides were rinsed 5 times at one minute each time with ice-cold buffer.

Slides were dried and made conductive by coating with a copper foil tape on the free side and then placed in the gas chamber [mixture of argon and triethylamine (Sigma-Aldrich, USA)] of a gaseous detector, the Beta Imager 2000Z Digital Beta Imaging System (Biospace, France). After the gas is well mixed and a homogenous state is reached, further exposure for 24 hours to 48 hours until high quality images are observed. [³H]Microscale (American Radiolabeled Chemicals, Inc., St. Louis, Mo.) was counted at the same time as a reference for total radioactivity quantitative analysis, i.e., to convert the cpm/mm2 to nCi/mg tissue. Quantitative analysis was performed with the program Beta-Image Plus (BioSpace, France) for the anatomical regions of interest (ROI), i.e., to obtain the quantitative radioactivity uptake (cpm/nlln2) in the regions of cortex and hippocampus. The binding density was normalized to fmol/mg tissue based on the specific activities of the corresponding radioligands and calibration curve from the standard [³H]Microscale. A series of dilutions of candidate compounds (10 nM, 100 nM, 1,000 nM and 10,000 nM) were tested for competing the binding sites using the quantitative autoradiography, for those four radioligands, [³H](+)-Pentazocine, [³H]RHM-1, [³H]DTG and [³H]Haloperidol, then the specific binding (% control) was analyzed to derive the binding affinity in the regions of the cortex and the hippocampus (dentate gyrus, hippocampal CA I and CA3).

Autoradiography at sigma-1 and sigma-2 receptors is shown at FIGS. 8A and 8B, respectively. FIG. 8C shows (A) [³H]-(+)-Pentazocine (a sigma-1 receptor ligand) autoradiography in human frontal cortex slices from normal patients, Lewy Body Dementia (DLB) patients, or Alzheimer's Disease (AD) patients and (B) a graph of specific binding compared to control. As shown in FIG. 8A, sigma-1 receptors are statistically downregulated in Alzheimer's disease and possibly DLB compared to normal control. This finding confirms that of Mishina et al. who reported low density of sigma-1 receptors in early Alzheimer's disease. Mishina et al., 2008, Low density of signal receptors in early Alzheimer's disease. Ann. Nucl Med 22: 151-156. FIG. 8B shows (A) [¹²⁵I]-RHM-4 (a sigma-2 receptor ligand) autoradiography in human frontal cortex slices from normal patients, Lewy Body Dementia (DLB) patients, or Alzheimer's Disease (AD) patients, and (B) a graph of specific binding compared to control. Sigma-2 receptors are not statistically downregulated in AD. FIG. 8C shows (A) displacement of 18.4 nM [³H]-RHM-1 in monkey frontal cortex, monkey hippocampus or human temporal cortex by sigma-2 ligands and (B) a graph of binding density of [³H]-RHM-1 with and without 1 μM each of siramesine and compounds IXA,IXB and J. Siramesine and compounds IXA,IXB and II partially displace [³H]-RHM-1 in the target tissues.

### Example 11. MTS Assay: Determination of Agonist or Antagonist Activity of Various Sigma-2 Ligands

The cytotoxicity of compounds shown below was determined using the CellTiter96 Aqueous One Solution Assay (Promega, Madison, Wis.). Briefly, MDA-MB-435 or MDA-MB231 or SKOV-3 cells were seeded in a 96-well plate at a density of 2000 cells/well on the day prior to treatment with sigma-2 receptor selective ligands. After a 24 hour treatment, the CellTiter 96 AQueous One Solution Reagent was added to each well, and the plate incubated for 2 hours at 37° C. The plate was read at 490 nm in a Victor3 plate reader (PerkinElmer Life and Analytical Sciences, Shelton, Conn.). The EC⁵⁰ value, defined as the concentration of the sigma ligand required to inhibit cell viability by 50% relative to untreated cells, was determined from the dose response curve for each cell line. Siramesine is accepted as an agonist. The agonists and antagonists of the sigma-2 ligands were defined as the following: If the EC50s of a sigma-2 ligand was less than 2 fold of EC50 of siramesine, this sigma-2 ligand is considered as an agonist. If the EC50 of a sigma-2 ligand was between 2 and 10 fold of EC50 of siramesine, this sigma-2 ligand was considered as a partial agonist. If the EC50 of a sigma-2 ligand is larger than 10 fold of EC50 of siramesine, this sigma-2 ligand is considered as an antagonist. The sigma-2 ligands used for the studies are: agonists (siramesine and SV 119), partial agonist (WC26), antagonist (RHM-1), and candidate compounds (II and IXa,IXb). Results are shown in FIG. 9A. Data from FIG. 9A is shown in Table 10.

TABLE 10
IC₅₀ values for TumorCell Viability assay.

| Compound | IC₅₀, 48 hrs. (uM) | Action |
|---|---|---|
| RHM-1 | 203 ± 13 | Antagonist |
| Siramesine | 11.8 ± 2.7 | Full agonist |
| SV-119 | 21.7 ± 2.9 | Full agonist |
| WC-26 | 65.6 ± 6.3 | Partial agonist |
| IXa, IXb | 169 ± 9 | Antagonist |
| II | 150 ± 12 | Antagonist, |

Neuronal cultures were treated with various concentrations of sigma compounds for 24 hours and nuclear intensity compared to vehicle was measured. Sigma-2 agonists (siramesine, SV-119, WC-26) caused significant abnormal nuclear morphology in neurons, as shown in FIG. 9B, in contrast to sigma-2 antagonists (RHM-1, IXa,IXb and II) which did not decrease nuclear intensity at the test concentrations. Therefore, sigma-2 receptor agonists were cytotoxic to the neuronal and cancer cells; however sigma-2 receptor antagonists were not toxic and further blocked the cytotoxicity caused by sigma-2 receptor agonists.

### Example 12. Caspase-3 Assays. Determination of Agonist or Antagonist Activity of Sigma-2 Ligands

As described herein, Xu et al. identified PGRMC1 protein complex as the putative sigma-2 receptor binding site. Xu et al., 2011. Nature Commun. 2, article number 380, incorporated herein by reference. Sigma-2 receptor agonists can induce Caspase-3-dependent cell death. Xu et al 2011 disclose functional assays to examine the ability of the PGRMC1 to regulate caspase-3 activation by sigma-2 receptor agonist WC-26.

Abeta oligomers cause low levels of caspase-3 activation and lead to LTD. High levels of Abeta oligomers and caspase-3 activation lead to cell death. Li et al., 2010; Olsen and Sheng 2012. It was demonstrated herein that sigma-2 receptor agonists (SV-119, siramesine) activate caspase-3 in tumor cells and neurons; see, for example, FIGS. 10A and 10B. Sigma-2 receptor antagonist RHM-1 inhibits the activation in tumor cells (FIG. 10A), but was not able to block activation by agonist SV-119 in neurons in this experiment (FIG. 10B). Test compounds II and IXa,IXb (all of which are sigma 2 receptor antagonists as shown below) were able to inhibit caspase-3 activation in tumor cells and block sigma-2 receptor agonist SV-119 activation of caspase-3 in neurons. Therefore, the test compounds II and IXa,IXb acted as sigma-2 receptor antagonists in caspase-3 assays in tumor cells and neurons, as demonstrated in this example.

The activation of endogenous caspase-3 by sigma-2 receptor ligands was measured using the Caspase-3 Colorimetric Activity Assay Kit (Milipore, Billerica, Mass.) according to the manufacture's protocol. Briefly, MDA-MB 435 or MDA-MB23I cells were plated at 0.5×10⁶ cells 100 mm dish. 24 hours after plating, sigma-2 ligands were added to the culture dishes to induce caspase 3 activation. The final concentration of the sigma-2 ligand was its EC50. 24 hours after treatment, cells were harvested, lysed in 300 uL of Cell Lysis Buffer, and centrifuged for 5 minutes at 10,000×g. Supernatant was collected and incubated with caspase-3 substrate, DEVD-pNA, for 2 hours at 37° C. The protein concentration was determined using Dc protein assay kit (Bio-Rad, Hercules, CA1. The resulting free pNA was measured using a Victor³ microplate reader (PerkinEliner Life and Analytical Sciences, Shelton, Conn.) at 405 nm. The ligands tested included: sigma-2 agonists (siramesine, SV119, WC26), and sigma-2 antagonist, RHMWU-I-102 (RHM-1), and candidate compounds (II and IXa,IXb). The ligands which activated caspase 3 were considered as agonists, whereas the ligands which did not activate caspase 3 were considered antagonists. As shown in FIG. 10A, the sigma-2 agonist siramesine induced caspase-3 activity, whereas sigma-2 antagonists RHM-1, and candidate compounds II and IXa,IXb did not induce caspase-3 activity. FIG. 10B shows activation of caspase-3 by sigma-2 agonist SV-119, that is blocked by compounds IXa,IXb and II. Compounds IXa,IXb and II behaved like sigma-2 antagonists in both cancer cells and neurons.

### Example 13. Therapeutic Phenotype

In some embodiments, the disclosure provides an in vitro assay platform predictive of behavioral efficacy. A compound that (1) selectively binds with high affinity to a sigma-2 receptor; and (2) acts as a functional antagonist in a neuron and is predicted to have behavioral efficacy if: it blocks Aβ-induced membrane trafficking deficits; blocks Aβ-induced synapse loss and does not affect trafficking or synapse number in the absence of Abeta oligomer. This pattern of activity in the in vitro assays is termed the “therapeutic phenotype”. The ability of a sigma-2 receptor antagonist to block Abeta oligomer effects in mature neurons without affecting normal function in the absence of Abeta oligomers is one criteria for the therapeutic phenotype. Compounds that affect trafficking or synapse number in the absence of oligomers are not behaviorally efficacious. Only those compounds that selectively block oligomers without affecting normal trafficking or altering synapse number are behaviorally efficacious in preventing and treating Abeta oligomer-induced memory loss. In one embodiment, the in vitro assay platform can predict behavioral efficacy. This pattern of activity in the platform assays is therefore a therapeutic phenotype.

For example, see Table 11A.

TABLE 11A
Therapeutic Phenotype.

| | Block Aβ- | | | |
|---|---|---|---|---|
| | induced | | | |
| | membrane | | | |
| | trafficking | Block Aβ- | Assay effects | |
| | deficits | induced | in the absence | Behavioral |
| Compound | EC50 (uM) | synapse loss | of Aβ | efficiency |
| II | 2.2 | ++ | No | Yes |
| Z | 6.1 | +++ | Yes | No |
| Z′ | 4.3 | +++ | Yes | No |
| IXa + IXb | 4.9 | +++ | No | Yes |

In summary; sigma-2 antagonists with high affinity (preferably Ki less than about 600 nM, 500 nM, 400 nM, 300 nM, 200 nM, 150 nM, 100 nM, or 70 nM) at sigma-2 receptors that have greater than about 20-fold, 30-fold, 50-fold, 70-fold, or preferably greater than 100-fold selectivity for sigma receptors compared to other non-sigma CNS or target receptors, have good drug-like properties including brain penetrability and good metabolic and/or plasma stability, and that possess the therapeutic phenotype, are predicted to have behavioral efficacy and can be used to treat Abeta oligomer-induced synaptic dysfunction in a patient in need thereof.

Functional neuronal phenotype for several Compound II analogs, predicted to have oral bioavailability, with in vitro assay characterization, is shown in Table 11B.

TABLE 11B
Functional Neuronal Phenotype

| | | Inhibition | | | | |
|---|---|---|---|---|---|---|
| | | Abeta | | | | |
| | | oligomer- | | | | |
| | | induced | S1 | S2 | | |
| | | Membrane | binding | binding | Block | Functional |
| | | Trafficking | Ki | Ki | synapse | Neuronal |
| Selectivity | Compound | EC50 (uM) | (nM) | (nM) | loss | Phenotype |
| Higher | II | 2.2 | 500 | 9 | 100% | Antagonist |
| affinity at | II (+) | 5.6 | 100 | 80 | 47% | Antagonist |
| sigma-2 | isomer | | | | | |
| | W | 8.7 | 110 | 36 | 43% | Antagonist |
| | S′ | >20 | 25 | 8 | 0% | Inactive |
| | P | >20 | 320 | 110 | 0% | Inactive |
| Higher | A | 3.4 | 3 | 13 | 100% | Antagonist |
| affinity at | B | 5.5 | 1.3 | 3.9 | 100% | Antagonist |
| sigma-1 | X | 6.1 | 3.5 | 16 | 100% | Antagonist |
| | E | 8.2 | 2 | 3.6 | 34% | Antagonist |
| | II (−) | 10.9 | 46 | 63 | 0% | Antagonist |
| | isomer | | | | | |
| Comparable | Y | 4.3 | 78 | 85 | 100% | Antagonist |
| affinity at | R′ | >20 | 11 | 16 | 33% | Inactive |
| sigma-2 | | | | | | |
| and sigma-1 | | | | | | |

### Therapeutic Phenotype

Table 11C shows known prior art compounds with high affinity for either sigma-2 or sigma-1 receptors. Several sigma-2 ligands fall into three functional neuronal phenotypes: antagonists (block Abeta signaling); agonists (block Abeta signaling with U-shaped dose-response curve and toxicity at high doses; and inactive (no effect in neuronal cultures). The known prior art sigma-1 receptor ligands fall into two categories: antagonists (block A beta signaling) and inactive (no effect in neuronal cultures). Most of the prior art compounds suffer from low selectivity in that they have significant affinity to other, non-sigma, receptors. Several of the prior art compounds may not be able to penetrate the blood brain barrier (BBB) and are likely substrates for oxidative metabolism, and thus would not fit the therapeutic profile.

TABLE 11C
Compounds-Selective Sigma-2 and Sigma-1 Receptor Ligands.

| | | Inhibition | | | |
|---|---|---|---|---|---|
| | | Abeta | | | |
| | | oligomer- | | | |
| | | induced | | | |
| | | Membrane | S1 | S2 | Functional |
| Selec- | | Trafficking | binding | binding | Neuronal |
| tivity | Compound | EC50 (uM) | Ki (nM) | Ki (nM) | Phenotype |
| Higher | PB 28 | 1 | 15 | 0.8 | Antagonist |
| Affinity | Siramesine | 1.3 | 19 | 0.19 | Agonist |
| at | WC-26 | 1.6 | 1,400 | 3 | Agonist |
| Sigma-2 | SM-21 | 2.2 | 1050 | 145 | Antagonist |
| | SV119 | 2.6 | 1,400 | 8 | Agonist |
| | M-14 | 6.2 | 12,900 | 8 | Antagonist |
| | Ifenprodil | >20 | 26 | 4.9 | Inactive |
| | threo- | >20 | 59 | 0.9 | Inactive |
| | ifenprodil | | | | |
| | DTG | >20 | 88 | 35 | Inactive |
| Higher | NE 100 | 1.3 | 1.1 | 170 | Antagonist |
| Affinity | BD1008 | 1.5 | 2.2 | 8 | Antagonist |
| at | BD1047 | 2.4 | 0.9 | 47 | Antagonist |
| Sigma-1 | Fluvoxamine | 2.5 | 13 | 710 | Antagonist |
| | Pentazocine | 7.6 | 3 | >5,000 | Antagonist |
| | PPBP | 10.9 | 0.8 | 1 | Antagonist |
| | Haloperidol | 11 | 5 | 110 | Antagonist |
| | PRE-84 | >30 | 2.2 | 13,091 | Inactive |
| | BD1063 | >30 | 8.8 | 625 | Inactive |
| | SKF 10,047 | >30 | 149 | >10,000 | Inactive |

Although several clinical compounds have the desired functional phenotype, they do not meet the desired therapeutic profile. Known prior art compounds with the desired antagonist functional neuronal phenotype, but that fail the criteria for therapeutic profile, either by being non-selective, or by failing to cross the BBB, or by being predicted to be an oxidative substrate and having metabolic instability, are shown in Table 11D.

TABLE 11 D
Characterization of Certain Known Compounds.

| | Membrane | Functional | Sigma-1 | Sigma-2 | Selectivity | Drug- |
|---|---|---|---|---|---|---|
| | trafficking | Neuronal | Receptor | Ki (nM) | other | like |
| Cpd | IC50 (uM) | Phenotype | Ki (nM) | Receptor | activities | properties |
| PB 28 | 1 | Antagonist | 15 | 0.8 | Not | Oxidative |
| | | | | | Reported | substr. |
| SM-21 | 2.2 | Antagonist | 1050 | 145 | Muscarinic | Oxidative |
| | | | | | antag | substr. |
| M-14 | 6.2 | Antagonist | 12,900 | 8 | D3 | Not |
| | | | | | | BBB, |
| | | | | | | Ox |
| | | | | | | Sub. |
| NE 100 | 1.3 | Antagonist | 1.1 | 170 | Not | Oxidative |
| | | | | | Reported | substr. |
| BD1008 | 1.5 | Antagonist | 2.2 | 8 | Not | Oxidative |
| | | | | | reported | substr. |
| BD1047 | 2.4 | Antagonist | 0.9 | 47 | D2, β₃-AR | Oxidative |
| | | | | | | substr. |
| Fluvoxamine | 2.5 | Antagonist | 13 | 710 | 5HT, DA, | Yes |
| | | | | | AR | |
| | | | | | transporters | |
| (+) | 7.6 | Antagonist | 3 | >5,000 | NMDAR, | Yes |
| Pentazocine | | | | | PCP | |
| PPBP | 10.9 | Antagonist | 0.8 | 1 | Not | Oxidative |
| | | | | | reported | substr. |
| Haloperidol | 11 | Antagonist | 5 | 110 | D2, D3, | Yes |
| | | | | | D4, | |
| | | | | | 5HT2A, | |
| | | | | | a1B, | |
| | | | | | a2A, a2B, | |
| | | | | | a2C, | |
| | | | | | NMDAR, | |
| | | | | | Ach, | |
| | | | | | GluR, | |
| | | | | | Ca2+ | |

In addition, many clinical compounds that bind to the sigma-2 and or sigma-1 receptors are not highly selective as illustrated in Table 11E. In fact, 10-fold, 20-fold, and particularly 100-fold selectivity for sigma receptors compared to other non-sigma receptors is rare.

TABLE 11E
Clinical Compound Selectivity.

| | Therapeutic | Sigma Affinity | |
|---|---|---|---|
| Drug | Effect | (S1/S2) nM | Other Targets |
| Fluoxetine | SSRI | 75/260 | 5-HT2C (33 nM), |
| | antidepressant | | 2A (141 nM), |
| | | | muscarinic M1-M4 |
| | | | (500 nM), |
| | | | serotonin |
| | | | transporter (1-270 nM) |
| Fluvoxamine | SSRI | 13/710 | Dopamine |
| | antidepressant | | transporter (1.5 nM), |
| | | | Serotonin (12 nM), |
| | | | Adrenergic |
| | | | transporter (299 nM) |
| Pentazocine | Opioid | 1.2/5,000 | NMDAR2B (2.5 |
| | analgesic | | uM), 2A (2.7 uM), |
| | | | PCP (2.9 uM) |
| Opipramol | Antidepressant, | 7/56 | 5-HT2A,B,C (120 nM), |
| | anxyolytic | | alpha |
| | | | 1A, 1B (200 nM) |
| Siramesine | Antidepressant, | 19/0.19 | D2 (800 nM), alpha |
| | anxyolytic | | 1A, 1B (300 nM) |
| Cutamesine | Antidepressant, | 17/>1800 | VAChT (50 nM), |
| | stroke | | muscarinic M1, 2, |
| | | | D2 (2 uM), alpha1 |
| | | | AR, 5-HT1a/2, H1 |
| | | | (2 uM) |
| Anavex 1-41 | Alzheimer's | 7/>10,000 | Muscarinic M1-M4 |
| | | | (18-114 nM), Na+ |
| | | | ch2 (>10 uM) |
| Anavex 2-73 | Alzheimer's | 860/>20,000 | Muscarinic M1-M4 |
| | | | (3-5 uM), |
| | | | NMDAR2B (8 |
| | | | uM), Na+ ch2 |
| | | | (5uM) |
| II | Alzheimer's | 500/9 | D3 (4 uM), Mu |
| | | | opioid (4 uM), |
| | | | Na + ch2 (2 uM) |
| IXa, IXb | Alzheimer's | 31/54 | DA transporter (1.5 |
| | | | uM) |

### Example 14: In Vitro Toxicity

Representative sigma-2 antagonists II and IXa,IXb did not induce neuronal or glial toxicity with acute or chronic dosing in vitro. The sigma-2 receptor antagonists eliminated or reduced Abeta oligomer-induced changes in membrane trafficking. No significant effect of compounds on membrane trafficking occurred when dosed without oligomers. There was no toxicity relative to neuron number, glial number, nuclear size, nuclear morphology, neurite length, cytoskeletal morphology when tested to 10 times the EC50 concentration (up to 50 μM II or IXa,IXb) for three days. See Table 12.

TABLE 12

| Compound | IXa, IXb | II |
|---|---|---|
| EC50 (uM) | 4.9 | 2.2 |
| Max Inhib. of Abeta (Conc) | 100% (14) | 85% (10) |
| Calculated Ki* | 0.58 | 0.26 |
| Cpd alone at Ki | +9% | +1% |

*Km for Abeta = 0.4 uM; assay concentr. 3 μM total Abeta.

In vitro toxicity for Compound II was tested in a number of standard assays. Testing in vitro tox studies reveals there is no genotoxicity at 10 μM (AMES, micronucleus, bacterial cytotox). HepG2 toxicity of 66% at 10 μM (100-fold above affinity at receptor x) may be due to compound lipophilicity or receptor overexpression in HepG2 tumor cell line. Partial inhibition (46-73%) of CYP 450 enzymes 2D6, 3A4, and 2C19 occurred at 10 uM. Moderate hERG inhibition (24%) was seen at 100 nM. Compound II exhibited very weak (IC50>30 uM) activity at PGP.

### Example 15: Separation and Activities of Enantiomers of Compound II in the Membrane Trafficking Assay

Compound II was separated into its (+) and (−) enantiomers. The racemic mixture was applied to a chiral column CHIRALPAK AD-H (amylose tris (3,5-dimethylphenylcarbamate) coated on silica-gel; 4.6×250 mm). The sample was injected into the column in a 15 microliter volume. The eluent was Hexane/EtOH/TEA (95/5/0.1) with a flow rate of 1 ml/min at 25 degrees Celsius. The two enantiomers were separated in distinct peaks. The (+) enantiomer eluted in a first peak at approximately 16 minutes and the (−) enantiomer eluted in a second peak eluting at approximately 20 minutes. The enantiomers were at least 98% pure. The (+) enantiomer had a specific rotation of +10.1 (c 1.80 in MeOH) and the (−) enantiomer had a specific rotation of −7.2 (c 1.80 in MeOH). The (+) enantiomer was more potent in the membrane trafficking assay described in Example 6 than the (−) enantiomer. In one sample, the (+) enantiomer had an EC50 of 5.6 and the (−) enantiomer has an EC50 of 10.9 μM in inhibiting amyloid beta induced deficits in the membrane trafficking assay.

### Example 16. Behavioral Efficacy of Orally Available Compounds-Improvement of Memory Deficits in Transgenic Alzheimer's Mouse Model

Male hAPP Swe/Ldn transgenic (Tg) mice were utilized as a TG model of AD. Transgenic mice that were treated with vehicle, 10 or 30 mg/kg/day of CB or CF for 5.5 months p.o., as well as non-transgenic vehicle-treated littermates were subjected to a standard fear conditioning paradigm. Vehicle-treated 9 month old male hAPP Swe/Ldn transgenic (Tg) mice that were treated p.o. for 5.5 months with vehicle exhibited significant memory deficits vs. vehicle-treated non-transgenic littermates in contextual fear conditioning.

When the animals were tested for associative memory 24 hours after training, two-way (genotype and time) ANOVA with repeated measures did not detect a significant difference in total freezing time between transgenic and nontransgenic vehicle-treated mice. However, the more sensitive analysis of freezing behavior during individual timed intervals indicates that transgenic mice performed significantly worse during the 1-3-minute interval compared to the non-transgenic vehicle-treated animals (Mann-Whitney U test, p<0.05). During this interval, transgenic animals that were treated with 10 and 30 mg/kg/day of CB (p<0.05) and 30 mg/kg/day of CF (p<0.005) significantly improved performance compared to vehicle (Mann-Whitney U test). Results are shown in FIG. 12, both doses of CB significantly reversed memory deficits in AD mice; and the higher dose of CF revered memory deficits in AD mice. Treatment of Tg animals with CB at 10 and 30 mg/kg/day or CF at 30 mg/kg/day improves the deficits at measured brain concentrations of 394±287, 793+325, or 331-373 nM respectively (AVG±S.D.). Brain/trough plasma and brain/peak plasma ratios for orally available compounds.

are shown in the Table 13.

TABLE 13
Brain/trough plasma and brain/peak plasma ratios for orally available
compounds.

| | | Brain/Trough | Brain/Peak |
|---|---|---|---|
| Compound | Dose (p.o.) | Plasma Ratio | Plasma Ratio |
| CF | 30 mg/kg | 13 | 0.5 |
| CF | 10 mg/kg | 11 | 0.2 |
| CB | 30 mg/kg | 14 | 0.4 |
| CB | 10 mg/kg | 17 | 0.7 |

Therefore, both compounds CB and CF are orally bioavailable, capable of achieving significant brain penetration and reversing established memory deficits in aged transgenic Alzheimer's mouse models animals following chronic long-term administration. No adverse behavioral effects observed.

Both CB and CF are selective, high affinity sigma-2 receptor antagonist compounds. Both CB and CF bind to the sigma-2 and sigma-1 receptors with high affinity as shown in Table 14. Counterscreening was performed against a panel of 40 brain receptors and results indicated that CB and CF are highly selective for sigma receptors, as shown in the Table 14.

TABLE 14
Receptor Affinities for Orally Bioavailable Compounds.

| | | Sigma Receptor | |
|---|---|---|---|
| | | Affinity (sigma- | |
| | Therapeutic | 1/sigma-2) | Other Receptor Affinities |
| Drug | Effect | (Ki, nM) | (Ki, nM) |
| CB | Alzheimer's | 19/48 | Muscarinic M1 (1.5 uM), |
| | | | M2 (1.5 uM), M3 (1.8 uM) |
| | | | kappa opioid (1.5 uM) |
| | | | Ca++ ch-L-type (860 nM) |
| | | | Transporters: NE (1.4 uM), |
| | | | DA (220 nM), 5-HT (970 nM) |
| CF | Alzheimer's | 180/50 | Muscarinic M1 (1.1 uM), M2 |
| | | | (2.5 uM), M3 (3.7 uM) |
| | | | kappa opioid (6.1 uM) |
| | | | Ca++ ch-L-type (2.5 uM) |
| | | | Transporters: NE (1.9 uM), |
| | | | DA (940 nM), 5-HT (3.2 uM) |

CB at a 10 mg/kg/day dose results in compound brain levels that are at or above the Ki for sigma and dopamine transporters, 30 mg/kg/day dose hits those plus Ca++ ch and 5-HT transporter. Subsequent studies can be used to determine the minimum effective dose of this compound. CF at the 30 mg/kg/day dose results in compound brain levels that are selective for sigma receptors only, therefore its affinity at sigma receptors accounts for its behavioral efficacy at these brain concentrations.

### SYNTHETIC EXAMPLES

### Synthesis Example 1: Synthesis of Compounds by Reductive Amination

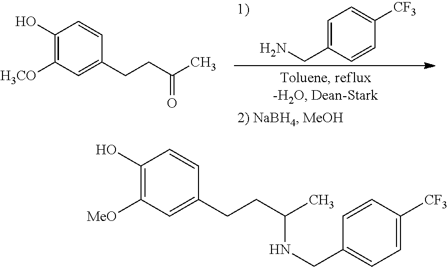 [SMILES: COc1cc(CCC(C)=O)ccc1O ; NCc1ccc(C(F)(F)F)cc1 ; COc1cc(CCC(C)NCc2ccc(C(F)(F)F)cc2)ccc1O]

Vanillylacetone (5.00 g, 25.7 mmol) was dissolved in toluene (250 mL) and 4-trifluoromethylbenzylamine (4.73 g, 27.0 mmol) was added. The mixture was maintained under an atmosphere of nitrogen and heated at reflux with removal of water by Dean-Stark distillation for 16 hours. At this time the Dean-Stark trap was removed and the reaction mixture was cooled to 0° C. on an ice bath. A solution of sodium borohydride (5 g) in methanol (100 mL) was added portion-wise over 30 minutes with vigorous stirring. When the addition was complete the mixture was heated at reflux for 16 hours. At this time the reaction mixture was cooled to room temperature and poured into saturated aqueous sodium bicarbonate solution (300 mL). The resulting mixture was concentrated by rotary evaporation and the aqueous residue was partitioned between water and chloroform. The chloroform layer was dried over anhydrous sodium sulfate and then filtered and concentrated. The product was then purified using silica gel column chromatography employing a mobile phase of 5% ammonia-methanol in chloroform. Product-containing fractions were combined and concentrated then dried under high vacuum overnight to provide a light brown oil (6.72 g, 74%). ¹H NMR (500 MHz, CDCl₃) δ: 7.57 (d, J=7.8 Hz, 2H), 7.43 (d, J=7.9 Hz, 2H), 6.82 (d, J=7.3 Hz, 1H), 6.65 (m, 2H), 5.16-4.42 (br s, 2H), 3.90 (d, J=13.7 Hz, 1H), 3.84 (s, 3H), 3.80 (d, J=13.7 Hz, 1H), 2.76-2.70 (m, 1H), 2.67-2.55 (m, 2H), 1.84-1.77 (m, 1H), 1.69-1.63 (m, 1H), 1.17 (d, J=6.3 Hz, 3H). ¹³C NMR (125 MHz, CDCl₃) δ: 146.7, 144.6, 143.9, 134.0, 129.1, 128.4, 127.5, 125.4, 125.3, 123.2, 120.8, 114.6, 111.0, 55.7, 52.1, 50.6, 38.8, 32.0, 20.1. MS (CI) m/z 353 (M⁺).

The chemical shift measure by ¹H NMR may vary, for example, up to 0.2 ppm. The chemical shift measure by ¹³H NMR may vary, for example, up to 0.5 ppm. The analytical Mass Spectrum may have an experimental error of +/−0.3.

### Purity Determination

The purity of the product was measured by HPLC. The major peak of retention time of 2.22 minutes indicating greater than about 80%, 85%, 90, or 95% of purity. The HPLC conditions used are as follows.

### HPLC Conditions:

Mobile Phase A: 13.3 mM ammonium formate/6.7 mM formic acid in water
Mobile Phase B: 6 mM ammonium formate/3 mM formic acid in water/CH₃CN (1/9, v/v)
Column: Synergi Fusion-RP 100A Mercury, 2×20 mm, 2.5 micron

### (Phenomenex Part No 00M-4423-B0_CE)

Gradient Program: RT=2.22 minutes

| Time, minute | % Phase B | Flow rate, ml/min |
|---|---|---|
| 0 | 100 | 0.5 |
| 1 | 100 | 0.5 |
| 2.5 | 40 | 0.5 |
| 3.4 | 40 | 0.5 |
| 3.5 | 100 | 0.5 |
| 4.5 | 100 | 0.5 |

The purity of the product was also measure by ¹H NMR indicating it to be a single compound of a purity of greater than 90% or 95%. The synthesis described herein can be modified depending upon the final-product to be synthesized.

### Synthesis Example 2: Synthesis of Compounds by Reductive Amination

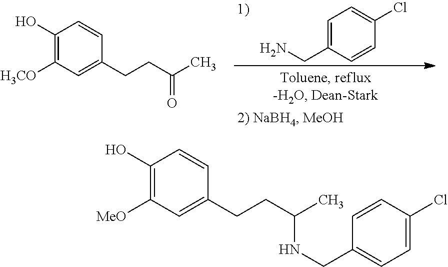 [SMILES: COc1cc(CCC(C)=O)ccc1O ; NCc1ccc(Cl)cc1 ; COc1cc(CCC(C)NCc2ccc(Cl)cc2)ccc1O]

Vanillylacetone (5.00 g, 25.7 mmol) was dissolved in toluene (250 mL) and 4-chlorobenzylamine (4.73 g, 27.0 mmol) was added. The mixture was maintained under an atmosphere of nitrogen and heated at reflux with removal of water by Dean-Stark distillation for 16 hours. At this time the Dean-Stark trap was removed and the reaction mixture was cooled to 0° C. on an ice bath. A solution of sodium borohydride (5 g) in methanol (100 mL) was added portion-wise over 30 minutes with vigorous stirring. When the addition was complete the mixture was heated at reflux for 16 hours. At this time the reaction mixture was cooled to room temperature and poured into saturated aqueous sodium bicarbonate solution (300 mL). The resulting mixture was concentrated by rotary evaporation and the aqueous residue was partitioned between water and chloroform. The chloroform layer was dried over anhydrous sodium sulfate and then filtered and concentrated. The product was then purified using silica gel column chromatography employing a mobile phase of 5% ammonia-methanol in chloroform. Product-containing fractions were combined and concentrated then dried under high vacuum overnight to provide a light brown oil (6.16 g, 75%). ¹H NMR (500 MHz, CDCl₃) δ: 7.30-7.24 (m, 4H), 6.81 (d, J=7.8 Hz, 1H), 6.66-6.62 (m, 2H), 4.25 (br s, 2H), 3.82 (s, 3H), 3.82 (d, J=13.2 Hz, 1H), 3.72 (d, J=13.2 Hz, 1H), 2.73 (m, 1H), 2.66-2.51 (m, 1H), 1.86-1.78 (m, 1H), 1.72-1.63 (m, 1H), 1.62-1.51 (m, 1H), 1.17 (d, J=6.3 Hz, 3H). ¹³C NMR (125 MHz, CDCl₃) δ: 146.6, 143.8, 133.9 132.8, 129.9, 129.7, 128.6, 120.8, 114.5, 110.9, 55.8, 51.9, 50.2, 38.5, 31.9, 31.6, 29.7, 26.9, 22.6, 19.9. MS (MH⁺): m/z 320.

The chemical shift measure by ¹H NMR may vary, for example, up to 0.2 ppm. The chemical shift measure by ¹³H NMR may vary, for example, up to 0.5 ppm. The analytical Mass Spectrum may have an experimental error of +/−0.3.

### Purity Determination

The purity of the product was measured by HPLC. The major peak of retention time of 2.22 minutes indicating greater than about 80%, 85%, 90, or 95% of purity. The HPLC conditions used are as follows.

### HPLC Conditions:

Mobile Phase A: 13.3 mM ammonium formate/6.7 mM formic acid in water
Mobile Phase B: 6 mM ammonium formate/3 mM formic acid in water/CH₃CN (1/9, v/v)
Column: Synergi Fusion-RP 100A Mercury, 2×20 mm, 2.5 micron

### (Phenomenex Part No 00M-4423-B0_CE)

Gradient Program: RT=2.22 minutes

| Time, minute | % Phase B | Flow rate, ml/min |
|---|---|---|
| 0 | 100 | 0.5 |
| 1 | 100 | 0.5 |
| 2.5 | 40 | 0.5 |
| 3.4 | 40 | 0.5 |
| 3.5 | 100 | 0.5 |
| 4.5 | 100 | 0.5 |

The purity of the product was also measure by ¹H NMR indicating it to be a single compound of a purity of greater than 90% or 95%. The synthesis described herein can be modified depending upon the final-product to be synthesized.

### Synthesis Example 3

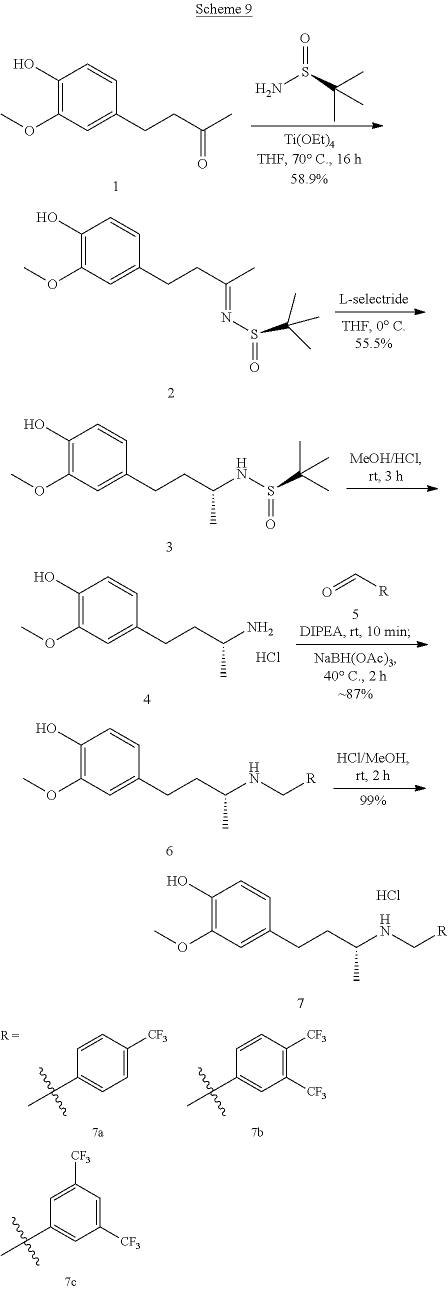

Step 1:

To a solution of 4-(4-hydroxy-3-methoxy-phenyl)-butan-2-one (38.8 g, 200 mmol) in THF (600 mL) was added Ti(OEt)₄ (136.9 g, 600 mmol) and (S)-(−)-tert-butylsulfinamide (29 g, 240 mmol). The mixture was stirred at 70° C. for 16 h, quenched by ice water, extracted with EA (3×300 mL), dried over Na₂SO₄, concentrated to obtain a crude product, which was purified by column chromatography (PE/EA:3/1) to give the title compound 2 (35 g, 59%).

Step 2:

To a solution of compound 2 (18 g, 60 mmol) in THF (180 mL) was added L-Selectride (180 mL, 1.0 M in THF, 180 mmol) at 0° C. The reaction was allowed to warm to rt over a 3 h period. Analysis of the reaction mixture by TLC showed complete consumption of the starting imine 2. The solution was then quenched by adding water and extracted by EA (3×200 mL). The combined organic layers were washed with brine, dried over Na₂SO₄ and concentrated under vacuum to give a residue, which was purified by column chromatography (PE/EA:2/1) to provide product. The product continued purified by recrystallization with PE/EA (1:1) to got product 3 (9.9 g, 55%). The ee value was determined by HPLC.

Step 3:

To a solution of 3 (7.0 g, 23.4 mmol) in MeOH (20 mL), HCl (2 M in MeOH, 20 mL) was added and the resulting solution was stirred at rt over a 3 h period. TLC analysis of the reaction mixture showed complete consumption of compound 3. The solvent was then removed in vacuum, and the resulting residue 4 was used directly for the next step.

Step 4:

To a solution of the crude compound 4 (5.4 g, 23.4 mmol) in THF (100 μmL) were added DIPEA (4.53 g, 35.1 mmol) and 4-trifluoromethylbenzaldehyde 5 (4.28 g, 24.6 mmol). The resulting solution was stirred at rt for 10 min. Then NaBH(OAc)₃ (14.9 g, 70.2 mmol) was added and the mixture was stirred at 40° C. for 2 h. The mixture was quenched by water at 0° C., filtered and extracted by EtOAc. The organic layer was washed by brine, dried over sodium sulfate, filtered and the filtrate was concentrated under reduced pressure to afford a residue. The residue was purified by column chromatography (PE/EA=1:2) to give product 6 (7.0 g, 87%).

Step 5:

To a solution of 6 (1.0 g, 2.8 mmol) in MeOH (5 mL), HCl (2 M in MeOH, 20 mL) was added and the resulting solution was stirred at rt for 30 min. The solvent was removed to give the product 7a (1.1 g, 99%) as white solid. Compounds 7b and 7c were similarly made by substituting compound 5 with the appropriate benzaldehyde.

m/z (ESI+) (M+H)+: 7a [354.2]; 7b [422.2]; 7c [422.2].

### Synthesis Example 4

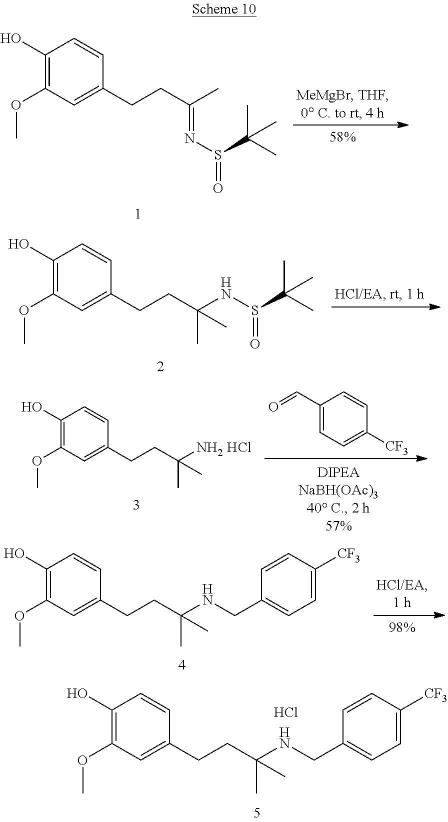

Step 1:

To a solution of methylmagnesium bromide in THF (5 mL) was added a solution of 1 (1.0 g, 3.3 mmol) in THF (5 mL) at 0° C. The mixture was stirred at rt for 4 h, quenched by adding ice-water, extracted with ethyl acetate (3×30 mL), dried by vacuum to afford a crude product, which was purified by column chromatography (PE/EA:3/1) to give compound 2 (0.6 g, 58%).

Step 2:

To a solution of 2 in EA (10 mL) at 0° C. was HCl (2 M in EA, 3 mL), and the resulting solution was stirred at rt for 1 h. Analysis of the reaction mixture by TLC showed complete consumption of 2. Concentrated under vacuum, the crude product was directly used in next step.

Step 3:

To a solution of 3 (0.4 g, 1.9 mmol) in THF (20 mL), DIPEA (0.6 g, 4.6 mmol) and trifluoromethylbenzaldehyde (0.4 g, 2.3 mmol) were added subsequently. The resulting solution was stirred at rt for 10 min. Sodium triacetoxylboronhydride (1.63 g, 7.7 mmol) was added and the mixture was stirred at 40° C. for 2 h. The mixture was quenched by water at 0° C., filtered and extracted by ethyl acetate (3×40 mL). The organic layer was washed by brine, dried over sodium sulfate, filtered and the filtrate was concentrated under reduced pressure to afford a residue. The residue was purified by column chromatography (PE/EA=1:1) to give 4 (0.4 g, 57%).

Step 4:

To a solution of 4 in EA (10 mL), HCl (2 M in MeOH, 2 mL) was added and the resulting solution was stirred at rt for 1 h. After concentrated by vacuum, the residue was washed with ethyl acetate to afford 5 (0.4 g, 98%).

m/z (ESI+) (M+H)+: 5 [368.2].

### Synthesis Example 5

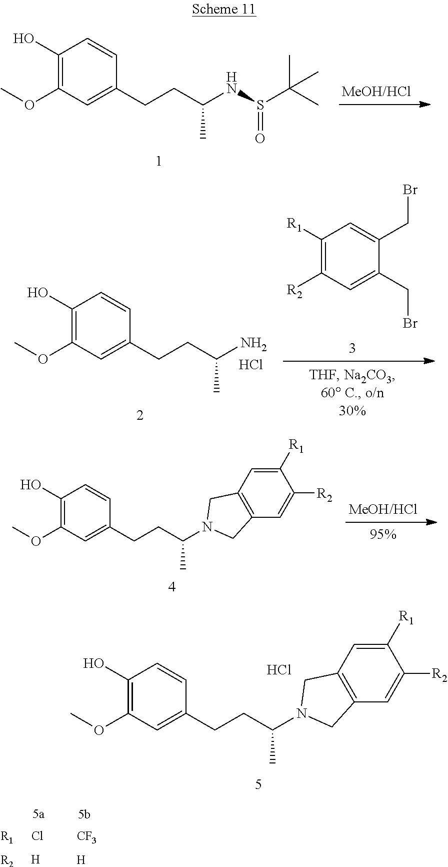

Step 1:

To a solution of compound 1 (2 g, 6 mmol) in MeOH (30 mL), HCl (2 M in MeOH, 30 mL) was added and the resulting solution was stirred at rt for 3 h. TLC analysis of the reaction mixture showed complete consumption of compound 1. The solvent was then removed in vacuum, and it was used directly for next step.

Step 2:

To a solution of 2 (0.4 g, 2 mmol) in THF (10 mL), compound 3a (0.54 g, 2 mmol) in THF (5 mL) was added. Na₂CO₃ (0.6 g, 6 mmol) was added, and the resulting solution was stirred at 60° C. overnight. After concentration, the residue was purified by FCC to give compound 4 (0.2 g, 30%).

Step 3:

To a solution of 4 in EA (5 mL), HCl (2 M in MeOH, 3 mL) was added and the resulting solution was stirred at rt for 1 h. After being concentrated in vacuo, the residue was washed by ethyl acetate to give compound 5 (0.2 g, 95%). Compound 5b was similarly made by substituting compound 3 with the appropriate dibenzyl bromide.

m/z (ESI+) (M+H)+: 5a [332.1]; 5b [366.1].

### Synthesis Example 6

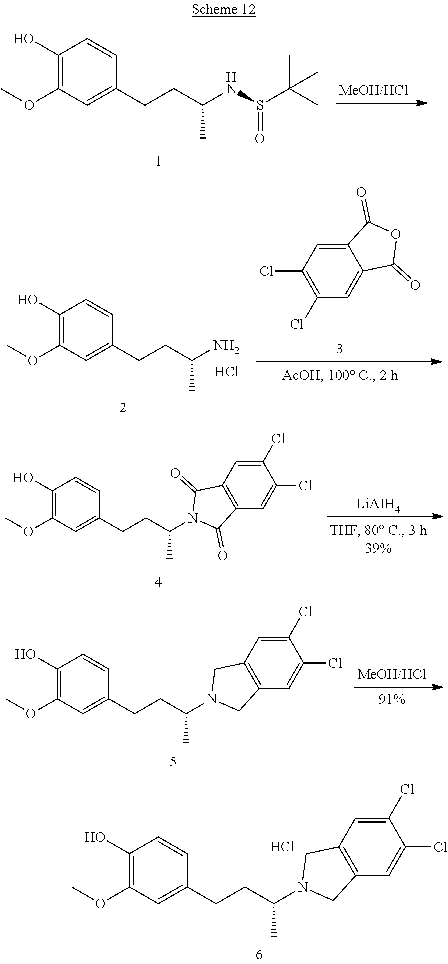

Step 1:

To a solution of compound 1 (0.4 g, 1.3 mmol) in MeOH (10 mL), HCl (2 M in MeOH, 10 mL) was added and the resulting solution was stirred at rt for 3 h. TLC analysis of the reaction mixture showed complete consumption of compound 1. The solvent was removed in vacuum, and it was used directly for next step.

Step 2:

Compound 2 (0.2 g, 1 mmol) and 3 (0.2 g, 1 mmol) was dissolved in acetic acid (10 mL), stirred at 100° C. for 2 h. The mixture was cooled to rt and quenched by water (10 mL), extracted by EtOAc (3×20 mL), dried, concentrated to give the title compound 4 (0.3 g, 76%).

Step 3:

To a solution of 4 (0.3 g, 0.7 mmol) in THF (10 mL) was added LAH (0.1 g, 3.5 mmol). The mixture was stirred at 80° C. for 3 h. The mixture was quenched by water (0.1 mL), 15% of NaOH (0.1 mL) and water (0.3 mL), filtered, concentrated. The crude product was purified by column chromatography (PE/EA=5:1) to give compound 5 (0.1 g, 39%).

Step 4:

To a solution of 5 in ethyl acetate (5 mL), HCl (2 M in MeOH, 3 mL) was added and the resulting solution was stirred at rt for 1 h. The reaction was concentrated by vacuum to afford the title compound 6 (0.16 g, 91%).

m/z (ESI+) (M+H)+: 6 [366.2].

### Synthesis Example 7

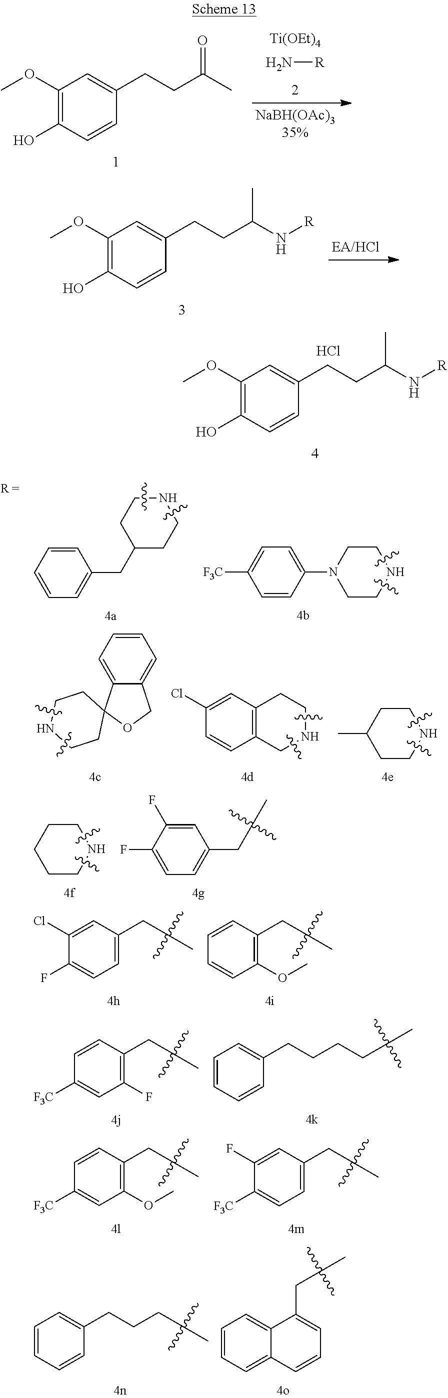

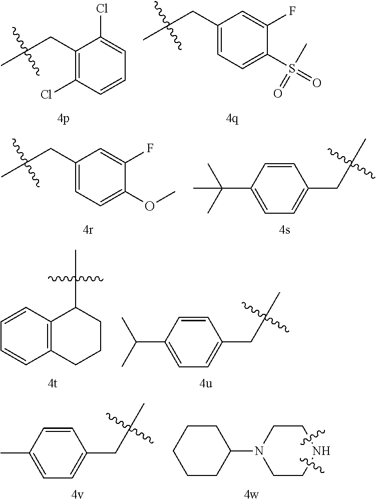

Step 1:

To a solution of 1 (0.278 g, 1.43 mmol) in THF (20 mL) was added Ti(OEt)₄ (2.1 g, 9.2 mmol) and (4-benzylpiperidine (0.34 g, 1.3 mmol). The mixture was stirred at 40° C. for one day, quenched by ice water, extracted with ethyl acetate (3×20 mL). After being concentrated in vacuo, the crude product was purified by column chromatography (PE/EA:1/1) to give 3 (205 mg, 35%).

Step 2:

To a solution of 3 (0.2 g, 0.47 mmol) in ethyl acetate (5 mL), HCl (2 M in MeOH, 3 mL) was added and the resulting solution was stirred at rt for 1 h. The reaction was concentrated by vacuum to get 4a (0.2 g, 95%). Compounds 4b-4w were similarly made by substituting amine compound 2 with the appropriate amine.

m/z (ESI+) (M+H)+: 4a [354.3]; 4b [409]; 4c [368.2]; 4d [346.1]; 4e 5 [278.50]; 4f [264.05]; 4g [322.10]; 4h [338.05]; 4i [316.15]; 4j [372.10]; 4k [328.25]; 41 [384.15]; 4m [372.10]; 4n [314.10]; 4o [336.15]; 4p [354.10]; 4q [382.20]; 4r [334.15]; 4s [342.15]; 4t [326.15]; 4u [328.20]; 4v [300.10]; 4w [347.6].

### Synthesis Example 8

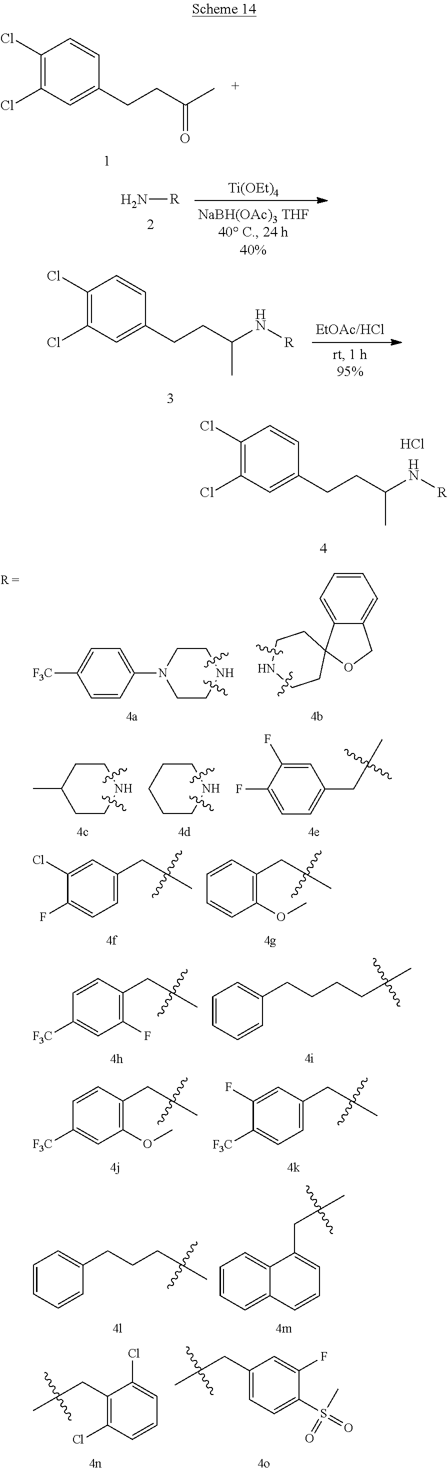

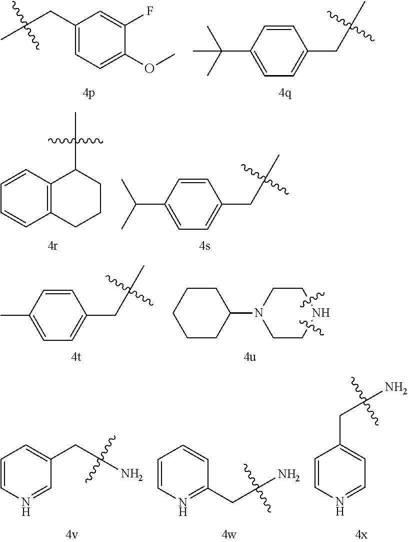

Step 1:

To a solution of 1 (0.31 g, 1.43 mmol) in THF (20 mL) was added Ti(OEt)₄ (0.595 g, 2.58 mmol) and N-(4-trifluoromethylphenyl)-piperazine 2 (0.3 g, 1.3 mmol). The mixture was stirred at 40° C. for 24h, quenched by adding ice-water, extracted with ethyl acetate (3×20 mL), dried. Purification by column chromatography (PE/EA:1/1) gave product 3 (0.25 g, 41%).

Step 2:

To a solution of compound 3 (0.25 g, 0.58 mmol) in ethyl acetate (5 mL) was added MeOH—HCl (2 N, 4 mL). The mixture was stirred at room temperature for 1 h. Concentration in vacuo gave compound 4 (0.25 g, 95%). Compounds 4b-4× were similarly made by substituting amine compound 2 with the appropriate amine.

m/z (ESI+) (M+H)+: 4a [431.2]; 4b [390.2]; 4c [300.05]; 4d 10 [286.00]; 4e [344.05]; 4f [362.00]; 4g [338.05]; 4h [394.10]; 4i [350.05]; 4j [406.05]; 4k [394.15]; 41 [336.05]; 4m [358.05]; 4n [378.05]; 40 [445.20]; 4p [356.10]; 4q [364.10]; 4r [348.05]; 4s [350.10]; 4t [322.10]; 4u [369.2]; 4v [309.00]; 4w [308.95]; 4x [309.00].

### Synthesis Example 9

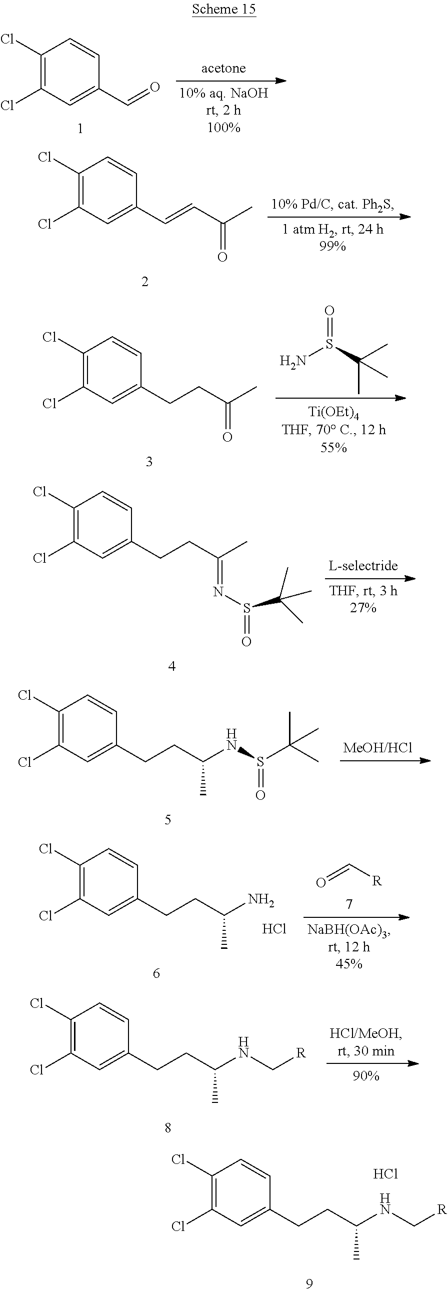

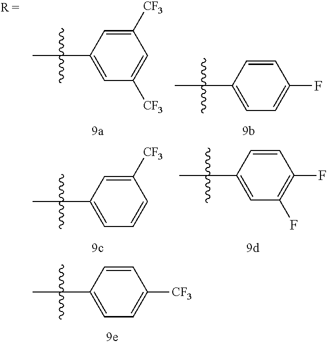 [SMILES: *c1cc(C(F)(F)F)cc(C(F)(F)F)c1 ; *c1ccc(F)cc1 ; *c1cccc(C(F)(F)F)c1 ; *c1ccc(F)c(F)c1 ; *c1ccc(C(F)(F)F)cc1]

Step 1:

To a solution of 1 (3.5 g, 20 mmol) in acetone (20 mL) and ethanol (2 mL) was added aqueous NaOH (10%, 15 mL) and water (80 mL). The mixture was stirred at rt for 2 h, extracted with EA (3×50 mL). The organic layers were dried and concentrated to give 2 (4.3 g, 100%).

Step 2:

To a solution of 2 (4.3 g, 20 mmol) in MeOH (50 mL) was added diphenylsulfide (0.15 mL) and Pd/C (10%, 0.43 g). The mixture was vigorously stirred at 25° C. under 1 atm of hydrogen for 24 h. The reaction mixture was filtered through a pad of Celite, washed with methanol, and the filtrate was concentrated to provide 3 (4.3 g, 99%).

Step 3:

To a solution of 3 (10 g, 46 mmol) in THF (100 mL) was added Ti(OEt)₄ (21 g, 92 mmol), and (S)-(−)-tert-butylsulfinamide (6.1 g, 50 mmol). The mixture was stirred at 70° C. for 12 h, quenched by ice water, extracted with ethyl acetate (3×250 mL). After being concentrated by vacuum, the crude product was purified by column chromatography (PE/EA:10/1) to afford compound 4 (8.1 g, 55%).

Step 4:

To a solution of compound 4 (3.3 g, 10 mmol) in THF (30 mL) was added L-Selectride (33 mL, 1.0 M in THF, 33 mmol) at 0° C. The reaction was allowed to warm to rt over a 3 h period. Analysis of the reaction mixture by TLC showed complete consumption of the starting imine 4. The solution was quenched by water and extracted by ethyl acetate (3×30 mL). The combined organic layer was washed with brine, dried over Na₂SO₄ and concentrated under vacuum to give a residue, which was purified by column chromatography (PE/EA:2/1) to provide product 5 (0.9 g, 27%).

Step 5:

To a solution of compound 5 (5 g, 15.5 mmol) in MeOH (10 mL), HCl (2 M in MeOH, 10 mL) was added and the resulting solution was stirred at rt for 3 h. TLC analysis of the reaction mixture showed complete consumption of compound 5. The solvent was removed in vacuum, and the crude 6 (3.95 g, 100%) was used directly for next step without further purification.

Step 6:

To a solution of 6 (0.6 g, 2.4 mmol) in THF (10 mL), DIPEA (0.4 g, 3.1 mmol) and 3-trifluoromethylbenzaldehyde (0.41 g, 2.4 mmol) were added subsequently. The resulting solution was stirred at rt for 10 min. NaBH(OAc)₃ (1.0 g, 4.7 mmol) was added and the mixture was stirred for 12 h. The mixture was quenched by water at 0° C., filtered and extracted by EtOAc (3×30 mL). The organic layer was washed by brine, dried over sodium sulfate, filtered and the filtrate was concentrated under reduced pressure to afford a residue. The residue was purified by column chromatography (PE/EA=1:1) to give compound 8 (0.4 g, 45%).

Step 7:

To a solution of 8 (0.4 g, 1.08 mmol) in MeOH (5 mL), HCl (2 M in MeOH, 4 mL) was added and the resulting solution was stirred at rt for 0.5 h. The reaction was concentrated to give amine 9a (0.4 g, 90%). Compounds 9b-9e were similarly made by substituting compound 7 with the appropriate benzaldehyde.

m/z (ESI+) (M+H)+: 9a[444.2]; 9b [326.25]; 9c [376.2]; 9d [344.2]; 9e [376.1].

### Synthesis Example 10

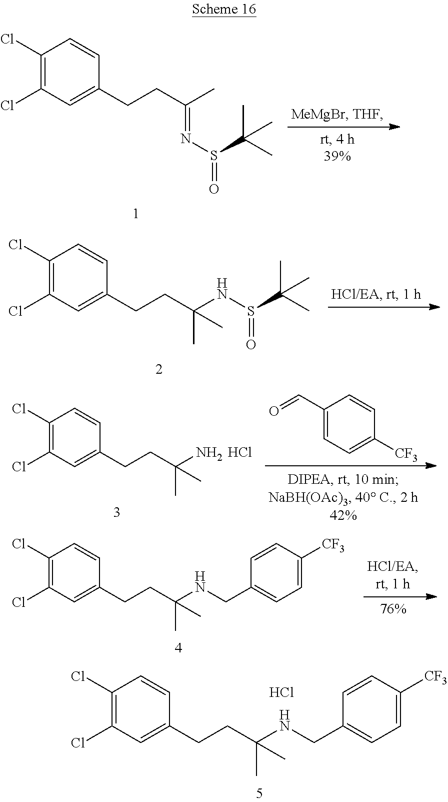

Step 1:

To a solution of methylmagnesium bromide in THF (3 M, 15 mL) was added a solution of 1 (1.5 g, 4.6 mmol) in THF (20 mL) at 0° C. The mixture was stirred at rt for 4 h, quenched by ice water, extracted with ethyl acetate (3×30 mL). After being concentrated, the crude product was purified by column chromatography (PE/EA: 3/1) to afford compound 2 (0.6 g, 39%).

Step 2:

To a solution of compound 2 (0.6 g, 1.8 mmol) in ethyl acetate (10 mL), HCl (2 M in MeOH, 3 mL) was added and the resulting solution was stirred at rt for 1 h. TLC analysis of the reaction mixture showed complete consumption of compound 2. The solvent was then removed in vacuum, and the crude compound 3 was used directly for next step.

Step 3:

To a solution of 3 (0.43 g, 1.8 mmol) in THF (20 mL), DIPEA (0.54 g, 4.0 mmol) and 4-trifluoromethylbenzaldehyde (0.36 g, 2.0 mmol) were added sequentially. The resulting solution was stirred at rt for 10 min. NaBH(OAc)₃ (1.57 g, 7.4 mmol) was added and the mixture was stirred at 40° C. for 2 h. The mixture was quenched by water at 0° C., filtered and extracted by EtOAc (3×30 mL). The organic layers were washed by brine, dried over sodium sulfate, filtered and the filtrate was concentrated under reduced pressure to afford a residue, which was purified by column chromatography (PE/EA=3:1) to give compound 4 (0.3 g, 43%).

Step 4:

To a solution of 4 (0.3 g, 0.8 mmol) in ethyl acetate (10 mL), HCl (2 M in MeOH, 2 mL) was added and the resulting solution was stirred at rt for 1 h. The precipitate was filtered to obtain compound 5 (0.25 g, 76%).

m/z (ESI+) (M+H)+: 5 [390.14].

### Synthesis Example 11

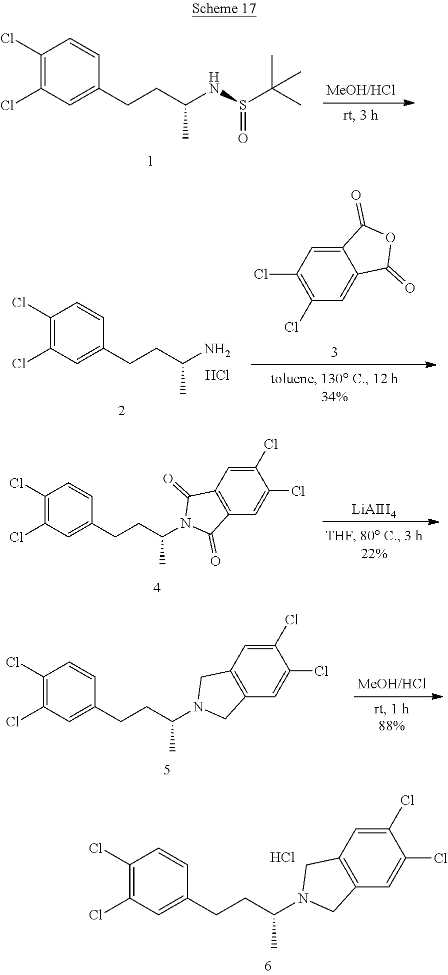

Step 1:

A mixture of compound 1 (1.75 g, 5.43 mmol) in MeOH—HCl (2 M, 10 mL) was stirred at rt for 3 h. The reaction mixture was concentrated in vacuo to give a crude 2, which was used for next step without further purification.

Step 2:

A solution of compound amine 2 (1.4 g, 5.43 mmol) and anhydride 3 (1.18 g, 5.43 mmol) in toluene (12 mL) was heated at 130° C. for 12 h. The mixture was cooled to rt and water (10 mL) was added, extracted by EtOAc (3×20 mL), dried, concentrated. The crude product was purified by column chromatography (PE/EA=10:1) to give product 4 (0.78 g, 34%).

Step 3:

To a solution of compound 4 (0.78 g, 1.87 mmol) in THF (20 mL) was added LAH (0.36 g, 9.1 mmol). The mixture was stirred at 80° C. for 3 h. The cooled mixture was quenched by water (3.46 mL), 15% of NaOH (3.46 mL) and water (13.5 mL). The reaction mixture was filtered, concentrated. The crude product was purified by column chromatography (PE/EA=5:1) to give product 5 (0.16 g, 23%).

Step 4:

Compound 5 (0.3 g, 0.8 mmol) was dissolved in ethyl acetate (5 mL), MeOH—HCl (2N, 3 mL) was added. The mixture was stirred at room temperature for 1 h, concentrated to give compound 6 (0.16 g, 89%).

m/z (ESI+) (M+H)+: 6 [390.0].

### Synthesis Example 12

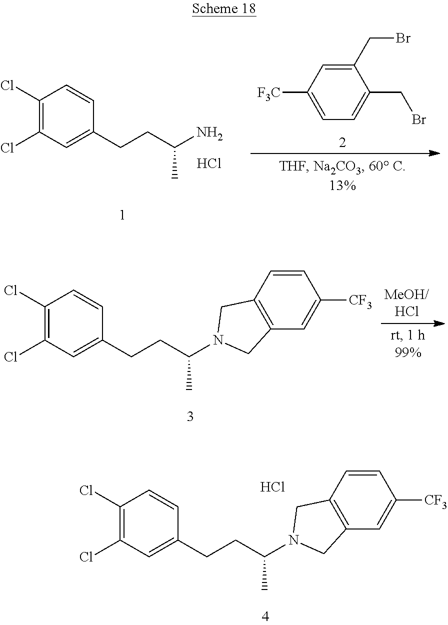 [SMILES: C[C@@H](N)CCc1ccc(Cl)c(Cl)c1 ; Cl ; FC(F)(F)c1ccc(CBr)c(CBr)c1 ; C[C@H](CCc1ccc(Cl)c(Cl)c1)N1Cc2ccc(C(F)(F)F)cc2C1 ; C[C@H](CCc1ccc(Cl)c(Cl)c1)N1Cc2ccc(C(F)(F)F)cc2C1 ; Cl]

Step 1:

To a solution of compound 1 (0.4 g, 2 mmol) in DMF (6 mL) was added dibromide 2 (0.6 g, 2 mmol). The resulting solution was stirred at 80° C. overnight, concentrated, purified by preparative-HPLC to give compound 3 (0.1 g, 13%).

Step 2:

Compound 3 (0.1 g, 0.2 mmol) was dissolved in ethyl acetate (5 mL), MeOH—HCl (2 N, 3 mL) was added. The mixture was stirred at room temperature for 1 h. The mixture was filtered to give compound 4 (0.11 g, 99%).

m/z (ESI+) (M+H)+: 4 [388.1].

### Synthesis Example 13

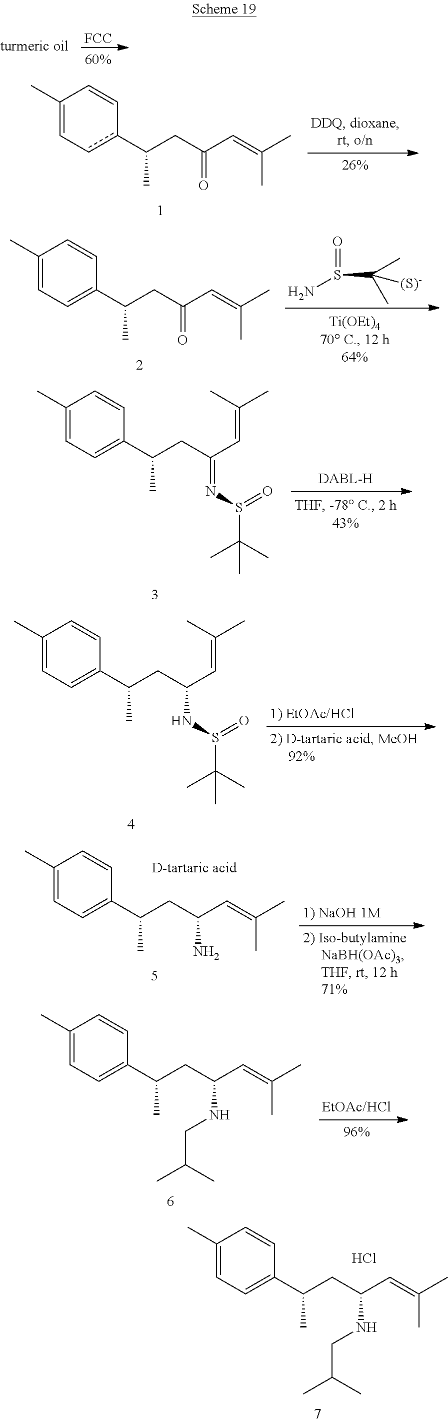

Step 1:

Turmeric oil (100 g) was purified by column chromatography (PE:EA/100:1) to provide crude product 1 (60 g).

Step 2: The crude product 1 (60 g) was dissolved in dioxane (200 mL), DDQ (81.7 g, 360 mmol) was added. The mixture was stirred at rt overnight, then quenched by water (500 mL), filtered through a pad of Celite. The filtrate was extracted by ethyl acetate (3×200 mL). The organic layers were dried, concentrated and purified by column chromatography (PE:EA/30:1) to provide compound 2 (15.6 g, 26%).

Step 3:

To a solution of 2 (7.4 g, 34.2 mmol) in Ti(OEt)₄ (23.4 g, 102.6 mmol) was added (S)-(−)-2-methyl-2-propanesulfinamide. The mixture was stirred at 70° C. for 12 h, quenched by ic-water, extracted with ethyl acetate (3×100 mL), and dried to give a residue, which was purified by column chromatography (PE/EA:5/1) to give product 3 (7.1 g, 64%).

Step 4:

To a solution of compound 3 (7.0 g, 21.9 mmol) in THF (70 mL) at −78° C. was added DIBAL-H (22 mL, 1.5 M in THF, 33 mmol). The resulting solution was stirred at −78° C. for 2 h. Analysis of the reaction mixture by TLC showed complete consumption of the starting imine to give sulfinamide compound 4. The solution was quenched by water and extracted by ethyl acetate (3×200 mL). The combined organic layers were washed with brine, dried over Na₂SO₄, and concentrated to furnish an orange oil. The crude product was subjected to column chromatography (PE:EA/3:1) to provide product 4 (3.1 g, 43%).

Step 5:

To a solution of compound 4 (1.6 g, 5.0 mmol) in ethyl acetate (10 mL) was added HCl-ethyl acetate (2 N, 10 mL), and the resulting solution was stirred at room temperature for 3 h. TLC analysis of the reaction mixture showed complete consumption of compound 3. The solvent was removed in vacuum. The residue was dissolved in water (10 mL), and pH was adjusted to 9-10 by a saturation aqueous solution of K₂CO₃, extracted by ethyl acetate (3×20 mL), dried, and concentrated to give a free amine. The free amine (1.1 g, 5.0 mmol) was dissolved in methanol (15 mL). D-tartaric acid (0.75 g, 5.0 mmol) was added to the solution. The mixture was stirred under reflux for 1 h. The solution was slowly cooled to rt. The formed crystals were filtered to give product 5 (1.7 g, 93%).

Mp. 172-174° C. The absolute stereochemistry of the compound 5 was determined by X-ray crystallography.

Step 6:

A solution of compound 5 (1.7 g, 4.6 mmol) in water (20 mL) was adjusted to pH 9-10 by 1M NaOH. The product was extracted with ethyl acetate (3×20 mL). The organic layers were dried, concentrated to give a free amine. The free amine was dissolved in THF (10 mL), iso-butylamine (0.40 g, 5.5 mmol) and NaBH(OAc)₃ (3.90 g, 18.4 mmol) was added. The mixture was stirred at rt for 12 h, quenched with water, extracted by ethyl acetate (3×30 mL). The organic layers were dried, concentrated and purified by column chromatography (PE:EA/3:1) to provide product 6 (0.9 g, 72%).

Step 7:

To a solution of compound 6 (0.9 g, 3.2 mmol) in ethyl acetate (10 mL) was added ethyl acetate-HCl (2 N, 5 mL). The mixture stirred at room temperature for 1 h. Ethyl acetate was removed in vacuo to afford compound 7 (0.95 g, 96%).

m/z (ESI+) (M+H)+: 7 [274.20].

### Synthesis Example 14

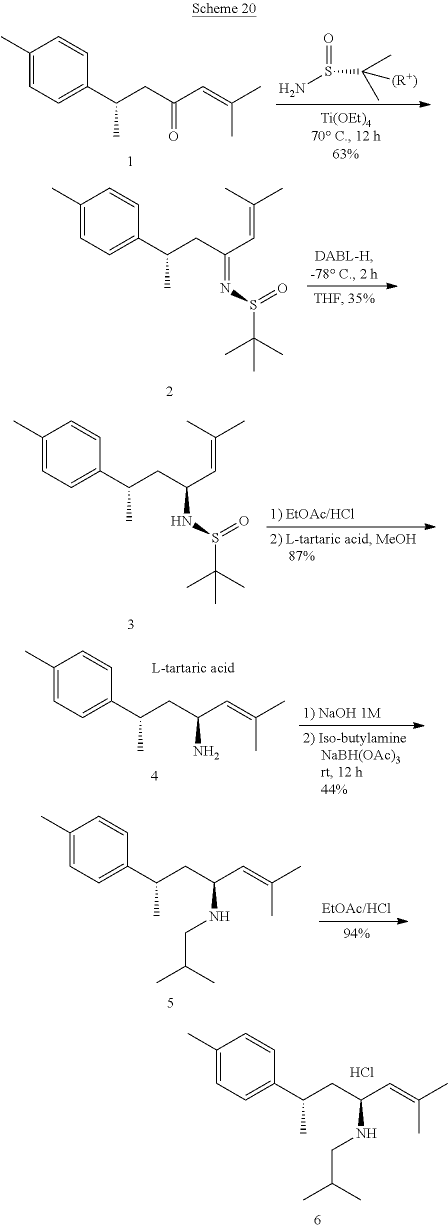

Step 1:

To a solution of 1 (7.4 g, 34.2 mmol) in Ti(OEt)₄ (23.4 g, 102.6 mmol) was added (R)-(+)-2-methyl-2-propanesulfinamide. The mixture was stirred at 70° C. for 12 h, quenched by ice-water, extracted with ethyl acetate (3×100 mL), dried. Purified by column chromatography (PE/EA:5/1) to afford product 2 (6.9 g, 63%).

Step 2:

Compound 2 (7.0 g, 21.9 mmol) was dissolved in THF (70 mL) and cooled to −78° C. To the vessel was then added DIBAL-H (22 mL, 1.5 M in THF, 33 mmol), and the resulting solution was stirred at −78° C. for 2 h. Analysis of the reaction mixture by TLC showed complete consumption of the starting imine to give sulfinamide compound 3. The solution was then quenched by water and extracted by ethyl acetate (3×200 mL). The combined organic layers were washed with brine, dried over Na₂SO₄ and concentrated under vacuum to furnish an orange oil. The crude product was subjected to column chromatography (PE:EA/3:1) to provide product 3 (2.5 g, 36%).

Step 3:

To a solution of 3 (2.5 g, 7.8 mmol) in ethyl acetate (10 mL), 2M HCl in ethyl acetate (10 mL) was added and the resulting solution was stirred at room temperature for 3 h. TLC analysis of the reaction mixture showed complete consumption of compound 3. The solvent was removed with vacuum. The residue was dissolved in water (10 mL), whose pH was adjusted to 9-10 by adding saturated K₂CO₃. The mixture was extracted by ethyl acetate (3×20 mL), dried, concentrated to get free amine 4. The free amine 4 was dissolved in methanol (15 mL), L-trataric acid (1.17 g, 7.8 mmol) was added. The mixture was stirred under reflux for 1 h, cooled to rt, filtered to get crystalline salt 4 (2.5 g, 87%).

Step 4:

L-trataric acid salt 4 (2.5 g, 6.8 mmol) was dissolved in water (20 mL), whose pH was adjusted to 9-10 by adding 1 M NaOH. The mixture was then extracted by ethyl acetate (3×50 mL), dried, concentrated to get free amine 4. The free amine 4 was redissolved in THF, iso-butylamine (0.60 g, 8.2 mmol) and NaBH(OAc)₃ (5.85 g, 27.6 mmol) was added. The mixture was stirred at rt for 12 h, quenched by water, extracted by ethyl acetate (3×30 mL). The organic layer was dried, concentrated and purified by column chromatography (PE:EA/3:1) to provide product 5 (0.83 g, 45%).

Step 5:

To a solution of 5 (0.83 g, 3.0 mmol) in ethyl acetate (10 mL), HCl (2 M in ethyl acetate, 5 mL) was added and the resulting solution was stirred at rt for 1 h. The solvent was removed to give the product 6 (0.88 g, 94%).

m/z (ESI+) (M+H)+: 6 [274.20]

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the invention, in addition to those described herein, will be apparent to those skilled in the art from the foregoing description. Such modifications are also intended to fall within the scope of the appended claims.

All publications mentioned herein are incorporated by reference in their entirety. Nothing herein is to be construed as an admission that the invention is not entitled to antedate such disclosure by virtue of prior invention.

1-32. (canceled)

33. A method for treating neurodegenerative disease comprising administering an effective amount of a sigma-2 antagonist having an inhibitory constant (Kᵢ) of less than 500 nM for sigma-2 receptor binding.

34. The method of claim 33, wherein the inhibitory constant (Ki) is less than 150 nM for sigma-2 receptor binding.

35. The method of claim 33, wherein the inhibitory constant (Ki) is less than 10 nM for sigma-2 receptor binding.

36. The method of claim 33, wherein the sigma-2 antagonist has a half maximal effective concentration (EC₅₀) of less than 20 μM.

37. The method of claim 33, wherein the sigma-2 antagonist has a half maximal effective concentration (EC₅₀) of less than 1 μM.

38. The method of claim 33, wherein the sigma-2 antagonist exhibits a brain/plasma ratio of greater than 2.

39. The method of claim 33, wherein the sigma-2 antagonist exhibits a brain/plasma ratio of greater than 10.

40. The method of claim 33, wherein the sigma-2 antagonist has a metabolic stability of greater than 20 minutes.

41. The method of claim 33, wherein the sigma-2 antagonist has a metabolic stability of greater than 30 minutes.

42. The method of claim 33, wherein the sigma-2 antagonist exhibits a brain maximum concentration (Cₘₐₓ) of greater than 600 ng/mL.

43. The method of claim 33, wherein the sigma-2 antagonist exhibits a brain maximum concentration (Cₘₐₓ) of greater than 1000 ng/mL.

44. The method of claim 33, wherein the sigma-2 antagonist exhibits a brain maximum concentration (Cₘₐₓ) of greater than 1600 ng/mL.

45. The method of claim 33, wherein the sigma-2 receptor antagonist is selected from the group consisting of small molecules, antibodies, and antibody fragments.

46. The method of claim 33, wherein the sigma-2 receptor is an antibody or antibody fragment conjugated to immunoglobulin G (IgG), insulin receptor (HIR), or the transferrin receptor (TfR).

47. The method of claim 33, wherein administering comprises administering to a human about 1 mg/Kg to about 300 mg/Kg of body weight per day.

48. The method of claim 33, wherein the neurodegenerative disease is Alzheimer's disease.

49. The method of claim 33 where the sigma-2 antagonist is selected from a compound of Formula I: 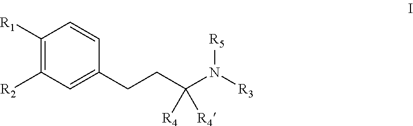
or pharmaceutically acceptable salts thereof,
wherein
R₁ and R₂ are independently selected from H, OH, halo, C₁₋₆ alkoxy, C₁₋₆ haloalkyl, C₁₋₆ haloalkoxy, (R₁₆)(R₁₇)N—C₁₋₄ alkylene-O—, or R₁ and R₂ are linked together to form a —O—C₁₋₂ methylene-O— group, wherein
R₁₆ and R₁₇ are independently C₁₋₄ alkyl or benzyl, or R₁₆ and R₁₇ together with nitrogen form a ring selected from 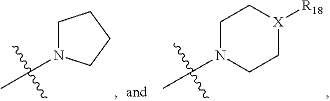
wherein
X is N or O and R₁₈ is H or unsubstituted phenyl; and
wherein at least one of R₁ and R₂ is not H;
R₄ is C₁₋₆ alkyl;
R_{4′} is H or C₁₋₆ alkyl; and
R₃ and R₅ together with nitrogen form a ring selected from 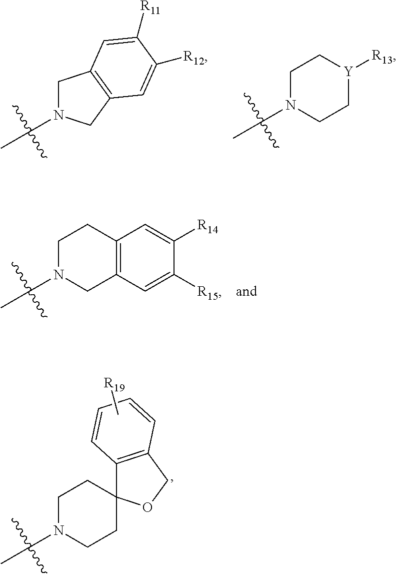
wherein
R₁₁ and R₁₂, are independently selected from H, halo, and C₁₋₆ haloalkyl, and
Y is CH or N;
R₁₃ is H, C₁₋₆ alkyl, C₃₋₆ cycloalkyl, unsubstituted phenyl or phenyl substituted with C₁₋₆ haloalkyl or unsubstituted benzyl;
R₁₄ and R₁₅ are independently selected from H, and halo; and
R₁₉ is H.
